SEQUENCE LISTING (151 sequences)

SEQ ID NO: 1 - protein, 613 aa, synthetic construct
  source 1..613: note = FMC63 TCR_CAR_Split_VL_TRBC_VH_TRAC Construct with GM-CSF leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 2 - protein, 613 aa, synthetic construct
  source 1..613: note = FMC63 TCR_CAR_Split_VH_TRBC_VL_TRAC Construct with GM-CSF leader sequences
MLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 3 - protein, 631 aa, synthetic construct
  source 1..631: note = FMC63 TCR_CAR_Full_TRBC_VL_VH_TRAC Construct with GM-CSF leader sequences
MLLLVTSLLLCELPHPAFLLIPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 4 - protein, 631 aa, synthetic construct
  source 1..631: note = FMC63 TCR_CAR_Full_VL_VH_TRBC_TRAC Construct with GM-CSF leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 5 - protein, 631 aa, synthetic construct
  source 1..631: note = FMC63 TCR_CAR_Full_TRBC_VH_VL_TRAC Construct with GM-CSF leader sequences
MLLLVTSLLLCELPHPAFLLIPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 6 - protein, 631 aa, synthetic construct
  source 1..631: note = FMC63 TCR_CAR_Full_VH_VL_TRBC_TRAC Construct with GM-CSF leader sequences
MLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 7 - DNA, 544 bp, synthetic construct
  source 1..544: mol_type = other DNA; note = EF1a_promoter
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 8 - DNA, 386 bp, synthetic construct
  source 1..386: mol_type = other DNA; note = MNDu3_promoter
tgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggc

SEQ ID NO: 9 - protein, 1271 aa, synthetic construct
  source 1..1271: note = Bispecific_TCR_CAR_GPRC5D scFv_TRBC_BCMA scFv_TRAC_tEGFR Construct with GM-CSF leader sequences
MLLLVTSLLLCELPHPAFLLIPQSALTQPASVSGSPGQSITISCTGTSSDVGGYNYVSWYQQHPGKAPKLMIYDVSKRPSGVSNRFSGSKSGNTASLTISGLQAEDEADYYCSSYTRSSTEVFGGGTKLTVLGSRGGGGSGGGGSGGGGSLEMAEVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSKKWSGEKWRRENFDYWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSSRAKRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 10 - protein, 621 aa, synthetic construct
  source 1..621: note = TCR_CAR_TRBC_Nanobody-AAA-Nanobody_TRAC Construct with GM-CSF leader sequences
MLLLVTSLLLCELPHPAFLLIPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 11 - protein, 621 aa, synthetic construct
  source 1..621: note = TCR_CAR_Nanobody-AAA-Nanobody_TRBC_TRAC Construct with GM-CSF leader sequences
MLLLVTSLLLCELPHPAFLLIPQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 12 - protein, 856 aa, synthetic construct
  source 1..856: note = TCR_CAR_Nanobody-AAA-Nanobody_TRBC_Nanobody-AAA-Nan body_TRAC Construct with GM-CSF leader sequences
MLLLVTSLLLCELPHPAFLLIPQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 13 - protein, 587 aa, synthetic construct
  source 1..587: note = TCR_CAR_TRBC_FLAG-HA_Bcl2_Ectodomain_TRAC Construct with GM-CSF and murine IgG, kappa leader sequences
MLLLVTSLLLCELPHPAFLLIPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMETDTLLLWVLLLWVPGSTGDYKDEYPYDVPDYAGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 14 - protein, 587 aa, synthetic construct
  source 1..587: note = TCR_CAR_FLAG_HA_Bcl2_Ectodomain_TRBC_TRAC Construct with GM-CSF and murine IgG, kappa leader sequences
METDTLLLWVLLLWVPGSTGDYKDEYPYDVPDYAGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMLLLVTSLLLCELPHPAFLLIPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 15 - protein, 789 aa, synthetic construct
  source 1..789: note = TCR_CAR_Bcl2_Ectodomain_TRBC_Bcl2_Ectodomain_TRAC Construct with murine IgG, kappa leader sequences
METDTLLLWVLLLWVPGSTGDYKDEYPYDVPDYAGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMETDTLLLWVLLLWVPGSTGDYKDEEQKLISEEDLGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 16 - RNA, 20 nt, synthetic construct
  source 1..20: mol_type = other RNA; note = sgRNA_CRISPR_TRAC_1
agagtctctcagctggtaca

SEQ ID NO: 17 - RNA, 20 nt, synthetic construct
  source 1..20: mol_type = other RNA; note = sgRNA_ CRISPR_TRAC_2
gagaatcaaaatcggtgaat

SEQ ID NO: 18 - RNA, 20 nt, synthetic construct
  source 1..20: mol_type = other RNA; note = sgRNA_ CRISPR_TRBC_1
caaacacagcgaccttgggt

SEQ ID NO: 19 - RNA, 20 nt, synthetic construct
  source 1..20: mol_type = other RNA; note = sgRNA_ CRISPR_TRBC_2
ggctctcggagaatgacgag

SEQ ID NO: 20 - RNA, 20 nt, synthetic construct
  source 1..20: mol_type = other RNA; note = sgRNA_Base_Editor_TRAC_1
ttcgtatctgtaaaaccaag

SEQ ID NO: 21 - RNA, 20 nt, synthetic construct
  source 1..20: mol_type = other RNA; note = sgRNA_ Base_Editor _TRAC_2
cttacctgggctggggaaga

SEQ ID NO: 22 - RNA, 20 nt, synthetic construct
  source 1..20: mol_type = other RNA; note = sgRNA_ Base_Editor _TRBC_2
aggtcctctggaaagggaag

SEQ ID NO: 23 - RNA, 20 nt, synthetic construct
  source 1..20: mol_type = other RNA; note = sgRNA_ Base_Editor _TRBC_2
ccactcacctgctctacccc

SEQ ID NO: 24 - protein, 975 aa, synthetic construct
  source 1..975: note = FMC63 TCR_CAR_Split_CCR_CD226mut Construct with GM-CSF and CD226 leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQAAPNNYRSPISTSQPTNQSMDDTREDIYVNYPTFSRRPATRVRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 25 - protein, 1030 aa, synthetic construct
  source 1..1030: note = FMC63 TCR_CAR_Split_CCR_CD226_CD2 Construct with GM-CSF and CD226 leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 26 - protein, 990 aa, synthetic construct
  source 1..990: note = FMC63 TCR_CAR_Split_CCR_CD226_CD2t Construct with GM-CSF and CD226 leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLKRKKQHPPPPPGHRSQAPSHRPPPPGHRVQHQPQKRPPAPSGTQVHQQKGPPLPRPRVQPKPPHGAAENSLSPSSNRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 27 - protein, 955 aa, synthetic construct
  source 1..955: note = FMC63 TCR_CAR_Split_CCR_CD226_CD28 Construct with GM-CSF and CD226 leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLRSKRSRLLHSDYMNMTPRRPGPTRKHYQPYAPPRDFAAYRSRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 28 - protein, 956 aa, synthetic construct
  source 1..956: note = FMC63 TCR_CAR_Split_CCR_CD226_4-1BB Construct with GM-CSF and CD226 leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLKRGRKKLLYIFKQPFMRPVQTTQEEDGCSCRFPEEEEGGCELRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 29 - protein, 917 aa, synthetic construct
  source 1..917: note = FMC63 TCR_CAR_Split_CCR_TIGIT_CD2 Construct with GM-CSF and TIGIT leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVAKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 30 - protein, 877 aa, synthetic construct
  source 1..877: note = FMC63 TCR_CAR_Split_CCR_TIGIT_CD2t Construct with GM-CSF and TIGIT leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVAKRKKQHPPPPPGHRSQAPSHRPPPPGHRVQHQPQKRPPAPSGTQVHQQKGPPLPRPRVQPKPPHGAAENSLSPSSNRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 31 - protein, 842 aa, synthetic construct
  source 1..842: note = FMC63 TCR_CAR_Split_CCR_TIGIT_CD28 Construct with GM-CSF and TIGIT leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVARSKRSRLLHSDYMNMTPRRPGPTRKHYQPYAPPRDFAAYRSRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 32 - protein, 843 aa, synthetic construct
  source 1..843: note = FMC63 TCR_CAR_Split_CCR_TIGIT_4-1BB Construct with GM-CSF and TIGIT leader sequences
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRGSGATNFSLLKQAGDVEENPGPMRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVAKRGRKKLLYIFKQPFMRPVQTTQEEDGCSCRFPEEEEGGCELRAKRGSGATNFSLLKQAGDVEENPGPMLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 33 - RNA, 20 nt, synthetic construct
  source 1..20: mol_type = other RNA; note = sgRNA_ Base_Editor _TIGIT_1
ggtaccttccacaagatcgg

SEQ ID NO: 34 - RNA, 20 nt, synthetic construct
  source 1..20: mol_type = other RNA; note = sgRNA_ Base_Editor _TIGIT_2
ggtggtaccttccacaagat

SEQ ID NO: 35 - RNA, 20 nt, synthetic construct
  source 1..20: mol_type = other RNA; note = sgRNA_ Base_Editor _TIGIT_3
caggaatacctgagctttct

SEQ ID NO: 36 - RNA, 20 nt, synthetic construct
  source 1..20: mol_type = other RNA; note = sgRNA_ Base_Editor _TIGIT_4
ctgggcccaggggctgaggc

SEQ ID NO: 37 - RNA, 20 nt, synthetic construct
  source 1..20: mol_type = other RNA; note = sgRNA_ Base_Editor _TIGIT_5
aggttccagattccattgct

SEQ ID NO: 38 - RNA, 20 nt, synthetic construct
  source 1..20: mol_type = other RNA; note = sgRNA_ Base_Editor _TIGIT_6
aagatcggtggcttgtacac

SEQ ID NO: 39 - RNA, 20 nt, synthetic construct
  source 1..20: mol_type = other RNA; note = sgRNA_ Base_Editor _TIGIT_7
tctgcttcctgtaggccctc

SEQ ID NO: 40 - RNA, 20 nt, synthetic construct
  source 1..20: mol_type = other RNA; note = sgRNA_ Base_Editor _TIGIT_8
tctcctcctgatctgggccc

SEQ ID NO: 41 - RNA, 20 nt, synthetic construct
  source 1..20: mol_type = other RNA; note = sgRNA_ Base_Editor _TIGIT_9
ccactcgatccttgaaggat

SEQ ID NO: 42 - protein, 15 aa, synthetic construct
EPKSCDKTHTCPPCP

SEQ ID NO: 43 - protein, 23 aa, synthetic construct
EPKSCDKTHTCPPCPAPELLGGP

SEQ ID NO: 44 - protein, 23 aa, synthetic construct
EPKSCDKTHTCPPCPAPEFLGGP

SEQ ID NO: 45 - protein, 15 aa, synthetic construct
EPKSCDKTHTCPSCP

SEQ ID NO: 46 - protein, 23 aa, synthetic construct
EPKSCDKTHTCPSCPAPELLGGP

SEQ ID NO: 47 - protein, 23 aa, synthetic construct
EPKSCDKTHTCPSCPAPEFLGGP

SEQ ID NO: 48 - protein, 12 aa, synthetic construct
ESKYGPPCPPCP

SEQ ID NO: 49 - protein, 20 aa, synthetic construct
ESKYGPPCPPCPAPELLGGP

SEQ ID NO: 50 - protein, 20 aa, synthetic construct
ESKYGPPCPPCPAPEFLGGP

SEQ ID NO: 51 - protein, 12 aa, synthetic construct
ESKYGPPCPSCP

SEQ ID NO: 52 - protein, 20 aa, synthetic construct
ESKYGPPCPSCPAPELLGGP

SEQ ID NO: 53 - protein, 20 aa, synthetic construct
ESKYGPPCPSCPAPEFLGGP

SEQ ID NO: 54 - protein, 15 aa, synthetic construct
GGGGSGGGGSGGGGS

SEQ ID NO: 55 - protein, 12 aa, synthetic construct
ESKYGPPCPSCP

SEQ ID NO: 56 - protein, 140 aa, Homo sapiens
  source 1..140: note = human TRAC from UniProt KB P01848
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 57 - protein, 140 aa, synthetic construct
  source 1..140: note = human TRAC with Thr->Cys and LVL mutations
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 58 - protein, 176 aa, Homo sapiens
  source 1..176: note = human TRBC1 from UniProt KB P01850
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 59 - protein, 176 aa, synthetic construct
  source 1..176: note = human TRBC1 with Ser-->Cys mutation
DLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 60 - protein, 178 aa, Homo sapiens
  source 1..178: note = human TRBC2 from UniProt KB A0A5G9
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 178 aa, synthetic construct
  source 1..178: note = human TRBC2 with Ser-->Cys mutation
DLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 62 - protein, 6 aa, synthetic construct
  source 1..6: note = TRAC sequence motif
LSVIGF

SEQ ID NO: 63 - protein, 6 aa, synthetic construct
  source 1..6: note = TRAC sequence motif with L-V-L mutations
LLVIVL

SEQ ID NO: 64 - protein, 6 aa, synthetic construct
  source 1..6: note = TRBC1 intracellular sequence
VKRKDF

SEQ ID NO: 65 - protein, 11 aa, synthetic construct
  source 1..11: note = TRBC2 intracellular sequence
MAMVKRKDSRG

SEQ ID NO: 66 - protein, 9 aa, synthetic construct
  source 1..9: note = WT-1 126 peptide
RMFPNAPYL

SEQ ID NO: 67 - protein, 9 aa, synthetic construct
  source 1..9: note = NY-ESO-1 157 peptide
SLLMWITQC

SEQ ID NO: 68 - protein, 12 aa, synthetic construct
  source 1..12: note = Townsend linker
GSGGSGGSGGTG

SEQ ID NO: 69 - protein, 18 aa, synthetic construct
  source 1..18: note = Whitlow linker (linker 218)
GSTSGSGKPGSGEGSTKG

SEQ ID NO: 70 - protein, 10 aa, synthetic construct
  source 1..10: note = linker
GSGKPGSGEG

SEQ ID NO: 71 - protein, 8 aa, synthetic construct
  source 1..8: note = linker
GKPGSGEG

SEQ ID NO: 72 - protein, 8 aa, synthetic construct
  source 1..8: note = linker
SGKPGSGE

SEQ ID NO: 73 - protein, 14 aa, synthetic construct
  source 1..14: note = linker
EGKSSGSGSESKVD

SEQ ID NO: 74 - protein, 6 aa, synthetic construct
  source 1..6: note = linker motif
  VARIANT 1: note = a positively charged amino acid
  VARIANT 3..5: note = or serine (S)
  VARIANT 6: note = or negatively charge amino acid
XPGGGG

SEQ ID NO: 75 - protein, 336 aa, Homo sapiens
  source 1..336: note = CD226 precursor (UniProt KB Q15762)
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 76 - protein, 18 aa, synthetic construct
  source 1..18: note = CD226 leader sequence
MDYPTLLLALLHVYRALC

SEQ ID NO: 77 - protein, 236 aa, synthetic construct [sequence omitted]

SEQ ID NO: 78 - protein, 21 aa, synthetic construct
  source 1..21: note = CD226 transmembrane sequence
GGTVLLLLFVISITTIIVIFL

SEQ ID NO: 79 - protein, 61 aa, synthetic construct
  source 1..61: note = CD226 endodomain sequence with K295A and K333A mutations
NRRRRRERRDLFTESWDTQAAPNNYRSPISTSQPTNQSMDDTREDIYVNYPTFSRRPATRV

SEQ ID NO: 80 - protein, 244 aa, Homo sapiens
  source 1..244: note = TIGIT precursor (UniProt KB Q495A1)
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 81 - protein, 21 aa, synthetic construct
  source 1..21: note = TIGIT leader sequence
MRWCLLLIWAQGLRQAPLASG

SEQ ID NO: 82 - protein, 120 aa, synthetic construct
  source 1..120: note = TIGIT ectodomain sequence
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 83 - protein, 21 aa, synthetic construct
  source 1..21: note = TIGIT transmembrane domain sequence
LLGAMAATLVVICTAVIVVVA

SEQ ID NO: 84 - protein, 116 aa, synthetic construct
  source 1..116: note = CD2 endodomain sequence
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 85 - protein, 76 aa, synthetic construct
  source 1..76: note = truncated CD2 endodomain sequence
KRKKQHPPPPPGHRSQAPSHRPPPPGHRVQHQPQKRPPAPSGTQVHQQKGPPLPRPRVQPKPPHGAAENSLSPSSN

SEQ ID NO: 86 - protein, 41 aa, synthetic construct
  source 1..41: note = CD28 co-stimulatory domain sequence
RSKRSRLLHSDYMNMTPRRPGPTRKHYQPYAPPRDFAAYRS

SEQ ID NO: 87 - protein, 42 aa, synthetic construct
  source 1..42: note = 4-1BB co-stimulatory domain sequence
KRGRKKLLYIFKQPFMRPVQTTQEEDGCSCRFPEEEEGGCEL

SEQ ID NO: 88 - protein, 318 aa, synthetic construct
  source 1..318: note = CD226-based (CD226mut) CCR sequence
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQAAPNNYRSPISTSQPTNQSMDDTREDIYVNYPTFSRRPATRV

SEQ ID NO: 89 - protein, 373 aa, synthetic construct
  source 1..373: note = CD226-based (CD2) CCR sequence
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 90 - protein, 332 aa, synthetic construct
  source 1..332: note = CD226-based (CD2t) CCR sequence
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLKRKKQHPPPPPGHRSQAPSHRPPPPGHRVQHQPQKRPPAPSGTQVHQQKGPPLPRPRVQPKPPHGAAENSLSPSS

SEQ ID NO: 91 - protein, 298 aa, synthetic construct
  source 1..298: note = CD226-based (CD28) CCR sequence
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLRSKRSRLLHSDYMNMTPRRPGPTRKHYQPYAPPRDFAAYRS

SEQ ID NO: 92 - protein, 299 aa, synthetic construct
  source 1..299: note = CD226-based (4-1BB) CCR sequence
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLKRGRKKLLYIFKQPFMRPVQTTQEEDGCSCRFPEEEEGGCEL

SEQ ID NO: 93 - protein, 257 aa, synthetic construct
  source 1..257: note = TIGIT-based (CD2) CCR sequence
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVAKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 94 - protein, 217 aa, synthetic construct
  source 1..217: note = TIGIT-based (CD2t) CCR sequence
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVAKRKKQHPPPPPGHRSQAPSHRPPPPGHRVQHQPQKRPPAPSGTQVHQQKGPPLPRPRVQPKPPHGAAENSLSPSSN

SEQ ID NO: 95 - protein, 182 aa, synthetic construct
  source 1..182: note = TIGIT-based (CD28) CCR sequence
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVARSKRSRLLHSDYMNMTPRRPGPTRKHYQPYAPPRDFAAYRS

SEQ ID NO: 96 - protein, 183 aa, synthetic construct
  source 1..183: note = TIGIT-based (4-1BB) CCR sequence
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVAKRGRKKLLYIFKQPFMRPVQTTQEEDGCSCRFPEEEEGGCEL

SEQ ID NO: 97 - protein, 107 aa, synthetic construct
  source 1..107: note = FMC63 antibody VL sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 98 - protein, 120 aa, synthetic construct
  source 1..120: note = FMC63 antibody VH sequence
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 99 - protein, 245 aa, synthetic construct
  source 1..245: note = FMC63 antibody scFv (VL-linker-VH) sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 100 - protein, 245 aa, synthetic construct
  source 1..245: note = FMC63 antibody scFv (VH-linker-VL) sequence
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 101 - protein, 113 aa, synthetic construct
  source 1..113: note = anti-GPRC5D antibody VL sequence
QSALTQPASVSGSPGQSITISCTGTSSDVGGYNYVSWYQQHPGKAPKLMIYDVSKRPSGVSNRFSGSKSGNTASLTISGLQAEDEADYYCSSYTRSSTEVFGGGTKLTVLGSR

SEQ ID NO: 102 - protein, 127 aa, synthetic construct
  source 1..127: note = anti-GPRC5D antibody VH sequence
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSKKWSGEKWRRENFDYWGQGTLVTVSS

SEQ ID NO: 103 - protein, 259 aa, synthetic construct
  source 1..259: note = anti-GPRC5D antibody scFv (VL-linker-VH) sequence
QSALTQPASVSGSPGQSITISCTGTSSDVGGYNYVSWYQQHPGKAPKLMIYDVSKRPSGVSNRFSGSKSGNTASLTISGLQAEDEADYYCSSYTRSSTEVFGGGTKLTVLGSRGGGGSGGGGSGGGGSLEMAEVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSKKWSGEKWRRENFDYWGQGTLVTVSS

SEQ ID NO: 104 - protein, 117 aa, synthetic construct
  source 1..117: note = anti-BCMA antibody (C11D5.3) VH sequence
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 105 - protein, 111 aa, synthetic construct
  source 1..111: note = anti-BCMA antibody (C11D5.3) VL sequence
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 106 - protein, 243 aa, synthetic construct
  source 1..243: note = anti-BCMA antibody (C11D5.3) scFv (VH-linker-VL) sequence
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 107 - protein, 116 aa, synthetic construct
  source 1..116: note = VHH sequence
QVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSS

SEQ ID NO: 108 - protein, 235 aa, synthetic construct
  source 1..235: note = VHH sequence - AAA linker - VHH sequence
QVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSS

SEQ ID NO: 109 - protein, 165 aa, synthetic construct
  source 1..165: note = Bcl2 ectodomain sequence
AHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMR

SEQ ID NO: 110 - protein, 283 aa, synthetic construct
  source 1..283: note = FMC63 VL-TRBC
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 111 - protein, 260 aa, synthetic construct
  source 1..260: note = FMC63 VH-TRAC
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 112 - protein, 296 aa, synthetic construct
  source 1..296: note = FMC63 VH-TRBC
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 113 - protein, 247 aa, synthetic construct
  source 1..247: note = FMC63 VL-TRAC
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 114 - protein, 385 aa, synthetic construct
  source 1..385: note = FMC63 scFv (VL-linker-VH) TRAC
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 115 - protein, 421 aa, synthetic construct
  source 1..421: note = FMC63 scFv (VL-linker-VH) TRBC
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 116 - protein, 385 aa, synthetic construct
  source 1..385: note = FMC63 scFv (VH-linker-VL) TRAC
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 117 - protein, 421 aa, synthetic construct
  source 1..421: note = FMC63 scFv (VH-linker-VL) TRBC
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 118 - protein, 435 aa, synthetic construct
  source 1..435: note = anti-GPRC5D scFv (VL-linker-VH) TRBC
QSALTQPASVSGSPGQSITISCTGTSSDVGGYNYVSWYQQHPGKAPKLMIYDVSKRPSGVSNRFSGSKSGNTASLTISGLQAEDEADYYCSSYTRSSTEVFGGGTKLTVLGSRGGGGSGGGGSGGGGSLEMAEVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSKKWSGEKWRRENFDYWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 119 - protein, 383 aa, synthetic construct
  source 1..383: note = anti-BCMA scFv (VH-linker-VL) TRAC
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 120 - protein, 375 aa, synthetic construct
  source 1..375: note = VHH-AAA-VHH TRAC
QVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 121 - protein, 411 aa, synthetic construct
  source 1..411: note = VHH-AAA-VHH TRBC
QVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSAAAQVKLVQSGGGLVQTGGSLRLSCAASEITFDMYSMGWYREAPGKARDAVASITNRGNTYYADSVKGRFTISRDNAKKTMYLQMNSLKPEDTAVYYCNVYRTGFSDYWGQGTQVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 122 - protein, 327 aa, synthetic construct
  source 1..327: note = Bcl2-linker-hinge-TRAC
AHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 123 - protein, 343 aa, synthetic construct
  source 1..343: note = Flag/HA-tag-Bcl2-linker-hinge-TRAC
DYKDEYPYDVPDYAGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 124 - protein, 363 aa, synthetic construct
  source 1..363: note = Bcl2-linker-hinge-TRBC
AHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 125 - protein, 379 aa, synthetic construct
  source 1..379: note = Flag/HA-tag-Bcl2-linker-hinge-TRBC
DYKDEYPYDVPDYAGSAHAGRTGYDNREIVMKYIHYKLSQRGYEWDAGDDAEENRTEAPEGTESEVVHRALRDAGDDFERRYRRDFAEMSSQLHLTPDTARQRFETVVEELFRDGVNWGRIVAFFEFGGVMCVESVNREMSPLVDNIAEWMTEYLNRHLHTWIQDNGGWDAFVELYGPSMRGGGGSGGGGSESKYGPPCPPCPDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 126 - protein, 22 aa, synthetic construct
  source 1..22: note = GM-CSF leader sequence
MLLLVTSLLLCELPHPAFLLIP

SEQ ID NO: 127 - protein, 21 aa, synthetic construct
  source 1..21: note = CD8alpha leader sequence
MALPVTALLLPLALLLHAARP

SEQ ID NO: 128 - protein, 21 aa, synthetic construct
  source 1..21: note = CD8beta leader sequence
MRPRLWLLLAAQLTVLHGNSV

SEQ ID NO: 129 - skipped

SEQ ID NO: 130 - protein, 5 aa, synthetic construct
  source 1..5: note = consensus minimal furin cleavage site sequence
RXKRR

SEQ ID NO: 131 - protein, 4 aa, synthetic construct
  source 1..4: note = minimal furin cleavage site sequence
RAKR

SEQ ID NO: 132 - protein, 4 aa, synthetic construct
  source 1..4: note = minimal furin cleavage site sequence
RARR

SEQ ID NO: 133 - protein, 22 aa, Teschovirus A
  source 1..22: note = Porcine teschovirus-1 2A (P2A) self-cleaving peptide with N-terminal GSG linker
GSGATNFSLLKQAGDVEENPGP

SEQ ID NO: 134 - protein, 24 aa, Thosea asigna virus
  source 1..24: note = Thoseaasigna virus 2A (T2A) self-cleaving peptide
LEGGGEGRGSLLTCGDVEENPGPR

SEQ ID NO: 135 - protein, 20 aa, Equine rhinitis A virus
  source 1..20: note = Equine rhinitis A virus (ERAV) 2A (E2A) self-cleaving peptide
QCTNYALLKLAGDVESNPGP

SEQ ID NO: 136 - protein, 25 aa, Foot-and-mouth disease virus
  source 1..25: note = Foot-and-Mouth disease virus 2A (F2A) self-cleaving peptide with N-terminal G-S-G linker
GSGVKQTLNFDLLKLAGDVESNPGP

SEQ ID NO: 137 - protein, 8 aa, synthetic construct
  source 1..8: note = Flag Tag
DYKDDDDK

SEQ ID NO: 138 - protein, 5 aa, synthetic construct
  source 1..5: note = Flag Tag variant
DYKDE

SEQ ID NO: 139 - protein, 10 aa, synthetic construct
  source 1..10: note = HA Tag
YPYDVDPDYA

SEQ ID NO: 140 - protein, 16 aa, synthetic construct
  source 1..16: note = Flag/HA Hybrid Tag
DYKDEYYPYDVDPDYA

SEQ ID NO: 141 - protein, 38 aa, synthetic construct
  source 1..38: note = ICOS intracellular sequence
CWLTKKKYSSSVHDPNGEYMFMRAVNTAKKSRLTDVTL

SEQ ID NO: 142 - protein, 42 aa, synthetic construct
  source 1..42: note = OX40 intracellular sequence
ALYLLRRDQRLPPDAHKPPGGGSFRTPIQEEQADAHSTLAKI

SEQ ID NO: 143 - protein, 58 aa, synthetic construct
  source 1..58: note = GITR intracellular sequence
QLGLHIWQLRSQCMWPRETQLLLEVPPSTEDARSCQFPEEERGERSAEEKGRLGDLWV

SEQ ID NO: 144 - protein, 48 aa, synthetic construct
  source 1..48: note = CD27 intracellular sequence
QRRKYRSNKGESPVEPAEPCHYSCPREEEGSTIPIQEDYRKPEPACSP

SEQ ID NO: 145 - protein, 60 aa, synthetic construct
  source 1..60: note = HVEM intracellular sequence
CVKRRKPRGDVVKVIVSVQRKRQEAEGEATVIEALQAPPDVTTVAVEETIPSFTGRSPNH

SEQ ID NO: 146 - protein, 24 aa, synthetic construct
  source 1..24: note = DAP10 intracellular sequence
LCARPRRSPAQEDGKVYINMPGRG

SEQ ID NO: 147 - protein, 77 aa, synthetic construct
  source 1..77: note = SLAMF1 intracellular sequence
QLRRRGKTNHYQTTVEKKSLTIYAQVQKPGPLQKKLDSFPAQDPCTTIYVAATEPVPESVQETNSITVYASVTLPES

SEQ ID NO: 148 - protein, 182 aa, synthetic construct
  source 1..182: note = LIGHT intracellular sequence
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 149 - protein, 189 aa, synthetic construct
  source 1..189: note = CD30 intracellular sequence
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 150 - protein, 20 aa, synthetic construct
  source 1..20: note = murine IgG, kappa leader sequence
METDTLLLWVLLLWVPGSTG

SEQ ID NO: 151 - protein, 10 aa, synthetic construct
  source 1..10: note = Myc tag
EQKLISEEDL

(19) United States
(12) Patent Application Publication
(10) Pub. No.: US 2026/0250405 A1
(43) Pub. Date: Aug. 27, 2026

(54) COMPOSITIONS AND METHODS FOR CELLULAR IMMUNOTHERAPY

(71) Applicant: FRED HUTCHINSON CANCER CENTER, Seattle, WA (US)

(72) Inventors: Sylvain SIMON, Seattle, WA (US); Stanley R. RIDDELL, Sammamish, WA (US); Grace BUGOS, Seattle, WA (US)

(21) Appl. No.: 18/861,869

(22) PCT Filed: May 1, 2023
(86) PCT No.: PCT/US2023/066466

§ 371 Date: Oct. 30, 2024

Related U.S. Application Data
(60) Provisional application No. 63/337,588, filed on May 2, 2022.

(51) Int. Cl. C07K 16/28; A61K 38/00; A61K 40/11; A61K 40/32; A61K 40/41; A61P 35/00; C07K 14/705; C12N 5/0783; C12N 15/86

(52) U.S. Cl. CPC C07K 16/2878; A61K 40/11; A61K 40/32; A61K 40/41; A61P 35/00; C07K 14/70503; C07K 14/70514; C07K 16/2803; C07K 16/2866; C12N 5/0636; C12N 15/86; A61K 38/00; C07K 2317/31; C07K 2317/622; C07K 2317/92; C07K 2319/03; C07K 2319/30; C12N 2510/00; C12N 2740/15043

(57) ABSTRACT

The present disclosure provides polypeptide dimers and polypeptides for improving cellular immunotherapy. Disclosed embodiments include polypeptide dimers (referred-to herein as TCR/CARs) that confer to a host cell (e.g. a T cell) target-specificity of a CAR while leveraging natural T cell signaling properties. TCR/CARs can be efficiently expressed at the surface of host cells and confer to host cells a diversified and highly sensitive signaling complex that acts with native host (e.g. T) cell signaling properties. Also provided are chimeric co-stimulatory receptor polypeptides (CCRs) that are capable of improving host cell (e.g. T cell) function when the host cell encounters PVR aka CD155. CCRs comprise an extracellular component (e.g. from CD226 or TIGIT) that is capable of binding to PVR and an intracellular component designed or selected to preserve normal CD226 signaling or disrupt or abrogate normal TIGIT signaling, such as by abrogating this signaling and/or by converting this signaling into an activating or co-stimulatory signal. Also provided are polynucleotides encoding the TCR/CARs and/or CCRs, and vectors that comprise the polynucleotides. Also provided are host cells, such as T cells, that encode or express the TCR/CARs and/or CCR. Also provided are compositions and methods for using the same the same.

### STATEMENT OF GOVERNMENT INTEREST

This invention was made with government support under CA18029 and CA114536 awarded by the National Institutes of Health and W81XWH-20-1-0230 awarded by the United States Army Medical Research and Development Command. The government has certain rights in the invention.

### REFERENCE TO AN ELECTRONIC SEQUENCE LISTING

The contents of the electronic sequence listing (360056_498WO_SeqListing.xml, Size: 186 kilobytes; and Date of Creation: Apr. 6, 2023) is herein incorporated by reference in its entirety.

### BACKGROUND

Genetically engineered T cells expressing transgenic T Cell Receptors (TCRs) or Chimeric Antigen Receptors (CARs) bave proven to be relevant options to treat certain cancers. CAR T therapies have notably been successful for treating patients with refractory hematologic malignancies. CARs include an extracellular domain comprising an antibody single-chain variable fragment (scFv) or other binding domain that recognizes antigen, such as antigen expressed on the surface of target cells. CARs also include an intracellular domain comprising one or more signaling domains to activate the T cell and typically include the CD3ζ cytoplasmic domain, often paired with a co-stimulatory signaling from, for example, CD28 or 4-1BB.

Despite clinical success, some limitations of CAR T cells have been observed. Namely, some CAR T therapies have been observed to induce toxicities that can be severe and potentially life-threatening such as Cytokine Release Syndrome (CRS). Antigen-independent CAR T cell activation, which has been referred-to as tonic signaling, is another observed limitation of some CARs as it fosters higher cytokine production, contributing to toxicities and driving early T cell exhaustion thus dampening long term functions and antitumor activity. Another observed limitation of CAR T cells to-date is a weak ability to sense target cells expressing low levels of antigen, which is thought to play a role in limiting clinical success by allowing escape of tumor cells with low levels of antigen. These limitations may originate from CAR-intrinsic signaling properties.

TCRs also have limitations. TCRs are MHC restricted-they recognize their antigen (peptides from intracellular proteins) presented on a given MHC molecule, thus limiting the number of patients eligible for each therapy to those that express both the antigen and the relevant MHC molecule. The antigen presentation pathway for T cell recognition involves multiple partners (MHC, TAP1/2, β2m) and is frequently altered in cancer cells as an immune escape mechanism (downregulation or mutations). Additionally, peripheral T cells are selected through thymic selection, a process where self-reactive, low affinity and high affinity TCRs are sequentially depleted. As a result, discovering high affinity TCRs (which have been described to provide preferred functions) specific for tumor antigens remains challenging.

Additional modalities for cellular immunotherapy that overcome these limitations of CARs and TCRs are needed.

### BRIEF DESCRIPTION OF THE DRAWINGS

FIGS. 1A-ID relate to certain embodiments of T cell receptor/chimeric antigen receptor hybrid (TCR/CAR) constructs of the present disclosure. (A) Top left: schematic of TCR/CAR in “split-scFv” format (schematic of expression vector shown at bottom left) expressed at a cell membrane; top right: schematic of TCR/CAR in “full-scFv” format (schematic of expression vector shown at bottom right) expressed at a cell membrane. A non-limiting example of an expression product of a split-scFv TCR/CAR vector as illustrated is provided in SEQ ID NO.: 1. A non-limiting example of an expression product of a full-scFv TCR/CAR vector as illustrated is provided in SEQ ID NO.: 3. As described further herein, TCR/CARs can include a (ny) target-binding domain (e.g., VH, VL, scFv, VHH, ligand, receptor ectodomain, fully synthetic (e.g., designed de novo) binding protein, or the like), such as for example a scFv comprising VH and VL from a tumor antigen-specific antibody.

In the illustrated “split-scFv” format, the VH and VL are not linked to one another by a peptide linker (as would be in a scFv)-though in some embodiments they may share one or more interchain disulfide bond-, but each is fused or linked to one of two TCR constant domains. For example, VH can be fused or linked to a T cell receptor beta-chain constant domain (TRBC) and VL can be fused or linked to a T cell receptor alpha chain-constant domain (TRAC), or VL can be fused or linked to a TRBC domain and VH can be fused or linked to a TRAC domain. Amino acid sequences of non-limiting examples of “split-scFv” expression products (vector-encoded amino acid sequence) are provided in SEQ ID NOs.: 1 (VL-TRBC_VH-TRAC) and 2 (VH-TRBC_VL-TRAC).

In the illustrated “full-scFv” format, a scFv includes a linker and can be in VH-linker-VL or VL-linker-VH orientation, and the scFv can be fused to either TCR constant domain (e.g. to TRAC or to TRBC) of a TCR constant domain dimer. Non-limiting examples of “full-scFv” expression products (vector-encoded amino acid sequence) are provided in SEQ ID NOs.: 3-6. As shown in, for example, FIG. 5C, each TCR constant domain can be fused to a binding domain, such as a scFv. Contemplated embodiments include polypeptide chains comprising two or more binding domains (e.g., scFv-linker-scFv-TCR constant domain). In embodiments comprising two or more scFvs (comprised in one or two polypeptide chains), two or more scFvs can be in the same orientation (e.g., two or more can be VH-linker-VL or VL-linker-VH) or one scFv can be in a VH-linker-VL and another scFv can be in VL-linker-VH. Likewise, a scFab can be VH-CH1-linker-VL-CL or VL-CL-linker-VH-CH1, and two or more scFabs can be in the same or different orientations.

In the illustrated expression vectors, a signal peptide (SP; also called a leader sequence) is shown disposed at the N-terminal end of each polypeptide chain, and a furin cleavage sequence (RAKR, SEQ ID NO.: 131) and a Thoseaasigna virus 2A (T2A) self-cleaving peptide (LEGGGEGRGSLLTCGDVEENPGPR; SEQ ID NO.: 134) separate the two polypeptides. An alternative 2A peptide sequence, such as a P2A self-cleaving peptide with N-terminal GSG linker (GSGATNFSLLKQAGDVEENPGP; SEQ ID NO.: 133), can be used. It will be understood that other self-cleaving peptides and cleavage (e.g. protease recognition) sequences may be used. Signal peptides are typically removed, in whole or in part, prior to expression of a polypeptide at a cell surface. TCR/CAR constructs can be delivered to host cells using, for example, a viral vector such as a lentiviral vector. A vector can include a promoter, such as, for example, an EF1a promoter (SEQ ID NO.: 7) or a MNDU3 promoter (SEQ ID NO.: 8). (B) Expression of CARs and TCR/CARs in primary T cells with knockout of endogenous TCRα and TCRβ. (C) Basal activation of TCRs, CD19/28z CAR, CD19/BBz CAR and TCR/CARs in Jurkat NFAT reporter cells. (D) Lysis of (left) CD19-negative and (right) CD19+ target cells by cells expressing a CAR or TCR/CAR as indicated.

FIG. 2 shows that RORI-specific TCR/CARs exhibit better recognition at low antigen density than a ROR 1-specific CAR.

FIGS. 3A-3C relate to certain embodiments of CD226-based or TIGIT-based chimeric costimulatory receptors (“CCRs”) (also referred-to herein as immunomodulatory fusion proteins “IFPs”), which can be co-expressed with TCR/CARs of the present disclosure, or with, for example, TCRs or CARs. (A (left, right)) Schematics showing TCR/CARs and CCRs. The extracellular and optionally the transmembrane components can be from CD226 (left) or TIGIT (right), and the intracellular component can comprise, for example, a mutated CD226 endodomain or portion thereof, a CD2 endodomain or portion thereof (e.g., a truncated CD2 endodomain), a CD28 endodomain or portion thereof, or a 4-1BB endodomain or portion thereof. (B) Co-expression of TCR/CARs and CCRs in primary CD8 T cells with knock-out of endogenous TCRa, TCRB and TIGIT expression. (C) CD226 (left) and TIGIT (right) expression in T cells expressing CD19-specific TCR/CAR alone (WT) or in T cells engineered with TCR/CARs as in (B) and with different CCRs with a CD226 or TIGIT ectodomain.

FIG. 4 shows embodiments of full-design and split-design TCR/CARs in association with CD3 proteins of a TCR complex at a cell membrane. ITAM=Immunoreceptor Tyrosine-based Activation Motif. (left) “Split-scFv” construct with antibody variable domains; (right) “full-scFv” construct. X=immunoreceptor tyrosine-based activation motifs (ITAMs) present in CD3 proteins.

FIGS. 5A-5E relate to certain embodiments of TCR/CAR constructs of the present disclosure. (A) Schematic representation of an example of a “split-scFv” construct as an expression vector (bottom) and TCR/CAR expressed at a cell membrane (top). In this example, VH is comprised in a single chain fusion with the TRAC domain, and VL is comprised in a single chain fusion with the TRBC domain. (B) Schematic representation of an example of a “full-scFv” construct as an expression vector (bottom) and TCR/CAR expressed at a cell membrane (top). In this example, a VL-linker-VH scFv is comprised in a single chain fusion with TRAC. (C) Schematic represenation of an example of a “bi-specific format” construct as an expression vector (bottom) and as TCR/CAR expressed at a cell membrane (top). In this example, a different VL-linker-VH scFv is comprised in a single chain fusion with each of the TRAC and TRBC domains. Alternatively, a scFv can be VH-linker-VL, and different scFvs present in a TCR/CAR can have different orientations. Each scFv can be specific for a different target (e.g. tumor antigen) to recognize cells expressing either or both antigens, or can target the same epitope or can target different epitopes within the same target to provide greater avidity. In other embodiments, a mono-specific TCR/CAR can comprise one (or more) scFv fused or linked to each of two TCR constant domains, wherein the scFvs are the same or bind the same epitope. In other words, a “full-scFv” TCR/CAR can comprise two scFvs while having single-target specificity. A non-limiting example of an expression product according to the illustrated bispecific full-scFv TCR/CAR vector is provided in SEQ ID NO.: 9. It will be understood that other binding domains, addtionally or alternatively to scFvs, may be employed in multi-specific (e.g., bispecific) or multivalent (e.g., bivalent) TCR/CARs.

Any of the presently disclosed TCR/CAR constructs or IFP constructs can be co-expressed with one another, and/or with a transduction marker; a transduction marker may also function as a suicide switch (e.g. targetable by an antibody or antigen-binding fragment that induces cell death of a cell expressing the suicide switch); non-limiting examples of suicide switch transduction markers include tEGFR, tCD19, INGFR, or the like. (D) Schematic representations of “VHH-based” (also referred-to as “nanobody-based”) TCR/CARs shown with a VHH linked or fused to one, the other, or both TCR constant domains of a TCR constant domain dimer. For example, a VHH can be linked or fused to TRAC (left; e.g. vector-encoded amino acid sequence of SEQ ID NO.: 10, wherein two copies of a VHH are comprised in a polypeptide chain further comprising TRAC) or to TRBC (center; e.g. vector-encoded amino acid sequence of SEQ ID NO.: 11, wherein two copies of a VHH are comprised in a polypeptide chain further comprising TRBC), or a VHH can be linked or fused to each of TRAC and TRBC (right; e.g. vector-encoded amino acid sequence of SEQ ID NO.: 12, wherein two copies of a VHH are comprised in a polypeptide chain further comprising TRAC and two copies of a VHH are comprised in a polypeptide chain further comprising TRBC). In embodiments where two (or more) VHH are present, the two (or more) VHH can be the same or can be different, and if different, may have specificity for different targets or for different epitopes of the same target. (E) Schematic representation of “protein-based” TCR/CARs with a N-terminal HA-tag and Bcl-2 ectodomain linked or fused to TRAC (left; e.g. vector-encoded amino acid sequence of SEQ ID NO.: 13) or TRBC (right; e.g. vector-encoded amino acid sequence of SEQ ID NO.: 14). Not shown: schematic of embodiment wherein a Bcl-2 ectodomain (with N-terminal HA-tag) is linked or fused to each of TRAC and TRBC (e.g., vector-encoded amino acid sequence of SEQ ID NO.: 15).

FIG. 6 provides non-limiting examples of targets targeted using TCR/CARs of the present disclosure.

FIG. 7 shows a schematic of certain sequence modifications that can be used to improve chain pairing and stability of TCR/CARs. Shown is an embodiment of a “split-scFv” TCR/CAR with certain amino acid mutations to improve pairing efficiency between the two chains of the construct and to increase its stability when expressed at the cell surface. The upper “C” in TRAC represents a threonine to cysteine mutation at position 48 (T48C), and the lower “C” in TRBC represents a serine to cysteine mutation at postion 57 (S57C). The other “C”s (bottom in TRAC, top in TRBC) are native cysteines. The T48C and S57C mutations permit formation of novel disulfide bonds bewteen TRAC and TRBC. The “LVL” found in the diagram of the plasma membrane represents mutations in the TRAC transmembrane portion that introduce leucine (L), valine (V), and leucine (L) amino acid residues (described further herein). These three hydrophobic residues counterbalance instability. The positions of the LVL mutations within TRAC sequence are known (see Haga-Friedman et al., J Immunol 188:5538-5546 (2012)) and discussed further herein (see SEQ ID NOS.: 57 and 63).

TCR/CARs comprising TRAC and TRBC domains can include any or all of the above-mentioned pairing efficiency and stabilizing mutations, and/or can include other mutations as described herein. Additionally or alternatively, TCR/CARs may be expressed in T cells in which one or more endogenous TCR locus (e.g., TRAC, TRBC) is knocked-out to prevent mispairing between a TCR/CAR constant domain and a potential cognate endogenous TCR constant domain.

FIGS. 8A-8C relate to certain embodiments in which endogenous TRAC and TRBC genes of T cells were targeted for knockout utilizing either CRISPR or base-editing technologies. (A) Schematic of experimental workflow (see e.g. Kluesner et al., Nature Communications 12:2437 (2021)) to generate TCR/CAR T cells. The exemplified strategy includes activating bulk T cells, transducing the activated T cells with lentivirus encoding a TCR/CAR, and knocking out endogenous TRAC and/or TRBC genes using a CRISPR/Cas or base editor system. (B) Results of Base Editor knockout of endogenous TRAC and/or TRBC in primary T cells. sgRNA TRAC-1, sgRNA TRAC-2, sgRNA TRBC-1, and sgRNA TRBC-2 refer to different base editor sgRNAs targeting either the TRAC or TRBC locus. See SEQ ID NOS.: 20-23. Each sgRNA was used individually (top row) or in the listed combinations (bottow row). (C) Summarizes efficiency of base-editing knockout (% of edited TCRs) in graphical format. sgRNA TRAC-1 is represented by the symbol α1, sgRNA TRAC-2 is represented by the symbol α2, sgRNA TRBC-1 is represented by the symbol β1, and sgRNA TRBC-2 is represented by β2.

FIGS. 9A and 9B relate to experiments for improving T cell transduction and base-editing efficiency. Timing and sequencing (ordering) of lentiviral transduction and base-editing was tested. (A) Six different tranduction and base-editing conditions were tested experimentally. D0, D1, D2, D3, and D6 refer to day zero (the time when T cells were placed into culture), day one, day two, day three, and day six of the culture period, respectively. “Td” represents the time, with reference to DO-D6, at which cells were transduced with lentiviral expression constructs. “BE” represents the time, with reference to DO-D6, at which base-editing was performed. (B) Cellular expression of TCR/CAR and knockout of endogencous TRAC and/or TRBC measured by flow cytometry. Cells were stimulated on either day zero (top row) or day one (bottom row). Left panels: cells were transduced with lentivirus, rested for six hours, and then base-edited. Center panels: cells were base-edited, rested for six hours, and then transduced with lentivirus. Right panels: cells were transduced with lentivirus on day two and base-edited on day three.

FIGS. 10A-10E relate to certain embodiments of TCR/CARs of the present disclosure. (A) Cell surface expression of CD19-specific CARs (with either CD28 costimulatory domain and CD35 effector domain or 4-1BB costimulatory domain and CD3 (effector domain), a CD19-specific “split-scFv” TCR/CAR, and a CD19-specific “full-scFv” TCR/CAR in primary CD4+ and CD8+ T cells. TCR/CARs are expressed in a similar frequency as CARs in primary T cells. (B) Transduction percentage (top row) and expression (quantified as geometric mean rCD19) (bottom row) of the CAR and TCR/CAR constructs shown in FIG. 10A, in primary CD4+ and CD8+ T cells. (C) Cell surface expression of RORI-specific CARs, a RORI-specific “split-scFv” TCR/CAR, and a RORI-specific “full-scFv” TCR/CAR in primary CD4+ and CD8+ T cells. (D) Transduction percentage (top row) and expression (quantified as geometric mean rROR1) (bottom row) of the constructs shown in FIG. 10C, in primary CD4+ and CD8+ T cells. (E) Cell surface expression of BCMA-specific CARs, a BCMA-specific “split-scFv” TCR/CAR, and a BCMA-specific “full-scFv” TCR/CAR in primary CD4+ and CD8+ T cells. These data show that TCR/CARs specific for different antigens and in either split-scFv or full-scFv format expressed efficiently in primary CD4+ and CD8+ T cells.

FIG. 11 shows data comparing cell surface expression levels of the indicated TCR/CARs when the encoding lentiviral vector contained either an EF1a promoter or a MNDU3 promoter. The MNDU3 promoter provided increased cell surface TCR/CAR expression and frequency of T cells that express the TCR/CAR as compared to the EFla promoter,

FIGS. 12A and 12B relate to certain embodiments of “bi-specific full-scFv” constructs recognizing different multiple myeloma antigens. (A) Schematic representations of “bi-specific full-scFv” constructs (top row). Expression of “bi-specific full-scFv” constructs (bottom row) in primary T cells. (B) Schematic representation of an anti-CD229×anti-BCMA “bi-specific full-scFv” construct (far left panel), cell surface expression of the anti-CD229×anti-BCMA “bi-specific full-scFv” construct (center left panel), binding by the anti-CD229×anti-BCMA “bi-specific full-scFv” TCR/CAR to biotinylated BCMA (center right panel), and binding by the anti-CD229×anti-BCMA “bi-specific full-scFv” TCR/CAR to biotinylated CD229 (far right panel). These data show that bispecific TCR/CARs, such as in full scFv format, can target different antigens and be expressed in primary T cells.

FIG. 13 relates to certain embodiments of “VHH-based” and “protein-based” constructs of the present disclosure. Left panel: expression (bottom) of an anti-RORI “VHH-based” TCR/CAR (schematic shown at top) in primary T cells. Right panel: expression of a “protein-based” TCR/CAR (schematic shown at top) in primary T cells. A “protein-based” TCR/CAR includes a TCR/CAR comprising a target-binding portion that is a native protein or portion thereof that is not an antigen-binding domain of an antibody and interacts with its native ligand (target).

FIG. 14A-14C: (A) Jurkat cells with a triple reporter system NFAT-eGFP, NFkB-CFP, AP-1-mCherry were unstimulated or were stimulated with PMA/Ionomycin demonstrating upregulation of reporter constructs with stimulation. (B) TCR, CAR, “split-scFv” TCR/CAR, and “full-scFv” TCR/CAR constructs were individually transduced into Jurkat NFAT-eGFP, NFkB-CFP, AP-1-mCherry triple reporter cells and the percentage of NFAT-reporter positive cells in unstimulated conditions was measured. TCRs, CARs, and TCR/CARs were specific for the indicated antigen. (C) TCR, CAR, “split-scFv” TCR/CAR, and “full-scFv” TCR/CAR constructs were individually transduced into Jurkat NFAT-cGFP, NFkB-CFP, AP-1-mCherry triple reporter cells and the percentage of NFkB-reporter positive cells in unstimulated conditions was measured. TCRs, CARs, and TCR/CARs were specific for the indicated antigen.

These data show that TCR/CAR constructs induce minimal antigen-independent signaling.

FIGS. 15A and 15B show results from western blot experiments. (A) Detection of TRAC or TRBC in cell lysate from RORI-specific TCR/CAR-positive T cells. Western blots were performed against TRAC (left) and TRBC (right) using total input samples or immunoprecipitation (IP) samples with biotinylated recombinant RORI protein coated on streptavidin-coated beads. (B) Detection of all 4 CD3 sub-unit proteins in cell lysate from ROR1-specific TCR/CAR+ T cells after immunoprecipitation with biotinylated recombinant RORI protein coated on streptavidin-coated beads.

These data show that unlike a CAR, TCR/CAR constructs assemble with all CD3 signaling complex proteins.

FIG. 16 shows cellular Ca²⁺ flux following CD19 antigen stimulation of T cells transduced with a CD19-specific CAR comprising CD28/CD3ζ (or 4-1BB/CD3ζ signaling domains, or with a CD19-specific TCR/CAR of the present disclosure.

These data show that T cells expressing TCR/CAR constructs flux Ca²⁺ following antigen-specific stimulation as well as or better than T cells expressing comparator CARS.

FIG. 17 shows the percentage of killing of cancer cell lines by T cells transduced with CD19-specific CARs or CD19-specific TCR/CARs at various effector to target (E:T) ratios. K562 and Nalm 6 refer to CD19-positive cancer cell lines. K562 CD19^{ko} and Nalm 6 CD19^{ko} refer to engineered versions of K562 and Nalm6 cell lines, respectively, in which CD19 has been knocked-out.

These data show that T cells expressing TCR/CARs specifically recognize and lyse tumor cells expressing a targeted antigen.

FIG. 18 shows cytokine production (IL-2; IFN-γ) by T cells transduced with a CD19-specific CAR or a CD19-specific TCR/CAR, in response to antigen-positive and antigen-negative cell lines.

FIG. 19 shows proliferation, by T cells transduced with a CD19-specific CAR or a CD19-specific TCR/CAR, in response to various cell lines. Top, representative histograms from flow cytometry of T cells cultured in media (CTL) or with the indicated cell lines using Cell Trace Violet. Bottom left, percentage of divided T cells; bottom right, Geometric mean of CTV intensity.

FIG. 20 shows that TCR/CAR+ T cells possess higher functional avidity (measured as percentage of bound T cells) for their target than CAR+ T cells do for their target, as measured by z-movi, which determines the acoustic force required to disrupt binding of T cells to target cells. The tested TCR/CARs and CARs used antigen-binding domains from the same CD19-specific antibody.

FIGS. 21A and 21B show that TCR/CARs have superior antigen sensitivity as compared to CARs. (A) Response of T cells transduced with CD19-specific TCR/CARs or CD19-specific CARs to increasing concentrations of recombinant CD19 antigen (x-axis). Cellular reponse is monitored by measuring IL-2, TNF-α, and IFN-g cytokine concentration (y-axis). (B) Half-maximal effective concentration (EC50) measurements of CD19-specific CARs and CD19-specific TCR/CARs with respect to IL-2 and IFN-g production in response to antigen.

FIG. 22 provides a schematic illustrating an in vivo experiment in which CD19+ Raji GFP-FfLuc cells were infused into NSG mice. Seven days after administration of CD19+ Raji GFP-FfLuc cells to mice, equal numbers of CD4+ and CD8+ T cells transduced with CARs or TCR/CARs were infused into the mice. Growth of the Raji GFP-FfLuc cells was measured by bioluminescence (radiance) and the survival of the mice was monitored over time. These data show that TCR/CARs have antitumor efficacy in a NSG mouse model.

FIGS. 23A-23E relate to certain IFPs (also called CCRs, chimeric costimulatory receptors) of the present disclosure. CD226 is an adhesion molecule that binds to CD155 and amplifies T cell functions following receptor triggering. CD226 is expressed on unstimulated T cells and can be gradually lost following chronic antigen stimulation, while TIGIT, which also binds CD155, is expressed following T cell activation and becomes constitutively expressed during T cell exhaustion (see, e.g. Ge et al., Front Immunol 2021; doi.org/10.3389/fimmu.2021.699895). IFPs leveraging CD226 or TIGIT extracellular domain were constructed to potentially manipulate the PVR/TIGIT/CD226 signaling axis, where the common ligand for TIGIT and CD226, PVR aka CD155, is widely expressed and is often overexpressed by tumor cells. TIGIT has a higher affinity for PVR (approximately 100-fold) than does CD226. These IFPs are also referred-to herein as chimeric costimulatory receptors “CCRs”. Alternatively or additionally, endogenous TIGIT can be knocked-out in the T cell to avoid competition for ligand binding with the CCR.

CCRs can be expressed in T cells (e.g. can be encoded by the same vector) in trans with TCR/CARs of the present disclosure, and function to provide a co-stimulatory signal to the cell. (A) Schematic representation of a CCR expressed in trans with a “split-scFv” TCR/CAR (top left) or with a “full-scFv” TCR/CAR (top right); (bottom) schematic representation of expression vector encoding a “split-scFv” TCR/CAR with a CCR (coding sequence shown as “Co-stim”). Tested constructs included an extracellular portion from CD226 or TIGIT, and an intracellular portion from a mutated CD226, or from CD2, truncated CD2, CD28, or 4-1BB. (B) Schematic illustration of a strategy for lentiviral transduction of T cells with expression TCR/CAR+CCR constructs of the present disclosure, along with a CRISPR or Base Editor knockout of endogenous TRAC, TRBC, and TIGIT genes. Eliminating endogenous TIGIT eliminates competition for ligand binding with the CCR and TIGIT-mediated inhibitor signaling, while the CCR provides a positive signal to the T cell. (C) Knockout of TIGIT expression in T cells using a base editor system. (first sheet) Indel frequency analysis in T cells edited for TIGIT using sgRNAs 5 and 7. (second sheet) TIGIT expression in non-edited T cells (non-targeting sgRNA) and T cells edited with the combination of TIGIT-targeting sgRNAs 5 and 7 measured by flow cytometry after overnight stimulation with anti-CD3 antibody. (D) TCR/CAR expression (split-scFv format, top; full-scFv format, bottom) assessed by anti-TCR antibody and in-house labelled recombinant CD19 protein in primary T cells transduced with TCR/CAR alone (leftmost panel, labeled “WT”) or co-transduced with different CCRs (other panels). The “CD226-based” CCRs tested in these experiments comprise a CCR ectodomain and either a: mutated CD226 intracellular domain; truncated CD2 intracellular domain; or 4-1BB intracellular domain, as indicated. The “TIGIT-based” CCRs tested in these experiments comprise a TIGIT ectodomain and either a: truncated CD2 intracellular domain; or 4-1BB intracellular domain, as indicated. (E) Shows expression of CD19-specific “split-scFv” and “full-scFv” TCR/CARs when a CCR is encoded in the same expression vector, as measured by percent positive cells and by geometric mean fluorescence intensity (MFI), Panels: frequency of TCR/CAR+ T cells (split-scFv format, 4 donors) transduced with a vector encoding the TCR/CAR alone or with the indicated CCR; geometric mean of recombinant CD19 protein (labelled with AF647) binding to TCR/CAR+ T cells in T cells co-expressing a CCR or not; frequency of TCR/CAR+ T cells (full-scFv format, 4 donors) transduced with a vector encoding the TCR/CAR alone or along with the indicated CCR; geometric mean of recombinant CD19 protein (labelled with AF647) binding to TCR/CAR+ T cells in T cells co-expressing a CCR or not. (D) and (E) show that TCR/CARs express well when a CCR is encoded in the same vector.

FIGS. 24A-24D: (A) First sheet: Expression of CD226 and TIGIT in T cells transduced with TCR/CAR (split-scFv format) and the indicated CCR using either CD226 or TIGIT as the extracellular binding domain. Controls (two left-most panels) used knockout of endogenous TCR or of endogenous TCR and TIGIT. Second sheet: histograms of TIGIT and CD226 expression showing overexpression of these molecules in T cells transduced with chimeric co-stimulation molecules using CD226 or TIGIT as their extracellular binding domain. (B) Frequency of CD226+ in T cells transduced with a vector encoding the TCR/CAR alone or along with the indicated CCR (4 donors); geometric mean (MFI) of CD226 in TCR/CAR+ T cells in T cells co-expressing the indicated CCR; frequency of TIGIT+ in T cells transduced with a vector encoding the TCR/CAR alone or along with the indicated CCR (4 donors); geometric mean of TIGIT in TCR/CAR+ T cells in T cells co-expressing the indicated CCR. (C) (First sheet) Expression of CD226 and TIGIT in T cells transduced with TCR/CAR (full format) and the CCRs using CD226 or TIGIT ectodomain as their extracellular binding domain. (Second sheet) Histograms of TIGIT and CD226 expression showing overexpression of these molecules in T cells transduced with CCRs using CD226 or TIGIT ectodomain as their extracellular binding domain. (D) Frequency of CD226+ in T cells transduced with a vector encoding the TCR/CAR alone or the TCR/CAR along with the CCR (4 donors); geometric mean of CD226 in TCR/CAR+ T cells in T cells co-expressing the different CCRs; frequency of TIGIT+ in T cells transduced with a vector encoding the TCR/CAR alone or the TCR/CAR along with the CCR (4 donors); geometric mean of TIGIT in TCR/CAR+ T cells in T cells co-expressing the different CCR.s.

FIG. 25 Four panels at left: concentration of IL-2 (top) and IFN-g (bottom) produced by TCR/CAR T cells (split-scFv format) co-cultured with Raji cell line WT (left) or overexpressing CD155 (right). Four panels at right: concentration of IL-2 (top) and IFN-g (bottom) produced by TCR/CAR T cells (full-scFv format) co-cultured with Raji cell line WT (left) or overexpressing CD155 (right).

These data show that co-expression with CCRs increases cytokine production by TCR/CAR+ T cells in response to antigen-expressing tumor cells.

FIG. 26 shows (top) non-limiting examples of hinge sequences (and their substituent components) that can be present in a TCR/CAR of the present disclosure, between the target-binding portion (e.g. scFv) and the TCR constant domain portion, and (bottom) that including the identified hinge sequence in a TCR/CAR does not compromise expression. “No hinge” refers to a construct without an additional hinge. Additional hinges and are tested and can be selected, combined, or engineered for preferred characteristics such as flexibility and length.

FIGS. 27A-27C relate to experiments knocking-out endogenous T cell coreceptor functions to potentiate TCR/CAR sensitivity. Without wishing to be bound by theory, endogenous CD4 and CD8 co-receptors may sequester Lck. Knocking-out endogenous expression of these co-receptors may “free-up” Lck and enhance TCR/CAR sensitivity. (A) CD4 (left) and CD8 (right) gene editing using base editors along with TRAC and TRBC genes in TCR/CAR T cells. (B) CD8 gene editing using base editors along with TRAC and TRBC genes in T cells expressing TCR/CARs (left and center panels) or a TCR (right panel). TCR/CARs and TCR are specific for NY-ESO-1157-165 HLA-A2. (C) (First sheet) CD19-specific TCR/CAR expression in primary T cells also edited for CD8 co-receptor or not; (second sheet) NY-ESO-1 antigen: HLA tetramer binding in primary T cells expressing TCR/CARs or a TCR specific for NY-ESO-1 antigen: HLA and edited for CD8 co-receptor or not. These data show that modulation of T cell functions in co-receptor-knockout T cells can rely on dependency to MHC. In these experiments, antigen-binding was unaffected in MHC-independent-specific TCR/CARS, but is reduced in CD8-knockout-T cells expressing TCR/CARs and CARs specific for peptide: MHC (these TCR/CARs and CARs comprise a scFv specific for NY-ESO-1: HLA-A2), as it is for “conventional” TCRs (MHC-dependent binding).

FIGS. 28A and 28B: (A) (left) Calcium flux measured by flow cytometry in CD19-specific CAR T cells edited for CD8 or not and stimulated with CD19 recombinant protein; (right) calcium flux measured by flow cytometry in CD19-specific TCR/CAR T cells edited for CD8 or not and stimulated with CD19 recombinant protein. Calcium flux increased in T cells expressing CARs or TCR/CARs specific for MHC-independent antigens when the co-receptor was knocked-out. (B) (left) Calcium flux measured by flow cytometry in NY-ESO-1-specific TCR T cells edited for CD8 and stimulated with NY-ESO-1 dextramer; (right) calcium flux measured by flow cytometry in NY-ESO-1-specific TCR T cells edited for CD8 and stimulated with OKT3. Calcium flux was abrogated in TCR T cells that are CD8-knockout when stimulated using MHC-peptide dextramer (MHC-dependent), but increased in TCR T cells that are CD8-knockout when stimulated through CD3 (MHC-independent). These data show that T cells expressing a binding protein (TCR/CAR or CAR) with MHC-independent binding to antigen have increased sensitivity to antigen when the endogenous T cell co-receptor is knocked-out.

FIGS. 29A-29Q show amino acid sequences encoded by certain TCR/CAR or TCR/CAR plus CCR-encoding constructs of the present disclosure.

FIGS. 30A and 30B relate to certain embodiments of “bi-specific format” TCR/CAR constructs. (A) Schematic representations of bi-specific constructs as TCR/CARs expressed at a cell membrane. The two scFvs in each TCR/CAR construct have a different specificity. Illustrated “Format #1” and “Format #2” differ in that the scFvs have swapped positions: the scFv that is in a fusion with Cα in Format #1 is in a fusion with Cβ in Format #2, and the scFv that is in a fusion with Cβ in Format #1 is in a fusion with Cα in Format #2. In illustrated Formats #1 and #2, both scFvs have a (N-terminal to C-terminal) orientation of VH-linker-VL. In illustrated “Format #3”, both scFvs have a VL-linker-VH orientation. In illustrated “Format #4”, the scFv in a fusion with Cα has a VH-linker-VL oriendation and the scFv in a fusion with Cβ has a VL-linker-VH orientation. (B) Schematic representation of an expression vector encoding a TCR/CAR according to Format #1, and further encoding a truncated EGFR transduction marker.

FIG. 31 shows (top row) schematic representations of the bi-specific format TCR/CAR constructs in FIG. 30 and expression of the constructs (bottom row) in T cells.

FIGS. 32A and 32B relate to knockout of endogenous SLAMF7 gene in T cells utilizing cytidine base editing. (A) Histograms of SLAMF7 expression following knockout with sgRNA. sgRNAs 1, 2, 3, 4, and 5 are different base editor sgRNAs targeting the SLAMF7 locus. Co sgRNA is a control. (B) Percentage of SLAMF7+ T cells following base editing. FIGS. 33A and 33B relate to knockout of endogenous CD229 gene in T cells utilizing cytidine base editing. (A) Histograms of CD229 expression following knockout with sgRNA. sgRNAs 1, 2, and 3 are different base editor sgRNAs targeting the CD229 locus. sgRNAs 2+3 were also tested as a combination. (B) Percentage of CD229+ cells following base editing. Data in FIGS. 32A-33B support that endogenous SLAMF7 and CD229 can be knocked-out along with endogenous TCR chain genes using cytidine base editing to prevent fratricide lysis.

FIGS. 34A and 34B show fold expansion and viability of T cells expressing bi-specific TCR/CAR constructs of the present disclosure. (A) Fold expansion (left) and cell viability (right) of T cells expressing a bi-specific (anti-BCMA×anti-SLAMF7) TCR/CAR as compared to that of T cells expressing a conventional anti-BCMA CAR (“BCMA BBz CAR”) or a conventional anti-SLAMF7 CAR (“SLAMF7 BBz CAR”). (B) Fold expansion (left) and viability (right) of T cells expressing a bi-specific (anti-CD229×anti-BCMA) TCR/CAR as compared to that of T cells expressing a conventional anti-BCMA CAR (“BCMA BBz CAR”) or a conventional anti-CD229 CAR (“CD229 BBz CAR”). These data show that bispecific TCR/CARs expand as well as conventional CAR T cells and are viable.

FIGS. 35A-35C relate to antigen bi-specificity and reactivity of certain TCR/CAR constructs of the present disclosure. (A) Expression of BCMA and SLAMF7 in INA-6 cell lines with a BCMA knock out (INA-6 BCMA^{ko}) or a SLAMF7 knock out (INA-6 SLAMF7^{ko}), and wild-type INA-6 cell line (INA-6^{WT}). (B) Cytokine production (IFN-γ) by T cells transduced with a conventional anti-BCMA CAR (“BCMA BBz-CAR”), a conventional anti-SLAMF7 CAR (“SLAMF7 BBz-CAR”), or an anti-SLAMF7×anti-BCMA bi-specific TCR/CAR, in response to the cell lines described in (A) and an unstimulated control. For each condition, the dots and bars are, from left to right, “BMCA BBz-CAR”, “SLAMF7 BBz-CAR”, “Bi-specific TCR/CAR”. (C) Proliferation, by T cells transduced with a conventional anti-BCMA CAR (“BCMA BBz-CAR”), a conventional anti-SLAMF7 CAR (“SLAMF7 BBz-CAR”), or an anti-SLAMF7×anti-BCMA bi-specific TCR/CAR, in response to the cell lines described in (A). Top, representative histograms from flow cytometry of T cells. Bottom, Geometric mean of CTV (Cell Trace Violet) intensity. These data show that bispecific TCR/CARs demonstrate antigen bi-specificity and reactivity. For each condition, the dots and bars are, from left to right, “BMCA BBz-CAR”, “SLAMF7 BBz-CAR”, “Bi-specific TCR/CAR”.

FIGS. 36A and 36B relate to antigen bi-specificity and reactivity of certain embodiments of bi-specific TCR/CAR constructs of the present disclosure. (A) Percentage of killing of the INA-6 cell lines depicted in FIG. 35A by T cells transduced with a conventional anti-BCMA CAR (“BCMA BBz-CAR”), a conventional anti-SLAMF7 CAR (“SLAMF7 BBz-CAR”), or an anti-SLAMF7×anti-BCMA TCR/CAR at various effector to target (E:T) ratios. (B) Graph shows percentage of killing of INA-6 cells at various ratios of INA-6 BCMA^{ko} to INA-6 SLAMF7^{ko} cells by T cells transduced with a conventional anti-BCMA CAR (“BCMA BBz-CAR”), a conventional anti-SLAMF7 CAR (“SLAMF7 BBz-CAR”), or an anti-SLAMF7×anti-BCMA TCR/CAR.

FIGS. 37A and 37B relate to antigen sensitivity of certain embodiments of bi-specific TCR/CAR constructs of the present disclosure. (A) Schematic of an in vitro experiment in which various concentrations of recombinant BCMA (rBCMA) and/or recombinant SLAMF7 (rSLAMF7) were incubated (15 minutes) with T cells transduced with a conventional anti-BCMA CAR (“BCMA BBz-CAR”), a conventional anti-SLAMF7 CAR (“SLAMF7 BBZ-CAR”), or an anti-SLAMF7×anti-BCMA TCR/CAR construct (“Bi-specific TCR/CAR”), and intracellular staining for phosphorylated extracellular signal-regulated kinase (“pERK”) was performed. (B) shows the % of pERK+ T cells measured under the indicated conditions. These data show that bi-specific TCR/CARs demonstrate superior antigen sensitivity as compared to conventional CARs.

FIGS. 38A and 38B relate to antigen sensitivity of certain embodiments of bi-specific TCR/CAR constructs of the present disclosure. (A) Schematic of an in vitro experiment in which various concentrations of recombinant BCMA (rBCMA) and/or recombinant SLAMF7 (rSLAMF7) were incubated (24 hours) with T cells transduced with a conventional anti-BCMA CAR (“BCMA BBz-CAR”), a conventional anti-SLAMF7 CAR (“SLAMF7 BBz-CAR”), or an anti-SLAM-F7×anti-BCMA TCR/CAR construct (“Bi-specific TCR/CAR”), followed by ELISA to measure IFN-γ levels. (B) Concentration of IFN-γ (left) and % max IFN-γ (right) produced by T cells transduced with the indicated conventional CAR or bi-specific TCR/CAR. construct in the persence of antigen. These data show that bispecific TCR/CARs demonstrate superior antigen sensitivity as compared to conventional CARs.

### DETAILED DESCRIPTION

The present disclosure provides compositions and methods for improving cellular immunotherapy, such as against cancer, infection, autoimmune disease, or neurodegenerative disease.

Disclosed embodiments include polypeptide dimers (referred-to herein as TCR/CARs) that confer to a host cell (e.g. a T cell) target-specificity (including, in some contexts, high binding affinity and/or non-MHC-restricted binding) of a CAR while leveraging natural T cell signaling properties. TCR/CARs can be efficiently expressed at the surface of, for example, host T cells and confer to host T cells a diversified and highly sensitive signaling complex that acts with native T cell signaling properties. In some embodiments, a T cell expressing a TCR/CAR has one or more of the following properties as compared to a T cell expressing a CAR that binds the same target(s): increased sensitivity to antigen; increased production of one or more cytokine (e.g., IFN-γ, IL-2); increased killing against cells expressing the target(s); increased proliferation when in the presence of the target(s); reduced basal or non-specific activation; increased survival of a model mammal comprising cells expressing the target(s) (e.g., target-expressing cancer cells); increased antitumor activity against tumor cells expressing the target(s); lower target EC50 for production of IL-2 and/or IFN-γ; higher avidity (e.g., a greater amount of TCR/CAR-expressing cells bound to target); and association of the TCR/CAR with one or more CD3 proteins.

In some embodiments, a TCR/CAR comprises (1) a first polypeptide comprising a first TCR constant domain and (2) a second polypeptide comprising a second TCR constant domain, wherein the first TCR constant domain and the second TCR constant domain associate with one another. The association can comprise native interactions between cognate TCR constant domains (e.g. a native disulfide bond), engineered interactions between the TCR constant domains (e.g. one or more disulfide bonds introduced by protein engineering, knob-into-hole-type interactions, and/or charge-pair interactions), or both. One or both of the first polypeptide and the second polypeptide further comprises a target-binding domain disposed N-terminal to the TCR constant domains, and the target-binding domain does not comprise TCR variable regions. In certain embodiments, one or both of the first polypeptide and the second polypeptide comprises a target-binding domain disposed N-terminal to the TCR constant domains, wherein the target binding domain is selected from the target binding domains described herein. In certain embodiments, one or both of the first polypeptide and the second polypeptide comprises a target-binding domain disposed N-terminal to the TCR constant domains, wherein the target binding domain is selected from the target binding domains described herein and one or both of the first polypeptide and the second polypeptide may further comprise a TCR variable domain, provided that one or both fo the first polypeptide and the second polypeptide comprise a target-binding domain that is not, or does not comprise, a TCR variable domain.

TCR/CARs are preferably heterodimers (i.e., the TCR constant domains of the two polypeptides are different to one another), though TCR/CARs wherein the TCR constant domains of the two polypeptides are the same or substantially the same (e.g. are homodimeric with respect to the constant domains; see e.g. Groettrup et al. EMBO J. 1/(7): 2735-2745 (1992)) are also contemplated. In some embodiments, the first TCR constant domain comprises a TCR alpha-chain constant domain (Cα) and the second TCR constant domain comprises a TCR beta-chain constant domain (Cβ). In other embodiments, the first TCR constant domain comprises a Cβ and the second TCR constant domain comprises a Cα.

In some embodiments, the first polypeptide and/or the second polypeptide of a TCR/CAR comprises an intracellular portion that consists essentially of or that consists of the intracellular portion of the respective TCR constant domain. In some embodiments, the first polypeptide and/or the second polypeptide (preferably, both) does not comprise an intracellular signaling component (e.g. effector domain) from a CD3 protein, such as CD3ζ. In some embodiments, the first polypeptide and/or the second polypeptide (preferably, both) does not comprise an intracellular costimulatory domain from a costimulatory protein, such as CD28, 4-1BB, ICOS, CD27, OX40, DAP10, or any combination thereof. In some embodiments, the first polypeptide and/or the second polypeptide (preferably, both) does not comprise an immunoglobulin CH1, an immunoglobulin CH2, an immunoglobulin CH3, and/or an immunoglobulin CL (light chain constant domain). In some embodiments, the first polypeptide and/or the second polypeptide (preferably, both) does not comprise an immunoglobulin CH1, an immunoglobulin CH2, an immunoglobulin CH3, and/or an immunoglobulin CL (light chain constant domain) disposed C-terminal of the TCR constant domain(s).

The target-binding domain can comprise any naturally occurring or engineered binding domain suitable for binding a target of interest, such as, for example, an antibody heavy chain variable domain (VH), an antibody light chain variable domain (VL), a VH and a VL, a single-chain variable fragment (scFv) comprising VH-linker-VL or VL-linker-VH, a fragment antigen-binding region (Fab), a single-chain Fab, an antigen-binding fragment of a heavy chain-only antibody (VHH, also referred-to as a nanobody), a killer immunoreceptor from a NK cell, a designed ankyrin repeat protein (DARPin (Binz et al., J. Mol. Biol. 332:489, 2003 and Binz et al., Nat. Biotechnol. 22:575, 2004)), a ¹⁰FNIII domain such as an Adnectin™ or monobody ((Richards et al., J. Mol. Biol. 326:1475, 2003; Parker et al., Protein Eng. Des. Selec. 18:435, 2005 and Hackel et al. (2008). J. Mol. Biol. 381:1238-1252)), a lectin binding domain, a receptor ectodomain or functional portion or fragment thereof, provided that the receptor ectodomain does not comprise a TCR variable domain, a ligand such as e.g. a cytokine, a fully synthetic polypeptide (e.g. designed in silico, such as using the AlphaFold modeling program), a fibrinogen domain (see, e.g., Weisel et al., Science 230:1388, 1985), Kunitz domains (see, e.g., U.S. Pat. No. 6,423,498), a cysteine-knot miniprotein (Vita et al. (1995) Proc. Nat'l. Acad. Sci. (USA) 92:6404-6408; Martin et al. (2002) Nat. Biotechnol. 21:71, 2002 and Huang et al. (2005) Structure 13:755, 2005; Lui et al. Nature Communications 11:295 (2020)), a tetratricopeptide repeat domain (Main et al., Structure 11:497, 2003 and Cortajarena et al., ACS Chem. Biol. 3:161, 2008), a leucine-rich repeat domain (Stumpp et al., J. Mol. Biol. 332:471, 2003), a lipocalin domain (see, e.g., WO 2006/095164, Beste et al., Proc. Nat'l. Acad. Sci. (USA) 96:1898, 1999 and Schönfeld et al., Proc. Nat'l. Acad. Sci. (USA) 106:8198, 2009), an armadillo repeat protein (see, e.g., Madhurantakam et al., Protein Sci. 21:1015, 2012; PCT Patent Application Publication No. WO 2009/040338), an affilin (Ebersbach et al., J. Mol. Biol. 372:172, 2007), an affibody, an avimer, a knottin, a fynomer, an atrimer, cytotoxic T-lymphocyte associated protein-4 (Weidle et al., Cancer Gen. Proteo. 10:155, 2013) or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011), a centyrin, or the like, or any combination thereof.

In some embodiments, a target-binding domain is “split” across the first polypeptide and the second polypeptide, meaning that the component parts of a target-binding domain are dispersed between the first polypeptide and the second polypeptide; for example, where a VH and a VL together function to bind to a target, the VH may be comprised in the first polypeptide and the VL is comprised in the second polypeptide, or vice versa. In some embodiments, a target-binding domain is fully comprised in one TCR constant domain-containing polypeptide. For example, a scFv (VH-linker-VL or VL-linker-VH) may be comprised in the first polypeptide or the second polypeptide of a TCR/CAR, such an arrangement can be described as “full-scFv”.

In some embodiments, both of the first polypeptide and the second polypeptide fully comprise a target-binding domain. For example, the first polypeptide can comprise a Cα linked or fused to a first scFv and the second polypeptide can comprise a Cβ linked or fused to a second scFv. A target binding domain can be fused directly to a TCR constant domain or can be linked thereto by a linker, such as, for example, a hinge sequence.

In some embodiments, a VH or a VL of an antibody is sufficient to confer specific binding to a target (e.g., binding interactions between the antibody and its target occur, or can occur, through only VH and the target or only VL and the target); accordingly, in certain embodiments, a TCR/CAR, or a first polypeptide and/or a second polypeptide of a TCR/CAR, comprises only a VH or a VL as a binding domain.

Certain embodiments provide multispecific (e.g. bispecific) TCR/CARs. In some contexts, a multispecific TCR/CAR binds to two or more antigens that are expressed by a cancer; for example, to target multiple myeloma, a multispecific TCR/CAR may target any two or more of: BCMA, GPRC5D, SLAMF7, and CD229. In some embodiments, a multispecific TCR/CAR binds to CD19 and CD22, or binds to CD19 and BCMA, or binds to BCMA and SLAMF7, or binds to BCMA and CD229, or binds to SLAMF7 and CD229. In certain embodiments, a target comprises a protein ligand and a binding domain is from a receptor for the ligand. For example, a binding domain can comprise a receptor ectodomain from Bcl2 and a target comprises BIM.

Also provided are chimeric co-stimulatory receptor polypeptides (CCRs) that are capable of improving host cell (e.g. T cell) function when the host cell encounters PVR aka CD155. PVR aka CD155 is often overexpressed by tumor cells and is a shared ligand for two T cell proteins, CD226 and TIGIT. CD226 can be gradually lost following chronic antigen stimulation, while TIGIT is expressed following T cell activation and becomes constitutively expressed during T cell exhaustion. TIGIT has a higher affinity for PVR (approximately 100-fold) than does CD226. Disclosed CCRs modulate PVR/TIGIT/CD226 signaling and improve one or more function of a host (e.g. T) cell in the presence of PVR (e.g. in the presence of tumor cells).

In some embodiments, a CCR comprises an extracellular binding domain from CD226 (e.g. can comprise a CD226 ectodomain (also referred-to as extracellular domain)), or a portion or variant thereof that is functional to bind PVR. The CCR can further comprise a transmembrane domain from CD226. In other embodiments, a CCR comprises an extracellular binding domain from TIGIT (e.g. can comprise a TIGIT ectodomain), or a portion or variant thereof that is functional to bind PVR. The CCR can further comprise a transmembrane domain from TIGIT. A CCR further comprises an intracellular component designed to preserve normal CD226 signaling or disrupt or abrogate normal TIGIT signaling, such as by abrogating this signaling and/or by converting this signaling into an activating or co-stimulatory signal. A CCR may comprise a signaling (e.g. costimulatory) domain from, for example, CD2, CD28, 4-1BB, OX40, CD27, CD3ε, CD3δ, CD3γ, CD3ζ, CD79A, CD79B, SLAMF1, ICOS, DAP10, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, PTCH2, or the like. Any of these signaling domains or any combination of these signaling domains may be present in a CCR.

Thus, a CCR may comprise a CD226 ectodomain and a signaling (e.g. costimulatory) domain from, for example, CD2, CD28, 4-1BB, OX40, CD27, CD3ε, CD3δ, CD3γ, CD3ζ CD79A, CD79B, SLAMF1, ICOS, DAP10, GITR, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, or PTCH2. A CCR may comprise a TIGIT ectodomain and a signaling (e.g. costimulatory) domain from, for example, CD2, CD28, 4-1BB, OX40, CD27, CD3ε, CD3δ, CD3γ, CD3ζ, CD79A, CD79B, SLAMF1, ICOS, DAP10, GITR, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, or PTCH2.

For example, a CCR can comprise: (i) a CD226 ectodomain and a mutated CD226 endodomain (also called intracellular domain) sequence (e.g. comprising a K295A mutation, a K333A mutation, or both; see Braun et al., Immunity 53 (4): 805-823.e15 (2020)); (ii) a CD226 ectodomain and a CD2 intracellular domain; (iii) a CD226 ectodomain and a truncated CD2 intracellular domain; (iv) a CD226 ectodomain and a CD28 co-stimulatory domain; (v) a CD226 ectodomain and a 4-1BB co-stimulatory domain; (vi) a TIGIT ectodomain and a CD2 intracellular domain; (vii) a TIGIT ectodomain and a truncated CD2 intracellular domain; (viii) a TIGIT ectodomain and a CD28 co-stimulatory domain; (ix) a TIGIT ectodomain and a 4-1BB co-stimulatory domain; (x) a CD226 ectodomain and a CD27 co-stimulatory domain; (xi) a CD226 ectodomain and an OX40 costimulatory domain; (xii) a CD226 ectodomain and an ICOS costimulatory domain; (xiii) a CD226 ectodomain and a DAP10 costimulatory domain; (xiv) a CD226 ectodomain and a HVEM costimulatory domain; (xv) a CD226 ectodomain and a LIGHT costimulatory domain; (xvi) a CD226 ectodomain and CD30 costimulatory domain; (xvii) a CD226 ectodomain and SLAMI costimulatory domain; (xviii) a TIGIT ectodomain and a CD27 co-stimulatory domain; (xix) a TIGIT ectodomain and an OX40 costimulatory domain; (xx) a TIGIT ectodomain and an ICOS costimulatory domain; (xxi) a TIGIT ectodomain and a DAP10 costimulatory domain; (xxii) a TIGIT ectodomain and a HVEM costimulatory domain; (xxiii) a TIGIT ectodomain and a LIGHT costimulatory domain; (xiv) a TIGIT ectodomain and CD30 costimulatory domain; (xv) a TIGIT ectodomain and SLAMI costimulatory domain; (xvi) a TIGIT ectodomain and a GITR costimulatory domain; or (xvii) a CD226 ectodomain and a GITR costimulatory domain. It will be appreciated that embodiments comprising a portion or variant of a CD226 or TIGIT ectodomain that is functional to bind PVR are contemplated.

A CCR comprising a TIGIT ectodomain can comprise a TIGIT transmembrane domain. A CCR comprising a CD226 ectodomain can comprise a CD226 transmembrane domain.

Any CCR of the present disclosure can be expressed in a host cell (e.g. T cell) with any TCR/CAR of the present disclosure (or with a TCR, or a CAR) and can provide an activating and/or co-stimulatory signal to the host cell; for example, a host T cell encounters a tumor cell that expresses an antigen and PVR binds to the antigen through the TCR/CAR (or TCR, or CAR), thus providing an antigen-binding signal to the T cell, and binds to PVR through the CCR, thus preventing a normal PVR-binding signal via endogenous CD226 or TIGIT and optionally converting the PVR-binding signal into an activating and/or co-stimulatory signal.

Also provided are polynucleotides that encode a disclosed TCR/CAR, a disclosed CCR, or both. A polynucleotide can be codon-optimized for expression in a host cell. A polynucleotide can be comprised in a vector, such as, for example, a viral vector, such as a lentiviral vector or a retroviral vector. A polynucleotide or vector can include one or more additional features to facilitate desired expression of the encoded polypeptide(s), such as one or more promoter, one or more sequence encoding a signal peptide (also known as a leader peptide or leader sequence or transit peptide), one or more sequence encoding a furin cleavage sequence, one or more sequence encoding a self-cleaving peptide, or any combination thereof.

Also provided are fusion polypeptides that comprise a binding-domain-containing TCR/CAR polypeptide of the present disclosure. Any of the presently disclosed first or second TCR/CAR polypeptides may be provided as an isolated polypeptide, provided that the polypeptide comprises a binding domain, and not accompanied by a cognate TCR/CAR polypeptide. Polynucleotides and vectors that encode the fusion polypeptides are also provided.

Any of the presently disclosed polypeptide dimers, polypeptides, or fusion polypeptides can be expressed as membrane-bound molecule or dimer at a cell surface. In some embodiments, a polypeptide dimer associates with one or more CD3 proteins of a host T cell expressing the polypeptide dimer, e.g. when the polypeptide dimer binds to its target(s).

Also provided are host cells that express a presently disclosed TCR/CAR, a presently disclosed CCR, or both. A TCR/CAR (polypeptide dimer) and/or a CCR can be expressed as a membrane-bound protein or protein dimer at a cell surface of a host cell. Also provided are host cells that comprise a polynucleotide or vector encoding a a presently disclosed TCR/CAR, a presently disclosed CCR, or both. A bost cell expressing or encoding a CCR may further express or encode a TCR or a CAR. In certain embodiments, a host cell comprises a hematopoietic progenitor cell, a hematopoeitic stem cell, or an immune system cell, such as a human immune system cell. In certain embodiments, an immune system cell comprises a T cell, a NK-T cell, or a macrophage. In certain embodiments, a T cell comprises a CD4+ T cell, a CD8+ T cell, a CD4-CD8-double negative T cell, an αβ+ T cell, a γδ+ T cell, or any combination thereof. In certain embodiments, a T cell comprises a naïve T cell, a central memory T cell, a stem cell memory T cell, an effector memory T cell, or any combination thereof.

In some embodiments, a host cell comprises a chromosomal knockout of TIGIT, of a TCR locus (e.g. TRAC, TRBC), of a CD8 locus, of a CD4 locus, of a PD-1 locus, of a LAG-3 locus, of a TIM3 locus, of an HLA locus (e.g. a gene that encodes an α1 macroglobulin, an α2 macroglobulin, an α3 macroglobulin, a β1 microglobulin, or a β2 microglobulin), of a TGFβR1 locus, of a TGFβR2 locus, of a LAT locus, of an A2AR locus, of a Fas locus, of a FasL locus, of a B7-H3 locus, of a B7-H4 locus, of an IDO locus, of a VISTA locus, of a SIGLEC7 locus, of a SIGLEC9 locus, of a CBLB locus, of a RASA2 locus, of a UBASH3A locus, of a CISH locus, or of any combination thereof.

In some embodiments, a host cell expresses a TCR/CAR and comprises a chromosomal knockout of one or more genes expressing a protein that is recognized by the TCR/CAR. For example, in some embodiments, a host cell expresses a TCR/CAR that binds SLAMF7 and comprises a chromosomal knockout of a SLAMF7 locus. In some embodiments, a host cell expresses a TCR/CAR that binds CD229 and comprises a chromosomal knockout of a CD229 locus. Adminstering a plurality of such host cells to a subject may reduce fratricidal lysis as between the host cells. In certain further embodiments, the host cell is a T cell. The T cell can further comprise a chromosomal gene knockout of a CD4 gene locus and/or of a CD8 gene locus.

Also provided is a T cell comprising a chromosomal gene knockout of a CD4 gene locus and/or of a CD8 gene locus. In certain embodiments, the T cell expresses a CAR or a TCR. In some embodiments, the TCR is capable of CD4-independent binding to an antigen: MHC complex and/or is capable of CD8-independent binding to an antigen: MHC complex.

Compositions that comprise the host cells (e.g. T cells) (including any combination thereof), polynucleotides, TCR/CARs, fusion polypepties, or vectors, and optionally a pharmaceutically acceptable carrier, excipient, or diluent, are also provided. Also provided are methods of making a host cell, wherein the methods comprise introducing a polynucleotide or vector encoding (i) a TCR/CAR of the present disclosure and/or (ii) a CCR of the present disclosure. Also provided are methods of making a host cell, wherein the methods comprise generating chromosomal CD4 or CD8 gene knockout in a T cell that comprises a polynucleotide encoding a CAR or a TCR, or in a T cell into which is to be introduced a polynucleotide encoding a CAR or a TCR.

Also provided are methods of using any of the presently disclosed TCR/CARs, CCRs, fusion polypeptides, polynucleotides, vectors, host cells, and compositions to treat a disease or disorder, such as, for example, a cancer, such as, for example, a solid cancer or a hematological malignancy.

Also provided are methods of using any of the presently disclosed TCR/CARs, CCRs, fusion polypeptides, polynucleotides, vectors, host cells, and compositions in the preparation of a medicament to treat a disease or disorder, such as, for example, a cancer, such as, for example, a solid cancer or a hematological malignancy.

Prior to setting forth this disclosure in more detail, it may be helpful to an understanding thereof to provide additional definitions of certain terms to be used herein. Still more definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. “About” includes ±15%, ±10%, and ±5%. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a protein domain, linker, signal peptide) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” or “fusion polypeptide” refers to a protein that, in a single chain, has at least two distinct domains and/or motifs, wherein the domains or motifs are not naturally found together (e.g., in the given arrangement, order, or number, or at all) in a protein. In certain embodiments, a fusion protein comprises at least two distinct domains and/or motifs that are not found together in a single naturally occurring peptide or polypeptide. In certain embodiments, a fusion protein comprises amino acid sequences from two or more distinct polypeptides. TCR/CARs include one or more fusion polypeptides. CCRs comprise a fusion polypeptide. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to the cell surface, where the fusion protein can be anchored to the cell membrane.

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co-or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. In some embodiments, a composition of the present disclosure can be “isolated” in the sense that it is physically separated from and not comprised within a subject to whom the composition can be, was, or is to be administered.

The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (Kp) constant) or avidity; or an assay measuring TCR signaling or an activity stimulated thereby (e.g. as exemplified herein, such as measuring IFN-g production, IL-2 production, intracellular calcium flux, cellular avidity as determined by the percentage of cells in a sample that bind to antigen, proliferation, specific cytotoxicity against a target cell, NFAT expression, NFkB expression, AP-1 expression, Nur77 expression) optionally in the presence of PVR or PVR-expressing cells.))

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., T cell signaling and/or activity following binding to antigen). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release).

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). It will be appreciated that in the case of a host cell that comprises a heterologous polynucleotide, the polynucleotide is “heterologous” to progeny of the host cell, whether or not the progeny were themselves manipulated to, for example, introduce the polynucleotide.

The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell (e.g., T cell) or microorganism targeted for genetic modification with a heterologous nucleic acid molecule to produce a polypeptide of interest (e.g., a fusion protein of the present disclosure). In certain embodiments, a host cell may optionally possess or be modified to include other genetic modifications that confer desired properties related or unrelated to, e.g., biosynthesis of the heterologous protein (e.g., inclusion of a detectable marker; deleted, altered or truncated endogenous host cell protein; expression of an antigen-binding protein).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“T cell receptor” (TCR) refers to a multi-protein complex (each component protein having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane.

Non-limiting examples of TCR constant domain amino acid sequences are provided herein. The variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). The source of a TCR or TCR binding domain as used in the present disclosure may be from various animal species, such as a human, mouse, rat, rabbit, non-human primate, or other mammal.

TCR/CARs of the present disclosure are structurally distinct from TCRs.

“CD3” is a multi-protein complex of six chains (see, Abbas and Lichtman, 2003; Janeway et al., p. 172 and 178, 1999). In mammals, the complex generally comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is thought to allow these chains to associate with positively charged regions of T cell receptor chains. The intracellular tails of the CD3 complex proteins contain immunoreceptor tyrosine-based activation motifs or ITAMs, which are thought to be important for T cell signaling in response to antigen binding.

CD3, as well as the protein subunits, domains, and sequences therefrom, may be from various animal species, including human, mouse, rat, or other mammals.

In certain embodiments, a TCR is found on the surface of T cells (also referred to as T lymphocytes) and associates with the CD3 complex. In certain embodiments, a TCR complex comprises a TCR or a functional portion thereof; a dimer comprising two CD3ζ chains, or functional portions or variants thereof; a dimer comprising a CD3δ chain and a CDε chain, or functional portions or variants thereof, and a dimer comprising a CD3γ chain and a CDε chain, or functional portions or variants thereof, any one or more of which may be endogenous or heterologous to the T cell. TCR/CARs of the present disclosure associate with the CD3 complex.

“Major histocompatibility complex molecules” (MHC molecules) refer to glycoproteins that deliver peptide antigens to a cell surface. MHC class I molecules are heterodimers consisting of a membrane spanning a chain (with three α domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide: MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

“CD4” refers to an immunoglobulin co-receptor glycoprotein that can assist the TCR in binding to antigen: MHC and communicating with antigen-presenting cells (see, Campbell & Reece, Biology 909 (Benjamin Cummings, Sixth Ed., 2002); UniProtKB P01730). CD4 is found on the surface of immune cells such as T helper cells, monocytes, macrophages, and dendritic cells, and includes four immunoglobulin domains (D1 to D4) that are expressed at the cell surface. During antigen recognition, CD4 is recruited, along with the TCR complex, to bind to different regions of the MHCII molecule (CD4 binds MHCII β2, while the TCR complex binds antigen: MHCII α1/β1).

As used herein, the term “CD8 co-receptor” or “CD8” means the cell surface glycoprotein CD8, either as an alpha-alpha homodimer or an alpha-beta heterodimer. The CD8 co-receptor can assist in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1: 81-88, 2004). In humans, there are five (5) different CD8 beta chains (see UniProtKB identifier P10966) and a single CD8 alpha chain (see UniProtKB identifier P01732).

“Chimeric antigen receptor” (CAR) refers to a fusion protein engineered to contain two or more amino acid sequences (which may be naturally occurring amino acid sequences) linked together in a way that does not occur naturally or does not occur naturally in a host cell, which fusion protein can function as an antigen-specific receptor when present on a surface of a cell. CARs of the present disclosure include an extracellular portion comprising an antigen-binding domain (e.g., obtained or derived from an immunoglobulin or immunoglobulin-like molecule, such as a scFv or scTCR derived from an antibody or TCR (respectively) specific for a cancer antigen, or an antigen-binding domain derived or obtained from a killer immunoreceptor from an NK cell, a designed ankyrin repeat protein (DARPin), an engineered fibronectin type three domain (also referred-to as a monobody) such as an Adnectin™, a ligand (e.g., a cytokine, if the target is a cytokine receptor), a receptor ectodomain (e.g., a cytokine receptor, if the target is a cytokine) or the like) linked to a transmembrane domain and one or more intracellular signaling domains (optionally containing co-stimulatory domain(s)) (see, e.g., Sadelain et al., Cancer Discov., 3 (4): 388 (2013); see also Harris and Kranz, Trends Pharmacol. Sci., 37 (3): 220 (2016); Stone et al., Cancer Immunol. Immunother., 63 (11): 1163 (2014)). In certain embodiments, a CAR comprises an antigen-specific TCR binding domain (see, e.g., Walseng et al., Scientific Reports 7:10713, 2017; it will be understood that TCR/CARs of the present disclosure possess a distinct structure to CARs).

The term “variable region” or “variable domain” refers to the domain an antibody heavy or light chain (or, for TCRs, of a TCR of a TCR α-chain or β-chain (or γ-chain and δ-chain for γδ TCRs)), that is involved in binding to antigen (i.e., contains amino acids and/or other structures that contact antigen and result in binding). The variable domains of cognate chains generally have similar structures, with each domain comprising four generally conserved framework regions (FRs) and three CDRs. In both TCRs and antibodies, framework regions separate CDRs and CDRs are situated between framework regions (i.e., in primary structure).

The terms “complementarity determining region,” and “CDR,” are synonymous with “hypervariable region” or “HVR,” and refer to sequences of amino acids within TCR or antibody variable regions, which, in general, confer antigen specificity and/or binding affinity and are separated from one another in primary structure by framework sequence. In some cases, framework amino acids can also contribute to binding, e.g., may also contact the antigen or antigen-containing molecule. In general, there are three CDRs in each variable region (e.g., three CDRs in each of the antibody heavy chain and light chain variable regions). Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” or “Ag” as used herein refers to an immunogenic molecule that can provoke an immune response. This immune response may involve antibody production, activation of specific immunologically competent cells (e.g., T cells), secretion of cytokines, or any combination thereof. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen. In any of the presently disclosed embodiments, a target can be, or can comprise, an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a fusion protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof. In some embodiments, a benefit of a cellular immunotherapy of this disclosure can further include a reduction (e.g., in number or severity) or absence of a cytokine-related toxicity, such as a cytokine release syndrome.

A “therapeutically effective amount” or “effective amount” of a composition (fusion protein, host cell expressing a fusion protein, polynucleotide, vector, polypeptide dimer, or the like) of this disclosure, refers to an amount of the composition sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. In the case of cancers, benefits can include, for example, a reduction in the size, area, volume, and/or density of a tumor, and/or a reduction or reversal in the rate of tumor growth or spread of cancer,

When referring to an individual active ingredient, administered alone, a therapeutically effective amount refers to the effects of that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

### TCR/CARs, Polypeptides, and CCRs

Disclosed embodiments include hybrid receptor polypeptides (also referred-to herein as TCR/CARs) that confer to a host cell (e.g. a T cell) target-specificity (including, in some contexts, high binding affinity and/or non-MHC-restricted binding) of a CAR while leveraging natural T cell signaling properties. TCR/CARs can be efficiently expressed at the surface of host cells and confer to host cells a diversified and highly sensitive signaling complex that acts with native T cell signaling properties. In some embodiments, a TCR/CAR comprises (1) a first polypeptide comprising a first TCR constant domain and (2) a second polypeptide comprising a second TCR constant domain, wherein the first TCR constant domain and the second TCR constant domain associate to form a dimer. The association can comprise one or more native interaction between cognate TCR constant domains (e.g. a native disulfide bond), one or more engineered interaction between the TCR constant domains (e.g. one or more disulfide bonds introduced by protein engineering), or both. One or both of the first polypeptide and the second polypeptide further comprises a target-binding domain N-terminal to the TCR constant domain(s).

In some embodiments, the first TCR constant domain comprises a TCR alpha-chain constant domain (Cα) and the second TCR constant domain comprises a TCR beta-chain constant domain (Cβ). In other embodiments, the first TCR constant domain comprises a Cβ and the second TCR constant domain comprises a Cα.

In other embodiments, the first TCR constant domain comprises a TCR gamma-chain constant domain (Cγ) and the second TCR constant domain comprises a TCR delta-chain constant domain (Cδ). In other embodiments, the first TCR constant domain comprises a Cδ and the second TCR constant domain comprises a Cγ.

It will be understood that the terms “TRAC” and “TRBC”, when referring to a TCR/CAR, may be used interchangeably with the terms TCR Cα and TCR Cβ, respectively, and include embodiments comprising the variant sequences and modifications described herein. A TRAC or TRBC may, but need not necessarily, comprise the native amino acid sequence encoded by a (n e.g. human) TRAC or TRBC, respectively. When describing an endogenous gene locus encoding a TCR alpha chain constant domain or beta chain constant domain, the term TRAC or TRBC, respectively, may be used.

TCR constant domain sequences may be from, for example, human, mouse, marsupial (e.g. opossum, bandicoot, wallaby), shark, or non-human primate. In certain preferred embodiments, TCR constant domain sequences are human or comprise engineered variants of human sequences. TCR constant domains may be engineered to improve pairing, expression, stability, or any combination of these. See, e.g., Cohen et al., Cancer Res, 2007; Kuball et al., Blood 2007; and Haga-Friedman et al., Journal of Immunology 2009. Examples of engineering in TCR Cα and Cβ are illustrated in FIG. 7; these can include mutation of a native amino acid to a cysteine so that a disulfide bond forms between the introduced cysteine of one TCR constant domain and a native cysteine of the other TCR constant domain. Such mutations can include T48C in Cα, T57C in Cβ, or both. Mutations to improve stability can include a mutation in the Cα transmembrane domain from the sequence LSVIGF (SEQ ID NO.: 62) to the sequence LLVIVL (SEQ ID NO.: 63) (“L-V-L” mutation; see Haga-Friedman et al., J Immunol 188:5538-5546 (2012), the TCR mutations and mutant TCR constant domain sequences of which are incorporated herein by reference). Also contemplated are embodiments wherein cognate TCR constant domains comprise mutations so that, for example, one TCR constant domain (e.g., one of Cα and Cβ) comprises an introduced “cavity” (e.g., obtainable by replacing one or more native amino acid with one or more amino acids having smaller side chains) and the other (e.g., the other of Cα and Cβ) comprises a compensatory “protuberance” (e.g., obtainable by replacing one or more native amino acid with one or more amino acids having larger side chains), similar to a “knob-into-hole” configuration used to promote preferential pairing of antibody heavy chains. Also contemplated are embodiments wherein TCR constant domain amino acids are mutated to introduce charge properties that favor pairing of the mutated constant domains. Examples of mutations that may be made in Cα and Cβ to promote specific pairing by a knobs-into-holes-type mechanism or by a charge-pairing mechanism are provided in Voss et al., J. Immunol 180 (1): 391-401 (2008) doi.org/10.4049/jimmunol.180.1.391; see also U.S. Pat. No. 9,062,127. The TCR constant domain mutations, mutated TCR constant domains, and methods used to identify sites for mutation, described in these documents, are incorporated herein by reference.

An example of a TCR Cα amino acid sequence is provided in UniProt KB P01848 (human TRAC):

(SEQ ID NO.: 56)
IQNPDPAVYQ LRDSKSSDKS VCLFTDFDSQ TNVSQSKDSD
VYITDKTVLD MRSMDFKSNS AVAWSNKSDF ACANAFNNSI
IPEDTFFPSP ESSCDVKLVE KSFETDTNLN FQNLSVIGFR
ILLLKVAGFN LLMTLRLWSS

An example of a TCR Cα amino acid sequence engineered to include threonine-to-cysteine and LVL mutations as described herein is provided in SEQ ID NO.: 57:

IQNPDPAVYQLRDSKSSDKSVCLFTDFDSQTNVSQSKDSDVYITDKCVLD
MRSMDFKSNSAVAWSNKSDFACANAFNNSIIPEDTFFPSPESSCDVKLVE
KSFETDTNLNFQNLLVIVLRILLLKVAGFNLLMTLRLWSS.

Two human TCR Cβ isoforms are TRBCI and TRBC2. An example of a TRBCI amino acid sequence is provided in UniProt KB P01850;

(SEQ ID NO.: 58)
DLNKVFPPEV AVFEPSEAEI SHTQKATLVC LATGFFPDHV
ELSWWVNGKE VHSGVSTDPQ PLKEQPALND SRYCLSSRLR
VSATFWQNPR NHFRCQVQFY GLSENDEWTQ DRAKPVTQIV
SAEAWGRADC GFTSVSYQQG VLSATILYEI LLGKATLYAV
LVSALVLMAM VKRKDF.

An example of a TRBCI amino acid sequence engineered to include a serine-to-cysteine mutation is provided in SEQ ID NO.: 59:

DLNKVFPPEVAVFEPSEAEISHTQKATLVCLATGFFPDHVELSWWVNGKE
VHSGVCTDPQPLKEQPALNDSRYCLSSRLRVSATFWQNPRNHFRCQVQFY
GLSENDEWTQDRAKPVTQIVSAEAWGRADCGFTSVSYQQGVLSATILYEI
LLGKATLYAVLVSALVLMAMVKRKDF.

An example of a TRBC2 amino acid sequence is provided in UniProt KB A0A5B9:

(SEQ ID NO.: 60)
DLKNVFPPKV AVFEPSEAEI SHTQKATLVC LATGFYPDHV
ELSWWVNGKE VHSGVSTDPQ PLKEQPALND SRYCLSSRLR
VSATFWQNPR NHFRCQVQFY GLSENDEWTQ DRAKPVTQIV
SAEAWGRADC GFTSESYQQG VLSATILYEI LLGKATLYAV
LVSALVLMAM VKRKDSRG.

An example of a TRBC2 amino acid sequence engineered to include a serine-to-cysteine mutation is provided in SEQ ID NO.: 61: DLKNVFPPKV AVFEPSEAEI SHTQKATLVC LATGFYPDHV ELSWWVNGKE VHSGVCTDPQ PLKEQPALND SRYCLSSRLR VSATFWQNPR NHFRCQVQFY GLSENDEWTQ DRAKPVTQIV SAEAWGRADC GFTSESYQQG VLSATILYEI LLGKATLYAV LVSALVLMAM VKRKDSRG.

In any of the presently disclosed embodiments, a TCR/CAR can comprise a TCR Cα having at least 90%, at least 91%, at least 92%, at least 93%, at least 94%, at least 95%, at least 96%, at least 97%, at least 98%, at least 99%, or 100% identity to any one of SEQ ID NOs.: 56-57, and a TCR Cβ having at least 90%, at least 91%, at least 92%, at least 93%, at least 94%, at least 95%, at least 96%, at least 97%, at least 98%, at least 99%, or 100% identity to any one of SEQ ID NOs.: 58-61. In certain embodiments, a TCR/CAR comprises a TCR Cα and a TCR Cβ having at least least 90%, at least 91%, at least 92%, at least 93%, at least 94%, at least 95%, at least 96%, at least 97%, at least 98%, or at least 99% to, or comprising or consisting of, the amino acid sequences set forth in SEQ ID NOs.: (1) 56 and 58, respectively; (ii) 56 and 59, respectively; (iii) 56 and 60, respectively; (iv) 56 and 61, respectively; (v) 57 and 58, respectively; (vi) 57 and 59, respectively; (vii) 57 and 60, respectively; or (viii) 57 and 61, respectively.

In preferred embodiments, a variant TCR Cα or Cβ maintains the same or substantially the same length and/or number of amino acids as compared to a native TCR Cα or Cβ, respectively, such that, in certain embodiments, the variation does not comprise a truncation in the length thereof as compared to a native TCR Cα or Cβ, respectively. In certain embodiments, a variant of a TCR Cβ (TRBC1) maintains the intracellular sequence VKRKDF (SEQ ID NO.: 64). In certain embodiments, a variant of a TCR Cβ (TRBC1) maintains the intracellular sequence MAMVKRKDSRG (SEQ ID NO.: 65). Variant TCR constant domains of the present disclosure are capable of associating with a cognate TCR constant domain and with one or more CD3 proteins. In other words, in certain embodiments, TCR/CARs can be assimilated into a TCR complex on a host (e.g. T) cell surface that comprises the TCR/CAR and CD3 proteins. In particular, TCR/CARs of the present disclosure, including those that comprise variant TCR constant domains, are capable of producing a TCR-CD3 complex signal in a host (e.g. T) cell when the TCR/CAR expressed by the host cell binds to its target(s).

In some embodiments, the first polypeptide and/or the second polypeptide (preferably both) of a TCR/CAR comprises an intracellular portion that consists essentially of or that consists of the intracellular portion of the respective TCR constant domain. In some embodiments, the first polypeptide and/or the second polypeptide (preferably both) does not comprise an intracellular signaling component (e.g. effector domain) from a CD3 protein, such as CD3. In some embodiments, the first polypeptide and/or the second polypeptide (preferably both) does not comprise an intracellular costimulatory domain from a costimulatory protein, such as CD28, 4-1BB, ICOS, CD27, OX40, DAP10, or any combination thereof.

In some embodiments, the first polypeptide and/or the second polypeptide (preferably both) of a TCR/CAR does not comprise an immunoglobulin CH2 domain and/or an immunoglobulin CH3 domain and/or an immunoglobulin light chain constant domain. In some embodiments, the first polypeptide and/or the second polypeptide (preferably both) of a TCR/CAR does not comprise an immunoglobulin CH2 domain and/or an immunoglobulin CH3 domain and/or an immunoglobulin light chain constant domain disposed C-terminal to the TCR constant domain. In some embodiments, the polypeptide dimer does not comprise an immunoglobulin CH2-CH3 or an immunoglobulin CH2-CH3: CH2-CH3 dimer.

In some embodiments, a polypeptide dimer comprises a target-binding domain comprising (i) a VH comprised in the first polypeptide or the second polypeptide and (ii) a cognate VL comprised in the other of the first and the second polypeptide, wherein the target is not 2,4,6-trinitrophenyl (TNP), digoxin, or phosphorylcholine.

A “binding domain” (also referred to as a “binding region” or “binding moiety”), as used herein, refers to a molecule or portion thereof (e.g., peptide, oligopeptide, polypeptide) that possesses the ability to specifically and non-covalently associate, unite, or combine with a target (e.g. antigen). A binding domain includes any naturally occurring, synthetic, semi-synthetic, or recombinantly produced binding partner for a biological molecule, a molecular complex (i.e., complex comprising two or more biological molecules), or other target of interest.

Herein, a “binding domain” also includes a subunit of a complete binding domain; e.g., if VH and VL together are required for binding to a target, VH alone or VL alone may be referred-to as a “binding domain”. It will be understood that TCR/CARs are capable of binding to a target. Exemplary binding domains useful in TCR/CARs include single chain immunoglobulin variable regions (e.g., scFv, scFab), Fabs, Fv, sdAbs such as nanobodies/VHH, VNAR, receptor ectodomains, ligands (e.g., cytokines, chemokines), or (other) synthetic polypeptides selected for their specific ability to bind to a biological molecule, a molecular complex or other target of interest (e.g., DARPins, ¹⁰NIII domains). In certain embodiments, the binding domain comprises a scFv, or ligand. In certain embodiments, the binding domain is chimeric, human, or humanized.

The target-binding domain can comprise any naturally occurring or engineered binding domain suitable for binding a target of interest, such as, for example, an antibody heavy chain variable domain (VH), an antibody light chain variable domain (VL), a VH and a VL, a single-chain variable fragment (scFv) comprising VH-linker-VL or VL-linker-VH, a fragment antigen-binding region (Fab), a single-chain Fab, an antigen-binding fragment of a heavy chain-only antibody (VHH, also referred-to as a nanobody), a killer immunoreceptor from a NK cell, a designed ankyrin repeat protein (DARPin (Binz et al., J. Mol. Biol. 332:489, 2003 and Binz et al., Nat. Biotechnol. 22:575, 2004)), a ¹⁰FNIII domain such as an Adnectin™ or monobody ((Richards et al., J. Mol. Biol. 326:1475, 2003; Parker et al., Protein Eng. Des. Selec. 18:435, 2005 and Hackel et al. (2008) J. Mol. Biol. 381:1238-1252)), a lectin binding domain, a receptor ectodomain or functional portion or fragment thereof, provided that the receptor ectodomain does not comprise a TCR variable domain, a ligand such as e.g. a cytokine, a fully synthetic polypeptide (e.g. designed in silico, such as using the AlphaFold modeling program), a fibrinogen domain (see, e.g., Weisel et al., Science 230:1388, 1985), Kunitz domains (see, e.g., U.S. Pat. No. 6,423,498), a cysteine-knot miniprotein (Vita et al. (1995) Proc. Nat'l. Acad. Sci. (USA) 92:6404-6408; Martin et al. (2002) Nat. Biotechnol. 21:71, 2002 and Huang et al. (2005) Structure 13:755, 2005; Lui et al. Nature Communications 11:295 (2020)), a tetratricopeptide repeat domain (Main et al., Structure 11:497, 2003 and Cortajarena et al., ACS Chem. Biol. 3:161, 2008), a leucine-rich repeat domains (Stumpp et al., J. Mol. Biol. 332:471, 2003), a lipocalin domain (see, e.g., WO 2006/095164, Beste et al., Proc. Nat'l. Acad. Sci. (USA) 96:1898, 1999 and Schönfeld et al., Proc. Nat'l. Acad. Sci. (USA) 106:8198, 2009), an armadillo repeat protein (see, e.g., Madhurantakam et al., Protein Sci. 21:1015, 2012; PCT Patent Application Publication No. WO 2009/040338), an affilin (Ebersbach et al., J. Mol. Biol. 372:172, 2007), an affibody, an avimer, a knottin, a fynomer, an atrimer, cytotoxic T-lymphocyte associated protein-4 (Weidle et al., Cancer Gen. Proteo. 10:155, 2013) or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol.22:849, 2011), a receptor ectodomain or functional portion or fragment thereof, provided that the receptor ectodomain does not comprise a TCR variable domain, a centyrin, or the like, or any combination thereof.

Binding domains of this disclosure can be generated as described herein or by a variety of methods known in the art (see, e.g., U.S. Pat. Nos. 6,291,161 and 6,291,158). For example, binding domains of this disclosure may be identified by screening a Fab phage library for Fab fragments that specifically bind to a target of interest (see Hoet et al., Nat. Biotechnol. 23:344, 2005). Additionally, traditional strategies for hybridoma development using a target of interest as an immunogen in convenient systems (e.g., mice, HuMAb Mouse®, TC Mouse™, KM-Mouse®, llamas, chicken, rats, hamsters, rabbits, etc.) can be used to develop binding domains of this disclosure. Binding domains can be isolated from a human protein (see e.g. Traggiai et al., Nature Medicine 10 (8): 871-875 (2004)), designed in silico (e.g. using AlphaFold or a like program), isolated or derived a rat, a mouse, a hamster, or other rodent, can be from an avian source, can be from a bovine source, can be from a canine source, can be from a camelid (e.g. from camels, dromedaries, or llamas; Ghahroudi et al., FEBS Lett. 414:521, 1997; Vincke et al., J. Biol. Chem. 284:3273, 2009; Hamers-Casterman et al., Nature 363:446, 1993 and Nguyen et al., J. Mol. Biol. 275:413, 1998), a shark such as a nurse shark (Roux et al., Proc. Nat'l. Acad. Sci. (USA) 95:11804, 1998), spotted ratfish (Nguyen et al., Immunogen. 54:39, 2002), or lamprey (Herrin et al., Proc. Nat'l. Acad. Sci. (USA) 105:2040, 2008 and Alder et al. Nat. Immunol. 9:319, 2008). A binding domain can include sequences from a library that encodes random peptides or sequences from a library that encodes an engineered diversity of amino acids in loop regions of alternative non-antibody scaffolds.

In some contexts, a VH alone is sufficient to confer binding (i.e. a target-binding domain can comprise a VH and need not comprise a VL). In some contexts, a VL alone is sufficient to confer binding (i.e. a target-binding domain can comprise a VL and need not comprise a VH).

In some embodiments, a target-binding domain is “split” across the first polypeptide and the second polypeptide; for example, where a VH and a VL together function to bind to a target, the VH is comprised in the first polypeptide and the VL is comprised in the second polypeptide, or vice versa. The same may be the case, for example, with an antibody Fab; VH—CH1 may be comprised in the first polypeptide, and VL-CL may be comprised in the second polypeptide, or vice versa. Regarding Fab-type molecules that function as binding domains in the presently disclosed TCR/CARs, it will be understood that CH1 and CL may be swapped (i.e. VH-CL, VL-CH1), and that other immunoglobulin constant domains can be utilized in place of CH1 and CL (e.g., CH2-CH2 or CH3-CH3 may replace CH1-CL; see e.g. Wozniak-Knopp et al. PLOS One 13 (4): c0195442 (2018))

Accordingly, certain embodiments refer to a “split” format. It will be understood that in a “split-scFv” design, “scFv” refers to the VH and VL components that would form a scFv if linked by a linker; however, “split-scFv” designs typically do not include a peptide linker disposed between and connecting the VH and the VL. Thus, “split-scFv” refers to an arrangement wherein VH is comprised in a first polypeptide of a TCR/CAR and VL is comprised in a second polypeptide of a TCR/CAR, and VH and VL function to form a target-binding domain. In “split” formats, VH and VL can be engineered to introduce one or more disulfide bond therebetween; see e.g. Reiter et al. Biochemistry 33:5451-5459 (1994), Brinkmann Antibody Engineering (2012) doi.org/10.1007/978-3-642-01147-4_14: Weatherill et al. PEDS 25 (7): 321-329 (2012), and Metz et al. PEDS 25 (10): 571-580 (2012)). Such disulfide bonds may be introduced in “full” scFv for full scFab formats, as well.

In some embodiments, a target-binding domain is fully comprised in one TCR constant domain-containing polypeptide. For example, a scFv (VH-linker-VL or VL-linker-VH) may be comprised in the first polypeptide or the second polypeptide of a TCR/CAR; such an arrangement can be described as “full-scFv”.

In some embodiments, both the first polypeptide and the second polypeptide fully comprise a target-binding domain. For example, the first polypeptide can comprise a Cα linked or fused to a first scFv and the second polypeptide can comprise a Cβ linked or fused to a second scFv. In other embodiments, the first polypeptide can comprise a Cα linked or fused to a first VHH and the second polypeptide can comprise a Cβ linked or fused to a second VHH. In other embodiments, the first polypeptide can comprise a Cα linked or fused to a first Fab or scFab and the second polypeptide can comprise a Cβ linked or fused to a second Fab or scFab, respectively. In other embodiments, the first polypeptide can comprise a Cα linked or fused to a first ligand and the second polypeptide can comprise a Cβ linked or fused to a second ligand. For example, the first polypeptide can comprise a Cα linked or fused to a first receptor ectodomain and the second polypeptide can comprise a Cβ linked or fused to a second receptor ectodomain.

When present, two target-binding domains (it will be understood that any arrangement and combination of binding domains is contemplated; for example, one polypeptide can comprise a scFv and the other polypeptide can comprise a VHH, or one polypeptide can comprise a Fab and the other polypeptide can comprise a scFv, or one polypeptide can comprise a receptor ectodomain and the other polypeptide can comprise a DARPin, or the like) may be the same (e.g. two copies of a same VHH may be present, one comprised in each of the first and the second polypeptide) or different. If different, the two binding domains may target different epitopes on the same target (e.g. two epitopes present within a tumor antigen), or may target different targets altogether (e.g., may target two different tumor antigens, or may target a tumor antigen and a cytokine). Accordingly, certain embodiments provide multispecific (e.g. bispecific) TCR/CARs. In some contexts, a multispecific TCR/CAR binds to two or more antigens that are expressed by a cancer; for example, to target multiple myeloma, a multispecific TCR/CAR may target any two or more of: BCMA, GPRC5D, SLAMF7, and CD229. In some contexts, a multispecific TCR/CAR binds to two or more antigens that are expressed by a cancer; for example, to target multiple myeloma, a multispecific TCR/CAR may target any two or more of: BCMA, GPRC5D, SLAMF7, CD229, CD19, and CD22.

Contemplated embodiments include those wherein a polypeptide chain comprises two or more binding domains (e.g., scFv-linker-scFv-TCR constant domain; VHH-linker-VHH-TCR constant domain; or the like).

A target can be a synthetic molecule or a biological antigen or other biomolecule. In some embodiments, a target is expressed on or by a cancer cell, a cell infected with a pathogen (e.g. virus, fungus, parasite, bacteria) or is otherwise associated with an an infection, or is associated with an autoimmune disease or a neurodegenerative disease (e.g., tau, amyloid-beta, alpha-synuclein), or is a cytokine (e.g. TNFα, IL-13, IL-10) or a chemokine. In some embodiments, a target is or comprises a cancer antigen selected from BCMA, GPRC5D, CD19, RORI, SLAMF7, CD229, PNE, EGFR, EGFRVIII, EGP-2, EGP-40, GD2, GD3, HPV E6, HPV E7, Her2, LI-CAM, Lewis A, Lewis Y, MUC1, MUC16, PSCA, PSMA, CD20, CD22, CD56, CD23, CD24, CD30, CD33, CD37, CD44v7/8, CD3δ, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Carbonic anhydrase 9, BRAF, α-fetoprotein, MAGE-A3, MAGE-A4, SSX-2, PRAME, HA-1, β2M, ETA, tyrosinase, KRAS, NRAS, a peptide: MHC complex, and CEA. In some embodiments, a TCR/CAR comprises a Bcl-2 ectodomain or portion or variant thereof and a target is a Bcl-2 ligand.

In certain embodiments, a TCR/CAR is bispecific and binds to: (i) BCMA and GPRC5D; (ii) BCMA and SLAMF7; (iii) BCMA and CD229; (iv) GPRCSD and SLAMF7; (v) GPRCSD and CD229; or (vi) SLAMF7 and CD229. In certain embodiments, a TCR/CAR is bispecific and binds to: CD19 and BCMA; or to CD19 and CD22. In some embodiments, the bispecific TCR/CAR comprises two scFvs. In some embodiments, a bispecific TCR/CAR comprises two VHH and each VHH binds to a different epitope on BCMA.

Non-limiting examples of binding domains include those that comprise the VH, the VL, the HCDRs, and/or the LCDRs of: trastuzumab; pertuzumab; rituximab; erbituxumab; ublituxumab; 1.5.3; a BMCA-specific antibody such as J22.0-xi, J22.9-xi, J6MO, J6M1, J6M2, J9M0, J9M1, J9M2, CA8, A7D12.2, C11 D5.3, C12A3.2, C13F12.1, 13C2, 17A5, 83A10, 13A4, 13D2, 14B11, 14E1, 29B11, 29F3, 13A7, CA7, SG1, S307118G03, S332121F02, S332126E04, S322110D07, S336105A07, S335115G01, S335122F05, ET140-3, ET140-24, ET140-37, ET140-40, ET140-54, TBL-CLN1, C4.E2.1, Vicky-1, pSCHLI333, pSCHLI372, pSCHLI373, and those other BCMA-specific antibodies and antigen-binding fragments disclosed in PCT Publication Nos. WO 2002/066516, WO 2007/062090, WO 2010/104949, WO 2011/108008, WO 2012/163805, WO 2014/068079, WO 2015/166073, WO 2014/122143, WO 2014/089335, WO 2016/090327, and WO 2016/079177; Ryan et al., Mol. Cancer. Ther. 6 (11): 3009, 2007; and Abbas et al., Blood 128:1688, 2016; a RORI-specific antibody such as R11, R12, Y4, Y13, Y27, or Y31; a CD19-specific antibody such as FMC63; a CD33-specific antibody such as gemtuzumab; a GPRC5D-specific antibody; a RORI-specific VHH; a VHH such as MB14; 3F8; alemtuzumab; XMAB-5574; pembrolizumab; nivolumab; a PD-1-specific antibody; elotuzomab; a SLAMF-specific antibody; a CD229-specific antibody; a PD-L1-specific antibody; a SARS-COV-2-specific antibody, or an (e.g. cancer antigen-specific, pathogen-specific, autoimmune disease antigen-specific, or neurodegenerative-disease-specific) antibody or antigen-binding fragment approved for therapeutic and/or diagnostic use in humans by the US Food and Drug Administration, the European Medicines Agency, or both. In some embodiments, a binding domain comprises the VH, the VL, the HCDRs, and/or the LCDRs of: 3F8, 8H9, Abagovomab, Abciximab, Abituzumab, Abrilumab, Actoxumab, Adalimumab, Adecatumumab, Aducanumab, Afasevikumab, Afelimomab, Afutuzumab, Alacizumab pegol, ALDS18, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cergutuzumab amunaleukin, Certolizumab pegol, Cetuximab, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Crotedumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dinutuximab, Donanemab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erenumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Idarucizumab, Igovomab, IMAB362, Imalumab, Imciromab, Imgatuzumab, Inclacumab, Indatuximab ravtansine, Indusatumab vedotin, Inebilizumab, Infliximab, Inolimomab, Inotuzumab ozogamicin, Intetumumab, Ipilimumab, Iratumumab, Isatuximab, Itolizumab, Ixekizumab, Keliximab, Labetuzumab, Lampalizumab, Lanadelumab, Landogrozumab, Laprituximab emtansine, Lebrikizumab, Lemalesomab, Lendalizumab, Lenzilumab, Lerdelimumab, Lecanemab, Lexatumumab, Libivirumab, Lifastuzumab vedotin, Ligelizumab, Lilotomab satetraxetan, Lintuzumab,Lirilumab, Lodelcizumab, Lokivetmab, Lorvotuzumab mertansine, Lucatumumab, Lulizumab pegol, Lumiliximab, Lumretuzumab, MABpl, Mapatumumab, Margetuximab, Maslimomab, Matuzumab, Mavrilimumab, Mepolizumab, Metelimumab, Milatuzumab, Minretumomab, Mirvetuximab soravtansine, Mitumomab, Mogamulizumab, Monalizumab, Morolimumab, Motavizumab, Moxetumomab pasudotox, Muromonab-CD3, Nacolomab tafenatox, Namilumab, Naptumomab estafenatox, Naratuximah emtansine, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanczumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Prezalizumab, Priliximab, Pritoxaximab, Pritumumab, PRO 140 aka leronlimab, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab tesirine, Rovelizumab, Ruplizumab, Sacituzumab govitecan, Samalizumab, Sapelizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Sotrovimab, Stamulumab, Sulesomab, Suvizumab, Tabalumab, Tacatuzumab tetraxetan, Tadocizumab, Talizumab, Tamtuvetmab, Tanezumab, Taplitumomab paptox, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tigatuzumab, Tildrakizumab, Timolumab, Tisotumab vedotin, TNX-650, Tocilizumab, Toralizumab, Tosatoxumab, Tositumomab, Tovetumab, Tralokinumab, Trastuzumab, Trastuzumab emtansine, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab mafodotin, Votumumab, Xentuzumab, Zalutumumab, Zanolimumab, Zatuximab, Ziralimumab, or Zolimomab aritox.

In certain embodiments, a TCR/CAR comprises VH, the VL, the HCDRs, and/or the LCDRs of an antibody or antigen-binding fragment that is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor I), U.S. Pat. Nos. 7,566,772 and 7,964,193 (interleukin-1B), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publication No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (0V064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain and CDR polypeptides, variable domain and CDR-encoding nucleic acids, host cells, vectors, methods of making the antibodies or antigen-binding fragments polypeptides encoding said variable domains, pharmaceutical compositions, and methods of treating diseases associated with the respective target of the variable domain-containing antibody or antigen-binding fragment.

In certain embodiments, a TCR/CAR comprises VH, the VL, the HCDRs, and/or the LCDRs of an antibody or antigen-binding fragment that is one of: Muromonab-CD3 (product marketed with the brand name Orthoclone Okt3®), Abciximab (product marketed with the brand name Reopro®.), Rituximab (product marketed with the brand name Mab Thera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-IHR), Adalimumab (product marketed with the brand name Humira®), Tositumomab-1131 (product marketed with the brand name Bexxar®), Efalizumab (product marketed with the brand name Raptiva®), Cetuximab (product marketed with the brand name Erbitux®), l'Ibritumomab tiuxetan (product marketed with the brand name Zevalin®), l'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), l'Eculizumab (product marketed with the brand name Soliris®), Certolizumab pegol (product marketed with the brand name Cimzia®), Golimumab (product marketed with the brand name Simponi®), Canakinumab (product marketed with the brand name Ilarise), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1.beta. antibodies such as canakinumab; anti-IL12/23 (p40) antibodies such as ustekinumab and briakinumab; and anti-IL2R antibodies, such as daclizumab. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CDS2 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies such as siltuximab; and anti-VEGF antibodies such as bevacizumab; and anti-IL6 receptor antibodies such as tocilizumab.

In certain embodiments, a target comprises a protein ligand and a binding domain is from a receptor for the ligand. For example, a binding domain can comprise a receptor ectodomain from Bcl2 and a target comprises BIM.

In some embodiments, a binding domain comprises a “TCR-mimic” antibody fragment (e.g. scFv or VH and VL from a TCR-mimic antibody) and a target comprises a peptide antigen in complex with a MHC (e.g. HLA) molecule. In some embodiments, the MHC molecule is a Class I MHC molecule. In other embodiments, the MHC molecule is a Class II MHC molecule. In certain further embodiments, a peptide antigen: HLA complex comprises WT1126 (RMFPNAPYL; SEQ ID NO.: 66)/HLA-A*0201 or NY-ESO-1157 (SLLMWITQC; SEQ ID NO.: 67)/HLA-A*0201. TCR-mimic antibodies may be prepared by the hybridoma methodology described by Kohler et al., Nature 256:495 (1975), or may be made using recombinant DNA methods in bacterial, eukaryotic animal, or plant cells (see, e.g., U.S. Pat. No. 4,816,567). TCR-mimic antibodies may also be isolated from phage antibody libraries using the techniques described in Clackson et al., Nature, 352:624-628 (1991) and Marks et al., J. Mol. Biol., 222:581-597 (1991), and Noy et al. Expert Rev. Anticancer Ther. 5 (3): 523-536 (2005); these techniques are incorporated herein in their entireties), for example. TCR-mimic antibodies may also be obtained using methods disclosed in PCT Publication No. WO 2004/076677A2.

In some embodiments, the binding domain is capable of specifically binding to the target. As used herein, “specifically binds” or “specific for” refers to an association or union of an target-binding protein or a binding domain (or fusion protein comprising the same) to a target molecule with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Target-binding proteins or binding domains may be classified as “high-affinity” binding proteins or binding domains or as “low-affinity” binding proteins or binding domains. “High-affinity” binding proteins or binding domains refer to those binding proteins or binding domains having a Kₐ of at least 10⁷M⁻¹, at least 10⁸ M⁻¹, at least 10⁹ M¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, or up to 10⁵ M⁻¹. Alternatively, affinity may be defined as an equilibrium dissociation constant (K_{D}) of a particular binding interaction with units of M (e.g., 10⁻⁵ M to 10⁻¹³ M).

In some embodiments, a binding domain is engineered have a preferred affinity for the target. For example, without wishing to be bound by theory, higher binding affinities (e.g., picomolar or femtomolar Kd) may in some contexts initiate or contribute to a more intense signal than is desired upon binding to antigen (e.g., binding by a TCR/CAR comprising such a binding domain may contribute to T cell signaling that is more intense, longer, or both, than may be desired, and may contribute to tonic signaling and/or cell exhaustion). Accordingly, certain embodiments provide binding domains which are selected for or are modified to have affinity in a preferred range, such as in the range of about 1 nM to about 100 nM Kd, as determined by surface plasmon resonance, which may be comparable to physiological affinity of a native TCR for antigen: MHC. By way of illustration, if a binding domain from an antibody with picomolar or femtomolar Kd for its antigen is selected for use in a TCR/CAR, the binding domain may first be engineered to decrease affinity for antigen to a range of about 1 nM to about 100 nM Kd, such as by rational mutagenesis in one or more CDRs (e.g., by mutation from an amino acid with a larger side-chain (e.g. tryptophan or phenylalanine) to an amino acid with a smaller side-chain (e.g. alanine or serine))

A variety of assays are known for identifying binding domains that specifically bind a particular target, as well as determining binding domain or antigen-binding protein affinities, such as Western blot, ELISA, analytical ultracentrifugation, spectroscopy, isothermal titration calorimetry (ITC), and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for apparent affinity or relative affinity are also known. In certain examples, apparent affinity for a target-binding protein is measured by assessing binding to various concentrations of tetramers, for example, by flow cytometry using labeled tetramers. In some examples, apparent K_{D} of a binding protein or binding domain is measured using 2-fold dilutions of labeled tetramers at a range of concentrations, followed by determination of binding curves by non-linear regression, apparent K_{D} being determined as the concentration of ligand that yielded half-maximal binding.

Non-limiting examples of binding domain amino acid sequences are provided in SEQ ID NOs.: 97-109. In some embodiments, a TCR/CAR comprises a binding domain comprising: (i) SEQ ID NO.: 97 and SEQ ID NO.: 98, optionally comprised in a scFv, such as having the sequence of SEQ ID NO.: 99 or SEQ ID NO.: 100; (ii) SEQ ID NO.: 101 and 102, optionally in a scFv, such as having the sequence of SEQ ID NO.: 103; (iii) SEQ ID NO.: 104 and SEQ ID NO.: 105, optionally in a scFv, such as having the sequence of SEQ ID NO.: 106; (iv) SEQ ID NO.: 107, optionally comprised in SEQ ID NO.: 108; or (v) SEQ ID NO.: 109. In certain further embodiments, the binding domain comprising (i), (ii), (iii), (iv), and/or (v) above is fused or linked to a TCR Cα, to a TCR Cβ, or to both of a TCRα and a TCRβ (e.g., where a VH and a VL are present, in a “split” format or in a “full format”). In some embodiments, the TCR Co has at least 90%, at least 91%, at least 92%, at least 93%, at least 94%, at least 95%, at least 96%, at least 97%, at least 98%, at least 99%, or 100% identity to SEQ ID NO.: 56. In some embodiments, the TCR Cα has at least 90%, at least 91%, at least 92%, at least 93%, at least 94%, at least 95%, at least 96%, at least 97%, at least 98%, at least 99%, or 100% identity to SEQ ID NO.: 57. In some embodiments, the TCR Cβ has at least 90%, at least 91%, at least 92%, at least 93%, at least 94%, at least 95%, at least 96%, at least 97%, at least 98%, at least 99%, or 100% identity to SEQ ID NO.: 58. In some embodiments, the TCR Cβ has at least 90%, at least 91%, at least 92%, at least 93%, at least 94%, at least 95%, at least 96%, at least 97%, at least 98%, at least 99%, or 100% identity to SEQ ID NO.: 59. In some embodiments, the TCR Cβ has at least 90%, at least 91%, at least 92%, at least 93%, at least 94%, at least 95%, at least 96%, at least 97%, at least 98%, at least 99%, or 100% identity to SEQ ID NO.: 60. In some embodiments, the TCR Cβ has at least 90%, at least 91%, at least 92%, at least 93%, at least 94%, at least 95%, at least 96%, at least 97%, at least 98%, at least 99%, or 100% identity to SEQ ID NO.: 61.

The target-binding domain or a component thereof may be directly fused to a TCR constant domain (i.e., there is no intervening amino acid or amino acid sequence between the target-binding domain or component thereof and the TCR constant domain) or may be linked to a TCR constant domain by a linker, which may function as a hinge. Accordingly, in some embodiments, a hinge sequence is disposed between and connects the target-binding domain or portion thereof and a TCR constant domain. A hinge sequence may be from an immunoglobulin superfamily molecule (e.g. an antibody hinge or a CD8 hinge, or an engineered variant hinge based thereon, or a synthetic flexible linker such as a glycine-serine linker) and can confer desired structure and flexibility for binding to the target. A hinge sequence can be selected and/or engineered for preferred characteristics, such as, for example, a desired length, a desired flexibility, a desired reduced interaction or lack of interaction of interaction with a Fc receptor (e.g. a FcγR.).

Non-limiting examples of hinge sequences are provided in SEQ ID NOs.: 42-55. These include a (G₄S) ₃ linker (SEQ ID NO.: 54); it will be understood that this linker and other synthetic linkers may function as a hinge, or may be present elsewhere in a TCR/CAR or CCR of the present disclosure (e.g., between adjacent domains of a polypeptide, such as between VH and VL in a scFv). Linkers include an (A)ₙ linker, wherein n is 1 or more, a GS linker, a GSG linker, a GPP linker, a (GlyₓSer_{y})ₙ linker wherein X, Y, and N are not zero, and may each independently be from 1-10, a Townsend linker (GSGGSGGSGGTG; SEQ ID NO.: 68), a Whitlow linker aka linker 218 (GSTSGSGKPGSGEGSTKG; SEQ ID NO.: 69), or a linker comprising or consisting of any one of the following amino acid sequences: GSGKPGSGEG (SEQ ID NO.: 70); GKPGSGEG (SEQ ID NO.: 71); SGKPGSGE (SEQ ID NO.: 72); EGKSSGSGSESKVD (SEQ ID NO.: 73), or BPXXXZ, wherein each X is independently a glycine (G) or serine(S), Bis a positively charged amino acid and Z is glycine (G) or a negatively charged amino acid (SEQ ID NO.: 74), or the like).

Non-limiting examples of TCR/CAR polypeptide amino acid sequences are provided in SEQ ID NOs.: 110-125. In certain embodiments, a TCR/CAR comprises a first polypeptide and a second polypeptide, wherein the first polypeptide and the second polypeptide comprise, consist essentially of, or consist of, amino acid sequences having at least least 90%, at least 91%, at least 92%, at least 93%, at least 94%, at least 95%, at least 96%, at least 97%, at least 98%, or at least 99% to, or comprising or consisting of, the amino acid sequences set forth in SEQ ID NOs.: (i) 110 and 111, respectively; (ii) 112 and 113, respectively; (iii) 118 and 119; (iv) 116 and 117; (v) 120 and 121; or (vi) 122 or 123 and 124 or 125, respectively. In some embodiments, a TCR/CAR comprises a first polypeptide and a second polypeptide, wherein the first polypeptide comprises or consists of the amino acid sequence set forth in SEQ ID NO.: 114 and the second polypeptide consists essentially of or consists of a TCR Cβ. In some embodiments, a TCR/CAR comprises a first polypeptide and a second polypeptide, wherein the first polypeptide comprises or consists of the amino acid sequence set forth in SEQ ID NO.: 115 and the second polypeptide consists essentially of or consists of a TCR Cα. In some embodiments, a TCR/CAR comprises a first polypeptide and a second polypeptide, wherein the first polypeptide comprises or consists of the amino acid sequence set forth in SEQ ID NO.: 115 and the second polypeptide consists essentially of or consists of a TCR Cβ. In some embodiments, a TCR/CAR comprises a first polypeptide and a second polypeptide, wherein the first polypeptide comprises or consists of the amino acid sequence set forth in SEQ ID NO.: 116 or SEQ ID NO.: 117 and the second polypeptide consists essentially of or consists of a TCR Cα. In some embodiments, a TCR/CAR comprises a first polypeptide and a second polypeptide, wherein the first polypeptide comprises or consists of the amino acid sequence set forth in SEQ ID NO.: 118 and the second polypeptide consists essentially of or consists of a TCR Cα. In some embodiments, a TCR/CAR comprises a first polypeptide and a second polypeptide, wherein the first polypeptide comprises or consists of the amino acid sequence set forth in SEQ ID NO.: 119 and the second polypeptide consists essentially of or consists of a TCR Cβ. In some embodiments, a TCR/CAR comprises a first polypeptide and a second polypeptide, wherein the first polypeptide comprises or consists of the amino acid sequence set forth in SEQ ID NO.: 120 and the second polypeptide consists essentially of or consists of a TCR Cβ. In some embodiments, a TCR/CAR comprises a first polypeptide and a second polypeptide, wherein the first polypeptide comprises or consists of the amino acid sequence set forth in SEQ ID NO.: 121 and the second polypeptide consists essentially of or consists of a TCR Cα. In some embodiments, a TCR/CAR comprises a first polypeptide and a second polypeptide, wherein the first polypeptide comprises or consists of the amino acid sequence set forth in SEQ ID NO.: 122 or SEQ ID NO.: 123 and the second polypeptide consists essentially of or consists of a TCR Cβ. In some embodiments, a TCR/CAR comprises a first polypeptide and a second polypeptide, wherein the first polypeptide comprises or consists of the amino acid sequence set forth in SEQ ID NO.: 124 or SEQ ID NO.: 125 and the second polypeptide consists essentially of or consists of a TCR Cα.

In some embodiments, a first polypeptide, the second polypeptide, or both, of a TCR/CAR has at least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 114, 115, 116, 117, 118, 119, 120, 121, 122, 123, 124, or 125.

Table 1 provides non-limiting examples of certain TCR/CAR designs. It will be understood that a scFv can be in VH-linker-VL or VL-linker-VH orientation. It will be understood that a scFab can be in VH-CH1-linker-VL-CL orientation or VL-CL-linker-VH-CH1 orientation (or, alternatively, that CH1 and CL can be exchanged or CH3 can replace CH1 and CL; e.g., VH-CL-linker-VL-CH1, VL-CH1-linker-VH-CL, VH-CH3-linker-VL-CH3, VL-CH3-linker-VH-VH3). It will be understood that any TCR/CAR can comprise a hinge disposed between a binding domain and a TCR constant domain. It will be understood that where two or more binding domains are present, they may be the same or different. If different, they may comprise a different amino acid sequence to one another but still bind a same (or overlapping) epitope or a same target, or they may bind different epitopes on a same target or may bind different targets. It will be understood that in Table 1, “none” refers to the absence of a target or antigen-binding domain; the absence of an antigen-binding domain does not exclude the presence of amino acid sequence N-terminal to the TCR constant domain. For example, in some embodiments of the present disclosure, one of the first polypeptide and the second polypeptide comprises, N-terminal to the TCR constant domain thereof, one or more tag and optionally one or more linker. A tag can include, for example, any tag or combinations of tags known in the art, including those tags and combinations of tags described herein. It will be understood that a polypeptide dimer (TCR/CAR) can comprise one or more additional binding domain further to the binding domain(s) specified in Table 1; for example, a polypeptide of a polypeptide dimer may comprise two, three, four, or more binding domains, e.g. linked in a series.

TABLE 1

| | Certain TCR/CAR designs | |
|---|---|---|
| | First polypeptide (e.g. | Second polypeptide (e.g. comprising cognate |
| | comprising TCR Cα or Cβ) | TCR C domain to first polypeptide) |
| Binding | VH | Corresponding VL of first polypeptide VH |
| domain | | |
| Binding | VL | Corresponding VH of first polypeptide VL |
| domain | | |
| Binding | scFv | None |
| domain | | |
| Binding | None | scFv |
| domain | | |
| Binding | scFv | scFv; same or different to first polypeptide scFv |
| domain | | |
| Binding | scFab | None |
| domain | | |
| Binding | None | scFab |
| domain | | |
| Binding | scFab | scFab; same or different to first polypeptide |
| domain | | scFab |
| Binding | VHH | None |
| domain | | |
| Binding | None | VHH |
| domain | | |
| Binding | VHH | VHH; same or different to first polypeptide VHH |
| domain | | |
| Binding | VHH-linker-VHH | None |
| domain | | |
| Binding | None | VHH-linker-VHH |
| domain | | |
| Binding | VHH-linker-VHH | VHH-linker-VHH; same or different to first |
| domain | | polypeptide VHH-linker-VHH |
| Binding | Protein ligand-binding | None |
| domain | domain e.g. receptor | |
| | ectodomain (e.g.) from Bcl2 | |
| Binding | None | Protein ligand-binding domain e.g. receptor |
| domain | | ectodomain |
| Binding | Protein ligand-binding | Protein ligand-binding domain e.g. receptor |
| domain | domain e.g. receptor | ectodomain; same or different to first polypeptide |
| | ectodomain | protein ligand-binding domain |
| Binding | scFv | scFab |
| domain | | |
| Binding | scFab | scFv |
| domain | | |
| Binding | scFv | VHH or VHH-linker-VHH |
| domain | | |
| Binding | VHH or VHH-linker-VHH | scFv |
| domain | | |
| Binding | scFv | Protein ligand-binding domain |
| domain | | |
| Binding | Protein ligand-binding | scFv |
| domain | domain | |
| Binding | scFab | VHH or VHH-linker-VHH |
| domain | | |
| Binding | VHH or VHH-linker-VHH | scFab |
| domain | | |
| Binding | scFab | Protein ligand-binding domain |
| domain | | |
| Binding | Protein ligand-binding | scFab |
| domain | domain | |
| Binding | VHH or VHH-linker-VHH | Protein ligand-binding domain |
| domain | | |
| Binding | Protein ligand-binding | VHH or VHH-linker-VHH |
| domain | domain | |
| Binding | Protein ligand-binding | Protein ligand-binding domain |
| domain | domain | |

Additionally or alternatively, any first and/or second polypeptide of a TCR/CAR as in Table 1 can comprise, as a binding domain: a cytokine; a chemokine; a synthetic polypeptide selected for its specific ability to bind to a biological molecule, a molecular complex or other target of interest (e.g., DARPins, ¹⁰FNIII domains); a killer immunoreceptor from a NK cell; a designed ankyrin repeat protein (DARPin); a ¹⁰FNIII domain such as an Adnectin™ or monobody; a lectin binding domain; a fibrinogen domain; a cysteine-knot miniprotein; a tetratricopeptide repeat domain; a lipocalin domain; an armadillo repeat protein; an affibody; an avimer; a knottin; a fynomer; an atrimer; cytotoxic T-lymphocyte associated protein-4; a centyrin; or any combination thereof.

In some embodiments, a first and a second polypeptide of a TCR/CAR each comprise a scFv.

Also provided are embodiments wherein a first polypeptide of a TCR/CAR and a second polypeptide of a TCR/CAR comprise, as a binding domain: (i) a VNAR and a VNAR, respectively (wherein the VNARs can be the same or different); (ii) a VNAR and no binding domain, respectively; (iii) no binding domain and a VNAR, respectively; (iv) a VNAR and scFv, respectively; (v) a scFv and a VNAR, respectively; (vi) a VNAR and a scFab, respectively; (vii) a scFab and a VNAR, respectively; (viii) a VNAR and a VHH or a VHH-linker-VHH, respectively; (ix) a VHH or a VHH-linker-VHHH and a VNAR, respectively; (x) a VNAR and a protein ligand-binding domain, respectively; or (xi) a protein ligand-binding domain and a VNAR, respectively.

Additionally or alternatively, a TCR/CAR can comprise a VNAR and one or more of: a cytokine; a chemokine; a synthetic polypeptide selected for its specific ability to bind to a biological molecule, a molecular complex or other target of interest (e.g., DARPins, ¹⁰FNIII domains); a killer immunoreceptor from a NK cell; a designed ankyrin repeat protein (DARPin); a ¹⁰FNIII domain such as an Adnectin™ or monobody; a lectin binding domain; a fibrinogen domain; a cysteine-knot miniprotein; a tetratricopeptide repeat domain; a lipocalin domain; an armadillo repeat protein; an affibody; an avimer; a knottin; a fynomer; an atrimer; cytotoxic T-lymphocyte associated protein-4; a centyrin; or any combination thereof.

Also provided are embodiments wherein a VH or a VL of an antibody is sufficient for binding and a first polypeptide of a TCR/CAR comprises the VH or VL and the second polypeptide of the TCR/CAR does not comprise a binding domain, or comprises the cognate VL or VH of the first polypeptide, or comprises a different binding domain. In some embodiments, a first polypeptide of a TCR/CAR and a second polypeptide of a TCR/CAR comprise, as a binding domain: (i) an antigen-binding VH or VL and a VNAR, respectively; (ii) an antigen-binding VH or VL and no binding domain, respectively; (iii) no binding domain and an antigen-binding VH or VL, respectively; (iv) a VNAR and an antigen-binding VH or VL, respectively; (v) an antigen-binding VH or VL and an antigen-binding VH or VL, respectively; (vi) an antigen-binding VH or VL and a scFab, respectively; (vii) a scFab and an antigen-binding VH or VL, respectively; (viii) an antigen-binding VH or VL and a VHH or a VHH-linker-VHH, respectively; (ix) a VHH or a VHH-linker-VHHH and an antigen-binding VH or VL, respectively; (x) an antigen-binding VH or VL and a protein ligand-binding domain, respectively; or (xi) a protein ligand-binding domain and an antigen-binding VH or VL, respectively.

Additionally or alternatively, a TCR/CAR can comprise an antigen-binding VH or antigen-binding VL and one or more of: a cytokine; a chemokine; a synthetic polypeptide selected for its specific ability to bind to a biological molecule, a molecular complex or other target of interest (e.g., DARPins, ¹⁰FNIII domains); a killer immunoreceptor from a NK cell; a designed ankyrin repeat protein (DARPin); a ¹⁰FNIII domain such as an Adnectin™ or monobody; a lectin binding domain; a fibrinogen domain; a cysteine-knot miniprotein; a tetratricopeptide repeat domain; a lipocalin domain; an armadillo repeat protein; an affibody; an avimer; a knottin; a fynomer; an atrimer; cytotoxic T-lymphocyte associated protein-4; a centyrin; or any combination thereof.

It will be appreciated that either or both of the first polypeptide and the second polypeptide can comprise any binding domain, such as described herein. It will be appreciated that the first polypeptide can comprise a TCR Co. and the second polypeptide can comprise a TCR Cβ, or the first polypeptide can comprise a TCR Cβ and the second polypeptide can comprise a TCR Cα.

Any TCR constant domain-containing polypeptide of a TCR/CAR can also be provided as an isolated polypeptide. For example, a Cα-containing or Cβ-containing polypeptide can be provided, wherein the Cα-containing or Cβ-containing polypeptide comprises a binding domain as provided herein.

Also provided are chimeric co-stimulatory receptor polypeptides (CCRs) that are capable of improving host cell (e.g. T cell) function when the host cell encounters PVR aka CD155. PVR aka CDISS is often overexpressed by tumor cells and is a shared ligand for two T cell proteins, CD226 and TIGIT. CD226 can be gradually lost following chronic antigen stimulation, while TIGIT is expressed following T cell activation and becomes constitutively expressed during T cell exhaustion. TIGIT has a higher affinity for PVR (approximately 100-fold) than does CD226. Disclosed CCRs are capable of manipulating PVR/TIGIT/CD226 signaling and improve one or more function of a host cell (e.g. peristence, anti-tumor activity, activation, proliferation, or the like) in the presence of PVR (e.g. in the presence of tumor cells). Presently disclosed CCRs are single-chain fusion polypeptides.

In some embodiments, a CCR comprises an extracellular binding domain from CD226 (e.g. can comprise a CD226 ectodomain (also referred-to as extracellular domain or extracellular component)), or a portion or variant thereof that is functional to bind PVR. An example of a CD226 ectodomain amino acid sequence is provided in SEQ ID NO.: 77. A portion or variant of a CD226 ectodomain that is functional to bind PVR will preferably maintain an Ig-like C2-type 1 domain and an Ig-like C2-type 2 domain of CD226. The CCR can further comprise a transmembrane domain from CD226. An example of a CD226 transmembrane domain amino acid sequence is provided in SEQ ID NO.: 78. In some embodiments, a CD226-based CCR comprises an ectodomain comprisign an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 77 and, optionally, a transmembrane domain having least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 78.

In other embodiments, a CCR comprises an extracellular binding domain from TIGIT (e.g. can comprise a TIGIT ectodomain), or a portion or variant thereof that is functional to bind PVR. An example of a TIGIT ectodomain amino acid sequence is provided in SEQ ID NO.: 82. A portion or variant of a TIGIT ectodomain that is functional to bind PVR will preferably maintain an Ig-like V-type domain of TIGIT. The CCR can further comprise a transmembrane domain from TIGIT. An example of a TIGIT transmembrane domain amino acid sequence is provided in SEQ ID NO.: 83. In some embodiments, a TIGIT-based CCR comprises an ectodomain having at least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 82 and, optionally, a transmembrane domain having least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 83.

A CCR further comprises an intracellular component designed to preserve or augment normal CD226 signaling or to disrupt or abrogate TIGIT signaling, such as by abrogating this signaling and/or by converting this signaling into an activating or co-stimulatory signal. A CCR may comprise a signaling (e.g. costimlatory) domain from, for example, CD2, CD28, 4-1BB, or the like. For example, a CCR can comprise: (i) a CD226 ectodomain and a mutated CD226 endodomain (also called intracellular domain) sequence (e.g. comprising a K295A mutation, a K333A mutation, or both; see Braun et al., Immunity 53 (4): 805-823.e15 (2020)); (ii) a CD226 ectodomain and a CD2 intracellular domain; (iii) a CD226 ectodomain and a truncated CD2 intracellular domain; (iv) a CD226 ectodomain and a CD28 co-stimulatory domain; (v) a CD226 ectodomain and a 4-1BB co-stimulatory domain; (vi) a TIGIT ectodomain and a CD2 intracellular domain; (vii) a TIGIT ectodomain and a truncated CD2 intracellular domain; (viii) a TIGIT ectodomain and a CD28 co-stimulatory domain; (ix) a TIGIT ectodomain and a 4-1BB co-stimulatory domain; (x) a CD226 ectodomain and a CD27 co-stimulatory domain; (xi) a CD226 ectodomain and an OX40 costimulatory domain; (xii) a CD226 ectodomain and an ICOS costimulatory domain; (xiii) a CD226 ectodomain and a DAP10 costimulatory domain; (xiv) a CD226 ectodomain and a HVEM costimulatory domain; (xv) a CD226 ectodomain and a LIGHT costimulatory domain; (xvi) a CD226 ectodomain and CD30 costimulatory domain; (xvii) a CD226 ectodomain and SLAM1 costimulatory domain; (xviii) a TIGIT ectodomain and a CD27 co-stimulatory domain; (xix) a TIGIT ectodomain and an OX40 costimulatory domain; (xx) a TIGIT ectodomain and an ICOS costimulatory domain; (xxi) a TIGIT ectodomain and a DAP10 costimulatory domain; (xxii) a TIGIT ectodomain and a HVEM costimulatory domain; (xxiii) a TIGIT ectodomain and a LIGHT costimulatory domain; (xiv) a TIGIT ectodomain and CD30 costimulatory domain; or (xv) a TIGIT ectodomain and SLAMI costimulatory domain. It will be appreciated that embodiments comprising a a portion or variant of a CD226 or TIGIT ectodomain that is functional to bind PVR are contemplated.

A CCR comprising a TIGIT ectodomain can comprise a TIGIT transmembrane domain. A CCR comprising a CD226 ectodomain can comprise a CD226 transmembrane domain.

An example of a mutated CD226 endodomain amino acid sequence is provided in SEQ ID NO.: 79. An example of a CD2 endodomain amino acid sequence is provided in SEQ ID NO.: 84. An example of a truncated CD2 endodomain amino acid sequence is provided in SEQ ID NO.: 85. An example of a CD28 co-stimulatory domain amino acid sequence is provided in SEQ ID NO.: 86. An example of a 4-1BB co-stimulatory domain amino acid sequence is provided in SEQ ID NO.: 87. In certain embodiments, a CCR comprises an intracellular component comprising an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 79, 84, 85, 86, or 87.

In certain embodiments, a CCR comprises, consists essentially of, or consists of, an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 88-96.

Any CCR of the present disclosure can be expressed in a host cell (e.g. T cell) with any TCR/CAR of the present disclosure (or with a TCR, or a CAR) and can provide an activating and/or co-stimulatory signal to the host cell; for example, a host T cell encounters a tumor cell that expresses an antigen and PVR and binds to the antigen through the TCR/CAR (or TCR, or CAR), thus providing an antigen-binding signal to the T cell, and binds to PVR through the CCR, thus preserving a normal PVR-binding signal via CD226 and/or preventing or attenuating a normal PVR-binding signal via endogenous TIGIT and optionally converting the PVR-binding signal into an activating and/or co-stimulatory signal.

Also provided are polynucleotides that encode a disclosed TCR/CAR, a disclosed CCR, or both. Any CCR of the present disclosure can be encoded in a polynucleotide or vector with any TCR/CAR of the present disclosure (or with a TCR, or a CAR).

In some embodiments, a CCR comprises a CD226 ectodomain (i.e. any CD226-based CCR may be utilized, including a CCR that comprises a portion or variant of a CD226 ectodomain that is functional to bind PVR) and a CAR or TCR/CAR binds to: BCMA; GPRCSD; CD229; SLAMF7; BCMA and GPRC5D; BCMA and CD229; or BCMA and SLAMF7.

In some embodiments, a CCR comprises a TIGIT ectodomain (i.e. any TIGIT-based CCR may be utilized, including a CCR that comprises a portion or variant of a TIGIT ectodomain that is functional to bind PVR) and a CAR or TCR/CAR binds to: BCMA; GPRCSD; CD229; SLAMF7; BCMA and GPRC5D; BCMA and CD229; or BCMA and SLAMF7.

A polynucleotide can be codon-optimized for expression in a host cell. A polynucleotide can be comprised in a vector, such as, for example, a viral vector, such as a lentiviral vector or a retroviral vector. A polynucleotide or vector can include one or more additional features to facilitate desired expression of the encoded polypeptide(s), such as one or more promoter, one or more sequence encoding a signal peptide (also known as a leader peptide or leader sequence or transit peptide), one or more sequence encoding a furin cleavage sequence, one or more sequence encoding a self-cleaving peptide, or any combination thereof.

Non-limiting examples of promoters include an EF1a promoter (SEQ ID NO.: 7) and a MNDu3 promoter (SEQ ID NO.: 8).

Signal peptides target newly synthesized polypeptides to their appropriate location inside or outside the cell. A signal peptide may be removed, at least in part, from the polypeptide during or once localization or secretion is completed. Polypeptides that have a signal peptide are referred to herein as a “pre-protein” and polypeptides having their signal peptide removed are referred to herein as “mature” proteins or polypeptides. Signal peptides can be at the N-terminal or C-terminal end of an encoded polypeptide. Non-limiting examples of signal peptides include: the signal peptide MLLLVTSLLLCELPHPAFLLIP (SEQ ID NO.: 126; from GM-CSF), the signal peptide MALPVTALLLPLALLLHAARP (SEQ ID NO.: 127; from CD8g); the signal peptide MRPRLWLLLAAQLTVLHGNSV (SEQ ID NO.: 128; from CD8B); and the signal peptide METDTLLLWVLLLWVPGSTG (SEQ ID NO.: 150, from murine IgG, kappa light chain). It will be appreciated that any suitable naturally occurring or engineered signal peptide can be employed. In some contexts, a signal peptide that is native to the encoded polypeptide or ectodomain of a polypeptide is used; for example, a CD226 signal peptide (MDYPTLLLALLHVYRALC; SEQ ID NO.: 76) may be used with a CD226-based CCR, a TIGIT signal peptide (MRWCLLLIWAQGLRQAPLASG; SEQ ID NO.: 81) may be used with a TIGIT-based CCR, and a receptor ectodomain signal peptide may be used in a receptor ectodomain-containing TCR/CAR. Certain signal peptides and characteristics of these are described in Owji et al., European Journal of Cell Biology 97 (6): 422-441 (2018), and in Ling et al. Front. Immunol. (2020) doi.org/10.3389/fimmu.2020.604318; the signal peptides of which are incorporated herein by reference. A furin cleavage sequence (also referred-to as a furin recognition site) can have a minimal cleavage site of R-X-X-R (SEQ ID NO.: 129). In some embodiments, a furin cleavage sequence has a minimal cleavage site of R—X-K/R-R (SEQ ID NO.: 130). In some embodiments, a furin cleavage sequence has a minimal cleavage site of RAKR (SEQ ID NO.: 131) or RARR (SEQ ID NO.: 132).

A nucleotide sequence encoding a self-cleaving peptide can be disposed between sequences encoding two polypeptides of interest. Self-cleavage of the peptide can separate a single-chain polypeptide into two polypeptides of interest. For example, expression of a TCR Cβ-containing polypeptide and a TCR Cα-containing polypeptide of a TCR/CAR of the present disclosure can be coordinated by encoding both polypeptides as part of a fusion amino acid sequence that separates (such as by action of a self-cleaving peptide and optionally a furin cleavage sequence) following translation, permitting expression of the TCR Cβ-containing polypeptide and the TCR Cα-containing polypeptide as separate molecules at the cell surface. Non-limiting examples of self-cleaving peptides include: a porcine teschovirus-1 2A (P2A) self-cleaving peptide with N-terminal G-S-G linker (GSGATNFSLLKQAGDVEENPGP; SEQ ID NO.: 133); a Thoseaasigna virus 2A (T2A) self-cleaving peptide (LEGGGEGRGSLLTCGDVEENPGPR; SEQ ID NO.: 134); an Equine rhinitis A virus (ERAV) 2A (E2A) self-cleaving peptide (QCTNYALLKLAGDVESNPGP; SEQ ID NO.: 135); and a Foot-and-Mouth disease virus 2A (F2A) self-cleaving peptide with N-terminal G-S-G linker (GSGVKQTLNFDLLKLAGDVESNPGP; SEQ ID NO.: 136).

In some embodiments, a polynucleotide comprises nucleotide sequences encoding a first and a second TCR constant domain-containing polypeptide, and further comprises a nucleotide sequence encoding a self-cleaving peptide, a nucleotide sequence encoding a furin or other protease cleavage site, or both.

Certain embodiments of a TCR/CAR or CCR further include one or more tag peptide, typically located in an extracellular component of the TCR/CAR or CCR. An example of a tag peptide is a Flag tag (DYKDDDDK; SEQ ID NO.: 137) or a variant thereof (e.g. DYKDEY; SEQ ID NO.: 138). Other non-limiting examples of tag peptides include a Strep tag (which refers the original Strep® tag, Strep® tag II, or any variant thereof; see, e.g., U.S. Pat. No. 7,981,632, which Strep tags are incorporated herein by reference), His tag, Xpress tag, Avi tag, Calmodulin tag, Polyglutamate tag, an HA tag (YPYDVDPDYA; SEQ ID NO.: 139), Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof. See, e.g., PCT Publication No. WO 2015/095895, which tag peptides and the amino acid sequences thereof are incorporated by reference herein. A combination of a Flag tag variant and a HA tag comprises the amino acid sequence DYKDEYYPYDVDPDYA (SEQ ID NO.: 140). Tag peptides can be useful for identifying, sorting, enriching, tracking, or isolating polypeptides that comprise the tag peptide(s), and cells that express the same. For example, antibodies or other proteins (e.g. Streptactin) having specificity for a tag peptide can be used for these purposes; such antibodies (or binding fragments thereof) or other proteins may be soluble or can be conjugated to beads, a cell culture plate, agarose, or any other solid surface matrix. Cells can be sorted, enriched, or isolated using an affinity column. In some contexts, an antibody having specificity for a tag peptide can be used to induce cell death (e.g. by ADCC or CDC or ADCP) of a cell expressing a tag peptide. A tag peptide can be comprised N-terminal to a target-binding domain, N-terminal to a TCR constant domain (whether a target-binding domain is present in the polypeptide or not), in a target-binding domain (e.g., comprised in a linker of a scFv or scFab), C-terminal to a target-binding domain (e.g. comprised in a hinge), or any combination of the foregoing.

Non-limiting examples of amino acid sequences comprising TCR/CAR components are provided in SEQ ID NOs.: 1-6, 9-15, and 24-32. The amino acid sequences are of expression products from certain TCR/CAR coding constructs. SEQ ID NOs.: 24-32 further comprise an amino acid sequence of a CCR.

SEQ ID NO.: 1 is an amino acid sequence encoded by a “split-scFv” TCR/CAR coding construct (general design of VL-CB_VH-Ca). It includes, from N-terminal end to C-terminal end: a GM-CSF leader sequence; VL of CD19-specific antibody FMC63; TCR Cβ; a furin cleavage sequence; a P2A peptide with N-terminal GSG linker; a GM-CSF leader sequence; VH of FMC63; and TCR Cα.

SEQ ID NO.: 2 is an amino acid sequence encoded by a “split-scFv” TCR/CAR coding construct (general design of VH-Cβ_VL-Cα). It includes, from N-terminal end to C-terminal end: a GM-CSF leader sequence; VH of FMC63; TCR Cβ; a furin cleavage sequence; a P2A peptide with N-terminal GSG linker; a GM-CSF leader sequence; VL of FMC63; and TCR Cα.

SEQ ID NO.: 3 is an amino acid sequence encoded by a “full-scFv” TCR/CAR coding construct (general design of Cβ_VL-linker-VH-Cα). It includes, from N-terminal end to C-terminal end: a GM-CSF leader sequence; TCR Cβ; a furin cleavage sequence; a P2A peptide with N-terminal GSG linker; a GM-CSF leader sequence; VL of FMC63; a linker; VH of FMC63; and TCR Cα.

SEQ ID NO.: 4 is an amino acid sequence encoded by a “full-scFv” TCR/CAR coding construct (general design of VL-linker-VH—Cβ_Cα). It includes, from N-terminal end to C-terminal end: a GM-CSF leader sequence; VL of FMC63; a linker; VH of FMC63; TCR Cβ; a furin cleavage sequence; a P2A peptide with N-terminal GSG linker; a GM-CSF leader sequence; and TCR Cα.

SEQ ID NO.: 5 is an amino acid sequence encoded by a “full-scFv” TCR/CAR coding construct (general design of Cβ_VH-linker-VL-Cα). It includes, from N-terminal end to C-terminal end: a GM-CSF leader sequence; TCR Cβ; a furin cleavage sequence; a P2A peptide with N-terminal GSG linker; a GM-CSF leader sequence; VH of FMC63; a linker; VL of FMC63; and TCR Cα.

SEQ ID NO.: 6 is an amino acid sequence encoded by a “full-scFv” TCR/CAR coding construct (general design of VH-linker-VL-Cβ_Cα). It includes, from N-terminal end to C-terminal end: a GM-CSF leader sequence; VH of FMC63; a linker; VL of FMC63; TCR Cβ; a furin cleavage sequence; a P2A peptide with N-terminal GSG linker; a GM-CSF leader sequence; and TCR Cα.

SEQ ID NO.: 9 is an amino acid sequence encoded by a “bispecific full-scFv” TCR/CAR coding construct (general design of VL-linker-VH-TCR Cβ_VH-linker-VL_Cα). It includes, from N-terminal end to C-terminal end: a GM-CSF leader sequence; VL of a GPRCSD-specific antibody; a linker; VH of the GPRC5D-specific antibody; TCR Cβ; a furin cleavage sequence; a P2A peptide with N-terminal GSG linker; a GM-CSF leader sequence; VH of a BCMA-specific antibody; a linker; VL of the BCMA-specific antibody; TCR Cα; a furin cleavage sequence; a P2A peptide with N-terminal GSG linker; a GM-CSF leader sequence; and a truncated EGFR. The truncated EGFR functions as a marker of transduction; other transduction markers are known and may be utilized; e.g. tCD19, tCD34, or tNGFR (also referred-to as CD19t, CD34t, and NGFRt, respectively).

SEQ ID NO.: 10 is an amino acid sequence encoded by a nanobody-based TCR/CAR-coding construct (general design of TCR Cβ_VHH-linker-VHH-TCR Cα). It includes, from N-terminal end to C-terminal end: GM-CSF leader sequence; TCR Cβ; a furin cleavage sequence; a P2A peptide with N-terminal GSG linker; GM-CSF leader sequence; a first copy of a RORI-specific VHH; a three-alanine linker; a second copy of the ROR1-specific VHH; TCR Cα.

SEQ ID NO.: 11 is an amino acid sequence encoded by a nanobody-based TCR/CAR-coding construct (general design of VHH-linker-VHH-TCR Cβ_TCR Cα). It includes, from N-terminal end to C-terminal end: GM-CSF leader sequence; a first copy of a RORI-specific VHH; a three-alanine linker; a second copy of the ROR1-specific VHH, TCR Cβ; a furin cleavage sequence; a P2A peptide with N-terminal GSG linker; GM-CSF leader sequence; TCR Cα.

SEQ ID NO.: 12 is an amino acid sequence encoded by a nanobody-based TCR/CAR-coding construct (general design of VHH-linker-VHH-TCR Cβ_VHH-linker-VHH-TCR Cα). It includes, from N-terminal end to C-terminal end: GM-CSF leader sequence; a first copy of a VHH (“MB14”); a three-alanine linker; a second copy of the ROR1-specific VHH; TCR Cβ; a furin cleavage sequence; a P2A peptide with N-terminal GSG linker; GM-CSF leader sequence; a third copy of the VHH; a three-alanine linker; a fourth copy of the VHH; TCR Cα.

SEQ ID NO.: 13 is an amino acid sequence encoded by a Bc12-based TCR/CAR-coding construct (general design of TCR Cβ_Bcl2-linker-hinge-TCR Cα). It includes, from N-terminal end to C-terminal end: GM-CSF leader sequence; TCR Cβ; a furin cleavage sequence; a P2A peptide with N-terminal GSG linker; a murine IgG, kappa leader sequence; a mini-FLAG sequence; a HA-Tag sequence; a GS linker; a Bcl2 ectodomain sequence; a (G₄S)₂ linker; a hinge; TCR Cα.

SEQ ID NO.: 14 is an amino acid sequence encoded by a Bcl2-based TCR/CAR-coding construct (general design of Bcl2-linker-hinge-TCR Cβ_TCR Cα). It includes, from N-terminal end to C-terminal end: a murine IgG, kappa leader sequence; a mini-FLAG sequence; a HA-Tag sequence; a GS linker; a Bel2 ectodomain sequence; a (G₄S)₂ linker; a hinge; TCR Cβ; a furin cleavage sequence; a P2A peptide with N-terminal GSG linker; a GM-CSF leader sequence; TCR Cα.

SEQ ID NO.: 15 is an amino acid sequence encoded by a Bc12-based TCR/CAR-coding construct (general design of Bcl2-linker-hinge-TCR Cβ_Bcl2-linker-hinge-TCR Cα). It includes, from N-terminal end to C-terminal end: a murine IgG, kappa leader sequence; a mini-FLAG sequence; a HA-Tag sequence; a GS linker; a Bcl2_ectodomain; a (G₄S)₂ linker; a hinge; a TCR Cβ; a furin cleavage sequence; a P2A peptide with N-terminal GSG linker; a murine IgG, kappa leader sequence; a mini-FLAG sequence; a myc-tag; a GS linker; a Bcl2 ectodomain; a (G₄S)₂ linker; a hinge; a TCR Cα.

SEQ ID NO.: 24 is an amino acid sequence encoded by a TCR/CAR_CCR-coding construct (general design of VL-TCR Cβ_CD226-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a CD226 leader sequence, a CD226 ectodomain; a CD226 transmembrane domain; a CD226 endodomain comprising K295A and K333A mutations; a furin cleavage sequence; a P2A peptide with N-terminal GSG linker; a GM-CSF leader sequence; VH from FMC63; TCR Cα.

SEQ ID NO.: 25 is an amino acid sequence encoded by a TCR/CAR_CCR-coding construct (general design of VL-TCR Cβ_CD226-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a CD226 leader sequence; a CD226 ectodomain sequence; a CD226 transmembrane domain sequence; a CD2 endodomain sequence; a furin cleavage sequence; a P2A peptide with N-terminal GSG linker; a GM-CSF leader sequence; VH from FMC63; TCR Cα.

SEQ ID NO.: 26 is an amino acid sequence encoded by a TCR/CAR_CCR-coding construct (general design of VL-TCR Cβ_CD226-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a CD226 leader sequence; a CD226 ectodomain sequence; a CD226 transmembrane domain sequence; a truncated CD2 endodomain sequence; a furin cleavage sequence; a P2A peptide with N-terminal GSG linker; a GM-CSF leader sequence; VH from FMC63; TCR Cα.

SEQ ID NO.: 27 is an amino acid sequence encoded by a TCR/CAR_CCR-coding construct (general design of VL-TCR Cβ_CD226-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a CD226 leader sequence; a CD226 ectodomain sequence; a CD226 transmembrane domain sequence; a CD28 endodomain sequence; a furin cleavage sequence; a P2A peptide with N-terminal GSG linker; a GM-CSF leader sequence; VH from FMC63; TCR Cα.

SEQ ID NO.: 28 is an amino acid sequence encoded by a TCR/CAR_CCR-coding construct (general design of VL-TCR Cβ_CD226-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a CD226 leader sequence; a CD226 ectodomain sequence; a CD226 transmembrane domain sequence; a 4-1BB endodomain sequence, a furin cleavage sequence; a P2A peptide with N-terminal GSG linker; a GM-CSF leader sequence; VH from FMC63; TCR Cα.

SEQ ID NO.: 29 is an amino acid sequence encoded by a TCR/CAR_CCR-coding construct (general design of VL-TCR Cβ_TIGIT-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a TIGIT leader sequence; a TIGIT ectodomain sequence; a TIGIT transmembrane domain sequence; a CD2 endodomain sequence; a furin cleavage sequence; a P2A peptide with N-terminal GSG linker; a GM-CSF leader sequence; VH from FMC63; TCR Cα.

SEQ ID NO.: 30 is an amino acid sequence encoded by a TCR/CAR_CCR-coding construct (general design of VL-TCR Cβ_TIGIT-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a TIGIT leader sequence; a TIGIT ectodomain sequence; a TIGIT transmembrane domain sequence; a truncated CD2 endodomain sequence; a furin cleavage sequence; a P2A peptide with N-terminal GSG linker; a GM-CSF leader sequence; VH from FMC63; TCR Cα.

SEQ ID NO.: 31 is an amino acid sequence encoded by a TCR/CAR_CCR-coding construct (general design of VL-TCR Cβ_TIGIT-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker; a TIGIT leader sequence; a TIGIT ectodomain sequence; a TIGIT transmembrane domain sequence; a CD28 endodomain sequence; a furin cleavage sequence; a P2A peptide with N-terminal GSG linker; a GM-CSF leader sequence; VH from FMC63; TCR Cα.

SEQ ID NO.: 32 is an amino acid sequence encoded by a TCR/CAR_CCR-coding construct (general design of VL-TCR Cβ_TIGIT-based CCR_VH-TCR Cα). It includes, from N-terminal end to C-terminal end: a GM-CSF leader sequence; VL from FMC63; TCR Cβ; a furin cleavage sequence; a P2A peptide with N-terminal GSG linker, a TIGIT leader sequence; a TIGIT ectodomain sequence; a TIGIT transmembrane domain sequence; a 4-1BB endodomain sequence; a furin cleavage sequence; a P2A peptide with N-terminal GSG linker; a GM-CSF leader sequence; VH from FMC63; TCR Cα.

In some embodiments, a polynucleotide of the present disclosure encodes an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 1-6, 9-15, and 24-32. It will be appreciated that other self-cleaving peptide sequences, leader sequences, furin cleavage sequences, and arrangements of coding sequences are contemplated.

A polynucleotide (e.g., encoding a TCR/CAR, a polypeptide of a TCR/CAR, a CCR, or any combination thereof) can be comprised in a vector, such as an expression vector comprising a lentiviral vector or a retroviral vector.

Also provided are fusion polypeptides that comprise a binding-domain-containing TCR/CAR polypeptide of the present disclosure. Any of the presently disclosed first or second TCR/CAR polypeptides may be provided as an isolated polypeptide, provided that the polypeptide comprises a binding domain, and not accompanied by a cognate TCR/CAR polypeptide. Polynucleotides and vectors that encode the fusion polypeptides are also provided.

Also provided are host cells that express a presently disclosed TCR/CAR, a presently disclosed CCR, or both. Also provided are host cells that comprise a polynucleotide or vector encoding a a presently disclosed TCR/CAR, a presently disclosed CCR, or both. A host cell expressing or encoding a CCR may further express or encode a TCR or a CAR. In certain embodiments, a host cell comprises a hematopoietic progenitor cell, a hematopoeitic stem cell, or an immune system cell, such as a human immune system cell. In certain embodiments, an immune system cell comprises a T cell, a NK-T cell, or a macrophage. In certain embodiments, a T cell comprises a CD4+ T cell, a CD8+ T cell, a CD4−CD8− double negative T cell, an αβ+ T cell, a γδ+ T cell, or any combination thereof. In certain embodiments, a T cell comprises a naïve T cell, a central memory T cell, a stem cell memory T cell, an effector memory T cell, or any combination thereof.

In some embodiments, a host cell comprises a chromosomal knockout of TIGIT, of a TCR locus (e.g. TRAC, TRBC), of a CD8 locus, of a CD4 locus, of a PD-I locus, of a LAG-3 locus, of a TIM3 locus, of an HLA locus (e.g. a gene that encodes an α1 macroglobulin, an α2 macroglobulin, an α3 macroglobulin, a β1 microglobulin, or a β2 microglobulin), of a TGFβR1 locus, of a TGFβR2 locus, of a LAT locus, of an A2AR locus, of a Fas locus, of a FasL locus, of a B7-H3 locus, of a B7-H4 locus, of an IDO locus, of a VISTA locus, of a SIGLEC7 locus, of a SIGLEC9 locus, of a CBLB locus, of a RASA2 locus, of a UBASH3A locus, of a CISH locus, or of any combination thereof. In some embodiments, a host cell expresses a TCR/CAR of the present disclosure and comprises a chromosomal knockout of the target(s) bound by the TCR/CAR. For example, in some embodiments, a host cell expresses a TCR/CAR that binds SLAMF7 and comprise a chromosomal knockout of a SLAMF7 locus. In some embodiments, a host cell expresses a TCR/CAR that binds CD229 and comprise a chromosomal knockout of a CD229 locus. In certain embodiments, a host cell expresses a TCR/CAR of the present disclosure is a T cell and comprises (1) a chromosomal knockout of the target(s) bound by the TCR/CAR and (2) a chromosomal knockout of a CD8 locus and/or of a CD4 locus.

Non-limiting examples of sgRNA sequences targeting chromosomal TRAC and TRBC sequences are provided in SEQ ID NOs.: 16-23. Non-limiting examples of sgRNA sequences targeting chromosomal TIGIT sequences are provided in SEQ ID NOs.: 33-41.

Also provided is a T cell comprising a chromosomal gene knockout of a CD4 gene locus and/or of a CD8 gene locus. In certain embodiments, the T cell expresses a CAR or a TCR. In some embodiments, the TCR or CAR is capable of CD4-independent binding to an antigen: MHC complex. In some embodiments, the TCR or CAR is is capable of CD8-independent binding to an antigen: MHC complex.

Compositions that comprise the host cells (including any combination thereof), polynucleotides, TCR/CARs, fusion polypepties, or vectors, and optionally a pharmaceutically acceptable carrier, excipient, or diluent, are also provided. Also provided are methods of making a host cell, wherein the methods comprise introducing a polynucleotide or vector encoding (i) a TCR/CAR of the present disclosure and/or (ii) a CCR of the present disclosure. Also provided are methods of making a host cell, wherein the methods comprise generating chromosomal CD4 or CD8 gene knockout in a T cell that comprises a polynucleotide encoding a CAR or a TCR, or in a T cell into which is to be introduced a polynucleotide encoding a CAR or a TCR. Also provided are methods of using any of the presently disclosed TCR/CARs, CCRs, fusion polypeptides, polynucleotides, vectors, host cells, and compositions to treat a disease or disorder, such as, for example, a cancer, such as, for example, a solid cancer or a hematological malignancy.

Also provided are methods of using any of the presently disclosed TCR/CARs, CCRs, fusion polypeptides, polynucleotides, vectors, host cells, and compositions in the preparation of a medicament to treat a disease or disorder, such as, for example, a cancer, such as, for example, a solid cancer or a hematological malignancy.

An extracellular component and an intracellular component of a polypeptide of the present disclosure are connected by a transmembrane domain. In some contexts, a “transmembrane domain” is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In certain embodiments, the transmembrane domain of a target-binding protein comprises or is derived from a known transmembrane protein (e.g., a CD4 transmembrane domain, a CD8 transmembrane domain, a CD27 transmembrane domain, a CD28 transmembrane domain, or any combination thereof), and can be a functional portion or variant thereof; i.e., that retains or substantially retains a three-dimensional structure that is thermodynamically stable in a cell membrane and generally having a length from about 15 amino acids to about 30 amino acids. Preferably, TCR/CARs comprise (e.g. retain) the transmembrane domains of their respective TCR constant domains.

An intracellular component of a CCR can comprise a costimulatory domain or a functional portion or variant thereof.

In certain embodiments, the intracellular component of a CCR comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In certain embodiments, the intracellular component comprises a CD28 costimulatory domain or a functional portion or variant thereof (which may optionally include a LL→GG mutation at positions 186-187 of the native CD28 protein (see Nguyen et al., Blood 102:4320, 2003)), a 4-1BB costimulatory domain or a functional portion or variant thereof, or both.

In certain embodiments, one or more of an extracellular component, a binding domain, a linker, a transmembrane domain, an intracellular component, or a costimulatory domain or functional portion or variant thereof, of a target-binding protein can (or a fusion protein can) further comprise one or more junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix) or a fusion protein of this disclosure. Protein tags can be useful for detecting, identifying, isolating, tracking, purifying, enriching for, targeting, or biologically or chemically modifying tagged proteins of interest, particularly when a tagged protein is part of a heterogeneous population of cell proteins or cells (e.g., a biological sample like peripheral blood). In certain embodiments, a protein tag of a fusion protein or antigen-binding protein of this disclosure comprises a Myc tag, His tag, Flag tag, Xpress tag, Avi tag, Calmodulin tag, Polyglutamate tag, HA tag, Nus tag, S tag, X tag, SBP tag, Softag, VS tag, CBP, GST, MBP, GFP, Thioredoxin tag, Strep tags (e.g., Strep-Tag; Strep-Tag II; and variants thereof, including those disclosed in, for example, Schmidt and Skerra, Nature Protocols, 2:1528-1535 (2007), U.S. Pat. No. 7,981,632; and PCT Publication No. WO 2015/067768, the strep-tag peptides, step-tag-peptide-containing polypeptides, and sequences of the same, are incorporated herein by reference), or any combination thereof.

Methods for making fusion proteins are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; Brentjens et al., 2007, Clin. Cancer Res. 13:5426, and Walseng et al., Scientific Reports 7:10713, 2017, the techniques of which are herein incorporated by reference.

Methods useful for isolating and purifying recombinantly produced soluble fusion proteins and/or target-binding proteins, by way of example, may include obtaining supernatants from suitable host cell/vector systems that secrete the recombinant soluble fusion protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble fusion protein described herein include batch cell culture, which is monitored and controlled to maintain appropriate culture conditions. Purification of the soluble fusion protein may be performed according to methods described herein and known in the art and that comport with laws and guidelines of domestic and foreign regulatory agencies.

TCR/CARs and CCRs as described herein may be functionally characterized according to any of a large number of art-accepted methodologies for assaying host cell activity. For example, in the case of a host T cell, target-binding proteins can be functionally characterized by determination of T cell binding, activation or induction, as well as determination of T cell responses that are target (e.g., antigen)-specific. Examples include determination of T cell proliferation, T cell cytokine release, target-specific T cell stimulation, MHC-restricted T cell stimulation, CTL activity (e.g., by detecting ⁵¹Cr or Europium release from pre-loaded target cells), changes in T cell phenotypic marker expression, and other measures of T-cell functions. Procedures for performing these and similar assays are found, for example, in Lefkovits (Immunology Methods Manual: The Comprehensive Sourcebook of Techniques, 1998). See, also, Current Protocols in Immunology, Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an target-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL~13.

### Polynucleotides, Vectors, and Host Cells

In certain aspects, nucleic acid molecules (also referred-to as polynucleotides) are provided that encode any one or more of the polypeptide dimers (TCR/CARs), polypeptides (subunit of a TCR/CAR), or fusion polypeptides (CCR) as described herein., or any combination thereof. In some embodiments, a polynucleotide encodes, in 5′ to 3′ direction: [TCR/CAR Cβ-containing polypeptide-TCR/CAR Cα-containing polypeptide]; [TCR/CAR Cα-containing polypeptide-TCR/CAR Cβ-containing polypeptide]; [TCR/CAR Cα-containing polypeptide-CCR-TCR/CAR Cβ-containing polypeptide], [TCR/CAR Cβ-containing polypeptide-CCR-TCR/CAR Cα-containing polypeptide]; [CCR-TCR/CAR Cβ-containing polypeptide-TCR/CAR Cα-containing polypeptide]; [CCR-TCR/CAR Cα-containing polypeptide-TCR/CAR Cβ-containing polypeptide]; [TCR/CAR Cβ-containing polypeptide-TCR/CAR Cα-containing polypeptide-CCR]; or [TCR/CAR Cα-containing polypeptide-TCR/CAR Cβ-containing polypeptide-CCR]. It will be appreciated that polynucleotide sequences encoding the polypeptides can be separated by polynucleotides encoding: a furin or other protease cleavage site, a self-cleaving peptide, or both.

A polynucleotide encoding a desired fusion protein or polypeptide(s) of this disclosure can be inserted into an appropriate vector (e.g., viral vector or non-viral plasmid vector) for introduction into a host cell of interest (e.g., an immune cell, such as a T cell). A polynucleotide can further encode additional features, as described herein.

Exemplary markers (e.g., for transduction of a cell with a polynucleotide as provided herein) include green fluorescent protein, an extracellular domain of human CD2, a truncated human EGFR (huEGFRt, (see Wang et al., Blood 118:1255, 2011), a truncated human CD19 (huCD19t); a truncated human CD34 (huCD34t); or a truncated human NGFR (huNGFRt). In certain embodiments, an encoded marker comprises EGFRt, CD19t, CD34t, or NGFRt.

In any of presently disclosed embodiments, a protein-encoding polynucleotide can further comprise a polynucleotide that encodes a marker and a polynucleotide that encodes a self-cleaving polypeptide, wherein the polynucleotide encoding the self-cleaving polypeptide is located between the polynucleotide encoding the protein and the polynucleotide encoding the marker. When the protein-encoding polynucleotide, marker-encoding polynucleotide, and self-cleaving polypeptide are expressed by a host cell, the (fusion or antigen-binding) protein and the marker will be present on the host cell surface as separate molecules. In certain embodiments, a self-cleaving polypeptide comprises a 2A peptide from porcine teschovirus-1 (P2A, Thoseaasigna virus (T2A, equine rhinitis A virus (E2A), or foot-and-mouth disease virus (F2A)). Exemplary nucleic acid and amino acid sequences of 2A peptides are set forth in, for example, Kim et al. (PLOS One 6: e18556, 2011, which 2A nucleic acid and amino acid sequences are incorporated herein by reference in their entirety),

In any of the presently disclosed embodiments, a self-cleaving polypeptide encoded by a polynucleotide of this disclosure comprises a P2A, a T2A, an E2A, or a F2A. A self-cleaving peptide can comprise a short linker sequence (e.g., G-S-G) disposed at the N-terminal end thereof.

In any of the embodiments described herein, a polynucleotide of the present disclosure may be codon-optimized for expression in a host cell (see, e.g, Scholten et al., Clin. Immunol. 119:135-145 (2006). Codon optimization can be performed using known techniques and tools, e.g., using the GenScript® Optimum Gene™ tool, or the GeneArt™/GeneOptimizer™ tools. Codon-optimized sequences include sequences that are partially codon-optimized (i.e., one or more of the codons is optimized for expression in the host cell) and those that are fully codon-optimized.

In certain embodiments, polynucleotide encoding a polypeptide dimer, polypeptide, or fusion polypeptide further comprises a polynucleotide encoding a leader or signal sequence. An exemplary leader amino acid sequence is from GM-CSF, CD8α, or murine IgG kappa light chain.

In further aspects, expression constructs are provided, wherein the expression constructs comprise a polynucleotide of the present disclosure operably linked to an expression control sequence (e.g., a promoter). An exemplary promoter sequence includes an EF1a promoter or a MNDu3 promoter. In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus (e.g., adeno-associated viruses), parvovirus, coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLOS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009 Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Construction of an expression vector that is used for genetically engineering and producing a polypeptide dimer, polypeptide, or fusion polypeptide of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell). A host cell encoding and/or expressing a fusion protein as disclosed herein is, in certain embodiments, useful in adoptive transfer therapy (e.g., targeting a cancer antigen or targeting an adoptively transferred cell that expresses a tag peptide). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to fusion proteins of the present disclosure.

In certain embodiments, the host cell is a hematopoietic progenitor cell or a human immune system cell. A “hematopoietic progenitor cell”, as referred to herein, is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4⁻ CD8⁻ double negative T cell, a γδ T cell, a regulatory T cell, a stem cell memory T cell, a natural killer cell (e.g., a NK cell or a NK-T cell), a B cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs), though it will be understood that a T cell in which expression of a native TCR is (e.g., artificially) suppressed or abrogated is still a T cell. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD45RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refer to antigen-experienced CD8⁺ cytotoxic T lymphocytes that have decreased expression of CD62L, CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺CD25⁺ (Foxp3⁺) regulatory T cells and Treg17 cells, as well as Tr1, Th3, CD8⁺CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells.

In certain embodiments, the immune system cell is a CD4+ T cell, a CD8+ T cell, a CD4− CD8− double negative T cell, a γδ T cell, a natural killer cell (e.g., NK cell or NK-T cell), a dendritic cell, a B cell, or any combination thereof. In certain embodiments, the immune system cell is a CD4+ T cell. In certain embodiments, the T cell is a naïve T cell, a central memory T cell, an effector memory T cell, a stem cell memory T cell, or any combination thereof.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, a host cell that comprises a heterologous polynucleotide encoding a polypeptide dimer, polypeptide, or fusion polypeptide can be an immune cell which is modified to reduce or eliminate expression of one or more endogenous genes that encode a polypeptide product selected from a CD4 gene locus, a CD8 gene locus, a TGFβR1 gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, or any combination thereof. In some embodiments, a host cell is modified to reduce or eliminate expression of: TIGIT and one or both of TRAC and TRBC; TIGIT and one or both of CD4 and CD8; one or both of TRAC and TRBC and one or both of CD4 and CD8; or TIGIT, one or both of TRAC and TRBC, and one or both of CD4 and CD8.

Without wishing to be bound by theory, certain endogenously expressed immune cell proteins may downregulate the immune activity of a modified immune host cell (e.g., PD-1, LAG-3, CTLA4, TIGIT, CBLB, RASA2, UBASH3A, CISH, Fas), or may compete with a disclosed polypeptide dimer, polypeptide, or fusion polypeptide for resources (e.g. Lck), or any combination thereof. Further, endogenous proteins (e.g., immune host cell proteins, such as an HLA) expressed on a donor immune cell to be used in a cell transfer therapy may be recognized as foreign by an allogeneic recipient, which may result in elimination or suppression of the donor immune cell by the allogeneic recipient.

Accordingly, decreasing or eliminating expression or activity of such endogenous genes or proteins can improve the activity, tolerance, and persistence of the host cells in an autologous or allogeneic host setting, and can allow universal administration of the cells (e.g., to any recipient regardless of HLA type). In certain embodiments, a modified host immune cell is a donor cell (e.g., allogeneic) or an autologous cell. In certain embodiments, a modified immune host cell of this disclosure comprises a chromosomal gene knockout of one or more of a gene that encodes PD-1, LAG-3, CTLA4, TIM3, TIGIT, CD4, CD8, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119 (24): 5697 (2012); and Torikai et al., Blood 122 (8): 1341 (2013) the gene editing techniques, compositions, and adoptive cell therapies of which are herein incorporated by reference in their entirety), TGFβR1, TGFβR2, LAT, A2AR, Fas, FasL, B7-H3, B7-H4, IDO, VISTA, SIGLEC7, SIGLEC9, TRAC, TRBC, CBLB, RASA2, UBASH3A, and/or CISH. As used herein, the term “chromosomal gene knockout” refers to a genetic alteration in a bost cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZEN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crR.NA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system or base editing system (Komor, A. C.; Kim, Y. B.; Packer, M. S.; Zuris, J. A.; Liu, D. R. Nature 533, 420-424 (2016). Briefly, base editing is a genome-editing approach that uses components from CRISPR systems together with other enzymes to directly introduce point mutations into cellular DNA or RNA without making double-stranded DNA breaks. Certain DNA base editors comprise a catalytically disabled nuclease fused to a nucleobase deaminase enzyme and, in some cases, a DNA glycosylase inhibitor. RNA base editors function similarly, using components that target RNA. Base editors directly convert one base or base pair into another, enabling the efficient installation of point mutations in non-dividing cells without generating excess undesired editing by-products. See e.g. Rees H et al. Nature Reviews Genetics (2018).

Exemplary gRNA sequences and methods of using the same to knock out endogenous genes that encode immune cell proteins include those described in Ren et al., Clin. Cancer Res. 23 (9): 2255-2266 (2017), the gRNAs, CAS9 DNAs, vectors, and gene knockout techniques of which are hereby incorporated by reference in their entirety.

Alternative Cas nucleases may be used, including but not limited to, Cas 12, Cas 13, and Cas 14 nucleases, and variants thereof. For example, Cas nucleases disclosed in WO 2019/178427, which is hereby incorporated by reference in its entirety (including the Cas nucleases, CRISPR-Cas systems, and related methods disclosed therein), may be utilized.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HINH, His-Cys box and PD-(D/E) XK. Exemplary meganucleases include I-Scel, I-Ceul, PI-PspI, PI-Sce, I-SceIV, I-CsmI, I-PanI, I-Scell, I-PpoI, I-SceIII, I-Crel, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, an HLA-encoding gene, a CD4, a CD8, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

A chromosomal gene knockout can be performed using base-editing, as known in the art and described herein.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4, TIGIT, an HLA component, or a TCR component, a CD4, a CD8, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

Any of the foregoing gene-editing techniques can be used to introduce a polynucleotide of the present disclosure (e.g., encoding a fusion protein) into a host cell genome. In some embodiments, a heterologous polynucleotide is introduced into a locus encoding an endogenous TCR component, HLA component, PD-1, LAG-3, CTLA4, TIM3, or TIGIT, or a “safe harbor” locus such as Rosa26, AAVS1, CCR5, or the like.

In certain embodiments, a host cell (e.g., immune cell) of the present disclosure is engineered so that expression of polypeptide dimer, polypeptide, or fusion polypeptide is modulated (e.g., controlled) by binding of the host cell to a target (e.g. antigen) that is not the same target as the target to which the polypeptide dimer, polypeptide, or fusion polypeptide, respectively, binds.

For example, a host cell can comprise (i) a polynucleotide encoding an engineered (i.e., synthetic) Notch receptor comprising (a) an extracellular component comprising a binding domain that binds to an antigen, which is a different antigen than the antigen to which the antigen-binding protein binds, (b) a Notch core domain, or a functional portion or variant thereof; and (c) an intracellular component comprising a transcriptional factor (i.e., a polypeptide capable of activating or increasing, or inhibiting, repressing or reducing, transcription of a target nucleotide sequence (e.g., a gene) or set of target nucleotide sequences); and (ii) the heterologous polynucleotide encoding a polypeptide dimer, polypeptide, or fusion polypeptide as disclosed herein and comprising an expression control sequence that can be recognized or bound by the transcriptional factor, wherein binding of the engineered Notch receptor to antigen leads to release of the transcriptional factor from the engineered Notch receptor (e.g., by protease-driven cleavage), which can, in turn, drive transcription of the polypeptide dimer, polypeptide, or fusion polypeptide. See, e.g., Morsut et al., Cell 164:780-791 (2016) and PCT Published Application No. WO 2016/138034A1, which synthetic Notch constructs are incorporated herein by reference. Briefly, such “logic-gated” expression systems may be useful to modulate expression of an antigen-binding protein of this disclosure so that the expression occurs only, or preferentially, when the host cell encounters a first antigen (i.e., that can be bound by the synthetic Notch receptor) that is only expressed by, or is principally expressed by, or has a higher expression level on cancer cells as compared to healthy cells. Such embodiments may reduce “on-target off-tissue” recognition by a fusion protein in circumstances where the target recognized by the antigen-binding protein is expressed by healthy cells.

In other aspects, kits are provided comprising (a) a vector or an expression construct as described herein and (b) reagents for transducing the vector or the expression construct into a host cell.

### Uses

The present disclosure also provides methods for treating a disease or condition, wherein the methods comprise administering to a subject in need thereof an effective amount of a polypeptide dimer, polypeptide, fusion polypeptide, polynucleotide, vector, host cell, composition, or unit dose of the present disclosure. In some embodiments, the the disease or condition expresses or is otherwise associated with the target (e.g., antigen).

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre malignant cells, as well as non-neoplastic or non-malignant disorders.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In certain embodiments, a cancer treatable according to the presently disclosed methods and uses comprises a carcinoma, a sarcoma, a glioma, a lymphoma, a leukemia, a myeloma (such as, for example, multiple myeloma), or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In any of the presently disclosed embodiments, the host cell is an allogeneic cell, a syngeneic cell, or an autologous cell. Typically, the host cell will further express or encode an antigen-binding protein. Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a fusion protein as described herein is administered intravenously, intraperitoneally, intratumorally, into the bone marrow, into a lymph node, or into the cerebrospinal fluid so as to encounter the tagged cells to be ablated. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In any of the above embodiments, methods of the present disclosure comprise administering a host cell expressing a polypeptide dimer, polypeptide, or fusion polypeptide of the present disclosure. The amount of cells in a composition is at least one cell (for example, one fusion protein-modified CD8⁺ T cell subpopulation; one fusion protein-modified CD4⁺ T cell subpopulation) or is more typically greater than 10² cells, for example, up to 10⁶, up to 10⁷, up to 10⁸ cells, up to 10⁹ cells, or more than 10¹⁰ cells, such as about 10¹¹ cells/m². In certain embodiments, the cells are administered in a range from about 10⁵ to about 10¹¹ cells/m², preferably in a range of about 10⁵ or about 10⁶ to about 10⁹ or about 10¹⁰ cells/m². The number of cells will depend upon the ultimate use for which the composition is intended as well the type of cells included therein. For example, cells modified to contain a fusion protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁵, 10⁶, 10⁷, 10⁸, 10⁹, 10¹⁰, or 10¹¹ cells.

Unit doses are also provided herein which comprise a host cell (e.g., a modified immune cell comprising a polynucleotide of the present disclosure) or host cell composition of this disclosure. Typically, the host cell will further express or encoden an antigen-binding protein. In certain embodiments, a unit dose comprises (i) a composition comprising at least about 30% (e.g., including 30% or more), at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio (e.g., such as a 1:1 ratio), wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of engineered CD45RA CD3⁺CD8⁺ and engineered CD45RA⁻ CD3⁺CD4⁺ T_{M} cells.

Also contemplated are pharmaceutical compositions that comprise fusion proteins or cells expressing or encoding a fusion protein as disclosed herein, and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the fusion protein-expressing cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with the target (e.g., antigen). Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell comprises a hematopoietic progenitor cell or a human immune cell. In certain embodiments, the immune system cell comprises a CD4⁺ T cell, a CD8⁺ T cell, a CD4⁻ CD8⁻ double-negative T cell, a γδ T cell, a natural killer cell, a dendritic cell, or any combination thereof. In certain embodiments, the immune system cell comprises a naïve T cell, a central memory T cell, a stem cell memory T cell, an effector memory T cell, or any combination thereof. In particular embodiments, the cell comprises a CD4⁺ T cell. In particular embodiments, the cell comprises a CD8⁺ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., fusion protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks or more. In certain embodiments, the plurality of unit doses are administered at intervals between administrations of about two, three, four, five, six, seven, eight, or more weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition (e.g., host cell, fusion protein, unit dose, or composition) refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

Target (e.g., antigen)-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a fusion protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁵ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

In further aspects, kits are provided that comprise (a) a host cell, (b) a composition, or (c) a unit dose as described herein.

Methods according to this disclosure may further include administering one or more additional agents to treat the disease or disorder in a combination therapy. For example, in certain embodiments, a combination therapy comprises administering a polypeptide dimer, polypeptide, or fusion polypeptide (or an engineered host cell expressing the same) with (concurrently, simultaneously, or sequentially) an immune checkpoint inhibitor. In some embodiments, a combination therapy comprises administering fusion protein of the present disclosure (or an engineered host cell expressing the same) with an agonist of a stimulatory immune checkpoint agent. In further embodiments, a combination therapy comprises administering a polypeptide dimer, polypeptide, or fusion polypeptide of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVRIG (CD112R), PVRL2, adenosine, A2aR, immunosuppressive cytokines (e.g., IL-10, IL-4, IL-IRA, IL-35), IDO, arginase, VISTA, TIGIT, LAIRI, CEACAM⁻¹, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a composition of the present disclosure (e.g., a fusion protein, polynucleotide, vector, an host cell, or pharmaceutical composition) with one or more inhibitor of any one of the following immune suppression components, singly or in any combination.

In certain embodiments, a composition is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558, or any combination thereof. In further embodiments, a composition is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a composition is used in combination with a LAG3 inhibitor, such as LAG525, IMP321, IMP701, 9H12, BMS-986016, or any combination thereof.

In certain embodiments, a composition is used in combination with an inhibitor of CTLA4. In particular embodiments, a composition is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a composition is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a composition is used in combination with an inhibitor of CD244. In certain embodiments, a composition is used in combination with an inhibitor of BLTA, HVEM, CD160, or any combination thereof. Anti CD-160 antibodies are described in, for example, PCT Publication No. WO 2010/084158. In certain embodiments, a composition is used in combination with an inhibitor of TIM3. In certain embodiments, a composition is used in combination with an inhibitor of Gal9. In certain embodiments, a composition is used in combination with an inhibitor of adenosine signaling, such as a decoy adenosine receptor. In certain embodiments, a composition is used in combination with an inhibitor of A2aR. In certain embodiments, a composition is used in combination with an inhibitor of KIR, such as lirilumab (BMS-986015). In certain embodiments, a composition is used in combination with an inhibitor of an inhibitory cytokine (typically, a cytokine other than TGFβ) or Treg development or activity. In certain embodiments, a composition is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof. In certain embodiments, a composition is used in combination with an arginase inhibitor, such as N (omega)-Nitro-L-arginine methyl ester (L-NAME), N-omega-hydroxy-nor-l-arginine (nor-NOHA), L-NOHA, 2 (S)-amino-6-boronohexanoic acid (ABH), S-(2-boronoethyl)-L-cysteine (BEC), or any combination thereof. In certain embodiments, a composition of the present disclosure (or an engineered host cell expressing the same) is used in combination with an inhibitor of VISTA, such as CA-170 (Curis, Lexington, Mass.). In certain embodiments, a composition is used in combination with an inhibitor of TIGIT such as, for example, COM902 (Compugen, Toronto, Ontario Canada), an inhibitor of CD155, such as, for example, COM701 (Compugen), or both. In certain embodiments, a composition is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a composition is used in combination with a LAIRI inhibitor. In certain embodiments, a composition is used in combination with an inhibitor of CEACAM⁻¹, CEACAM-3, CEACAM-5, or any combination thereof. In certain embodiments, a composition is used in combination with an agent that increases the activity (i.e., is an agonist) of a stimulatory immune checkpoint molecule. For example, a composition of the present disclosure can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition with one or more agonist of a stimulatory immune checkpoint molecule, including any of the foregoing, singly or in any combination.

In certain embodiments, a combination therapy comprises a composition and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a composition and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering composition and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42 (4): 539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

In certain embodiments, the subject is receiving, has received, or will receive one or more of: (i) chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.

Also provided herein are uses of any of the presently disclosed polypeptide dimers, polypeptides, fusion polypeptides, polynucleotides, vectors, host cells, compositions, or unit doses, for use in the treatment of a disease or disorder in a subject, wherein the disease or condition is optionally characterized by the presence of an antigen (e.g. that is bound by the binding domain of an antigen-binding protein such as expressed in a host cell) (e.g., any target as disclosed herein).

Also provided herein are uses of any of the presently disclosed polypeptide dimers, polypeptides, fusion polypeptides, polynucleotides, vectors, host cells, compositions, or unit doses, for use in the manufacture of a medicament for the treatment of a disease or condition in a subject.

The present disclosure also provides the following non-limiting enumerated Embodiments.
Embodiment 1. A polypeptide dimer comprising (i) a first polypeptide comprising a first T cell receptor (TCR) constant domain and (ii) a second polypeptide comprising a second TCR constant domain, wherein the first TCR constant domain and the second TCR constant domain associate with one another (e.g., share one or more disulfide bond), and wherein first polypeptide, the second polypeptide, or both, further comprises a target-binding domain disposed amino-terminal to the first or the second TCR constant domain, respectively, wherein the target-binding domain does not comprise a TCR variable domain.
Embodiment 2. The polypeptide dimer of Embodiment 1, wherein the target-binding domain comprises: (i) an antibody heavy chain variable domain (VI); (ii) an antibody light chain variable domain (VL); (iii) a single-chain variable fragment (scFv); (iv) a fragment antigen-binding region (Fab); (v) a single-chain Fab; (vi) an antigen-binding fragment of a heavy chain-only antibody (VHH); (vii) a designed ankyrin repeat protein (DARPin); (viii) a ¹⁰FNIII domain; (ix) a lectin binding domain; (x) a protein ligand-binding domain, such as a receptor ectodomain or a functional portion or fragment thereof; (xi) a killer immunoreceptor from a NK cell; (xii) a fibrinogen domain; (xiii) a cysteine-knot miniprotein; (xiv) a tetratricopeptide repeat domain; (xv) a leucine-rich repeat domain; (xvi) a lipocalin domain; (xvii) an armadillo repeat protein; (xviii) an affibody; (xix) an avimer; (xx) a knottin; (xxi) a fynomer; (xxii) an atrimer; (xxiii) CTLA4; (xxiv) a synthetic protein designed to bind a natural ligand; (xxv) a ligand, such as for example a cytokine or a peptide tag; (xxvi) a centyrin; (xxvii) a VNAR; or (xxviii) any combination of (i)-(xxvii).
Embodiment 3. The polypeptide dimer of Embodiment 1 or Embodiment 2, wherein: (a) the first TCR constant domain comprises a TCR alpha-chain constant domain (Cα) and the second TCR constant domain comprises a TCR beta-chain constant domain (Cβ); (b) the first TCR constant domain comprises a Cβ and the second TCR constant domain comprises a Cα; (c) the first TCR constant domain comprises a TCR gamma-chain constant domain (Cγ) and the second TCR constant domain comprises a TCR delta-chain constant domain (C); or (d) the first TCR constant domain comprises a Cô and the second TCR constant domain comprises a CY, wherein, preferably, the first TCR constant domain comprises a Cβ and the second TCR constant domain comprises a Co or the first TCR constant domain comprises a Cα and the second TCR constant domain comprises a Cβ.
Embodiment 4. The polypeptide dimer of any one of Embodiments 1-3, wherein the first TCR constant domain, the second TCR constant domain, or both, comprises an amino acid substitution mutation that promotes pairing between the first TCR constant domain and the second TCR constant domain.
Embodiment 5. The polypeptide dimer of Embodiment 4, wherein the amino acid substitution mutation comprises a cysteine amino acid at a non-native position and the cysteine amino acid forms a disulfide bond with an amino acid at a corresponding position in the other of the second or the first TCR constant domain, respectively.
Embodiment 5a. The polypeptide dimer of any one of Embodiments 1-5, wherein: (i) the first TCR constant domain comprises one or more amino acid substitution to provide a cavity and the second TCR constant domain comprises one or more amino acid substitution to provide a compensatory protuberance; (ii) the second TCR constant domain comprises one or more amino acid substitution to provide a cavity and the first TCR constant domain comprises one or more amino acid substitution to provide a compensatory protuberance; or (iii) the first TCR constant domain and/or the second TCR constant domain comprise one or more amino acid substitutions that create a charge pair to facilitate preferred pairing of the first TCR constant domain with the second TCR constant domain.
Embodiment 6. The polypeptide dimer of any one of Embodiments 1-5a, wherein the first polypeptide or the second polypeptide comprises a TCR Cα domain comprising one or more mutations to improve stability of the polypeptide dimer when expressed at a surface of a host cell, wherein, optionally, the one or more mutations comprise an L-V-L mutation.
Embodiment 7. The polypeptide dimer of any one of Embodiments 1-6, wherein: (1) the first polypeptide comprises a TCR Cα having at least 90%, at least 91%, at least 92%, at least 93%, at least 94%, at least 95%, at least 96%, at least 97%, at least 98%, at least 99%, or 100% identity to any one of SEQ ID NOs.: 56-57, and the second polypeptide comprises a TCR Cp having at least 90%, at least 91%, at least 92%, at least 93%, at least 94%, at least 95%, at least 96%, at least 97%, at least 98%, at least 99%, or 100% identity to any one of SEQ ID NOs.: 58-61; or (2) the first polypeptide comprises a TCR Cβ having at least 90%, at least 91%, at least 92%, at least 93%, at least 94%, at least 95%, at least 96%, at least 97%, at least 98%, at least 99%, or 100% identity to any one of SEQ ID NOs.: 58-61; and the second polypeptide comprises a TCR Cα having at least 90%, at least 91%, at least 92%, at least 93%, at least 94%, at least 95%, at least 96%, at least 97%, at least 98%, at least 99%, or 100% identity to any one of SEQ ID NOs.: 56-57.
Embodiment 8. The polypeptide dimer of any one of Embodiments 1-7, comprising a TCR Cα and a TCR Cβ having at least least 90%, at least 91%, at least 92%, at least 93%, at least 94%, at least 95%, at least 96%, at least 97%, at least 98%, or at least 99% to, or comprising or consisting of, the amino acid sequences set forth in SEQ ID NOs.: (1) 56 and 58, respectively; (ii) 56 and 59, respectively; (iii) 56 and 60, respectively; (iv) 56 and 61, respectively; (v) 57 and 58, respectively; (vi) 57 and 59, respectively; (vii) 57 and 60, respectively; or (viii) 57 and 61, respectively.
Embodiment 9. The polypeptide dimer of any one of Embodiments 3-8, wherein the TCR Cβ comprises the intracellular sequence VKRKDF (SEQ ID NO.: 64) or MAMVKRKDSRG (SEQ ID NO.: 65).
Embodiment 10. The polypeptide dimer of any one of Embodiments 1-9, wherein the first polypeptide, the second polypeptide, or both, comprises an intracellular portion that consists essentially of or consists of the intracellular portion of a native TCR constant domain (e.g. if the polypeptide comprises a TCR Cα, the intracellular portion of the first polypeptide consists essentially of or consists of the intracellular portion of a native TCR Cα; if the polypeptide comprises a TCR Cβ, the intracellular portion of the first polypeptide consists essentially of or consists of the intracellular portion of a native TCR Cβ).
Embodiment 11. The polypeptide dimer of any one of Embodiments 1-10, wherein the first polypeptide, the second polypeptide, or both, does not comprise an intracellular signaling component from a CD3 protein, and/or does not comprise an intracellular costimulatory domain from a costimulatory protein.
Embodiment 12. The polypeptide dimer of any one of Embodiments 1-11, comprising a design according to Table 1.
Embodiment 13. The polypeptide dimer of any one of Embodiments 1-12, wherein the first polypeptide and the second polypeptide comprise, N-terminal to the first TCR constant domain and the second TCR constant domain, respectively: (i) a VH, and a cognate VL; (ii) a VL, and a cognate VH; (iii) a scFv, and no antigen-binding domain; (iv) no antigen-binding domain, and a scFv; (v) VH-CH1, and a cognate VL-CL; (vi) VL-CL, and a cognate VH-CH1; (vii) a scFab, and no antigen-binding domain; (viii) no antigen-binding domain, and a scFab; (ix) a scFv, and a scFv; (x) a scFab, and a scFv; (xi) a scFv, and a scFab; (xii) a VHH, and no antigen-binding domain; (xiii) no antigen-binding domain, and a VHH; (xiv) VHH-linker-VHH, and no binding domain; (xv) VHH-linker-VHH, and VHH-linker-VHH; (xvi) a scFv, and a VHH or a VHH-linker-VHH; (xvii) a VHH or a VHH-linker-VHH, and a scFv; (xvii) a scFab, and a VHH or a VHH-linker-VHH; (xviii) a VHH or a VHH-linker-VHH, and a scFab; (xix) a VHH, and a VHH; (xx) a natural or synthetic protein ligand-binding domain, and no antigen-binding domain; (xxi) no antigen-binding domain, and a natural or synthetic protein ligand-binding domain; (xxii) a scFv, and a natural or synthetic protein ligand-binding domain; (xxiii) a natural or synthetic protein ligand-binding domain, and a scFv; (xxiii) a scFab, and a natural or synthetic protein ligand-binding domain; (xxiv) a natural or synthetic protein ligand-binding domain, and a scFab; (xxv) a VHH or a VHH-linker-VHH, and a natural or synthetic protein ligand-binding domain; (xxvi) a natural or synthetic protein ligand-binding domain, and a VHH or a VHH-linker-VHH; or (xxvii) a natural or synthetic protein ligand-binding domain, and a protein ligand binding-domain.
Embodiment 13a. The polypeptide dimer of any one of Embodiments 1-12, wherein the first polypeptide and the second polypeptide comprise, N-terminal to the first TCR constant domain and the second TCR constant domain, respectively: (i) a VH sufficient for binding to a target, and no binding domain; (ii) a VH sufficient for binding to a target, and a binding domain; (iii) a VL sufficient for binding to a target, and no binding domain; or (iv) a VL sufficient for binding to a target, and a binding domain.
Embodiment 13b. The polypeptide dimer of any one of Embodiments 1-13a, wherein the first polypeptide, the second polypeptide, or both, comprises two or more binding domains, wherein, optionally, the two or more binding domains are different.
Embodiment 14. The polypeptide dimer of any one of Embodiments 1-13b, wherein (1) the first polypeptide comprises a target-binding domain amino-terminal to the first TCR constant domain and the second polypeptide consists essentially of or consists of the second TCR constant domain; or (2) the second polypeptide comprises a target-binding domain amino-terminal to the second TCR constant domain and the first polypeptide consists essentially of or consists of the first TCR constant domain.
Embodiment 15. The polypeptide dimer of any one of Embodiments 1-14, wherein the target is expressed on or by a cancer cell, or is expressed on or by a cell infected with a pathogen (e.g. virus, fungus, parasite, bacteria) or is otherwise associated with an an infection, or is associated with an autoimmune disease or a neurodegenerative disease (e.g., tau, amyloid-beta, alpha-synuclein), or is a cytokine (e.g. TNFα, IL-13, IL-10) or a chemokine.
Embodiment 16. The polypeptide dimer of any one of Embodiments 1-15, wherein the target is expressed by a cancer cell, wherein, optionally, the cancer cell is associated with a solid tumor or a hematological malignancy.
Embodiment 17. The polypeptide dimer of any one of Embodiments 1-16, wherein the target is or comprises a cancer antigen selected from BCMA, GPRCSD, CD19, ROR1, SLAMF7, CD229, PNE, EGFR, EGFRVIII, EGP-2, EGP-40, GD2, GD3, HPV E6, HPV E7, Her2, L1-CAM, Lewis A, Lewis Y, MUC1, MUC16, PSCA, PSMA, CD20, CD22, CD56, CD23, CD24, CD30, CD33, CD37, CD44v7/8, CD3δ, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Carbonic anhydrase 9, BRAF, α-fetoprotein, MAGE-A3, MAGE-A4, SSX-2, PRAME, HA-1, B2M, ETA, tyrosinase, KRAS, NRAS, a peptide: MHC complex, and CEA.
Embodiment 18. The polypeptide dimer of any one of Embodiments 1-17, wherein the polypeptide dimer comprises two or more target-binding domains and is multispecific.
Embodiment 19. The polypeptide dimer of any one of Embodiments 1-18, wherein the polypeptide dimer comprises two or more target-binding domains and is bispecific.
Embodiment 20. The polypeptide dimer of Embodiment 18 or 19, which binds to: (i) BCMA and GPRCSD; (ii) BCMA and SLAMF7; (iii) BCMA and CD229; (iv) GPRC5D and SLAMF7; (v) GPRC5D and CD229; (vi) SLAMF7 and CD229; (vii) CD19 and BCMA; or (viii) CD19 and CD229, wherein, optionally, the polypeptide dimer comprises two scFvs.
Embodiment 21. The polypeptide dimer of any one of Embodiments 1-20, comprising a target-binding domain that comprises the VH, the VL, the HCDRs, and/or the LCDRs of: trastuzumab; pertuzumab, rituximab; erbituxumab; ublituxumab; 1.5.3; a BMCA-specific antibody such as J22.0-xi, J22.9-xi, J6MO, J6M1, J6M2, J9M0, J9M1, J9M2, CA8, A7D12.2, C11 D5.3, C12A3.2, C13F12.1, 13C2, 17A5, 83A10, 13A4, 13D2, 14B11, 14E1, 29B11, 29F3, 13A7, CA7, SGI, S307118G03, S332121F02, S332126E04, S322110D07, S336105A07, S335115G01, S335122F05, ET140-3, ET140-24, ET140-37, ET140-40, ET140-54, TBL-CLNI, C4.E2.1, Vicky-1, pSCHLI333, pSCHLI372, pSCHLI373, and those other BCMA-specific antibodies and antigen-binding fragments disclosed in PCT Publication Nos. WO 2002/066516, WO 2007/062090, WO 2010/104949, WO 2011/108008, WO 2012/163805, WO 2014/068079, WO 2015/166073, WO 2014/122143, WO 2014/089335, WO 2016/090327, WO 2016/079177, Ryan et al., Mol. Cancer. Ther. 6 (11): 3009, 2007, and Abbas et al., Blood 128:1688, 2016; a RORI-specific antibody such as R11, R12, Y4, Y13, Y27, or Y31; a CD19-specific antibody such as FMC63; a CD33-specific antibody such as gemtuzumab; an GPRC5D-specific antibody; a RORI-specific VHH; 3F8; a BCMA-specific VHH; alemtuzumab; XMAB-5574; pembrolizumab; nivolumab; a PD-1-specific antibody; elotuzomab; a SLAMF-specific antibody; a CD229-specific antibody; a PD-LI-specific antibody; or an (e.g. cancer antigen-specific, pathogen-specific, autoimmune disease antigen-specific, or neurodegenerative-disease-specific) antibody or antigen-binding fragment approved for therapeutic and/or diagnostic use in humans by the US Food and Drug Administration, the European Medicines Agency, or both.
Embodiment 22. The polypeptide dimer of any one of Embodiments 1-21, comprising a target-binding domain that comprises a receptor ectodomain from Bcl2, or a portion or variant thereof that is functional to bind Bim.
Embodiment 23. The polypeptide dimer of any one of Embodiments 1-22, comprising a target-binding domain comprising: (i) SEQ ID NO.: 97 and SEQ ID NO.: 98, optionally comprised in a scFv, such as having the sequence of SEQ ID NO.: 99 or SEQ ID NO.: 100; (ii) SEQ ID NO.: 101 and 102, optionally in a scFv, such as having the sequence of SEQ ID NO.: 103; (iii) SEQ ID NO.: 104 and SEQ ID NO.: 105, optionally in a scFv, such as having the sequence of SEQ ID NO.: 106; (iv) SEQ ID NO.: 107, optionally comprsed in SEQ ID NO.: 108; or (v) SEQ ID NO.: 109.
Embodiment 24. The polypeptide dimer of any one of Embodiments 1-23, comprising a hinge sequence disposed between and connecting a target-binding domain and the first TCR constant domain or the second TCR constant domain.
Embodiment 24a. The polypeptide dimer of Embodiment 24, wherein: (1) the first polypeptide comprises a hinge sequence disposed between and connecting a target-binding domain and the first TCR constant domain; (2) the second polypeptide comprises a hinge sequence disposed between and connecting a target-binding domain and the second TCR constant domain; or (3) (1) and (2).
Embodiment 25. The polypeptide dimer of Embodiment 24 or Embodiment 24a, wherein the hinge sequence comprises, consists essentially of, or consists of, the amino acid sequence set forth in any one of SEQ ID NOs.: 42-55 and 68-74, is a (GlyₓSer_{y})ₙ linker wherein x, y, and n are not zero, is a (A)ₙ linker wherein n is one or more, is a GPP linker, or any combination thereof.
Embodiment 26. The polypeptide dimer of any one of Embodiments 1-25, wherein the first polypeptide and/or the second polypeptide does not comprise an immunoglobulin CH2 domain and/or an immunoglobulin CH3 domain and/or an immunoglobulin light chain constant domain, e.g. disposed C-terminal to the TCR constant domain.
Embodiment 27. The polypeptide dimer of any one of Embodiments 1-26, comprising a target-binding domain comprising (i) a VH comprised in the first polypeptide or the second polypeptide and (ii) a cognate VL comprised in the other of the first and the second polypeptide, wherein the target is not 2,4,6-trinitrophenyl (TNP), digoxin, or phosphorylcholine.
Embodiment 28. The polypeptide dimer of any one of Embodiments 1-27, wherein the first polypeptide and the second polypeptide comprise, consist essentially of, or consist of, amino acid sequences having at least least 90%, at least 91%, at least 92%, at least 93%, at least 94%, at least 95%, at least 96%, at least 97%, at least 98%, or at least 99% to, or comprising or consisting of, the amino acid sequences set forth in SEQ ID NOs.: (i) 110 and 111, respectively; (ii) 112 and 113, respectively; or (iii) 118 and 119, respectively.
Embodiment 29. The polypeptide dimer of any one of Embodiments 1-27, wherein the first polypeptide, the second polypeptide, or both, comprises an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 114, 115, 116, 117, 118, 119, 120, 121, 122, 123, 124, or 125.
Embodiment 30. The polypeptide dimer of any one of Embodiments 1-29, wherein the target-binding domain has a Kd for the target in the range of about 1 nM to about 100 nM, as determined by surface plasmon resonance.
Embodiment 31. A polypeptide comprising the first or the second polypeptide of the polypeptide dimer of any one of Embodiments 1-30, provided that the polypeptide comprises a target-binding domain disposed amino-terminal to the TCR constant domain, and the polypeptide is not a TCR chain comprising a TCR variable domain and a TCR constant domain.
Embodiment 32. A fusion polypeptide comprising an extracellular component, an intracellular component, and a transmembrane component disposed between and connecting the extracellular component and the intracellular component, wherein the extracellular component comprises a CD226 ectodomain or a portion or variant thereof that is functional to bind PVR, and wherein the intracellular component comprises: (i) a CD226 endodomain comprising one or more (e.g. substitution, e.g. non-conservative) mutation that (a) provides increased expression of the fusion polypeptide in a T cell exposed to PVR, as compared to expression of endogenous CD226 by the T cell exposed to PVR, and/or (b) disrupts a Src kinase phosphorylation site on the fusion polypeptide, and/or (c) reduces ubiquitination of the fusion polypeptide by CBL-B, wherein, optionally, the mutated CD226 endodomain comprises a substitution mutation at a position corresponding to one or more of positions K295, and Y319, and K333, further comprising optionally K295A, Y319F, and/or K333A mutations; (ii) a CD2 intracellular domain sequence (e.g. comprising a co-stimulatory domain); (iii) a truncated CD2 intracellular domain sequence (e.g. comprising a co-stimulatory domain); (iv) a co-stimulatory domain sequence from any one or more of 4-1BB, CD28, OX40, CD27, CD3€, CD36, CD3γ, CD32, CD79A, CD79B, SLAMF1, ICOS, DAP10, GITR, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, PTCH2; or (v) any combination of (i)-(iv).
Embodiment 33. The fusion polypeptide of Embodiment 32, wherein the CD226 ectodomain or portion or variant thereof has least 90%, at least 91%, at least 92%, at least 93%, at least 94%, at least 95%, at least 96%, at least 97%, at least 98%, or at least 99% to, or comprises or consists of, the amino acid sequence set forth in SEQ ID NO.: 77.
Embodiment 34. The fusion polypeptide of Embodiment 32 or 33, wherein the transmembrane component comprises a CD226 transmembrane domain.
Embodiment 35. The fusion polypeptide of Embodiment 32 or 33, wherein the transmembrane component has 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 78.
Embodiment 36. A fusion polypeptide comprising an extracellular component, an intracellular component, and a transmembrane component disposed between and connecting the extracellular component and the intracellular component, wherein the extracellular component comprises a TIGIT ectodomain or a portion or variant thereof that is functional to bind PVR, and wherein the intracellular component comprises: (i) a CD2 intracellular domain sequence (e.g. comprising a co-stimulatory domain); (ii) a truncated CD2 intracellular domain sequence (e.g. comprising a co-stimulatory domain); (iii) a co-stimulatory domain sequence from any one or more of 4-1BB, CD28, OX40, CD27, CD3δ, CD3δ, CD3γ, CD3ζ, CD79A, CD79B, SLAMF1, ICOS, DAP10, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, PTCH2; or (iv) any combination of (i)-(iii).
Embodiment 37. The fusion polypeptide of Embodiment 36, wherein the TIGIT ectodomain or portion or variant thereof has least 90%, at least 91%, at least 92%, at least 93%, at least 94%, at least 95%, at least 96%, at least 97%, at least 98%, or at least 99% to, or comprises or consists of, the amino acid sequence set forth in SEQ ID NO.: 82.
Embodiment 38. The fusion polypeptide of Embodiment 36 or 37, wherein the transmembrane component comprises a TIGIT transmembrane domain.
Embodiment 39. The fusion polypeptide of Embodiment 36 or 37, wherein the transmembrane component has 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 83.
Embodiment 40. The fusion polypeptide of any one of Embodiments 32-39, wherein the intracellular component comprises an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 79, 84, 85, 86, 87, and 141-149.
Embodiment 41. The fusion polypeptide of any one of Embodiments 32-40, wherein, when a host T cell expressing the fusion polypeptide binds to PVR, the host T cell produces: (i) a CD226 signal; (ii) a CD226 signal that is longer, more persistent, and/or more intense than the signal endogenous CD226 produces in a reference T cell expressing the endogenous CD226 and binding PVR; (iii) does not produce a TIGIT signal; and/or (iv) produces a TIGIT signal that is less intense, is attenuated, is shorter than, and/or is less persistent than the signal endogenous TIGIT produces in a reference T cell expressing the endogenous TIGIT and binding PVR.
Embodiment 42. The fusion polypeptide of any one of Embodiments 32-41, comprising, consisting essentially of, or consisting of, an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 88-96.
Embodiment 43. A polynucleotide encoding (i) the polypeptide dimer of any one of Embodiments 1-30, (ii) the polypeptide of Embodiment 31, (iii) the fusion polypeptide of any one of Embodiments 32-42, or (iv) any combination thereof.
Embodiment 44. The polynucleotide of Embodiment 43, encoding the polypeptide dimer of any one of Embodiments 1-30 and the fusion polypeptide of any one of Embodiments 32-42.
Embodiment 44a. The polynucleotide of Embodiment 43 or 44, comprising:
i) (i) (a) a sequence encoding the polypeptide dimer of any one of Embodiments 1-30, wherein the polypeptide dimer binds to: BCMA, GPRCSD, BCMA and GPRC5D, BCMA and CD229, BCMA and SLAMF7, GPRCSD and CD229, GPRCSD and SLAMF7, or CD229 and SLAMF7, and/or (1) (b) a sequence encoding a chimeric antigen receptor (CAR) that binds to BCMA, GPRC5D, BCMA and GPRC5D, BCMA and CD229, BCMA and SLAMF7, GPRC5D and CD229, GPRC5D and SLAMF7, CD229 and SLAMF7, CD19 and CD229, or CD19 and BCMA; and
(ii) a sequence encoding the fusion polypeptide of any one of Embodiments 32-42.
Embodiment 45. The polynucleotide of any one of Embodiments 43-44a, further comprising: (i) a promoter, optionally a MNDu3 promoter or a EFla promoter; (ii) one or more sequence encoding a signal peptide; (iii) one or more sequence encoding a furin cleavage sequence; (iv) one or more sequence encoding a self-cleaving peptide; (v) one or more sequence encoding a tag peptide; (vi) one or more sequence encoding a transduction marker; or (vii) any combination thereof.
Embodiment 46. The polynucleotide of Embodiment 45, comprising a sequence encoding a signal peptide disposed 5′ to: a sequence encoding the first polypeptide of the polypeptide dimer; a sequence encoding the second polypeptide of the polypeptide dimer; a sequence encoding the polypeptide of Embodiment 31; and/or a sequence encoding the fusion polypeptide of any one of Embodiments 32-42.
Embodiment 47. The polynucleotide of Embodiment 45 or 46, comprising (1) a sequence encoding a furin cleavage sequence site sequence and/or (2) a sequence encoding a self-cleaving peptide, disposed between: a sequence encoding the first polypeptide of the polypeptide dimer and a sequence encoding the second polypeptide of the polypeptide dimer; a sequence encoding the first polypeptide of the polypeptide dimer and a sequence encoding the fusion polypeptide; a sequence encoding the second polypeptide of the polypeptide dimer and a sequence encoding the fusion polypeptide; a sequence encoding the first polypeptide of the polypeptide dimer and a sequence encoding the polypeptide; a sequence encoding the second polypeptide of the polypeptide dimer and a sequence encoding the polypeptide; a sequence encoding the polypeptide and a sequence encoding the fusion polypeptide; or any combination thereof.
Embodiment 48. The polynucleotide of any one of Embodiments 43-47, comprising a sequence encoding the fusion polypeptide of any one of Embodiments 32-42 disposed between: (i) a sequence encoding the first polypeptide of the polypeptide dimer and (ii) a sequence encoding the second polypeptide of the polypeptide dimer.
Embodiment 49. The polynucleotide of any one of Embodiments 43-48, which is codon optimized for expression in a host cell, wherein the host cell is optionally a human host cell, further optionally a human immune system cell, still further optionally a human T cell (e.g. a CD4+ T cell, a CD8+ T cell, a CD4−CD8− double negative T cell, a γδ T cell, or any combination thereof), a human NK cell, or a human NK-T cell.
Embodiment 50. The polynucleotide of Embodiment 49, which is codon optimized for expression in a T cell (e.g. a CD4+ T cell, a CD8+ T cell, a CD4−CD8− double negative T cell, a γδ T cell, or any combination thereof), a NK cell, or a NK-T cell, wherein the cell is preferably human.
Embodiment 51. The polynucleotide of any one of Embodiments 43-50, encoding an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 1-6, 9-15, and 24-32.
Embodiment 52. A vector comprising the polynucleotide of any one of Embodiments 43-51.
Embodiment 53. The vector of Embodiment 52, wherein the vector is capable of delivering the polynucleotide to a hematopoietic progenitor cell or a human immune system cell.
Embodiment 54. The vector of Embodiment 53, wherein the human immune system cell comprises a CD4⁺ T cell, a CD8⁺ T cell, a CD4-CD8-double negative T cell, a γδ T cell, a natural killer cell, a natural killer T cell, a dendritic cell, or any combination thereof.
Embodiment 55. The vector of Embodiment 54, wherein the T cell comprises a naïve T cell, a central memory T cell, a stem cell memory T cell, an effector memory T cell, or any combination thereof.
Embodiment 56. The vector of any one of Embodiments 52-55, wherein the vector is a viral vector.
Embodiment 57. The vector of Embodiment 56, wherein the viral vector is a lentiviral vector or a γ-retroviral vector.
Embodiment 58. A host cell expressing: (i) the polypeptide dimer of any one of Embodiments 1-30; (ii) the polypeptide of Embodiment 31; (iii) the fusion polypeptide of any one of Embodiments 32-42; or (iv) any combination thereof.
Embodiment 59. A host cell comprising the polynucleotide of any one of Embodiments 43-51.
Embodiment 60. A host cell comprising the vector of any one of Embodiments 52-57.
Embodiment 61. The host cell of any one of Embodiments 58-60, wherein the host cell comprises a hematopoietic progenitor cell or a human immune system cell.
Embodiment 62. The host cell of any one of Embodiments 58-61, wherein the host cell comprises a CD4⁺ T cell, a CD8⁺ T cell, a CD4-CD8-double negative T cell, a γδ T cell, a natural killer cell, a natural killer T cell, a monocyte, or any combination thereof.
Embodiment 63. The host cell of Embodiment any one of Embodiments 58-62, wherein the host cell comprises a T cell.
Embodiment 64. The host cell of Embodiment 63, wherein the T cell comprises a naïve T cell, a central memory T cell, a stem cell memory T cell, an effector memory T cell, or any combination thereof.
Embodiment 65. The host cell of any one of Embodiments 58-64, comprising a chromosomal gene knockout or a mutation of: a TGFβR1 gene locus, a TGFβR2 gene locus, a PD-1 gene locus, a CTLA4 gene locus, a LAT gene locus, a TIM-3 gene locus, a PD-LI gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, a CD4 gene locus, a CD8 gene locus, or any combination thereof, such as a TIGIT locus, a TRAC gene locus, a TRBC gene locus, and/or one or both of a CD4 gene locus and a CD8 gene locus.
Embodiment 66. The host cell of any one of Embodiments 58-65, wherein the host cell is modified (e.g., having a chromosomal knockout mutation and/or a chromosomal missense mutation and/or a chromosomal splice junction mutation; encoding an inhibitory nucleic acid such as an siRNA or an antisense oligonucleotide) to have reduced protein expression (including null expression), of an endogenous TRAC, an endogenous TRBC, an endogenous TIGIT, endogenous CD4, endogenous CD8, or any combination thereof, as compared to the unmodified host cell.
Embodiment 67. The host cell of any one of Embodiments 58-66, expressing the polypeptide dimer of any one of Embodiments 1-30 and the fusion polypeptide of any one of Embodiments 32-42, wherein, optionally, the polypeptide dimer binds to: BCMA, GPRCSD, BCMA and GPRC5D, BCMA and CD229, BCMA and SLAMF7, GPRC5D and CD229, GPRC5D and SLAMF7, CD229 and SLAMF7, CD19 and BCMA, or CD19 and CD229.
Embodiment 67a. A host cell comprising a T cell comprising a chromosomal gene knockout of a CD4 and/or of a CD8, wherein the T cell expresses a T cell receptor (TCR) or a chimeric antigen receptor CAR), wherein the TCR or CAR is capable of binding to a peptide: MHC complex in the absence of CD4, in the absence of CD8, or in the absence of both.
Embodiment 68. A composition comprising: (i) the polypeptide dimer of any one of Embodiments 1-30; and/or (ii) the polypeptide of Embodiment 31; and/or (iii) the fusion polypeptide of any one of Embodiments 32-42; and/or (iv) the polynucleotide of any one of Embodiments 43-51; and/or (v) the vector of any one of Embodiments 52-57; and/or (vi) the host cell of any one of Embodiments 58-67a, and a pharmaceutically acceptable carrier, excipient, or diluent.
Embodiment 69. The composition of Embodiment 68, comprising (i) a composition comprising at least about 30% CD4⁺ T host cells, combined with (ii) a composition comprising at least about 30% CD8⁺ T host cells, in about a 1:1 ratio.
Embodiment 70. A method of treating a disease or condition in a subject, the method comprising administering to the subject an effective amount of: (i) the polypeptide dimer of any one of Embodiments 1-30; and/or (ii) the polypeptide of Embodiment 31; and/or (iii) the fusion polypeptide of any one of Embodiments 32-42; and/or (iv) the polynucleotide of any one of Embodiments 43-51; and/or (v) the vector of any one of Embodiments 52-57; and/or (vi) the host cell of any one of Embodiments 58-67a; and/or (vii) the composition of Embodiment 68 or 69.
Embodiment 71. The polypeptide dimer of any one of Embodiments 1-30, and/or the polypeptide of Embodiment 31, and/or the fusion polypeptide of any one of Embodiments 32-42, and/or the polynucleotide of any one of Embodiments 43-51, and/or the vector of any one of Embodiments 52-57, and/or the host cell of any one of Embodiments 58-67a, and/or the composition of Embodiment 68 or 69, for use in a method of treating a disease or condition in a subject.
Embodiment 72. The polypeptide dimer of any one of Embodiments 1-30, and/or the polypeptide of Embodiment 31, and/or the fusion polypeptide of any one of Embodiments 32-42, and/or the polynucleotide of any one of Embodiments 43-51, and/or the vector of any one of Embodiments 52-57, and/or the host cell of any one of Embodiments 58-67a, and/or the composition of Embodiment 68 or 69, for use in the preparation of a medicament for treating a disease or condition in a subject.
Embodiment 73. The method of Embodiment 70, or the polypeptide dimer, polypeptide, fusion polypeptide, polynucleotide, vector, host cell, or composition for use of any one of Embodiments 71-72, wherein the disease or condition comprises or is a hyperproliferative disease, a proliferative disease, an autoimmune disease, a neurodegenerative disease, or an infection.
Embodiment 74. The method of Embodiment 70 or 73 or the polypeptide dimer, polypeptide, fusion polypeptide polynucleotide, vector, host cell, or composition for use of any one of Embodiments 71-73, wherein the disease or condition is a cancer, such as a hematological cancer or a solid cancer.
Embodiment 75. The method of Embodiment 74 or the polypeptide dimer, polypeptide, fusion polypeptide, polynucleotide, vector, host cell, or composition for use of Embodiment 74, wherein the cancer comprises a myeloma (e.g. multiple myeloma), a carcinoma, a sarcoma, a glioma, a lymphoma, a leukemia, a myeloma, or any combination thereof.
Embodiment 76. The method of Embodiment 74 or 75, or the polypeptide dimer, polypeptide, fusion polypeptide, polynucleotide, vector, host cell, or composition for use of Embodiment 74 or 75, wherein the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.
Embodiment 77. The method of any one of Embodiments 74-76, or the polypeptide dimer, polypeptide, fusion polypeptide, polynucleotide, vector, host cell, or composition for use of any one of Embodiments 74-76, wherein the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.
Embodiment 78. The method of Embodiment 74 or the polypeptide dimer, polypeptide, fusion polypeptide, polynucleotide, vector, host cell, or composition for use of Embodiment 74, wherein the cancer comprises a solid tumor.
Embodiment 79. The method of Embodiment 78 or the polypeptide dimer, polypeptide, fusion polypeptide, polynucleotide, vector, host cell, or composition for use of Embodiment 78, wherein the solid tumor is a sarcoma or a carcinoma.
Embodiment 80. The method of Embodiment 79 or the polypeptide dimer, polypeptide, fusion polypeptide, polynucleotide, vector, host cell, or composition for use of Embodiment 79, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.
Embodiment 81. The method of any one of Embodiments 78-80 or the polypeptide dimer, polypeptide, fusion polypeptide, polynucleotide, vector, host cell, or composition for use of any one of Embodiments 78-80, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).
Embodiment 82. The method of any one of Embodiments 78-81, or the polypeptide dimer, polypeptide, fusion polypeptide, polynucleotide, vector, host cell, or composition for use of any one of Embodiments 78-81, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.
Embodiment 83. The method of any one of Embodiments 70 and 73-82, or the polypeptide dimer, polypeptide, fusion polypeptide, polynucleotide, vector, host cell, or composition for use of any one of Embodiments 71-82, wherein the host cell is an allogeneic cell, a syngeneic cell, or an autologous cell.
Embodiment 84. The method of any one of Embodiments 70 and 73-83, or the polypeptide dimer, polypeptide, fusion polypeptide, polynucleotide, vector, host cell, or composition for use of any one of Embodiments 71-83, wherein the method comprises administering a plurality of doses of the fusion protein, polynucleotide, vector, host cell, or composition to the subject.
Embodiment 85. The method of Embodiment 84 or the polypeptide dimer, polypeptide, fusion polypeptide, polynucleotide, vector, host cell, or composition for use of
Embodiment 84, wherein the plurality of doses are administered at intervals between administrations of about two, three, four, five, six, seven, eight, or more weeks.
Embodiment 86. The method of Embodiment 84 or 85, or the polypeptide dimer, polypeptide, fusion polypeptide, polynucleotide, vector, host cell, or composition for use of Embodiment 84 or 85, wherein a dose of the host cell comprises about 10⁵ cells/m2 to about 10¹¹ cells/m2.
Embodiment 87. The method of any one of Embodiments 70 and 73-86, or the polypeptide dimer, polypeptide, fusion polypeptide, polynucleotide, vector, host cell, or composition for use of any one of Embodiments 71-86, wherein the subject is receiving, has received, or will receive one or more of: (i) chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.
Embodiment 88. A method comprising introducing into a host cell (i) a polynucleotide of any one of Embodiments 43-51 or (ii) a vector of any one of Embodiments 52-57.
Embodiment 89. The method of Embodiment 88, wherein the host cell comprises a hematopoietic progenitor cell or an immune system cell, optionally a human immune system cell.
Embodiment 90. The method of Embodiment 88 or 89, wherein the host cell comprises a CD4+ T cell, a CD8+ T cell, a CD4-CD8-double negative T cell, a γδ T cell, a natural killer cell, a natural killer T cell, a monocyte, or any combination thereof.
Embodiment 91. The method of any one of Embodiments 88-90, wherein the host cell comprises a T cell.
Embodiment 92. The method of Embodiment 91, wherein the T cell comprises a naïve T cell, a central memory T cell, a stem cell memory T cell, an effector memory T cell, or any combination thereof.
Embodiment 93. The method of any one of Embodiments 88-92, further comprising introducing a chromosomal gene knockout of TIGIT, CD226, TRAC, TRBC, CD8, CD4, or any combination thereof.

LIST OF SEQUENCES
Sequence TCR/CAR Split and Full formats:
(SEQ ID NO.: 1) TCR_CAR_Split_VL_TRBC_VH_TRAC Construct (with GM-CSF leader
sequences underlined, VL and VH from FMC63)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMLLLVTSLLLCELPHPAFLLIPEVKLQESGPGLVAPSQSLSVTCTVSGVSLPDYGVSWIR
QPPRKGLEWLGVIWGSETTYYNSALKSRLTIIKDNSKSQVFLKMNSLQTDDTAIYYCAKHYYYG
GSYAMDYWGQGTSVTVSSIQNPDPAVYQLRDSKSSDKSVCLFTDFDSQTNVSQSKDSDVYITDK
CVLDMRSMDFKSNSAVAWSNKSDFACANAFNNSHIPEDTFFPSPESSCDVKLVEKSFETDTNLNF
QNLLVIVLRILLLKVAGFNLLMTLRLWSS
(SEQ ID NO.: 2) TCR_CAR_Split_VH_TRBC_VL_TRAC Construct (with GM-CSF leader
sequences underlined, VH and VL from FMC63)
MLLLVTSLLLCELPHPAFLLIPEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYAM
DYWGQGTSVTVSSDLNKVFPPEVAVFEPSEAEISHTQKATLVCLATGFFPDHVELSWWVNGKEV
HSGVCTDPQPLKEQPALNDSRYCLSSRLRVSATFWQNPRNHFRCQVQFYGLSENDEWTQDRAK
PVTQIVSAEAWGRADCGFTSVSYQQGVLSATILYEILLGKATLYAVLVSALVLMAMVKRKDFRA
KRGSGATNFSLLKQAGDVEENPGPMLLLVTSLLLCELPHPAFLLIPDIQMTQTTSSLSASLGDRVTI
SCRASQDISKYLNWYQQKPDGTVKLLIYHTSRLHSGVPSRFSGSGSGTDYSLTISNLEQEDIATYF
CQQGNTLPYTFGGGTKLEITIQNPDPAVYQLRDSKSSDKSVCLFTDFDSQTNVSQSKDSDVYITD
KCVLDMRSMDFKSNSAVAWSNKSDFACANAFNNSIIPEDTFFPSPESSCDVKLVEKSFETDTNLN
FQNLLVIVLRILLLKVAGFNLLMTLRLWSS
(SEQ ID NO.: 3) TCR_CAR_Full_TRBC_VL_VH_TRAC Construct (with GM-CSF leader
sequences underlined, VL and VH from FMC63)
MLLLVTSLLLCELPHPAFLLIPDLNKVFPPEVAVFEPSEAEISHTQKATLVCLATGFFPDHVELSW
WVNGKEVHSGVCTDPQPLKEQPALNDSRYCLSSRLRVSATFWQNPRNHFRCQVQFYGLSENDE
WTQDRAKPVTQIVSAEAWGRADCGFTSVSYQQGVLSATILYEILLGKATLYAVLVSALVLMAM
VKRKDFRAKRGSGATNFSLLKAAGDVEENPGPMLLLVTSLLLCELPHPAFLLIPDIQMTQTTSSLS
ASLGDRVTISCRASQDISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVKLQESGPGLVAPSQSLS
VTCTVSGVSLPDYGVSWIRQPPRKGLEWLGVIWGSETTYYNSALKSRLTHIKDNSKSQVFLKMNS
LQTDDTAIYYCAKHYYYGGSYAMDYWGQGTSVTVSSIQNPDPAVYQLRDSKSSDKSVCLFTDF
DSQTNVSQSKDSDVYITDKCVLDMRSMDFKSNSAVAWSNKSDFACANAFNNSIIPEDTFFPSPES
SCDVKLVEKSFETDTNLNFQNLLVIVLRILLLKVAGFNLLMTLRLWSS
(SEQ ID NO.: 4) TCR_CAR_Full_VL_VH_TRBC_TRAC Construct (with GM-CSF leader
sequences underlined, VL and VH from FMC63)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITGSTS
GSGKPGSGEGSTKGEVKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYYGGSYAMDYWGQGT
SVTVSSDLNKVFPPEVAVFEPSEAEISHTQKATLVCLATGFFPDHVELSWWVNGKEVHSGVCTD
PQPLKEQPALNDSRYCLSSRLRVSATFWQNPRNHFRCQVQFYGLSENDEWTQDRAKPVTQIVSA
EAWGRADCGFTSVSYQQGVLSATILYEILLGKATLYAVLVSALVLMAMVKRKDFRAKRGSGAT
NFSLLKQAGDVEENPGPMLLLVTSLLLCELPHPAFLLIPIQNPDPAVYQLRDSKSSDKSVCLFTDFD
SQINVSQSKDSDVYITDKCVLDMRSMDFKSNSAVAWSNKSDFACANAFNNSHIPEDTFFPSPESS
CDVKLVEKSFETDTNLNFQNLLVIVLRILLLKVAGENLLMTLRLWSS
(SEQ ID NO.: 5) TCR_CAR_Full_TRBC_VH_VL_TRAC Construct (with GM-CSF leader
sequences underlined, VH and VL from FMC63)
MLLLVTSLLLCELPHPAFLLIPDLNKVFPPEVAVFEPSEAEISHTQKATLVCLATGFFPDHVELSW
WVNGKEVHSGVCTDPQPLKEQPALNDSRYCLSSRLRVSATFWQNPRNHFRCQVQFYGLSENDE
WTQDRAKPVTQIVSAEAWGRADCGFTSVSYQQGVLSATILYEILLGKATLYAVLVSALVLMAM
VKRKDFRAKRGSGATNFSLLKQAGDVEENPGPMLLLVTSLLLCELPHPAFLLIPEVKLQESGPGLV
APSQSLSVTCTVSGVSLPDYGVSWIRQPPRKGLEWLGVIWGSETTYYNSALKSRLTIIKDNSKSQ
VFLKMNSLQTDDTAIYYCAKHYYYGGSYAMDYWGQGTSVTVSSGSTSGSGKPGSGEGSTKGDI
QMTQTTSSLSASLGDRVTISCRASQDISKYLNWYQQKPDGTVKLLIYHTSRLHSGVPSRFSGSGS
GTDYSLTISNLEQEDIATYFCQQGNTLPYTFGGGTKLEITIQNPDPAVYQLRDSKSSDKSVCLFTD
FDSQTNVSQSKDSDVYITDKCVLDMRSMDFKSNSAVAWSNKSDFACANAFNNSIIPEDTFFPSPE
SSCDVKLVEKSFETDTNLNFQNLLVIVLRILLLKVAGFNLLMTLRLWSS
(SEQ ID NO.: 6) TCR_CAR_Full_VH_VL_TRBC_TRAC Construct (with GM-CSF leader
sequences underlined, VH and VL from FMC63)
MLLLVTSLLLCELPHPAFLLIPEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYAM
DYWGQGTSVTVSSGSTSGSGKPGSGEGSTKGDIQMTQTTSSLSASLGDRVTISCRASQDISKYLN
WYQQKPDGTVKLLIYHTSRLHSGVPSRFSGSGSGTDYSLTISNLEQEDIATYFCQQGNTLPYTFG
GGTKLEITDLNKVFPPEVAVFEPSEAEISHTQKATLVCLATGFFPDHVELSWWVNGKEVHSGVCT
DPQPLKEQPALNDSRYCLSSRLRVSATFWQNPRNHFRCQVQFYGLSENDEWTQDRAKPVTQIVS
AEAWGRADCGFTSVSYQQGVLSATILYEILLGKATLYAVLVSALVLMAMVKRKDFRAKRGSGA
TNFSLLKQAGDVEENPGPMLLLVTSLLLCELPHPAFLLIPIQNPDPAVYQLRDSKSSDKSVCLFTDF
DSQTNVSQSKDSDVYITDKCVLDMRSMDFKSNSAVAWSNKSDFACANAFNNSIIPEDTFFPSPES
SCDVKLVEKSFETDTNLNFQNLLVIVLRILLLKVAGFNLLMTLRLWSS
Sequence promoters:
(SEQ ID NO.: 7) EF1a_promoter
ggatctgcgatcgctccggtgcccgtcagtgggcagagcgcacatcgcccacagtccccgagaagttggggggaggggtcggcaattgaaccggtg
cctagagaaggtggcgcggggtaaactgggaaagtgatgtcgtgtactggctccgcctttttcccgaggggggggagaaccgtatataagtgcagtag
tcgccgtgaacgttctttttcgcaacgggtttgccgccagaacacagctgaagcttcgaggggctcgcatctctccttcacgcgcccgccgccctacctga
ggccgccatccacgccggttgagtcgcgttctgccgcctcccgcctgtggtgcctcctgaactgcgtccgccgtctaggtaagtttaaagctcaggtcga
gaccgggcctttgtccggcgctcccttggagcctacctagactcagccggctctccacgctttgcctgaccctgcttgctcaactctacgtctttgtttcgtttt
ctgttctgcgccgtTACAGATCCAAGCTGTGACCGGCGCCTAC
(SEQ ID NO.: 8) MNDu3_promoter
tgaaagaccccacctgtaggtttggcaagctaggatcaaggttaggaacagagagacagcagaatatgggccaaacaggatatctgtggtaagcagttc
ctgccccggctcagggccaagaacagttggaacagcagaatatgggccaaacaggatatctgtggtaagcagttcctgccccggctcagggccaaga
acagatggtccccagatgcggtcccgccctcagcagtttctagagaaccatcagatgtttccagggtgccccaaggacctgaaatgaccetgtgccttatt
tgaactaaccaatcagttcgcttctcgcttctgttcgcgcgcttctgctccccgagctcaataaaagagcccacaacccctcactcggc
Sequence bispecific TCR/CAR:
(SEQ ID NO.: 9) TCR_CAR Bispecific_GPRCSD_TRBC_BCMA TRAC_tEGFR Construct (with
GM-CSF leader sequences underlined; scFvs from anti-GPRCSD (VL-linker-VH) and anti-BCMA
(VH-linker-VL) antibodies)
MLLLVTSLLLCELPHPAFLLIPQSALTQPASVSGSPGQSITISCTGTSSDVGGYNYVSWYQQHPGK
APKLMIYDVSKRPSGVSNRFSGSKSGNTASLTISGLQAEDEADYYCSSYTRSSTEVFGGGTKLTV
LGSRGGGGSGGGGSGGGGSLEMAEVQLVQSGAEVKKPGASVKVSCKASGYTFTSYYMHWVRQ
APGQGLEWMGIINPSGGSTSYAQKFQGRVTMTRDTSTSTVYMELSSLRSEDTAVYYCARGGSKK
WSGEKWRRENFDYWGQGTLVTVSSDLNKVFPPEVAVFEPSEAEISHTQKATLVCLATGFFPDHV
ELSWWVNGKEVHSGVCTDPQPLKEQPALNDSRYCLSSRLRVSATFWQNPRNHFRCQVQFYGLS
ENDEWTQDRAKPVTQIVSAEAWGRADCGFTSVSYQQGVLSATILYEILLGKATLYAVLVSALVL
MAMVKRKDFRAKRGSGATNFSLLKQAGDVEENPGPMLLLVTSLLLCELPHPAFLLIPQIQLVQSGP
ELKKPGETVKISCKASGYTFTDYSINWVKRAPGKGLKWMGWINTETREPAYAYDFRGRFAFSLE
TSASTAYLQINNLKYEDTATYFCALDYSYAMDYWGQGTSVTVSSGGGGSGGGGSGGGGSDIVL
TQSPPSLAMSLGKRATISCRASESVTILGSHLIHWYQQKPGQPPTLLIQLASNVQTGVPARFSGSG
SRTDFTLTIDPVEEDDVAVYYCLQSRTIPRTFGGGTKLEIKIQNPDPAVYQLRDSKSSDKSVCLFT
DFDSQTNVSQSKDSDVYITDKCVLDMRSMDFKSNSAVAWSNKSDFACANAFNNSIIPEDTFFPSP
ESSCDVKLVEKSFETDTNLNFQNLLVIVLRILLLKVAGFNLLMTLRLWSSRAKRGSGATNFSLLKQ
AGDVEENPGPMLLLVTSLLLCELPHPAFLLIPRKVCNGIGIGEFKDSLSINATNIKHFKNCTSISGDL
HILPVAFRGDSFTHTPPLDPQELDILKTVKEITGFLLIQAWPENRTDLHAFENLEIIRGRTKQHGQF
SLAVVSLNITSLGLRSLKEISDGDVIISGNKNLCYANTINWKKLFGTSGQKTKIISNRGENSCKATG
QVCHALCSPEGCWGPEPRDCVSCRNVSRGRECVDKCNLLEGEPREFVENSECIQCHPECLPQAM
NITCTGRGPDNCIQCAHYIDGPHCVKTCPAGVMGENNTLVWKYADAGHVCHLCHPNCTYGCTG
PGLEGCPTNGPKIPSIATGMVGALLLLLVVALGIGLFM
Sequence nanobody-based TCR/CARs:
(SEQ ID NO.: 10) TCR_CAR_TRBC_Nanobody_TRAC Construct (with GM-CSF leader sequences
underlined; two copies of nanobody MB14 separated by triple-alanine linker)
MLLLVTSLLLCELPHPAFLLIPDLNKVFPPEVAVFEPSEAEISHTQKATLVCLATGFFPDHVELSW
WVNGKEVHSGVCTDPQPLKEQPALNDSRYCLSSRLRVSATFWQNPRNHFRCQVQFYGLSENDE
WTQDRAKPVTQIVSAEAWGRADCGFTSVSYQQGVLSATILYEILLGKATLYAVLVSALVLMAM
VKRKDFRAKRGSGATNFSLLKQAGDVEENPGPMLLLVTSLLLCELPHPAFLLIPQVKLVQSGGGL
VQTGGSLRLSCAASEITFDMYSMGWYREAPGKARDAVASITNRGNTYYADSVKGRFTISRDNA
KKTMYLQMNSLKPEDTAVYYCNVYRTGFSDYWGQGTQVTVSSAAAQVKLVQSGGGLVQTGG
SLRLSCAASEITFDMYSMGWYREAPGKARDAVASITNRGNTYYADSVKGRFTISRDNAKKTMY
LQMNSLKPEDTAVYYCNVYRTGFSDYWGQGTQVTVSSIQNPDPAVYQLRDSKSSDKSVCLFTD
FDSQTNVSQSKDSDVYITDKCVLDMRSMDFKSNSAVAWSNKSDFACANAFNNSIIPEDTFFPSPE
SSCDVKLVEKSFETDTNLNFQNLLVIVLRILLLKVAGFNLLMTLRLWSS
(SEQ ID NO.: 11) TCR_CAR_Nanobody_TRBC TRAC Construct (with GM-CSF leader sequences
underlined; two copies of nanobody MB14 separated by triple-alanine linker)
MLLLVTSLLLCELPHPAFLLIPQVKLVQSGGGLVQTGGSLRLSCAASEITFDMYSMGWYREAPG
KARDAVASITNRGNTYYADSVKGRFTISRDNAKKTMYLQMNSLKPEDTAVYYCNVYRTGFSDY
WGQGTQVTVSSAAAQVKLVQSGGGLVQTGGSLRLSCAASEITEDMYSMGWYREAPGKARDAV
ASITNRGNTYYADSVKGRFTISRDNAKKTMYLQMNSLKPEDTAVYYCNVYRTGFSDYWGQGT
QVTVSSDLNKVFPPEVAVFEPSEAEISHTQKATLVCLATGFFPDHVELSWWVNGKEVHSGVCTD
PQPLKEQPALNDSRYCLSSRLRVSATFWQNPRNHFRCQVQFYGLSENDEWTQDRAKPVTQIVSA
EAWGRADCGFTSVSYQQGVLSATILYEILLGKATLYAVLVSALVLMAMVKRKDFRAKRGSGAT
NFSLLKQAGDVEENPGPMLLLVTSLLLCELPHPAFLLIPIQNPDPAVYQLRDSKSSDKSVCLFTDFD
SQTNVSQSKDSDVYITDKCVLDMRSMDFKSNSAVAWSNKSDFACANAFNNSIIPEDTFFPSPESS
CDVKLVEKSFETDTNLNFQNLLVIVLRILLLKVAGFNLLMTLRLWSS
(SEQ ID NO.: 12) TCR_CAR Nanobody_MB14(2x, separated by triple-alanine
linker)_TRBC_Nanobody_MB14(2x, separated by triple-alanine linker)_TRAC Construct (with
GM-CSF leader sequences underlined)
MLLLVTSLLLCELPHPAFLLIPQVKLVQSGGGLVQTGGSLRLSCAASEITFDMYSMGWYREAPG
KARDAVASITNRGNTYYADSVKGRFTISRDNAKKTMYLQMNSLKPEDTAVYYCNVYRTGFSDY
WGQGTQVTVSSAAAQVKLVQSGGGLVQTGGSLRLSCAASEITFDMYSMGWYREAPGKARDAV
ASITNRGNTYYADSVKGRFTISRDNAKKTMYLQMNSLKPEDTAVYYCNVYRTGFSDYWGQGT
QVTVSSDLNKVFPPEVAVFEPSEAEISHTQKATLVCLATGFFPDHVELSWWVNGKEVHSGVCTD
PQPLKEQPALNDSRYCLSSRLRVSATFWQNPRNHFRCQVQFYGLSENDEWTQDRAKPVTQIVSA
EAWGRADCGFTSVSYQQGVLSATILYEILLGKATLYAVLVSALVLMAMVKRKDFRAKRGSGAT
NFSLLKQAGDVEENPGPMLLLVTSLLLCELPHPAFLLIPQVKLVQSGGGLVQTGGSLRLSCAASEI
TFDMYSMGWYREAPGKARDAVASITNRGNTYYADSVKGRFTISRDNAKKTMYLQMNSLKPED
TAVYYCNVYRTGFSDYWGQGTQVTVSSAAAQVKLVQSGGGLVQTGGSLRLSCAASEITEDMYS
MGWYREAPGKARDAVASITNRGNTYYADSVKGRFTISRDNAKKTMYLQMNSLKPEDTAVYYC
NVYRTGFSDYWGQGTQVTVSSIQNPDPAVYQLRDSKSSDKSVCLFTDFDSQTNVSQSKDSDVYI
TDKCVLDMRSMDFKSNSAVAWSNKSDFACANAFNNSIIPEDTFFPSPESSCDVKLVEKSFETDTN
LNFQNLLVIVLRILLLKVAGFNLLMTLRLWSS
Sequence protein-based TCR/CARs:
(SEQ ID NO.: 13) TCR_CAR_TRBC_FLAG-HA_Bcl2_Ectodomain_TRAC Construct (with GM-
CSF and and murine IgG, kappa leader sequences underlined)
MLLLVTSLLLCELPHPAFLLIPDLNKVFPPEVAVFEPSEAEISHTQKATLVCLATGFFPDHVELSW
WVNGKEVHSGVCTDPQPLKEQPALNDSRYCLSSRLRVSATFWQNPRNHFRCQVQFYGLSENDE
WTQDRAKPVTQIVSAEAWGRADCGFTSVSYQQGVLSATILYEILLGKATLYAVLVSALVLMAM
VKRKDFRAKRGSGATNFSLLKQAGDVEENPGPMETDTLLLWVLLLWVPGSTGDYKDEYPYDVP
DYAGSAHAGRTGYDNREIVMKYIHYKLSQRGYEWDAGDDAEENRTEAPEGTESEVVHRALRD
AGDDFERRYRRDFAEMSSQLHLTPDTARQRFETVVEELFRDGVNWGRIVAFFEFGGVMCVESV
NREMSPLVDNIAEWMTEYLNRHLHTWIQDNGGWDAFVELYGPSMRGGGGSGGGGSESKYGPP
CPPCPIQNPDPAVYQLRDSKSSDKSVCLFTDFDSQTNVSQSKDSDVYITDKCVLDMRSMDFKSNS
AVAWSNKSDFACANAFNNSIIPEDTFFPSPESSCDVKLVEKSFETDTNLNFQNLLVIVLRILLLKV
AGFNLLMTLRLWSS
(SEQ ID NO.: 14) TCR_CAR_FLAG_HA_Bcl2_Ectodomain_TRBC_TRAC Construct (with GM-
CSF and murine IgG, kappa leader sequences underlined)
METDTLLLWVLLLWVPGSTGDYKDEYPYDVPDYAGSAHAGRTGYDNREIVMKYIHYKLSQRG
YEWDAGDDAEENRTEAPEGTESEVVHRALRDAGDDFERRYRRDFAEMSSQLHLTPDTARQRFE
TVVEELFRDGVNWGRIVAFFEFGGVMCVESVNREMSPLVDNIAEWMTEYLNRHLHTWIQDNGG
WDAFVELYGPSMRGGGGSGGGGSESKYGPPCPPCPDLNKVFPPEVAVFEPSEAFISHTQKATLVC
LATGFFPDHVELSWWVNGKEVHSGVCTDPQPLKEQPALNDSRYCLSSRLRVSATFWQNPRNHF
RCQVQFYGLSENDEWTQDRAKPVTQIVSAEAWGRADCGFTSVSYQQGVLSATILYEILLGKATL
YAVLVSALVLMAMVKRKDFRAKRGSGATNFSLLKQAGDVEENPGPMLLLVTSLLLCELPHPAFL
LIPIQNPDPAVYQLRDSKSSDKSVCLFTDFDSQTNVSQSKDSDVYITDKCVLDMRSMDFKSNSAV
AWSNKSDFACANAFNNSIIPEDTFFPSPESSCDVKLVEKSFETDTNLNFQNLLVIVLRILLLKVAGF
NLLMTLRLWSS
(SEQ ID NO.: 15) TCR_CAR_Bcl2_Ectodomain_TRBC_Bcl2_Ectodomain_TRAC Construct (with
murine IgG, kappa leader sequences underlined)
METDTLLLWVLLLWVPGSTGDYKDEYPYDVPDYAGSAHAGRTGYDNREIVMKYIHYKLSQRG
YEWDAGDDAEENRTEAPEGTESEVVHRALRDAGDDFERRYRRDFAEMSSQLHLTPDTARQRFE
TVVEELFRDGVNWGRIVAFFEFGGVMCVESVNREMSPLVDNIAEWMTEYLNRHLHTWIQDNGG
WDAFVELYGPSMRGGGGSGGGGSESKYGPPCPPCPDLNKVFPPEVAVFEPSEAEISHTQKATLVC
LATGFFPDHVELSWWVNGKEVHSGVCTDPQPLKEQPALNDSRYCLSSRLRVSATFWQNPRNHF
RCQVQFYGLSENDEWTQDRAKPVTQIVSAEAWGRADCGFTSVSYQQGVLSATILYEILLGKATL
YAVLVSALVLMAMVKRKDFRAKRGSGATNFSLLKQAGDVEENPGPMETDTLLLWVLLLWVPGS
TGDYKDEEQKLISEEDLGSAHAGRTGYDNREIVMKYIHYKLSQRGYEWDAGDDAEENRTEAPE
GTESEVVHRALRDAGDDFERRYRRDFAEMSSQLHLTPDTARQRFETVVEELFRDGVNWGRIVAF
FEFGGVMCVESVNREMSPLVDNIAEWMTEYLNRHLHTWIQDNGGWDAFVELYGPSMRGGGGS
GGGGSESKYGPPCPPCPIQNPDPAVYQLRDSKSSDKSVCLFTDFDSQTNVSQSKDSDVYITDKCV
LDMRSMDFKSNSAVAWSNKSDFACANAFNNSIIPEDTFFPSPESSCDVKLVEKSFETDTNLNFQN
LLVIVLRILLLKVAGFNLLMTLRLWSS
Sequence seRNAs targeting TRAC and TRBC chains:
(SEQ ID NO.: 16) sgRNA_CRISPR_TRAC_1
AGAGTCTCTCAGCTGGTACA
(SEQ ID NO.: 17) sgRNA_CRISPR_TRAC_2
GAGAATCAAAATCGGTGAAT
(SEQ ID NO.: 18) sgRNA_CRISPR_TRBC_1
CAAACACAGCGACCTTGGGT
(SEQ ID NO.: 19) sgRNA_CRISPR_TRBC_2
GGCTCTCGGAGAATGACGAG
(SEQ ID NO.: 20) sgRNA_Base_Editor_TRAC_1
TTCGTATCTGTAAAACCAAG
(SEQ ID NO.: 21) sgRNA_Base_Editor_TRAC_2
CTTACCTGGGCTGGGGAAGA
(SEQ ID NO.: 22) sgRNA_Base_Editor_TRBC_1
AGGTCCTCTGGAAAGGGAAG
(SEQ ID NO.: 23) sgRNA_Base_Editor_TRBC_2
CCACTCACCTGCTCTACCCC
Sequence TCR/CARs with co-stimulation molecules:
(SEQ ID NO.: 24) TCR_CAR_Split_CCR_CD226mut Construct (VL and VH from FMC63; GM-
CSF and CD226 leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMDYPTLLLALLHVYRALCEEVLWHTSVPFAENMSLECVYPSMGILTQVEWFKIGTQQD
SIAIFSPTHGMVIRKPYAERVYFLNSTMASNNMTLFFRNASEDDVGYYSCSLYTYPQGTWQKVIQ
VVQSDSFEAAVPSNSHIVSEPGKNVTLTCQPQMTWPVQAVRWEKIQPRQIDLLTYCNLVHGRNF
TSKFPRQIVSNCSHGRWSVIVIPDVTVSDSGLYRCYLQASAGENETFVMRLTVAEGKTDNQYTLF
VAGGTVLLLLFVISITTIIVIFLNRRRRRERRDLFTESWDTQAAPNNYRSPISTSQPTNQSMDDTRE
DIYVNYPTFSRRPATRVRAKRGSGATNFSLLKQAGDVEENPGPMLLLVTSLLLCELPHPAFLLIPEV
KLQESGPGLVAPSQSLSVTCTVSGVSLPDYGVSWIRQPPRKGLEWLGVIWGSETTYYNSALKSRL
TIKDNSKSQVFLKMNSLQTDDTAIYYCAKHYYYGGSYAMDYWGQGTSVTVSSIQNPDPAVYQ
LRDSKSSDKSVCLFTDFDSQTNVSQSKDSDVYITDKCVLDMRSMDFKSNSAVAWSNKSDFACA
NAFNNSIIPEDTFFPSPESSCDVKLVEKSFETDTNLNFQNLLVIVLRILLLKVAGFNLLMTLRLWSS
(SEQ ID NO.: 25) TCR_CAR_Split_CCR_CD226_CD2 Construct (VL and VH from FMC63; GM-
CSF and CD226 leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDY
EENPGPMDYPTLLLALLHVYRALCEEVLWHTSVPFAENMSLECVYPSMGILTQVEWFKIGTQQD
SIAIFSPTHGMVIRKPYAERVYFLNSTMASNNMTLFFRNASEDDVGYYSCSLYTYPQGTWQKVIQ
VVQSDSFEAAVPSNSHIVSEPGKNVTLTCQPQMTWPVQAVRWEKIQPRQIDLLTYCNLVHGRNF
TSKFPRQIVSNCSHGRWSVIVIPDVTVSDSGLYRCYLQASAGENETFVMRLTVAEGKTDNQYTLF
VAGGTVLLLLFVISITTIIVIFLKRKKQRSRRNDEELETRAHRVATEERGRKPHQIPASTPQNPATS
QHPPPPPGHRSQAPSHRPPPPGHRVQHQPQKRPPAPSGTQVHQQKGPPLPRPRVQPKPPHGAAEN
SLSPSSNRAKRGSGATNFSLLKQAGDVEENPGPMLLLVTSLLLCELPHPAFLLIPEVKLQESGPGLV
APSQSLSVTCTVSGVSLPDYGVSWIRQPPRKGLEWLGVIWGSETTYYNSALKSRLTIIKDNSKSQ
VFLKMNSLQTDDTAIYYCAKHYYYGGSYAMDYWGQGTSVTVSSIQNPDPAVYQLRDSKSSDKS
VCLFTDFDSQTNVSQSKDSDVYITDKCVLDMRSMDFKSNSAVAWSNKSDFACANAFNNSIIPED
TFFPSPESSCDVKLVEKSFETDTNLNFQNLLVIVLRILLLKVAGFNLLMTLRLWSS
(SEQ ID NO.: 26) TCR_CAR_Split_CCR_CD226_CD2t Construct (VL and VH from FMC63; GM-
CSF and CD226 leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMDYPTLLLALLHVYRALCEEVLWHTSVPFAENMSLECVYPSMGILTQVEWFKIGTQQD
SIAIFSPTHGMVIRKPYAERVYFLNSTMASNNMTLFFRNASEDDVGYYSCSLYTYPQGTWQKVIQ
VVQSDSFEAAVPSNSHIVSEPGKNVTLTCQPQMTWPVQAVRWEKIQPRQIDLLTYCNLVHGRNF
TSKFPRQIVSNCSHGRWSVIVIPDVTVSDSGLYRCYLQASAGENETFVMRLTVAEGKTDNQYTLF
VAGGTVLLLLFVISITTIIVIFLKRKKQHPPPPPGHRSQAPSHRPPPPGHRVQHQPQKRPPAPSGTQ
VHQQKGPPLPRPRVQPKPPHGAAENSLSPSSNRAKRGSGATNFSLLKQAGDVEENPGPMLLLVTS
LLLCELPHPAFLLIPEVKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYYGGSYAMDYWGQGT
SVTVSSIQNPDPAVYQLRDSKSSDKSVCLFTDFDSQTNVSQSKDSDVYITDKCVLDMRSMDFKSN
SAVAWSNKSDFACANAFNNSIIPEDTFFPSPESSCDVKLVEKSFETDTNLNFQNLLVIVLRILLLKV
AGFNLLMTLRLWSS
(SEQ ID NO.: 27) TCR_CAR_Split_CCR_CD226_CD28 Construct (VL and VH from FMC63;
GM-CSF and CD226 leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMDYPTLLLALLHVYRALCEEVLWHTSVPFAENMSLECVYPSMGILTQVEWFKIGTQQD
SIAIFSPTHGMVIRKPYAERVYFLNSTMASNNMTLFFRNASEDDVGYYSCSLYTYPQGTWQKVIQ
VVQSDSFEAAVPSNSHIVSEPGKNVTLTCQPQMTWPVQAVRWEKIQPRQIDLLTYCNLVHGRNF
TSKFPRQIVSNCSHGRWSVIVIPDVTVSDSGLYRCYLQASAGENETFVMRLTVAEGKTDNQYTLF
VAGGTVLLLLFVISITTIIVIFLRSKRSRLLHSDYMNMTPRRPGPTRKHYQPYAPPRDFAAYRSRA
KRGSGATNFSLLKQAGDVEENPGPMLLLVTSLLLCELPHPAFLLIPEVKLQESGPGLVAPSQSLSVT
CTVSGVSLPDYGVSWIRQPPRKGLEWLGVIWGSETTYYNSALKSRLTIIKDNSKSQVFLKMNSLQ
TDDTAIYYCAKHYYYGGSYAMDYWGQGTSVTVSSIQNPDPAVYQLRDSKSSDKSVCLFTDFDS
QTNVSQSKDSDVYITDKCVLDMRSMDFKSNSAVAWSNKSDFACANAFNNSIIPEDTFFPSPESSC
DVKLVEKSFETDTNLNFQNLLVIVLRILLLKVAGFNLLMTLRLWSS
(SEQ ID NO.: 28) TCR_CAR_Split_CCR_CD226_4-1BB Construct (VL and VH from FMC63;
GM-CSF and CD226 leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMDYPTLLLALLHVYRALCEEVLWHTSVPFAENMSLECVYPSMGILTQVEWFKIGTQQD
SIAIFSPTHGMVIRKPYAERVYFLNSTMASNNMTLFFRNASEDDVGYYSCSLYTYPQGTWQKVIQ
VVQSDSFEAAVPSNSHIVSEPGKNVTLTCQPQMTWPVQAVRWEKIQPRQIDLLTYCNLVHGRNF
TSKFPRQIVSNCSHGRWSVIVIPDVTVSDSGLYRCYLQASAGENETFVMRLTVAEGKTDNQYTLF
VAGGTVLLLLFVISITTIIVIFLKRGRKKLLYIFKQPFMRPVQTTQEEDGCSCRFPEEEEGGCELRA
KRGSGATNFSLLKQAGDVEENPGPMLLLVTSLLLCELPHPAFLLIPEVKLQESGPGLVAPSQSLSVT
CTVSGVSLPDYGVSWIRQPPRKGLEWLGVIWGSETTYYNSALKSRLTIIKDNSKSQVFLKMNSLQ
TDDTAIYYCAKHYYYGGSYAMDYWGQGTSVTVSSIQNPDPAVYQLRDSKSSDKSVCLFTDFDS
QTNVSQSKDSDVYITDKCVLDMRSMDFKSNSAVAWSNKSDFACANAFNNSIIPEDTFFPSPESSC
DVKLVEKSFETDINLNFQNLLVIVLRILLLKVAGFNLLMTLRLWSS
(SEQ ID NO.: 29) TCR_CAR_Split_CCR_TIGIT_CD2 Construct (VL and VH from FMC63; GM-
CSF and TIGIT_leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMRWCLLLIWAQGLRQAPLASGMMTGTIETTGNISAEKGGSIILQCHLSSTTAQVTQVN
WEQQDQLLAICNADLGWHISPSFKDRVAPGPGLGLTLQSLTVNDTGEYFCIYHTYPDGTYTGRIF
LEVLESSVAEHGARFQIPLLGAMAATLVVICTAVIVVVAKRKKQRSRRNDEELETRAHRVATEE
RGRKPHQIPASTPQNPATSQHPPPPPGHRSQAPSHRPPPPGHRVQHQPQKRPPAPSGTQVHQQKG
PPLPRPRVQPKPPHGAAENSLSPSSNRAKRGSGATNFSLLKQAGDVEENPGPMLLLVTSLLLCELP
HPAFLLIPEVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGTSVTVSSI
QNPDPAVYQLRDSKSSDKSVCLFTDFDSQTNVSQSKDSDVYITDKCVLDMRSMDFKSNSAVAW
SNKSDFACANAFNNSIIPEDTFFPSPESSCDVKLVEKSFETDTNLNFQNLLVIVLRILLLKVAGFNL
LMTLRLWSS
(SEQ ID NO.: 30) TCR_CAR_Split_CCR_TIGIT_CD2t Construct (VL and VH from FMC63; GM-
CSF and TIGIT_leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMRWCLLLIWAQGLRQAPLASGMMTGTIETTGNISAEKGGSIILQCHLSSTTAQVTQVN
WEQQDQLLAICNADLGWHISPSFKDRVAPGPGLGLTLQSLTVNDTGEYFCIYHTYPDGTYTGRIF
LEVLESSVAEHGARFQIPLLGAMAATLVVICTAVIVVVAKRKKQHPPPPPGHRSQAPSHRPPPPG
HRVQHQPQKRPPAPSGTQVHQQKGPPLPRPRVQPKPPHGAAENSLSPSSNRAKRGSGATNESLLK
QAGDVEENPGPMLLLVTSLLLCELPHPAFLLIPEVKLQESGPGLVAPSQSLSVTCTVSGVSLPDYG
VSWIRQPPRKGLEWLGVIWGSETTYYNSALKSRLTIIKDNSKSQVFLKMNSLQTDDTAIYYCAKH
YYYGGSYAMDYWGQGTSVTVSSIQNPDPAVYQLRDSKSSDKSVCLFTDFDSQTNVSQSKDSDV
YITDKCVLDMRSMDFKSNSAVAWSNKSDFACANAFNNSIIPEDTFFPSPESSCDVKLVEKSFETD
TNLNFQNLLVIVLRILLLKVAGFNLLMTLRLWSS
(SEQ ID NO.: 31) TCR_CAR_Split_CCR_TIGIT_CD28 Construct (VL and VH from FMC63; GM-
CSF and TIGIT_leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMRWCLLLIWAQGLRQAPLASGMMTGTIETTGNISAEKGGSIILQCHLSSTTAQVTQVN
WEQQDQLLAICNADLGWHISPSFKDRVAPGPGLGLTLQSLTVNDTGEYFCIYHTYPDGTYTGRIF
LEVLESSVAEHGARFQIPLLGAMAATLVVICTAVIVVVARSKRSRLLHSDYMNMTPRRPGPTRK
HYQPYAPPRDFAAYRSRAKRGSGATNFSLLKQAGDVEENPGPMLLLVTSLLLCELPHPAFLLIPEV
KLQESGPGLVAPSQSLSVTCTVSGVSLPDYGVSWIRQPPRKGLEWLGVIWGSETTYYNSALKSRL
TIKDNSKSQVFLKMNSLQTDDTAIYYCAKHYYYGGSYAMDYWGQGTSVTVSSIQNPDPAVYQ
LRDSKSSDKSVCLFTDFDSQTNVSQSKDSDVYITDKCVLDMRSMDFKSNSAVAWSNKSDFACA
NAFNNSIIPEDTFFPSPESSCDVKLVEKSFETDTNLNFQNLLVIVLRILLLKVAGFNLLMTLRLWSS
(SEQ ID NO.: 32) TCR_CAR_Split_CCR_TIGIT_4-1BB Construct (VL and VH from FMC63;
GM-CSF and TIGIT_leader sequences underlined)
MLLLVTSLLLCELPHPAFLLIPDIQMTQTTSSLSASLGDRVTISCRASQDISKYLNWYQQKPDGTV
KLLIYHTSRLHSGVPSRFSGSGSGTDYSLTISNLEQEDIATYFCQQGNTLPYTFGGGTKLEITDLNK
VFPPEVAVFEPSEAEISHTQKATLVCLATGFFPDHVELSWWVNGKEVHSGVCTDPQPLKEQPAL
NDSRYCLSSRLRVSATFWQNPRNHFRCQVQFYGLSENDEWTQDRAKPVTQIVSAEAWGRADCG
FTSVSYQQGVLSATILYEILLGKATLYAVLVSALVLMAMVKRKDFRAKRGSGATNFSLLKQAGDV
EENPGPMRWCLLLIWAQGLRQAPLASGMMTGTIETTGNISAEKGGSIILQCHLSSTTAQVTQVN
WEQQDQLLAICNADLGWHISPSFKDRVAPGPGLGLTLQSLTVNDTGEYFCIYHTYPDGTYTGRIF
LEVLESSVAEHGARFQIPLLGAMAATLVVICTAVIVVVAKRGRKKLLYIFKQPFMRPVQTTQEED
GCSCRFPEEEEGGCELRAKRGSGATNFSLLKQAGDVEENPGPMLLLVTSLLLCELPHPAFLLIPEVK
LQESGPGLVAPSQSLSVTCTVSGVSLPDYGVSWIRQPPRKGLEWLGVIWGSETTYYNSALKSRLT
IIKDNSKSQVFLKMNSLQTDDTAIYYCAKHYYYGGSYAMDYWGQGTSVTVSSIQNPDPAVYQL
RDSKSSDKSVCLFTDFDSQTNVSQSKDSDVYITDKCVLDMRSMDFKSNSAVAWSNKSDFACAN
AFNNSIIPEDTFFPSPESSCDVKLVEKSFETDTNLNFQNLLVIVLRILLLKVAGFNLLMTLRLWSS
Sequence sgRNAs targeting TIGIT:
(SEQ ID NO.: 33) sgRNA_Base_Editor_TIGIT_1
GGTACCTTCCACAAGATCGG
(SEQ ID NO.: 34) sgRNA_Base_Editor_TIGIT_2
GGTGGTACCTTCCACAAGAT
(SEQ ID NO.: 35) sgRNA_Base_Editor_TIGIT_3
CAGGAATACCTGAGCTTTCT
(SEQ ID NO.: 36) sgRNA_Base_Editor_TIGIT_4
CTGGGCCAGGGGCTGAGGC
(SEQ ID NO.: 37) sgRNA_Base_Editor_TIGIT_5
AGGTTCCAGATTCCATTGCT
(SEQ ID NO.: 38) sgRNA_Base_Editor_TIGIT_6
AAGATCGGTGGCTTGTACAC
(SEQ ID NO.: 39) sgRNA_Base_Editor_TIGIT_7
TCTGCTTCCTGTAGGCCCTC
(SEQ ID NO.: 40) sgRNA_Base_Editor_TIGIT_8
TCTCCTCCTGATCTGGGCCC
(SEQ ID NO.: 41) sgRNA_Base_Editor_TIGIT_9
CCACTCGATCCTTGAAGGAT
Sequence hinges:
SEQ ID NO.: 42: EPKSCDKTHTCPPCP
SEQ ID NO.: 43: EPKSCDKTHTCPPCPAPELLGGP
SEQ ID NO.: 44: EPKSCDKTHTCPPCPAPEFLGGP
SEQ ID NO.: 45: EPKSCDKTHTCPSCP
SEQ ID NO.: 46: EPKSCDKTHTCPSCPAPELLGGP
SEQ ID NO.: 47: EPKSCDKTHTCPSCPAPEFLGGP
SEQ ID NO.: 48: ESKYGPPCPPCP
SEQ ID NO.: 49: ESKYGPPCPPCPAPELLGGP
SEQ ID NO.: 50: ESKYGPPCPPCPAPEFLGGP
SEQ ID NO.: 51: ESKYGPPCPSCP
SEQ ID NO.: 52: ESKYGPPCPSCPAPELLGGP
SEQ ID NO.: 53: ESKYGPPCPSCPAPEFLGGP
SEQ ID NO.: 54: GGGGSGGGGSGGGGS
SEQ ID NO.: 55: ESKYGPPCPSCP
Other Sequences
(SEQ ID NO.: 56) human TRAC from UniProt KB P01848
IQNPDPAVYQ LRDSKSSDKS VCLFTDFDSQ TNVSQSKDSD VYITDKTVLD MRSMDFKSNS
AVAWSNKSDF ACANAFNNSI IPEDTFFPSP ESSCDVKLVE KSFETDTNLN FQNLSVIGFR
ILLLKVAGEN LLMTLRLWSS
(SEQ ID NO.: 57) human TRAC with Thr→Cys and LVL mutations
IQNPDPAVYQLRDSKSSDKSVCLFTDFDSQTNVSQSKDSDVYITDKCVLDMRSMDFKSNSAVAW
SNKSDFACANAFNNSIIPEDTFFPSPESSCDVKLVEKSFETDTNLNFQNLLVIVLRILLLKVAGFNL
LMTLRLWSS
(SEQ ID NO.: 58) human TRBC1 from UniProt KB P01850
DLNKVFPPEV AVFEPSEAEI SHTQKATLVC LATGFFPDHV ELSWWVNGKE VHSGVSTDPQ
PLKEQPALND SRYCLSSRLR VSATFWQNPR NHFRCQVQFY GLSENDEWTQ DRAKPVTQIV
SAEAWGRADC GFTSVSYQQG VLSATILYEI LLGKATLYAV LVSALVLMAM VKRKDF
(SEQ ID NO.: 59) human TRBCI with Ser→Cys mutation
DLNKVFPPEVAVFEPSEAEISHTQKATLVCLATGFFPDHVELSWWVNGKEVHSGVCTDPQPLKE
QPALNDSRYCLSSRLRVSATFWQNPRNHFRCQVQFYGLSENDEWTQDRAKPVTQIVSAEAWGR
ADCGFTSVSYQQGVLSATILYEILLGKATLYAVLVSALVLMAMVKRKDF
(SEQ ID NO.: 60) human TRBC2 from UniProt KB AOASG9
DLKNVFPPKV AVFEPSEAEI SHTQKATLVC LATGFYPDHV ELSWWVNGKE VHSGVSTDPQ
PLKEQPALND SRYCLSSRLR VSATFWQNPR NHFRCQVQFY GLSENDEWTQ DRAKPVTQIV
SAEAWGRADC GFTSESYQQG VLSATILYEI LLGKATLYAV LVSALVLMAM VKRKDSRG
(SEQ ID NO.: 61) human TRBC2 with Ser→Cys mutation
DLKNVFPPKV AVFEPSEAEI SHTQKATLVC LATGFYPDHV ELSWWVNGKE VHSGVCTDPQ
PLKEQPALND SRYCLSSRLR VSATFWQNPR NHFRCQVQFY GLSENDEWTQ DRAKPVTQIV
SAEAWGRADC GFTSESYQQG VLSATILYEI LLGKATLYAV LVSALVLMAM VKRKDSRG
(SEQ ID NO.: 62) TRAC sequence motif
LSVIGF
(SEQ ID NO.: 63) TRAC sequence motif with L-V-L mutations
LLVIVL
(SEQ ID NO.: 64) TRBC1 intracellular sequence
VKRKDF
(SEQ ID NO.: 65) TRBC2 intracellular sequence
MAMVKRKDSRG
(SEQ ID NO.: 66) WT-1 126 peptide
RMFPNAPYL
(SEQ ID NO.: 67) NY-ESO-1 157 peptide
SLLMWITQC
(SEQ ID NO.: 68) Townsend linker
GSGGSGGSGGTG
(SEQ ID NO.: 69) Whitlow linker (linker 218)
GSTSGSGKPGSGEGSTKG
(SEQ ID NO.: 70) linker
GSGKPGSGEG
(SEQ ID NO.: 71) linker
GKPGSGEG
(SEQ ID NO.: 72) linker
SGKPGSGE
(SEQ ID NO.: 73) linker
EGKSSGSGSESKVD
(SEQ ID NO.: 74) linker motif
BPXXXZ, wherein each X is independently a glycine (G) or serine (S), B is a positively charged amino
acid and Z is glycine (G) or a negatively charged amino acid
(SEQ ID NO.: 75) CD226 precursor (UniProt KB Q15762)
MDYPTLLLAL LHVYRALCEE VLWHTSVPFA ENMSLECVYP SMGILTQVEW
FKIGTQQDSI AIFSPTHGMV IRKPYAERVY FLNSTMASNN MTLFFRNASE
DDVGYYSCSL YTYPQGTWQK VIQVVQSDSF EAAVPSNSHI VSEPGKNVTL
TCQPQMTWPV QAVRWEKIQP RQIDLLTYCN LVHGRNFTSK FPRQIVSNCS
HGRWSVIVIP DVTVSDSGLY RCYLQASAGE NETFVMRLTV AEGKTDNQYT
LFVAGGTVLL LLFVISITTI IVIFLNRRRR RERRDLFTES WDTQKAPNNY
RSPISTSQPT NQSMDDTRED IYVNYPTFSR RPKTRV
(SEQ ID NO.: 76) CD226 leader sequence
MDYPTLLLALLHVYRALC
(SEQ ID NO.: 77) CD226 ectodomain sequence
EEVLWHTSVPFAENMSLECVYPSMGILTQVEWFKIGTQQDSIAIFSPTHGMVIRKPYAERVYFLN
STMASNNMTLFFRNASEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIVSNCSHGRWSVIVIPDV
TVSDSGLYRCYLQASAGENETFVMRLTVAEGKTDNQYTLFVA
(SEQ ID NO.: 78) CD226 transmembrane sequence
GGTVLLLLFVISITTIIVIFL
(SEQ ID NO.: 79) CD226 endodomain sequence with K295A and K333A mutations
NRRRRRERRDLFTESWDTQAAPNNYRSPISTSQPTNQSMDDTREDIYVNYPTFSRRPATRV
(SEQ ID NO.: 80) TIGIT precursor (UniProt KB Q495A1)
MRWCLLLIWA QGLRQAPLAS GMMTGTIETT GNISAEKGGS IILQCHLSST
TAQVTQVNWE QQDQLLAICN ADLGWHISPS FKDRVAPGPG LGLTLQSLTV
NDTGEYFCIY HTYPDGTYTG RIFLEVLESS VAEHGARFQI PLLGAMAATL
VVICTAVIVV VALTRKKKAL RIHSVEGDLR RKSAGQEEWS PSAPSPPGSC
VQAEAAPAGL CGEQRGEDCA ELHDYFNVLS YRSLGNCSFF TETG
(SEQ ID NO.: 81) TIGIT leader sequence
MRWCLLLIWAQGLRQAPLASG
(SEQ ID NO.: 82) TIGIT ectodomain sequence
MMTGTIETTGNISAEKGGSIILQCHLSSTTAQVTQVNWEQQDQLLAICNADLGWHISPSFKDRVA
PGPGLGLTLQSLTVNDTGEYFCIYHTYPDGTYTGRIFLEVLESSVAEHGARFQIP
(SEQ ID NO.: 83) TIGIT transmembrane domain sequence
LLGAMAATLVVICTAVIVVVA
(SEQ ID NO.: 84) CD2 endodomain sequence
KRKKQRSRRNDEELETRAHRVATEERGRKPHQIPASTPQNPATSQHPPPPPGHRSQAPSHRPPPPG
HRVQHQPQKRPPAPSGTQVHQQKGPPLPRPRVQPKPPHGAAENSLSPSSN
(SEQ ID NO.: 85) truncated CD2 endodomain sequence
KRKKQHPPPPPGHRSQAPSHRPPPPGHRVQHQPQKRPPAPSGTQVHQQKGPPLPRPRVQPKPPHG
AAENSLSPSSN
(SEQ ID NO.: 86) CD28 co-stimulatory domain sequence
RSKRSRLLHSDYMNMTPRRPGPTRKHYQPYAPPRDFAAYRS
(SEQ ID NO.: 87) 4-1BB co-stimulatory domain sequence
KRGRKKLLYIFKQPFMRPVQTTQEEDGCSCRFPEEEEGGCEL
(SEQ ID NO.: 88) CD226-based (CD226mut) CCR sequence
EEVLWHTSVPFAENMSLECVYPSMGILTQVEWFKIGTQQDSIAIFSPTHGMVIRKPYAERVYFLN
STMASNNMTLFFRNASEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIVSNCSHGRWSVIVIPDV
TVSDSGLYRCYLQASAGENETFVMRLTVAEGKTDNQYTLFVAGGTVLLLLFVISITTIIVIFLNRR
RRRERRDLFTESWDTQAAPNNYRSPISTSQPTNQSMDDTREDIYVNYPTFSRRPATRV
(SEQ ID NO.: 89) CD226-based (CD2) CCR sequence
EEVLWHTSVPFAENMSLECVYPSMGILTQVEWFKIGTQQDSIAIFSPTHGMVIRKPYAERVYFLN
STMASNNMTLFFRNASEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIVSNCSHGRWSVIVIPDV
TVSDSGLYRCYLQASAGENETFVMRLTVAEGKTDNQYTLFVAGGTVLLLLFVISITTIIVIFLKRK
KQRSRRNDEELETRAHRVATEERGRKPHQIPASTPQNPATSQHPPPPPGHRSQAPSHRPPPPGHRV
QHQPQKRPPAPSGTQVHQQKGPPLPRPRVQPKPPHGAAENSLSPSSN
(SEQ ID NO.: 90) CD226-based (CD2t) CCR sequence
EEVLWHTSVPFAENMSLECVYPSMGILTQVEWFKIGTQQDSIAIFSPTHGMVIRKPYAERVYFLN
STMASNNMTLFFRNASEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIVSNCSHGRWSVIVIPDV
TVSDSGLYRCYLQASAGENETFVMRLTVAEGKTDNQYTLFVAGGTVLLLLFVISITTIIVIFLKRK
KQHPPPPPGHRSQAPSHRPPPPGHRVQHQPQKRPPAPSGTQVHQQKGPPLPRPRVQPKPPHGAAE
NSLSPSS
(SEQ ID NO.: 91) CD226-based (CD28) CCR sequence
EEVLWHTSVPFAENMSLECVYPSMGILTQVEWFKIGTQQDSIAIFSPTHGMVIRKPYAERVYFLN
STMASNNMTLFFRNASEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIVSNCSHGRWSVIVIPDV
TVSDSGLYRCYLQASAGENETFVMRLTVAEGKTDNQYTLFVAGGTVLLLLFVISITTIIVIFLRSK
RSRLLHSDYMNMTPRRPGPTRKHYQPYAPPRDFAAYRS
(SEQ ID NO.: 92) CD226-based (4-1BB) CCR sequence
EEVLWHTSVPFAENMSLECVYPSMGILTQVEWFKIGTQQDSIAIFSPTHGMVIRKPYAERVYFLN
STMASNNMTLFFRNASEDDVGYYSCSLYTYPQGTWQKVIQVVQSDSFEAAVPSNSHIVSEPGKN
VTLTCQPQMTWPVQAVRWEKIQPRQIDLLTYCNLVHGRNFTSKFPRQIVSNCSHGRWSVIVIPDV
TVSDSGLYRCYLQASAGENETFVMRLTVAEGKTDNQYTLFVAGGTVLLLLFVISITTIIVIELKRG
RKKLLYIFKQPFMRPVQTTQEEDGCSCRFPEEEEGGCEL
(SEQ ID NO.: 93) TIGIT-based (CD2) CCR sequence
MMTGTIETTGNISAEKGGSIILQCHLSSTTAQVTQVNWEQQDQLLAICNADLGWHISPSFKDRVA
PGPGLGLTLQSLTVNDTGEYFCIYHTYPDGTYTGRIFLEVLESSVAEHGARFQIPLLGAMAATLV
VICTAVIVVVAKRKKQRSRRNDEELETRAHRVATEERGRKPHQIPASTPQNPATSQHPPPPPGHR
SQAPSHRPPPPGHRVQHQPQKRPPAPSGTQVHQQKGPPLPRPRVQPKPPHGAAENSLSPSSN
(SEQ ID NO.: 94) TIGIT-based (CD2t) CCR sequence
MMTGTIETTGNISAEKGGSIILQCHLSSTTAQVTQVNWEQQDQLLAICNADLGWHISPSFKDRVA
PGPGLGLTLQSLTVNDTGEYFCIYHTYPDGTYTGRIFLEVLESSVAEHGARFQIPLLGAMAATLV
VICTAVIVVVAKRKKQHPPPPPGHRSQAPSHRPPPPGHRVQHQPQKRPPAPSGTQVHQQKGPPLP
RPRVQPKPPHGAAENSLSPSSN
(SEQ ID NO.: 95) TIGIT-based (CD28) CCR sequence
MMTGTIETTGNISAEKGGSIILQCHLSSTTAQVTQVNWEQQDQLLAICNADLGWHISPSFKDRVA
PGPGLGLTLQSLTVNDTGEYFCIYHTYPDGTYTGRIFLEVLESSVAEHGARFQIPLLGAMAATLV
VICTAVIVVVARSKRSRLLHSDYMNMTPRRPGPTRKHYQPYAPPRDFAAYRS
(SEQ ID NO.: 96) TIGIT-based (4-1BB) CCR sequence
MMTGTIETTGNISAEKGGSIILQCHLSSTTAQVTQVNWEQQDQLLAICNADLGWHISPSFKDRVA
PGPGLGLTLQSLTVNDTGEYFCIYHTYPDGTYTGRIFLEVLESSVAEHGARFQIPLLGAMAATLV
VICTAVIVVVAKRGRKKLLYIFKQPFMRPVQTTQEEDGCSCRFPEEEEGGCEL
(SEQ ID NO.: 97) FMC63 antibody VL sequence
DIQMTQTTSSLSASLGDRVTISCRASQDISKYLNWYQQKPDGTVKLLIYHTSRLHSGVPSRFSGSG
SGTDYSLTISNLEQEDIATYFCQQGNTLPYTFGGGTKLEIT
(SEQ ID NO.: 98) FMC63 antibody VH sequence
EVKLQESGPGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSS
(SEQ ID NO.: 99) FMC63 antibody scFv (VL-linker-VH) sequence
DIQMTQTTSSLSASLGDRVTISCRASQDISKYLNWYQQKPDGTVKLLIYHTSRLHSGVPSRFSGSG
SGTDYSLTISNLEQEDIATYFCQQGNTLPYTFGGGTKLEITGSTSGSGKPGSGEGSTKGEVKLQESG
PGLVAPSQSLSVTCTVSGVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSS
(SEQ ID NO.: 100) FMC63 antibody scFv (VH-linker-VL) sequence
EVKLQESGPGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSSGSTSGSGKP
GSGEGSTKGDIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEIT
(SEQ ID NO.: 101) anti-GPRCSD antibody VL sequence
QSALTQPASVSGSPGQSITISCTGTSSDVGGYNYVSWYQQHPGKAPKLMIYDVSKRPSGVSNRFS
GSKSGNTASLTISGLQAEDEADYYCSSYTRSSTEVFGGGTKLTVLGSR
(SEQ ID NO.: 102) anti-GPRCSD antibody VH sequence
EVQLVQSGAEVKKPGASVKVSCKASGYTFTSYYMHWVRQAPGQGLEWMGIINPSGGSTSYAQK
FQGRVTMTRDTSTSTVYMELSSLRSEDTAVYYCARGGSKKWSGEKWRRENFDYWGQGTLVTV
SS
(SEQ ID NO.: 103) anti-GPRCSD antibody scFv (VL-linker-VH) sequence
QSALTQPASVSGSPGQSITISCTGTSSDVGGYNYVSWYQQHPGKAPKLMIYDVSKRPSGVSNRFS
GSKSGNTASLTISGLQAEDEADYYCSSYTRSSTEVFGGGTKLTVLGSRGGGGSGGGGSGGGGSLE
MAEVQLVQSGAEVKKPGASVKVSCKASGYTFTSYYMHWVRQAPGQGLEWMGIINPSGGSTSYA
QKFQGRVTMTRDTSTSTVYMELSSLRSEDTAVYYCARGGSKKWSGEKWRRENFDYWGQGTLV
TVSS
(SEQ ID NO.: 104) anti-BCMA antibody (C11D5.3) VH sequence
QIQLVQSGPELKKPGETVKISCKASGYTFTDYSINWVKRAPGKGLKWMGWINTETREPAYAYDF
RGRFAFSLETSASTAYLQINNLKYEDTATYFCALDYSYAMDYWGQGTSVTVSS
(SEQ ID NO.: 105) anti-BCMA antibody (C11DS.3) VL sequence
DIVLTQSPPSLAMSLGKRATISCRASESVTILGSHLIHWYQQKPGQPPTLLIQLASNVQTGVPARFS
GSGSRTDFTLTIDPVEEDDVAVYYCLQSRTIPRTFGGGTKLEIK
(SEQ ID NO.: 106) anti-BCMA antibody (C11DS.3) scFv (VH-linker-VL) sequence
QIQLVQSGPELKKPGETVKISCKASGYTFTDYSINWVKRAPGKGLKWMGWINTETREPAYAYDF
RGRFAFSLETSASTAYLQINNLKYEDTATYFCALDYSYAMDYWGQGTSVTVSSGGGGSGGGGS
GGGGSDIVLTQSPPSLAMSLGKRATISCRASESVTILGSHLIHWYQQKPGQPPTLLIQLASNVQTG
VPARFSGSGSRTDFTLTIDPVEEDDVAVYYCLQSRTIPRTFGGGTKLEIK
(SEQ ID NO.: 107) VHH sequence
QVKLVQSGGGLVQTGGSLRLSCAASEITFDMYSMGWYREAPGKARDAVASITNRGNTYYADSV
KGRFTISRDNAKKTMYLQMNSLKPEDTAVYYCNVYRTGFSDYWGQGTQVTVSS
(SEQ ID NO.: 108) VHH sequence - AAA linker - VHH sequence
QVKLVQSGGGLVQTGGSLRLSCAASEITFDMYSMGWYREAPGKARDAVASITNRGNTYYADSV
KGRFTISRDNAKKTMYLQMNSLKPEDTAVYYCNVYRTGFSDYWGQGTQVTVSSAAAQVKLVQ
SGGGLVQTGGSLRLSCAASEITFDMYSMGWYREAPGKARDAVASITNRGNTYYADSVKGRFTIS
RDNAKKTMYLQMNSLKPEDTAVYYCNVYRTGFSDYWGQGTQVTVSS
(SEQ ID NO.: 109) Bel2 ectodomain sequence
AHAGRTGYDNREIVMKYIHYKLSQRGYEWDAGDDAEENRTEAPEGTESEVVHRALRDAGDDF
ERRYRRDFAEMSSQLHLTPDTARQRFETVVEELFRDGVNWGRIVAFFEFGGVMCVESVNREMSP
LVDNIAEWMTEYLNRHLHTWIQDNGGWDAFVELYGPSMR
(SEQ ID NO.: 110) FMC63 VL-TRBC
DIQMTQTTSSLSASLGDRVTISCRASQDISKYLNWYQQKPDGTVKLLIYHTSRLHSGVPSRFSGSG
SGTDYSLTISNLEQEDIATYFCQQGNTLPYTFGGGTKLEITDLNKVFPPEVAVFEPSEAEISHTQKA
TLVCLATGFFPDHVELSWWVNGKEVHSGVCTDPQPLKEQPALNDSRYCLSSRLRVSATFWQNP
RNHFRCQVQFYGLSENDEWTQDRAKPVTQIVSAEAWGRADCGFTSVSYQQGVLSATILYEILLG
KATLYAVLVSALVLMAMVKRKDF
(SEQ ID NO.: 111) FMC63 VH-TRAC
EVKLQESGPGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSSIQNPDPAV
YQLRDSKSSDKSVCLFTDFDSQTNVSQSKDSDVYITDKCVLDMRSMDFKSNSAVAWSNKSDFA
CANAFNNSIIPEDTFFPSPESSCDVKLVEKSFETDTNLNFQNLLVIVLRILLLKVAGFNLLMTLRLW
SS
(SEQ ID NO.: 112) FMC63 VH-TRBC
EVKLQESGPGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSSDLNKVFPP
EVAVFEPSEAEISHTQKATLVCLATGFFPDHVELSWWVNGKEVHSGVCTDPQPLKEQPALNDSR
YCLSSRLRVSATFWQNPRNHFRCQVQFYGLSENDEWTQDRAKPVTQIVSAEAWGRADCGFTSV
SYQQGVLSATILYEILLGKATLYAVLVSALVLMAMVKRKDF
(SEQ ID NO.: 113) FMC63 VL-TRAC
DIQMTQTTSSLSASLGDRVTISCRASQDISKYLNWYQQKPDGTVKLLIYHTSRLHSGVPSRFSGSG
SGTDYSLTISNLEQEDIATYFCQQGNTLPYTFGGGTKLEITIQNPDPAVYQLRDSKSSDKSVCLFT
DFDSQTNVSQSKDSDVYITDKCVLDMRSMDFKSNSAVAWSNKSDFACANAFNNSIIPEDTFFPSP
ESSCDVKLVEKSFETDTNLNFQNLLVIVLRILLLKVAGENLLMTLRLWSS
(SEQ ID NO.: 114) FMC63 scFv (VL-linker-VH) TRAC
DIQMTQTTSSLSASLGDRVTISCRASQDISKYLNWYQQKPDGTVKLLIYHTSRLHSGVPSRFSGSG
SGTDYSLTISNLEQEDIATYFCQQGNTLPYTFGGGTKLEITGSTSGSGKPGSGEGSTKGEVKLQESG
PGLVAPSQSLSVTCTVSGVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSSIQNPDPAVYQLRDSKSS
DKSVCLFTDFDSQTNVSQSKDSDVYITDKCVLDMRSMDFKSNSAVAWSNKSDFACANAFNNSII
PEDTFFPSPESSCDVKLVEKSFETDTNLNFQNLLVIVLRILLLKVAGFNLLMTLRLWSS
(SEQ ID NO.: 115) FMC63 scFv (VL-linker-VH) TRBC
DIQMTQTTSSLSASLGDRVTISCRASQDISKYLNWYQQKPDGTVKLLIYHTSRLHSGVPSRESGSG
SGTDYSLTISNLEQEDIATYFCQQGNTLPYTFGGGTKLEITGSTSGSGKPGSGEGSTKGEVKLQES
GPGLVAPSQSLSVTCTVSGVSLPDYGVSWIRQPPRKGLEWLGVIWGSETTYYNSALKSRLTIIKD
NSKSQVFLKMNSLQTDDTAIYYCAKHYYYGGSYAMDYWGQGTSVTVSSDLNKVFPPEVAVFEP
SEAEISHTQKATLVCLATGFFPDHVELSWWVNGKEVHSGVCTDPQPLKEQPALNDSRYCLSSRL
RVSATFWQNPRNHFRCQVQFYGLSENDEWTQDRAKPVTQIVSAEAWGRADCGFTSVSYQQGV
LSATILYEILLGKATLYAVLVSALVLMAMVKRKDF
(SEQ ID NO.: 116) FMC63 scFy (VH-linker-VL) TRAC
EVKLQESGPGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSSGSTSGSGKP
GSGEGSTKGDIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITIQNPDPAVYQLRDSKS
SDKSVCLFTDFDSQTNVSQSKDSDVYITDKCVLDMRSMDFKSNSAVAWSNKSDFACANAFNNSI
IPEDTFFPSPESSCDVKLVEKSFETDTNLNFQNLLVIVLRILLLKVAGFNLLMTLRLWSS
(SEQ ID NO.: 117) FMC63 scFv (VH-linker-VL) TRBC
EVKLQESGPGLVAPSQSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTSVTVSSGSTSGSGKP
GSGEGSTKGDIQMTQTTSSLSASLGDRVTISCRASQDISKYLNWYQQKPDGTVKLLIYHTSRLHS
GVPSRFSGSGSGTDYSLTISNLEQEDIATYFCQQGNTLPYTFGGGTKLEITDLNKVFPPEVAVFEPS
EAEISHTQKATLVCLATGFFPDHVELSWWVNGKEVHSGVCTDPQPLKEQPALNDSRYCLSSRLR
VSATFWQNPRNHFRCQVQFYGLSENDEWTQDRAKPVTQIVSAEAWGRADCGFTSVSYQQGVLS
ATILYEILLGKATLYAVLVSALVLMAMVKRKDF
(SEQ ID NO.: 118) anti-GPRC5D scFv (VL-linker-VH) TRBC
QSALTQPASVSGSPGQSITISCTGTSSDVGGYNYVSWYQQHPGKAPKLMIYDVSKRPSGVSNRFS
GSKSGNTASLTISGLQAEDEADYYCSSYTRSSTEVFGGGTKLTVLGSRGGGGSGGGGSGGGGSL
EMAEVQLVQSGAEVKKPGASVKVSCKASGYTFTSYYMHWVRQAPGQGLEWMGIINPSGGSTS
YAQKFQGRVTMTRDTSTSTVYMELSSLRSEDTAVYYCARGGSKKWSGEKWRRENFDYWGQGT
LVTVSSDLNKVFPPEVAVFEPSEAEISHTQKATLVCLATGFFPDHVELSWWVNGKEVHSGVCTD
PQPLKEQPALNDSRYCLSSRLRVSATFWQNPRNHFRCQVQFYGLSENDEWTQDRAKPVTQIVSA
EAWGRADCGFTSVSYQQGVLSATILYEILLGKATLYAVLVSALVLMAMVKRKDF
(SEQ ID NO.: 119) anti-BCMA scFv (VH-linker-VL) TRAC
QIQLVQSGPELKKPGETVKISCKASGYTFTDYSINWVKRAPGKGLKWMGWINTETREPAYAYDF
RGRFAFSLETSASTAYLQINNLKYEDTATYFCALDYSYAMDYWGQGTSVTVSSGGGGSGGGGS
GGGGSDIVLTQSPPSLAMSLGKRATISCRASESVTILGSHLIHWYQQKPGQPPTLLIQLASNVQTG
VPARFSGSGSRTDFTLTIDPVEEDDVAVYYCLQSRTIPRTFGGGTKLEIKIQNPDPAVYQLRDSKS
SDKSVCLFTDFDSQTNVSQSKDSDVYITDKCVLDMRSMDFKSNSAVAWSNKSDFACANAFNNSI
IPEDTFFPSPESSCDVKLVEKSFETDTNLNFQNLLVIVLRILLLKVAGENLLMTLRLWSS
(SEQ ID NO.: 120) VHH-AAA-VHH TRAC
QVKLVQSGGGLVQTGGSLRLSCAASEITFDMYSMGWYREAPGKARDAVASITNRGNTYYADSV
KGRFTISRDNAKKTMYLQMNSLKPEDTAVYYCNVYRTGFSDYWGQGTQVTVSSAAAQVKLVQ
SGGGLVQTGGSLRLSCAASEITFDMYSMGWYREAPGKARDAVASITNRGNTYYADSVKGRFTIS
RDNAKKTMYLQMNSLKPEDTAVYYCNVYRTGFSDYWGQGTQVTVSSIQNPDPAVYQLRDSKS
SDKSVCLFTDFDSQTNVSQSKDSDVYITDKCVLDMRSMDFKSNSAVAWSNKSDFACANAFNNSI
IPEDTFFPSPESSCDVKLVEKSFETDTNLNFQNLLVIVLRILLLKVAGENLLMTLRLWSS
(SEQ ID NO.: 121) VHH-AAA-VHH TRBC
QVKLVQSGGGLVQTGGSLRLSCAASEITFDMYSMGWYREAPGKARDAVASITNRGNTYYADSV
KGRFTISRDNAKKTMYLQMNSLKPEDTAVYYCNVYRTGFSDYWGQGTQVTVSSAAAQVKLVQ
SGGGLVQTGGSLRLSCAASEITFDMYSMGWYREAPGKARDAVASITNRGNTYYADSVKGRFTIS
RDNAKKTMYLQMNSLKPEDTAVYYCNVYRTGFSDYWGQGTQVTVSSDLNKVFPPEVAVFEPS
EAEISHTQKATLVCLATGFFPDHVELSWWVNGKEVHSGVCTDPQPLKEQPALNDSRYCLSSRLR
VSATFWQNPRNHFRCQVQFYGLSENDEWTQDRAKPVTQIVSAEAWGRADCGFTSVSYQQGVLS
ATILYEILLGKATLYAVLVSALVLMAMVKRKDF
(SEQ ID NO.: 122) Bcl2-linker-hinge-TRAC
AHAGRTGYDNREIVMKYIHYKLSQRGYEWDAGDDAEENRTEAPEGTESEVVHRALRDAGDDF
ERRYRRDFAEMSSQLHLTPDTARQRFETVVEELFRDGVNWGRIVAFFEFGGVMCVESVNREMSP
LVDNIAEWMTEYLNRHLHTWIQDNGGWDAFVELYGPSMRGGGGSGGGGSESKYGPPCPPCPIQ
NPDPAVYQLRDSKSSDKSVCLFTDFDSQTNVSQSKDSDVYITDKCVLDMRSMDFKSNSAVAWS
NKSDFACANAFNNSIIPEDTFFPSPESSCDVKLVEKSFETDTNLNFQNLLVIVLRILLLKVAGENLL
MTLRLWSS
(SEQ ID NO.: 123) Flag/HA-tag-Bcl2-linker-hinge-TRAC
DYKDEYPYDVPDYAGSAHAGRTGYDNREIVMKYIHYKLSQRGYEWDAGDDAEENRTEAPEGT
ESEVVHRALRDAGDDFERRYRRDFAEMSSQLHLTPDTARQRFETVVEELFRDGVNWGRIVAFFE
FGGVMCVESVNREMSPLVDNIAEWMTEYLNRHLHTWIQDNGGWDAFVELYGPSMRGGGGSG
GGGSESKYGPPCPPCPIQNPDPAVYQLRDSKSSDKSVCLFTDFDSQTNVSQSKDSDVYITDKCVL
DMRSMDFKSNSAVAWSNKSDFACANAFNNSIIPEDTFFPSPESSCDVKLVEKSFETDTNLNFQNL
LVIVLRILLLKVAGFNLLMTLRLWSS
(SEQ ID NO.: 124) Bcl2-linker-hinge-TRBC
AHAGRTGYDNREIVMKYIHYKLSQRGYEWDAGDDAEENRTEAPEGTESEVVHRALRDAGDDF
ERRYRRDFAEMSSQLHLTPDTARQRFETVVEELFRDGVNWGRIVAFFEFGGVMCVESVNREMSP
LVDNIAEWMTEYLNRHLHTWIQDNGGWDAFVELYGPSMRGGGGSGGGGSESKYGPPCPPCPDL
NKVFPPEVAVFEPSEAEISHTQKATLVCLATGFFPDHVELSWWVNGKEVHSGVCTDPQPLKEQP
ALNDSRYCLSSRLRVSATFWQNPRNHFRCQVQFYGLSENDEWTQDRAKPVTQIVSAEAWGRAD
CGFTSVSYQQGVLSATILYEILLGKATLYAVLVSALVLMAMVKRKDF
(SEQ ID NO.: 125) Flag/HA-tag-Bcl2-linker-hinge-TRBC
DYKDEYPYDVPDYAGSAHAGRTGYDNREIVMKYIHYKLSQRGYEWDAGDDAEENRTEAPEGT
ESEVVHRALRDAGDDFERRYRRDFAEMSSQLHLTPDTARQRFETVVEELFRDGVNWGRIVAFFE
FGGVMCVESVNREMSPLVDNIAEWMTEYLNRHLHTWIQDNGGWDAFVELYGPSMRGGGGSG
GGGSESKYGPPCPPCPDLNKVFPPEVAVFEPSEAEISHTQKATLVCLATGFFPDHVELSWWVNGK
EVHSGVCTDPQPLKEQPALNDSRYCLSSRLRVSATFWQNPRNHFRCQVQFYGLSENDEWTQDR
AKPVTQIVSAEAWGRADCGFTSVSYQQGVLSATILYEILLGKATLYAVLVSALVLMAMVKRKD
F
(SEQ ID NO.: 126) GM-CSF leader sequence
MLLLVTSLLLCELPHPAFLLIP
(SEQ ID NO.: 127) CD8a leader sequence
MALPVTALLLPLALLLHAARP
(SEQ ID NO.: 128) CD88 leader sequence
MRPRLWLLLAAQLTVLHGNSV
(SEQ ID NO.: 129) consensus minimal furin cleavage site sequence
R-X-X-R
(SEQ ID NO.: 130) consensus minimal furin cleavage site sequence
R-X-K/R-R
(SEQ ID NO.: 131) minimal furin cleavage site sequence
RAKR
(SEQ ID NO.: 132) minimal furin cleavage site sequence
RARR
(SEQ ID NO.: 133) Porcine teschovirus-1 2A (P2A) self-cleaving peptide with N-terminal GSG
linker
GSGATNFSLLKQAGDVEENPGP
(SEQ ID NO.: 134) Thoseaasigna virus 2A (T2A) self-cleaving peptide
LEGGGEGRGSLLTCGDVEENPGPR
(SEQ ID NO.: 135) Equine rhinitis A virus (ERAV) 2A (E2A) self-cleaving peptide
QCTNYALLKLAGDVESNPGP
(SEQ ID NO.: 136) Foot-and-Mouth disease virus 2A (F2A) self-cleaving peptide with N-terminal G-
S-G linker
GSGVKQTLNFDLLKLAGDVESNPGP
(SEQ ID NO.: 137) Flag Tag
DYKDDDDK
(SEQ ID NO.: 138) Flag Tag variant
DYKDE
(SEQ ID NO.: 139) HA Tag
YPYDVDPDYA
(SEQ ID NO.: 140) Flag/HA Hybrid Tag
DYKDEYYPYDVDPDYA
(SEQ ID NO.: 141) ICOS intracellular sequence
CWLTKKKYSSSVHDPNGEYMFMRAVNTAKKSRLTDVTL
(SEQ ID NO.: 142) OX40 intracellular sequence
ALYLLRRDQRLPPDAHKPPGGGSFRTPIQEEQADAHSTLAKI
(SEQ ID NO.: 143) GITR intracellular sequence
QLGLHIWQLRSQCMWPRETQLLLEVPPSTEDARSCQFPEEERGERSAEEKGRLGDLWV
(SEQ ID NO.: 144) CD27 intracellular sequence
QRRKYRSNKGESPVEPAEPCHYSCPREEEGSTIPIQEDYRKPEPACSP
(SEQ ID NO.: 145) HVEM intracellular sequence
CVKRRKPRGDVVKVIVSVQRKRQEAEGEATVIEALQAPPDVTTVAVEETIPSFTGRSPNH
(SEQ ID NO.: 146) DAP10 intracellular sequence
LCARPRRSPAQEDGKVYINMPGRG
(SEQ ID NO.: 147) SLAMF1 intracellular sequence
QLRRRGKINHYQTTVEKKSLTIYAQVQKPGPLQKKLDSFPAQDPCTTIYVAATEPVPESV
QETNSITVYASVTLPES
(SEQ ID NO.: 148) LIGHT intracellular sequence
LQLHWRLGEMVTRLPDGPAGSWEQLIQERRSHEVNPAAHLTGANSSLTGSGGPLLWETQL
GLAFLRGLSYHDGALVVTKAGYYYIYSKVQLGGVGCPLGLASTITHGLYKRTPRYPEELE
LLVSQQSPCGRATSSSRVWWDSSFLGGVVHLEAGEKVVVRVLDERLVRLRDGTRSYFGAF
MV
(SEQ ID NO.: 149) CD30 intracellular sequence
CHRRACRKRIRQKLHLCYPVQTSQPKLELVDSRPRRSSTQLRSGASVTEPVAEERGLMSQ
PLMETCHSVGAAYLESLPLQDASPAGGPSSPRDLPEPRVSTEHTNNKIEKIYIMKADTVI
VGTVKAELPEGRGLAGPAEPELEEELEADHTPHYPEQETEPPLGSCSDVMLSVEEEGKED
PLPTAASGK
(SEQ ID NO.: 150) murine IgG, kappa leader sequence
METDTLLLWVLLLWVPGSTG
(SEQ ID NO.: 151) Myc tag
EQKLISEEDL

### EXAMPLES

### Example 1

### Design and Testing of Tcr-Car Hybrid Molecules

Lentiviral vectors were designed that encode synthetic TCR/CARs comprising TCRα and β constant domains fused to the VH and VL of scFvs specific for CD19 and RORI. The TCR/CAR molecules were designed in both a “split scFv” format where the VH is fused to TCRβ and VL to TCRα, and a “full scFv” format where VH and VL are fused in tandem to the TCRα or TCRβ, respectively. The full scFv design can allow for dual specificity from a single receptor as one scFv can be fused to TCRα and the other scFv can be fused to TCRβ. All TCR/CAR formats were expressed in primary T cells and >90% of the cells were edited for both TCRα and TCRβ using base editing to prevent mispairing.

The affinity of an scFv is typically higher than that of a TCR, which could lead to prolonged signaling and AICD, although this was not observed with CD19 or RORI TCR/CARs. Some TCR/CAR constructs included scFv with reduced affinity via point mutations that were predicted by available algorithms²⁹; reducing affinity may achieve a “TCR-like” level which may be beneficial in promoting serial triggering in the TCR/CAR format and further improve sensitivity to antigen.

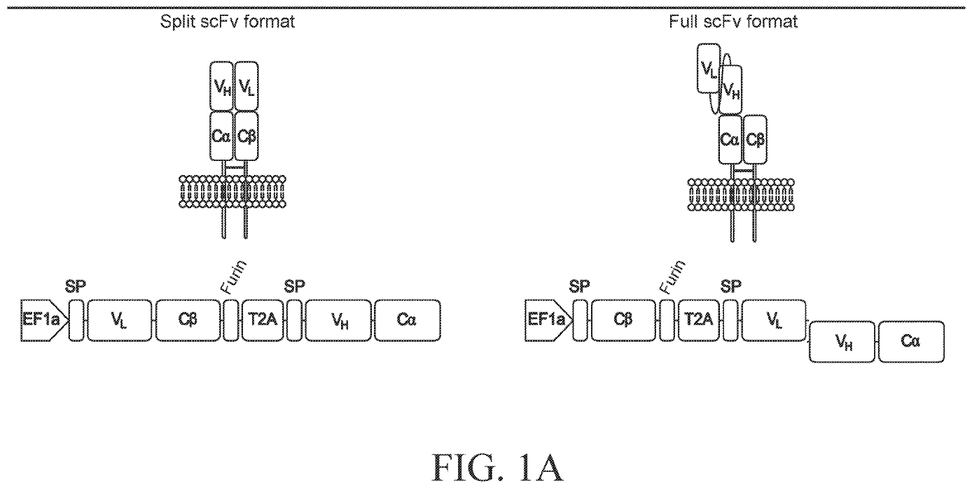

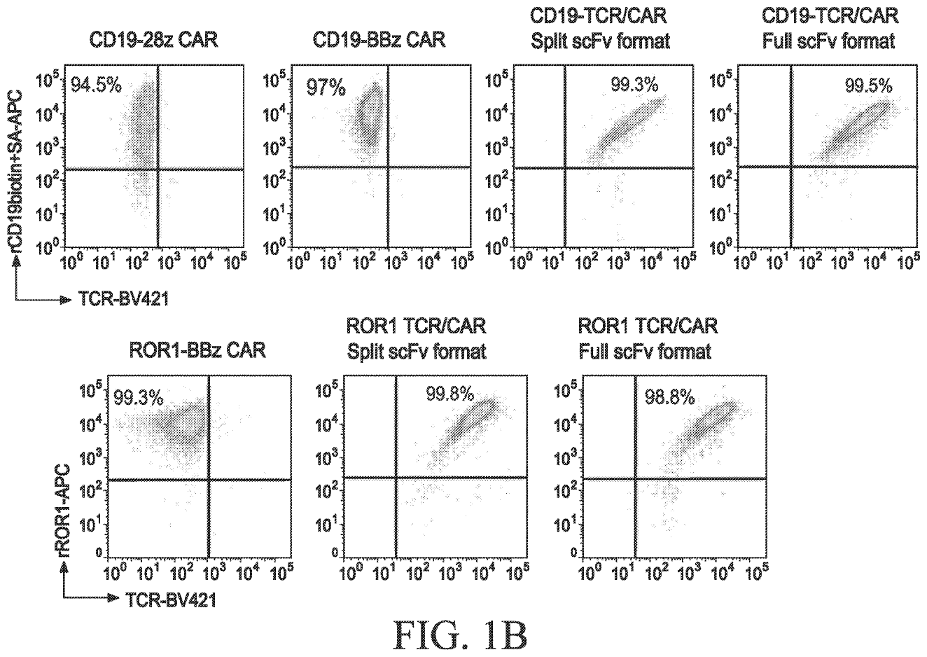

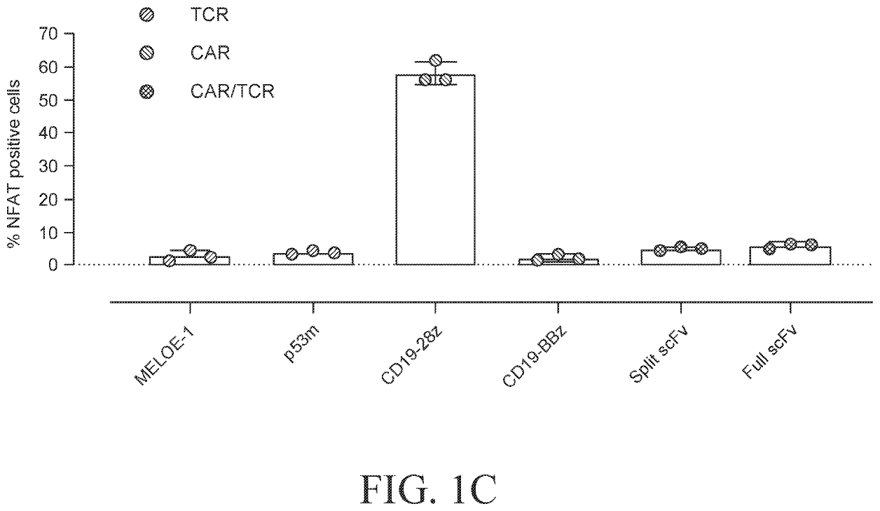

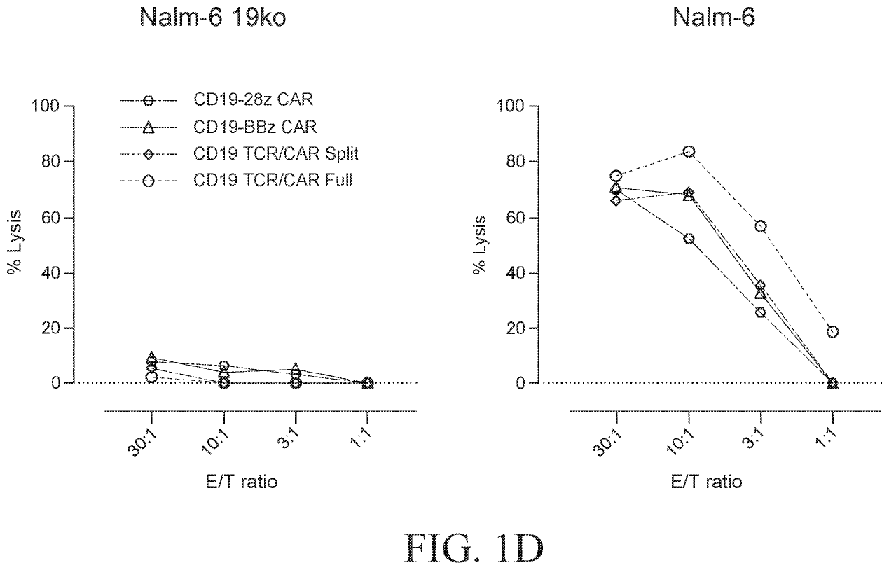

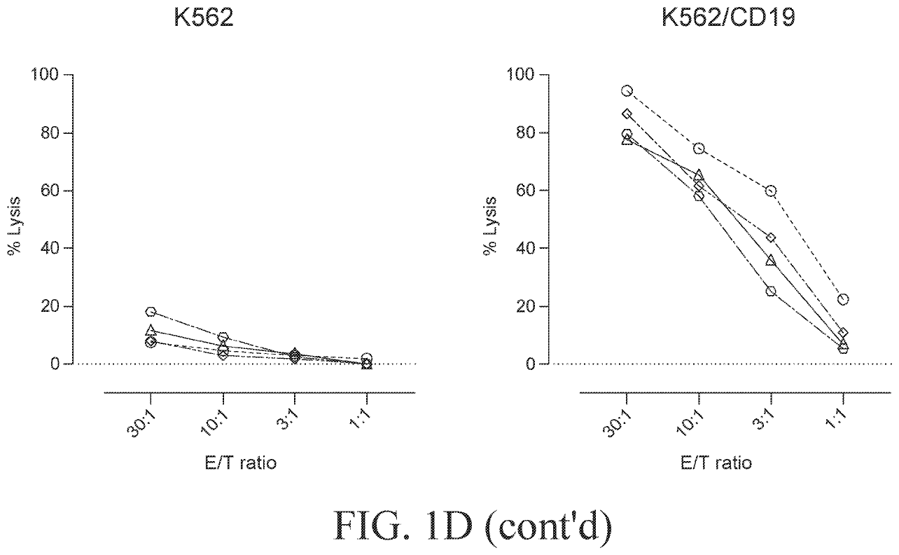

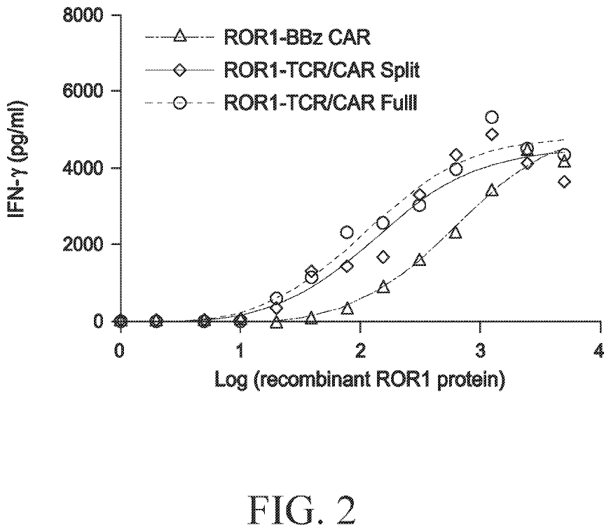

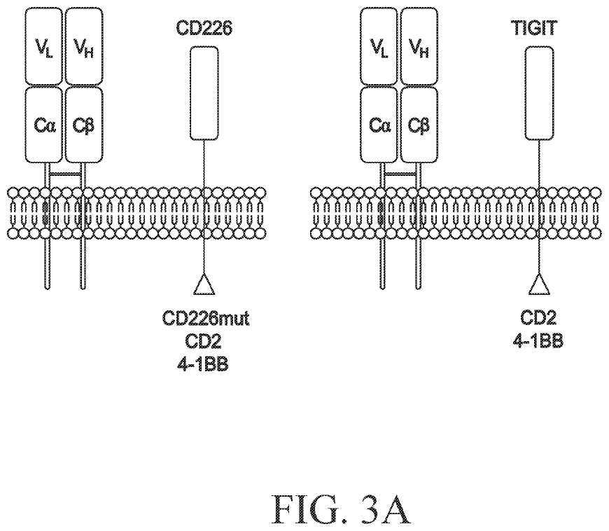

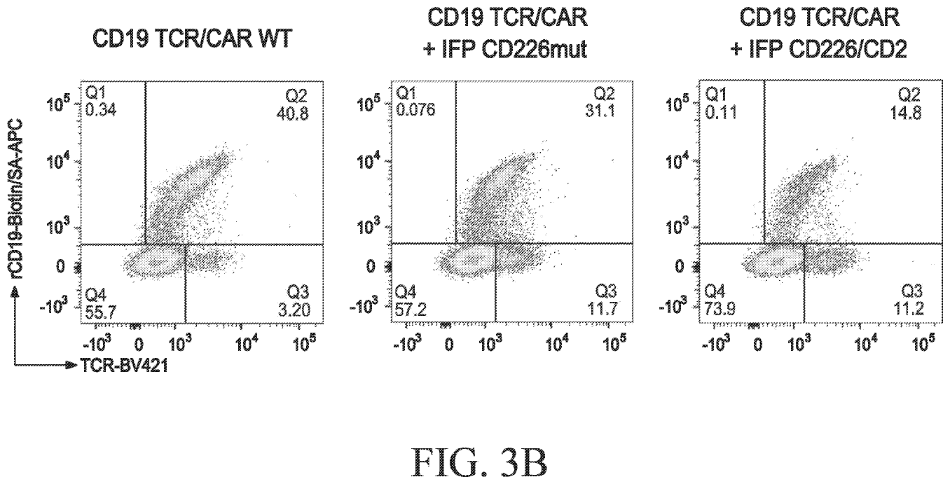

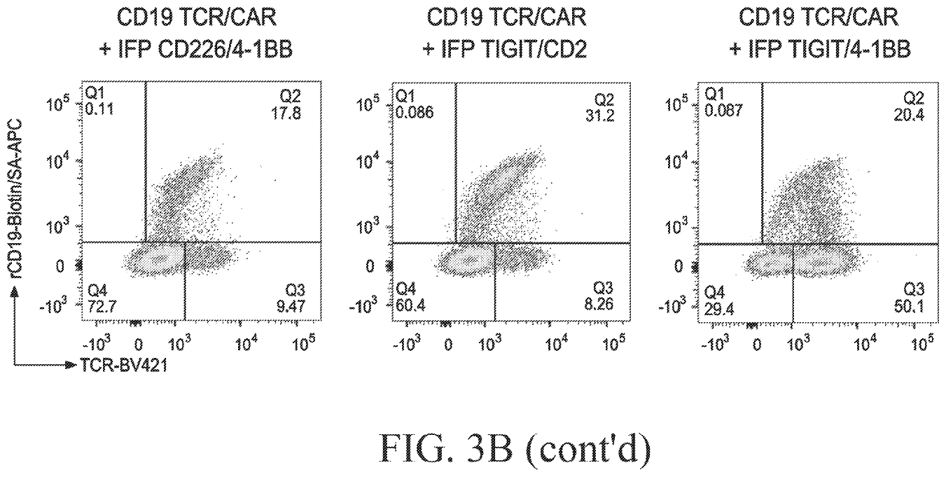

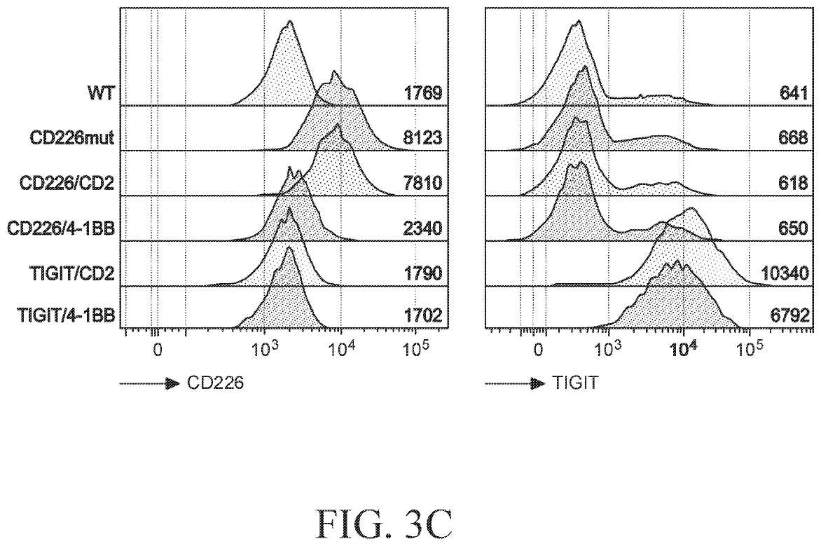

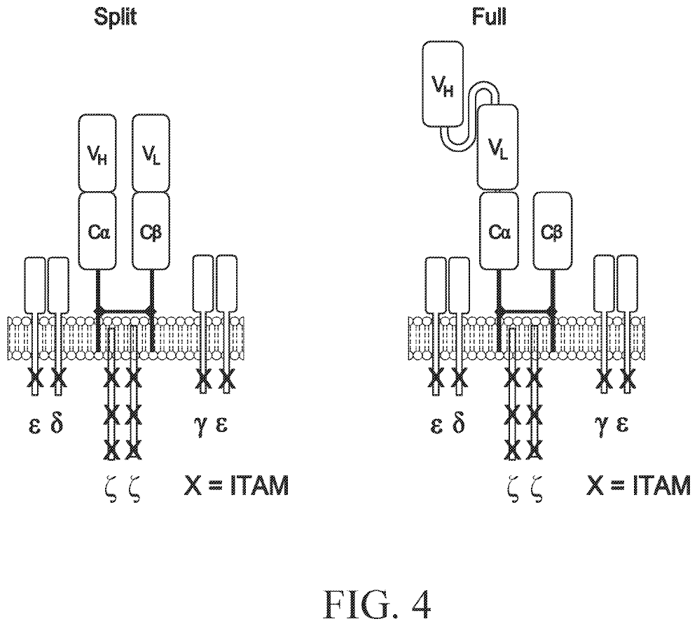

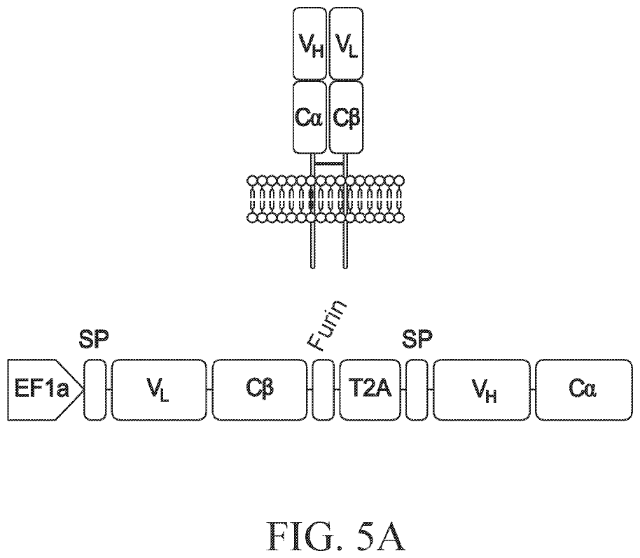

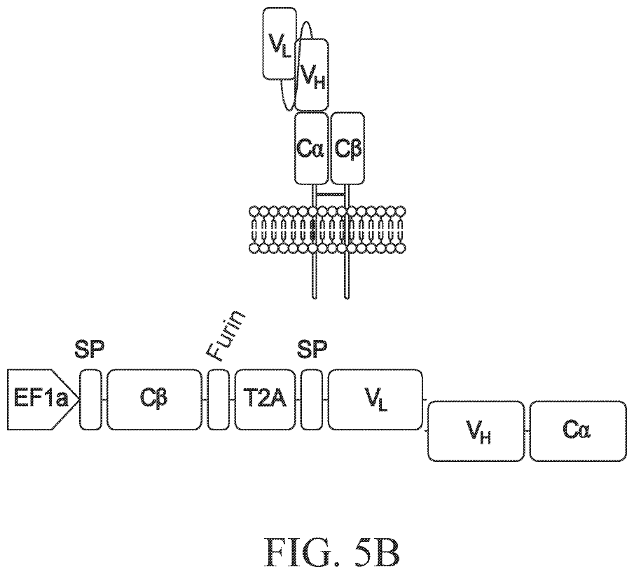

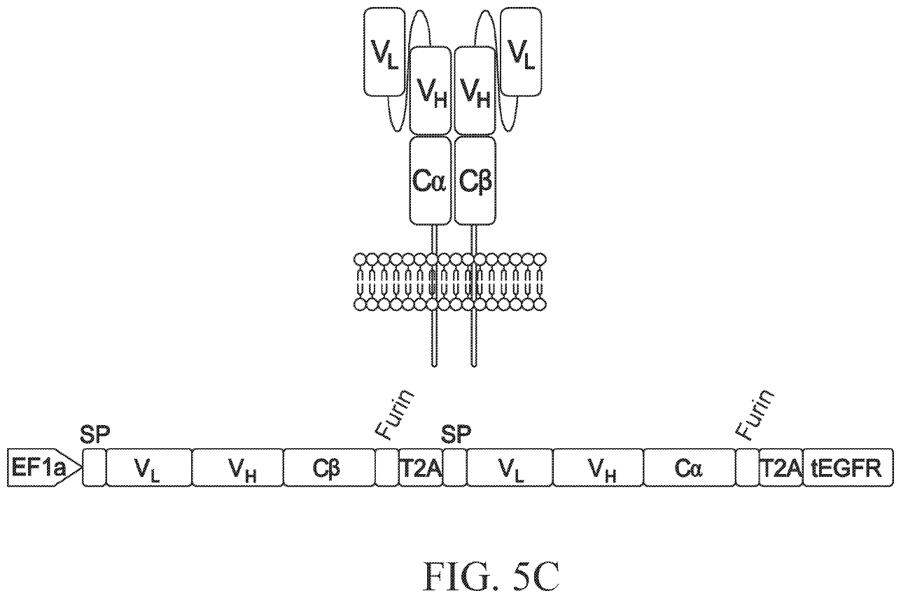

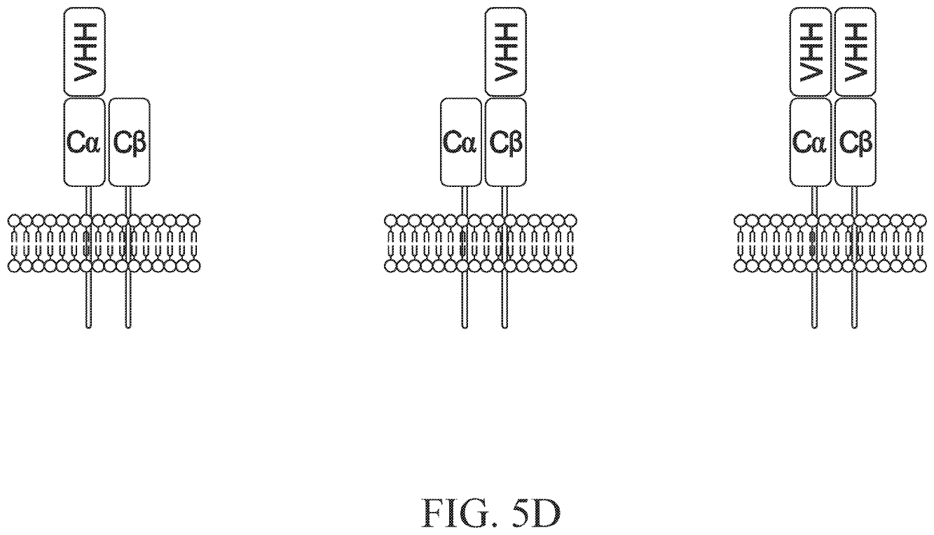

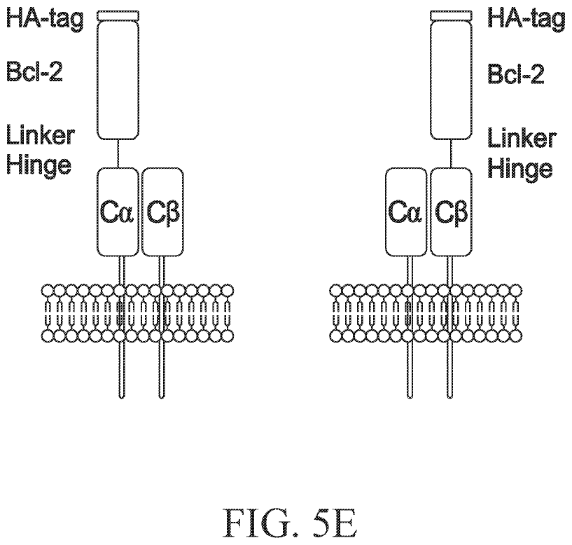

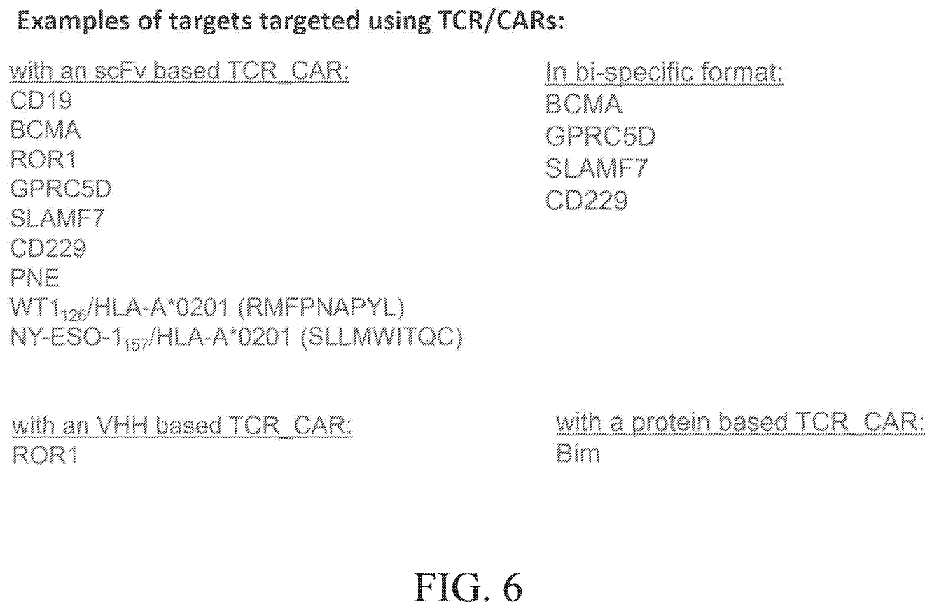

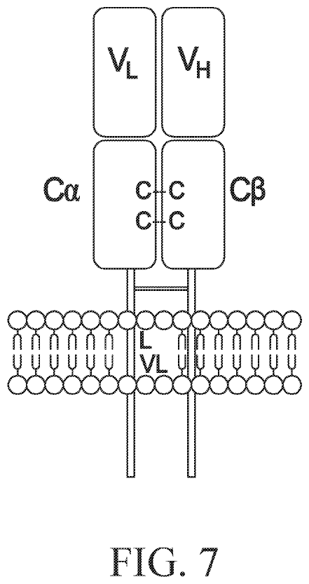

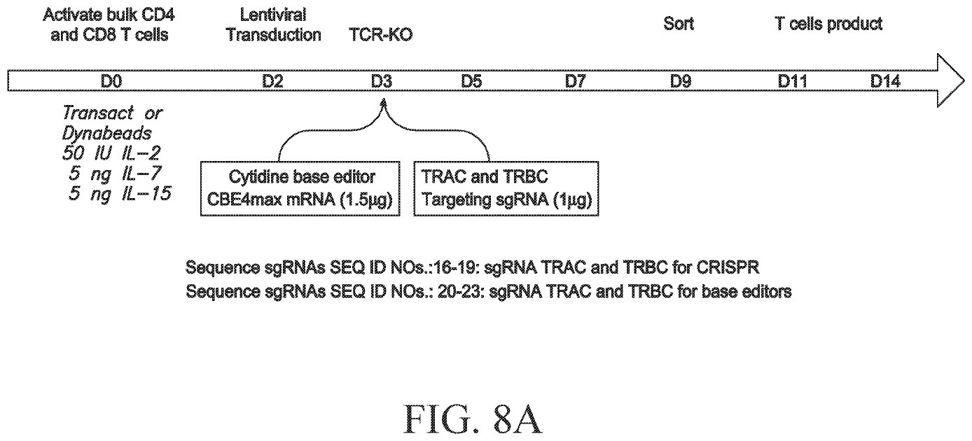

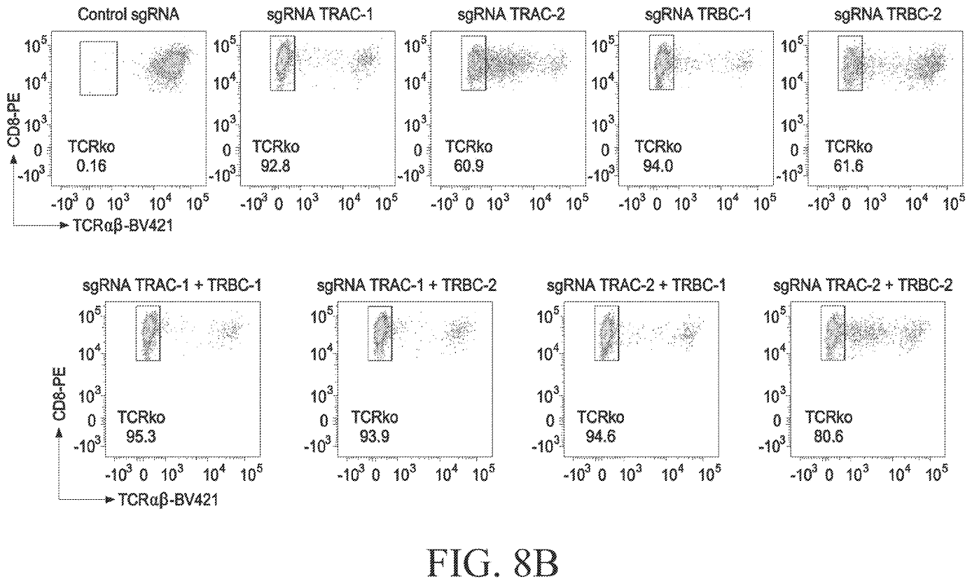

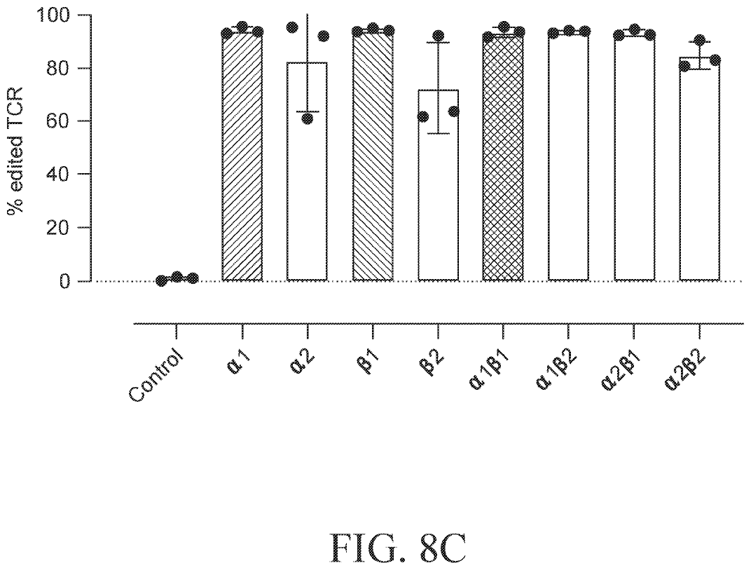

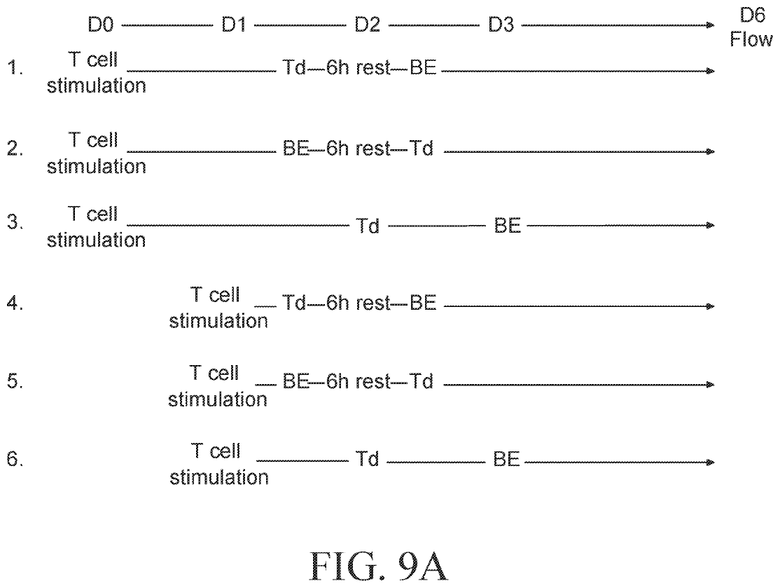

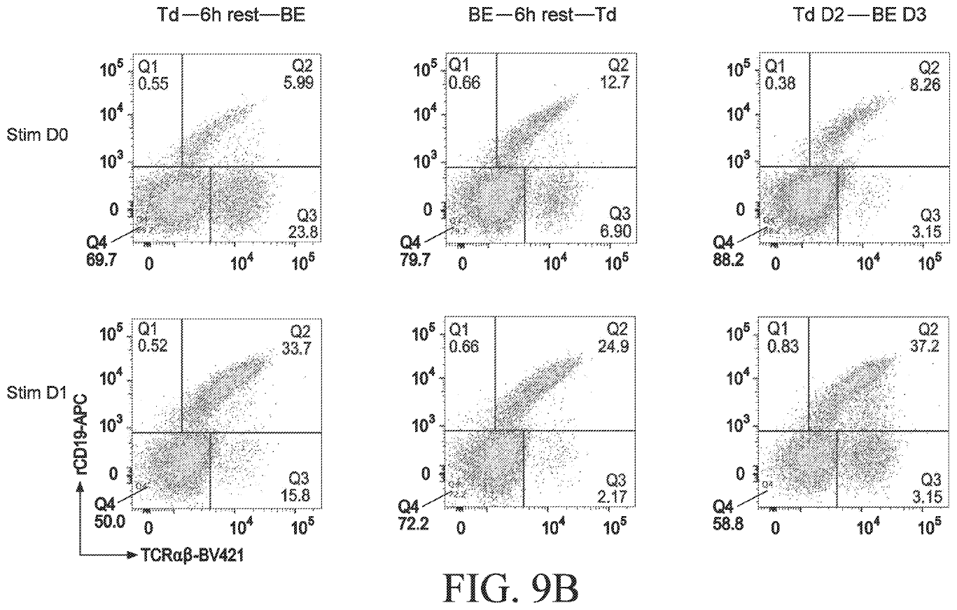

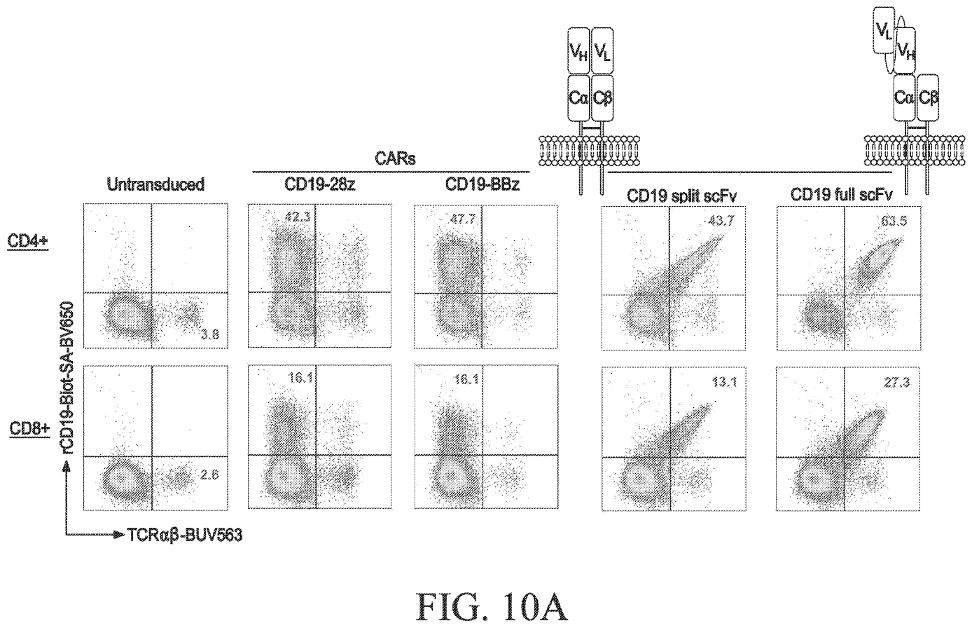

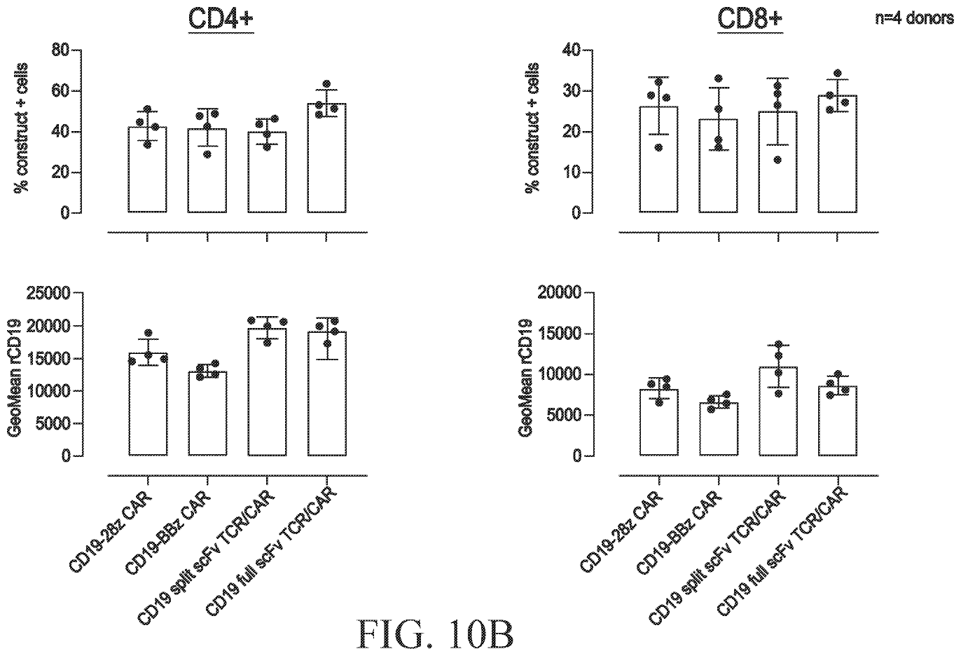

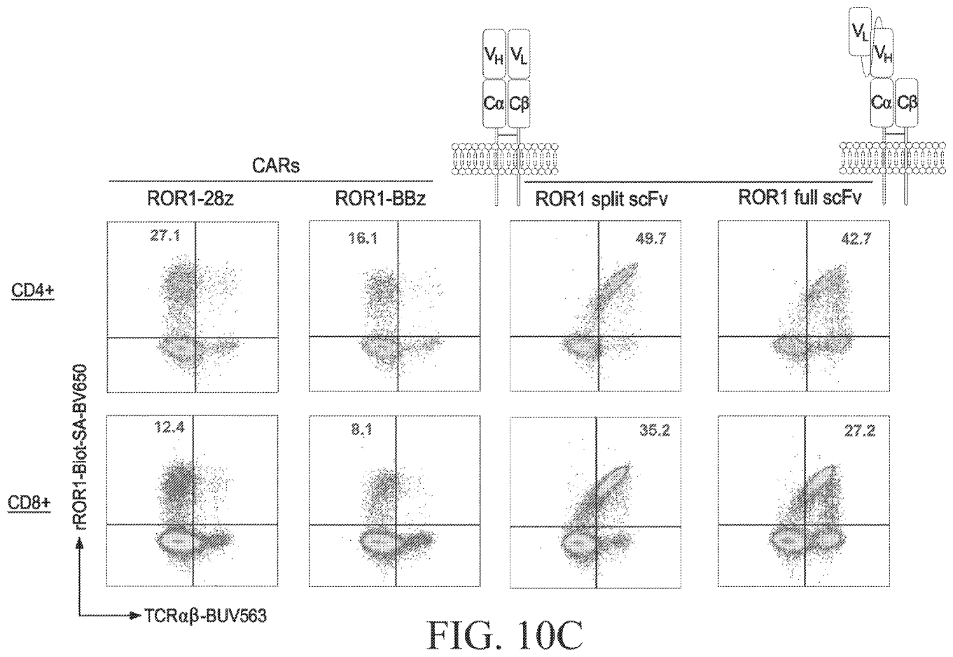

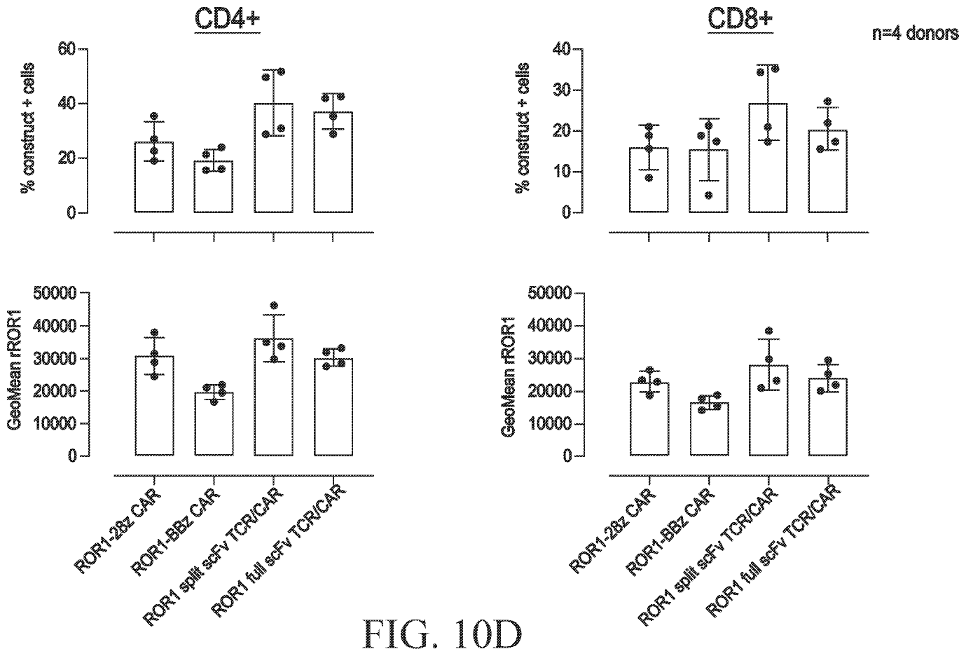

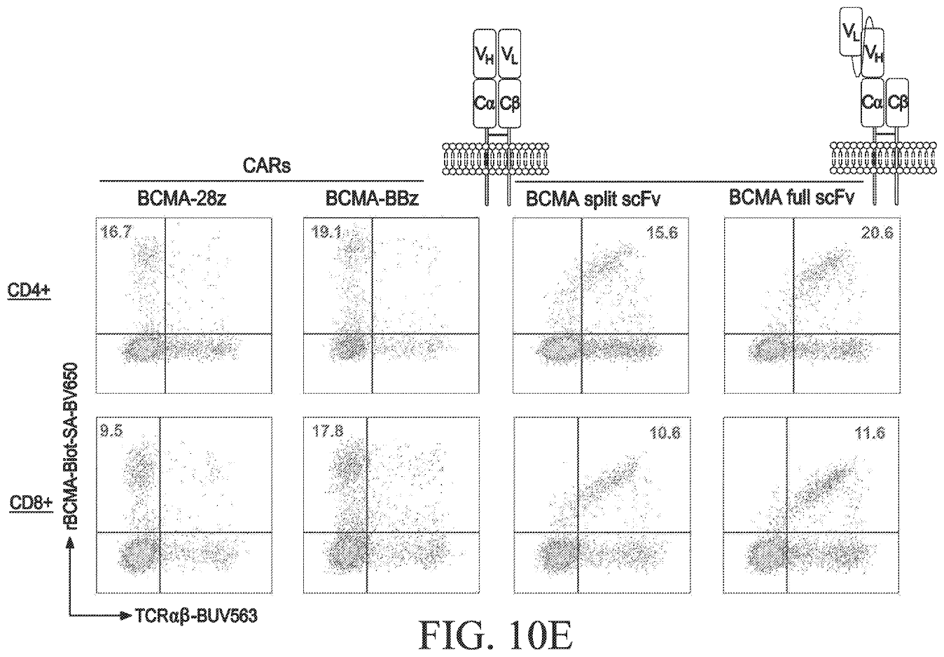

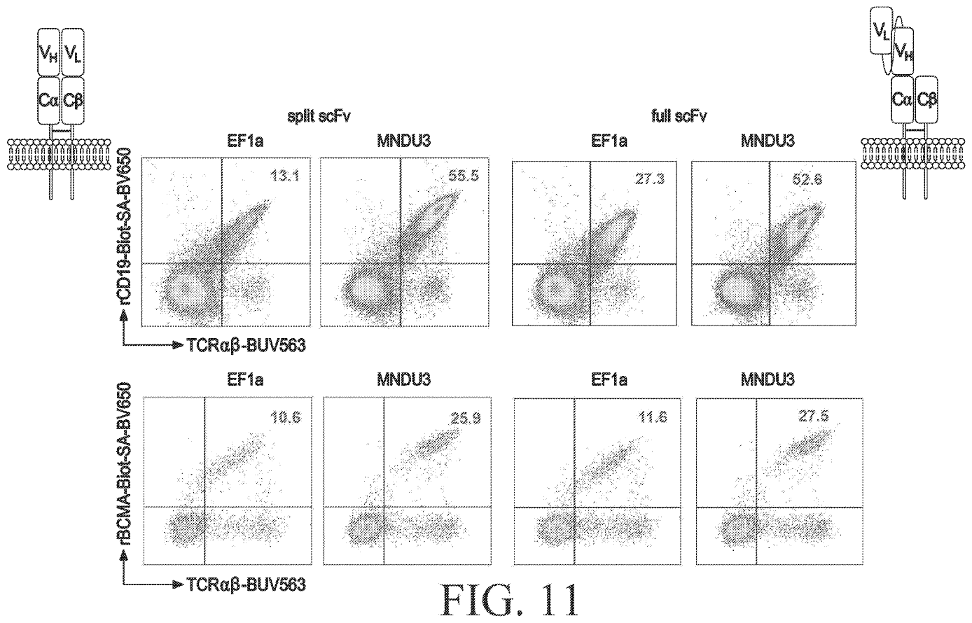

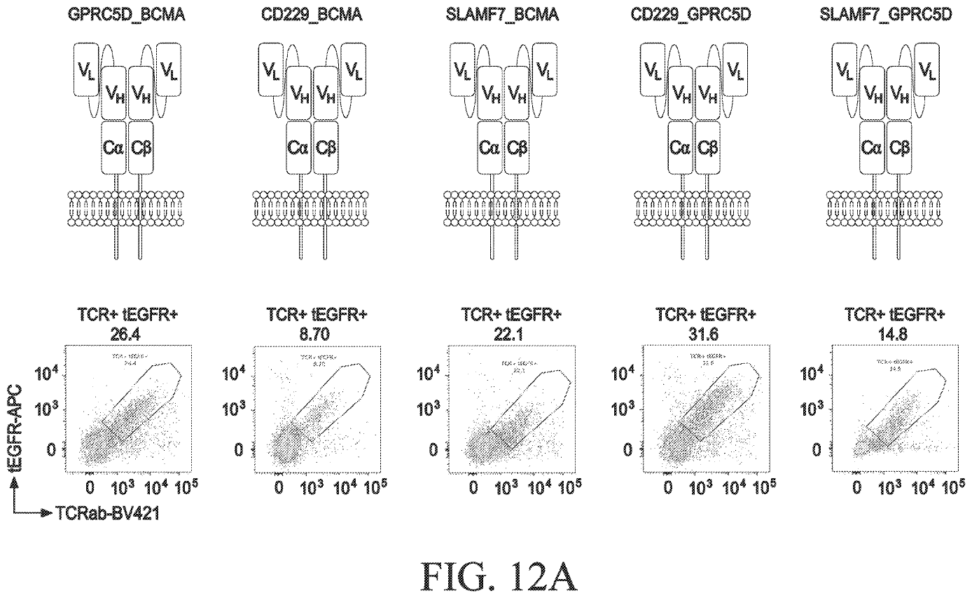

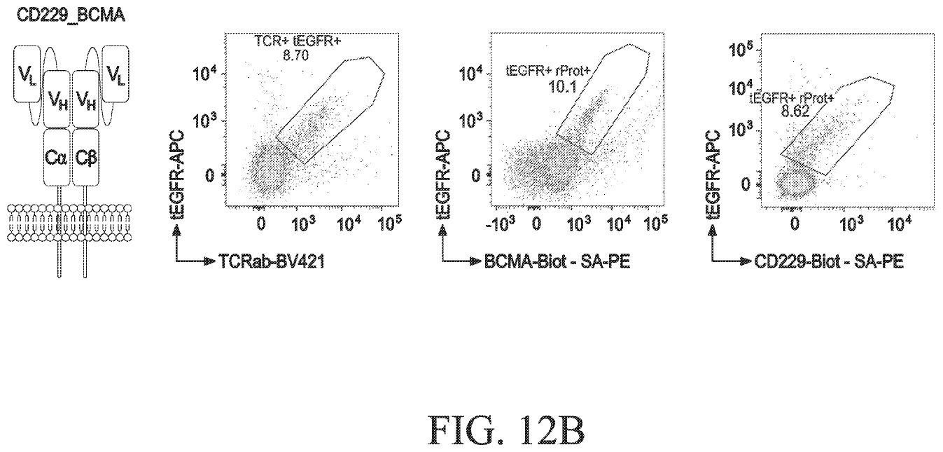

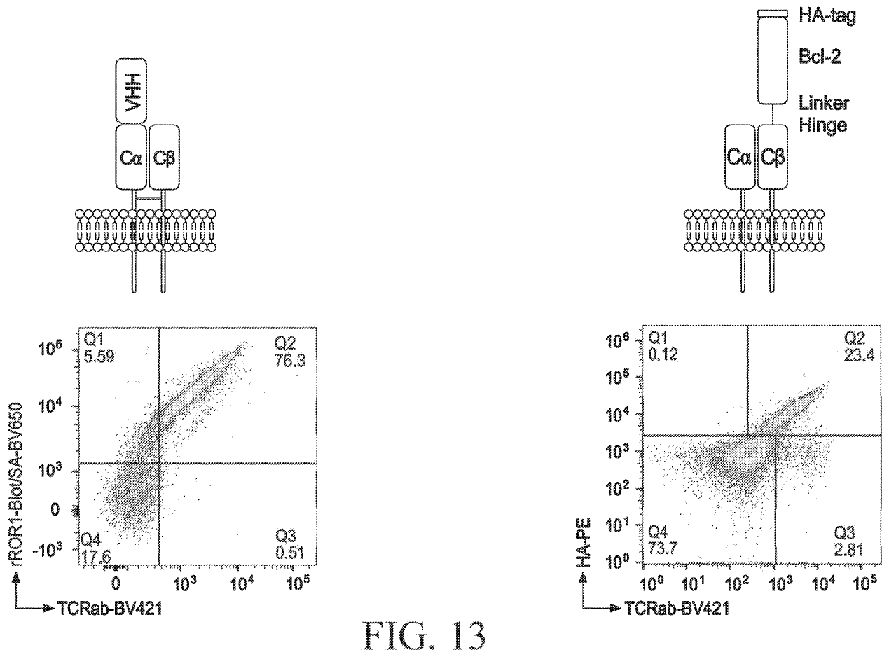

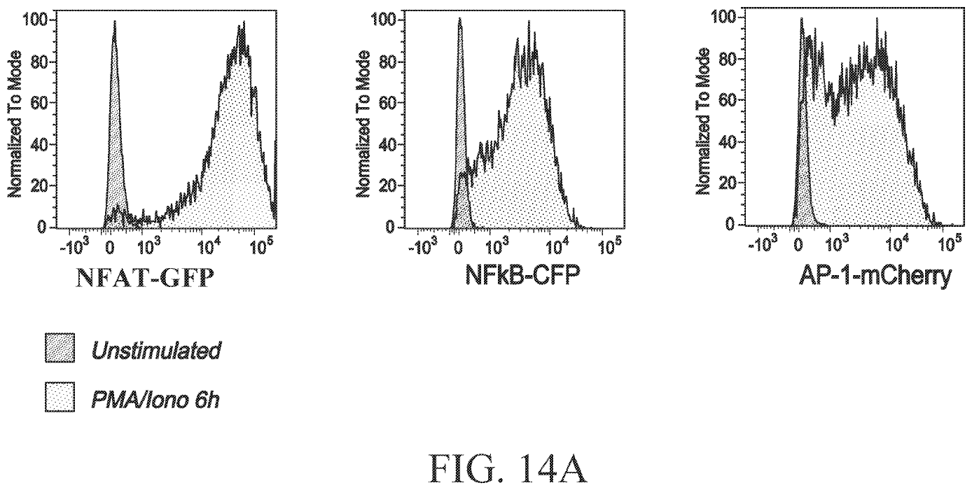

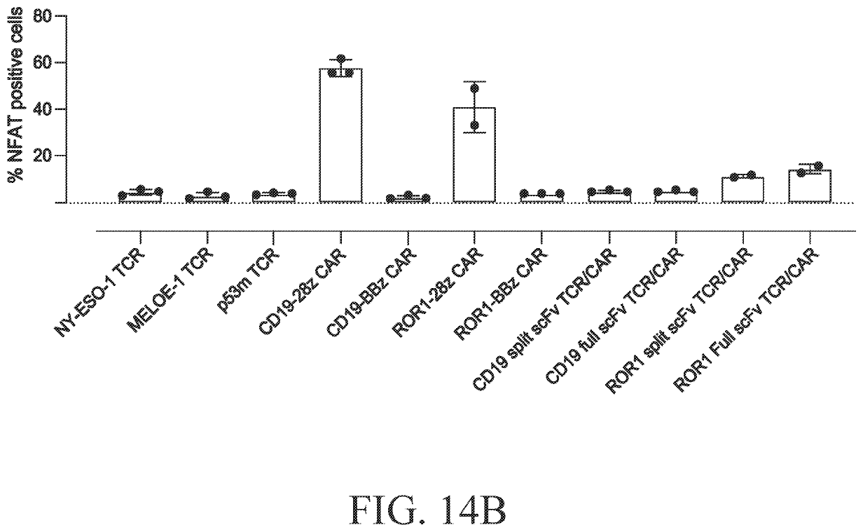

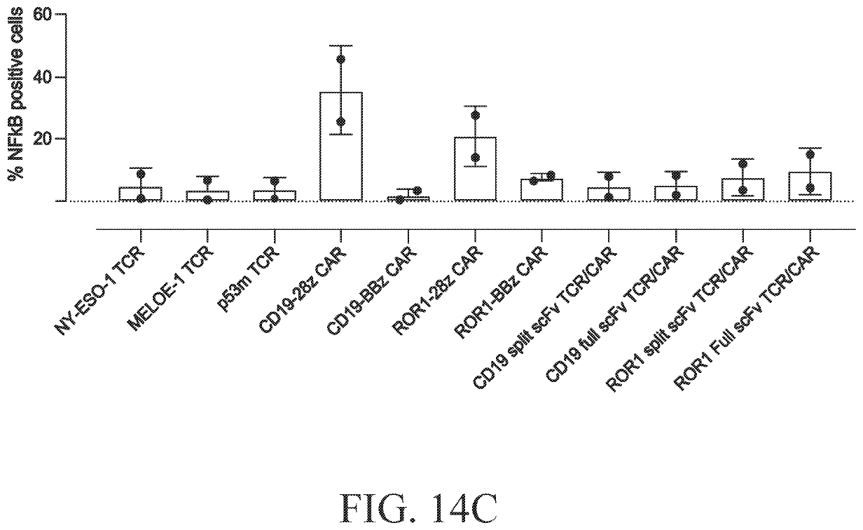

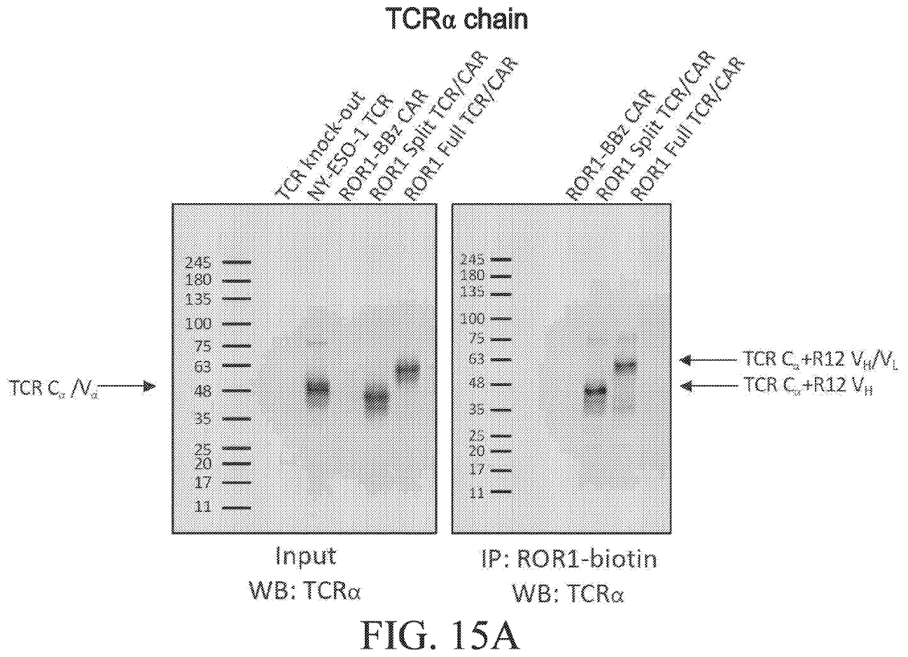

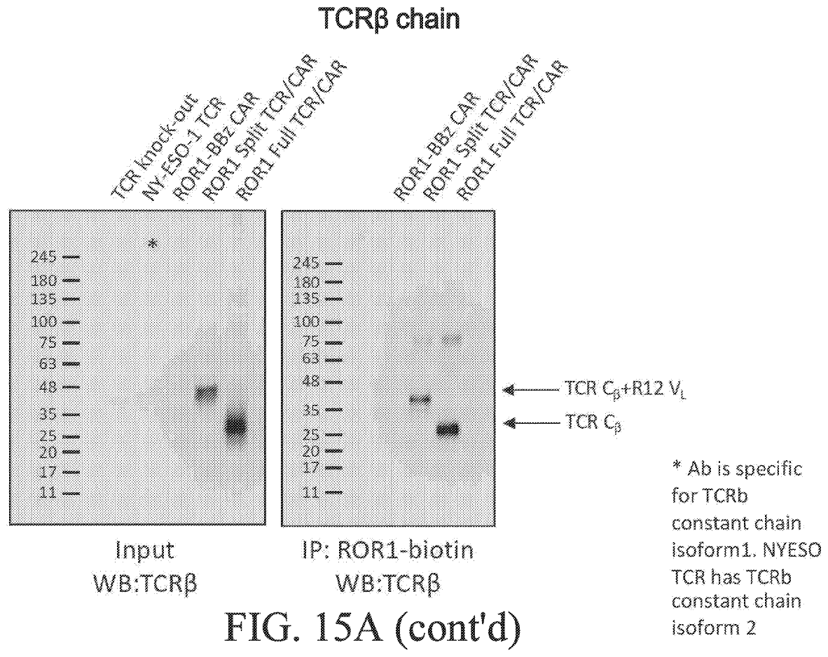

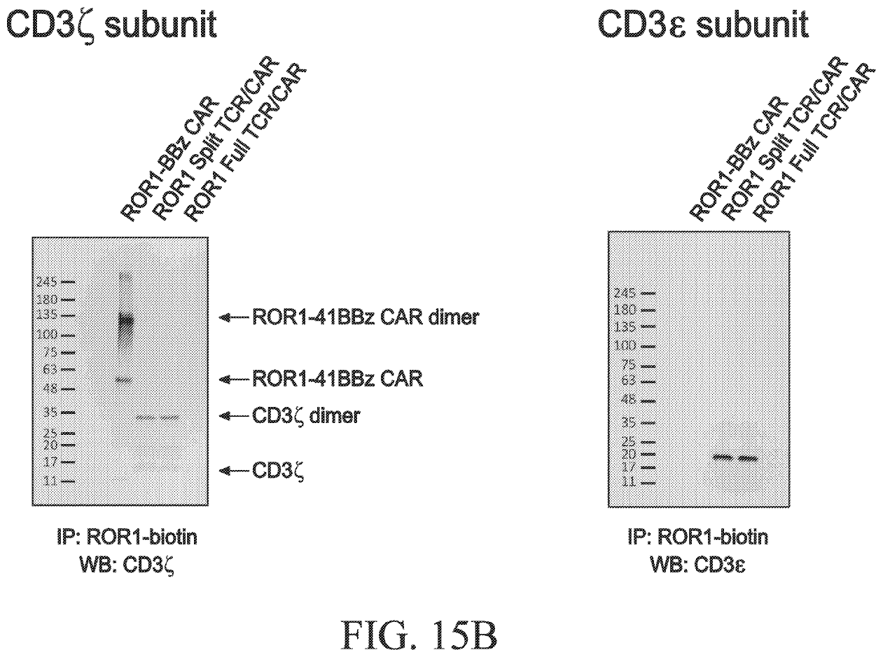

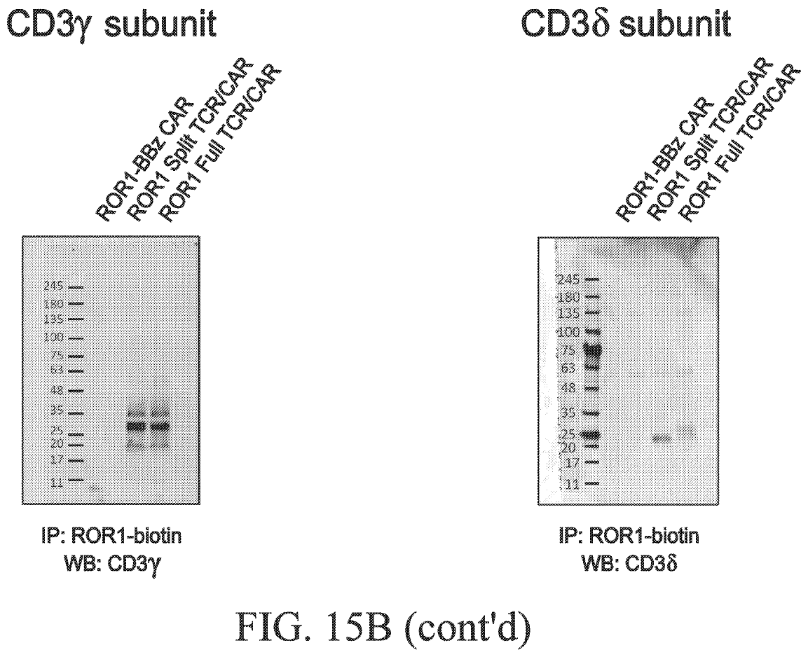

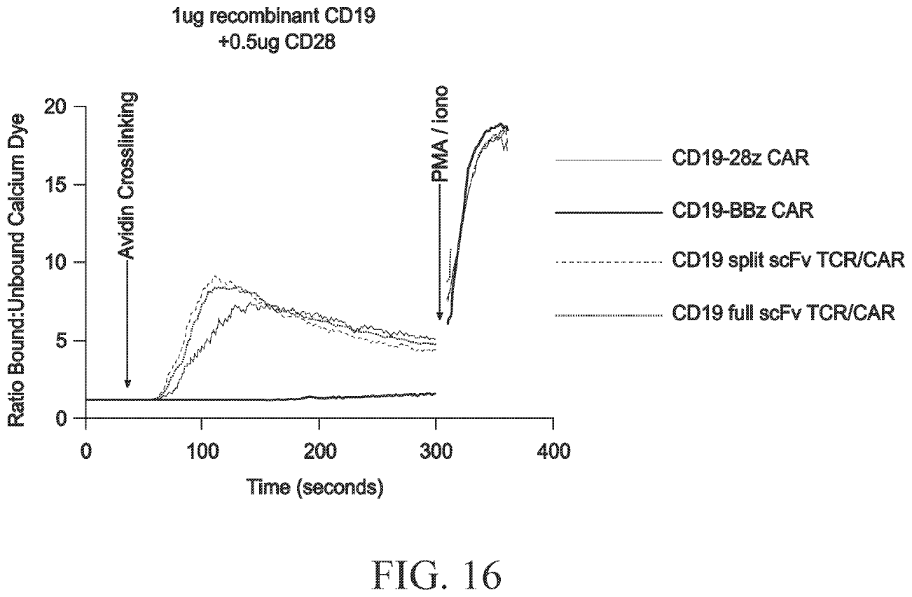

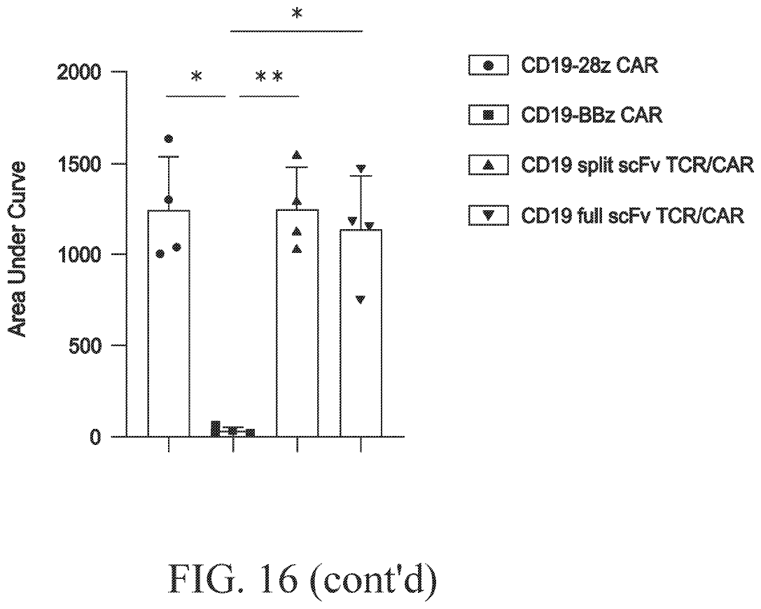

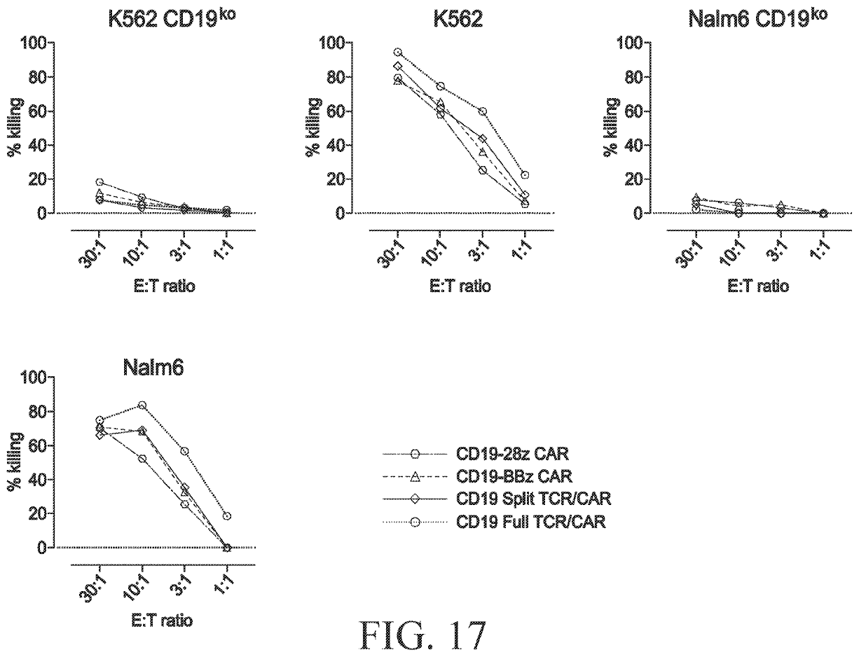

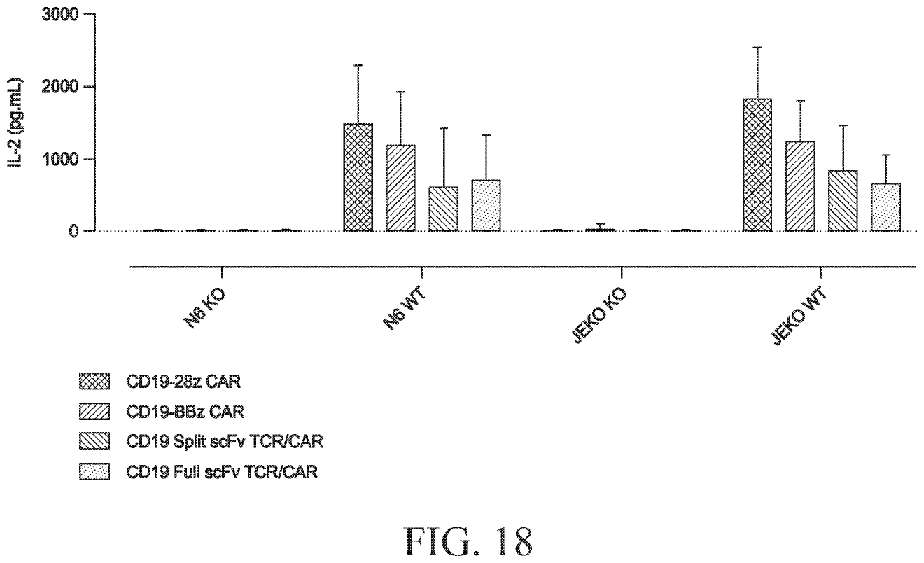

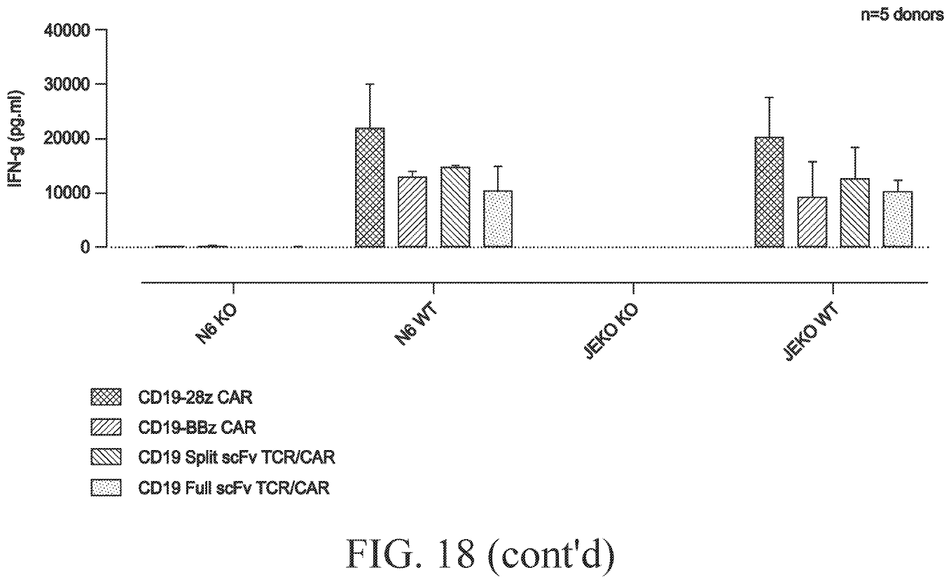

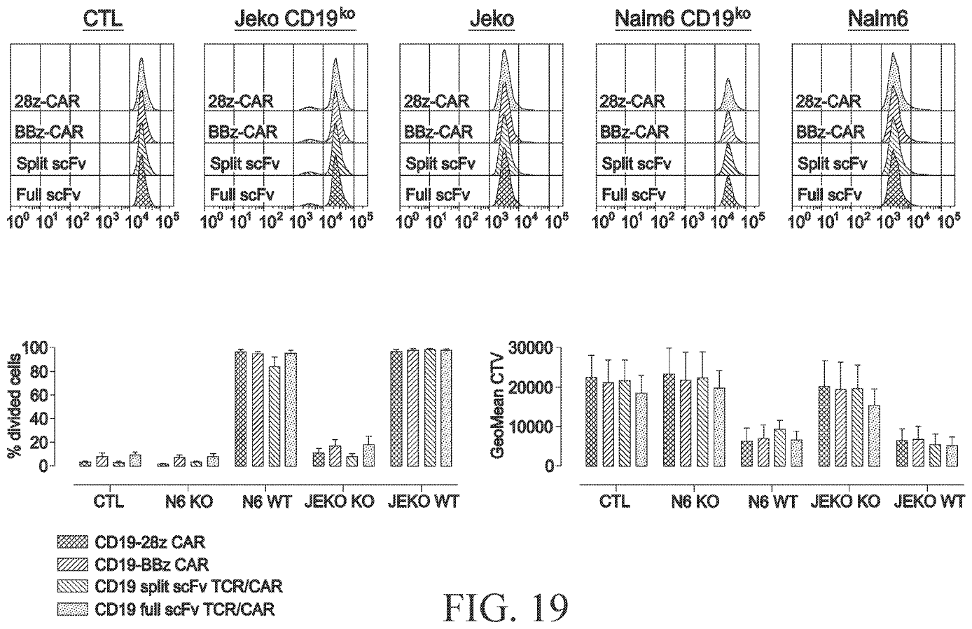

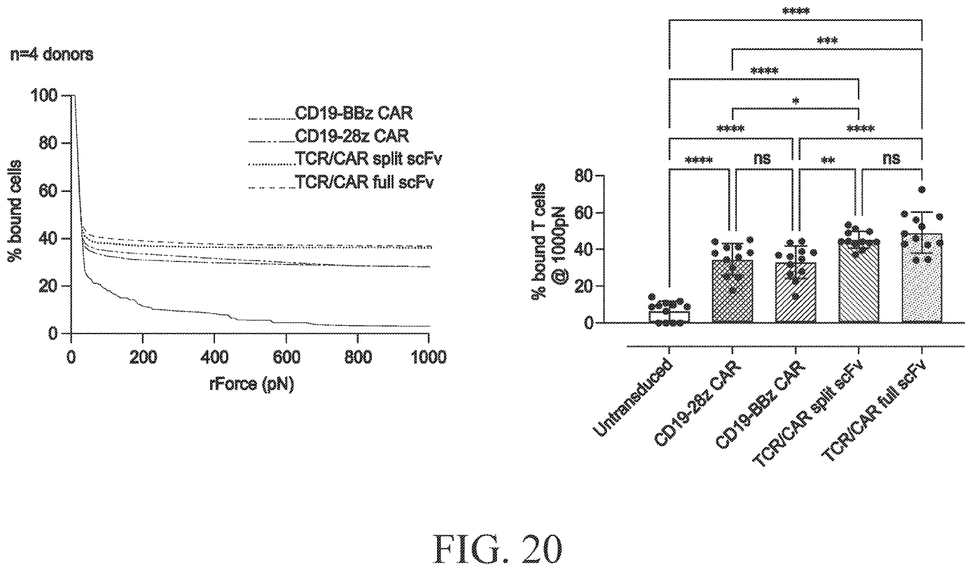

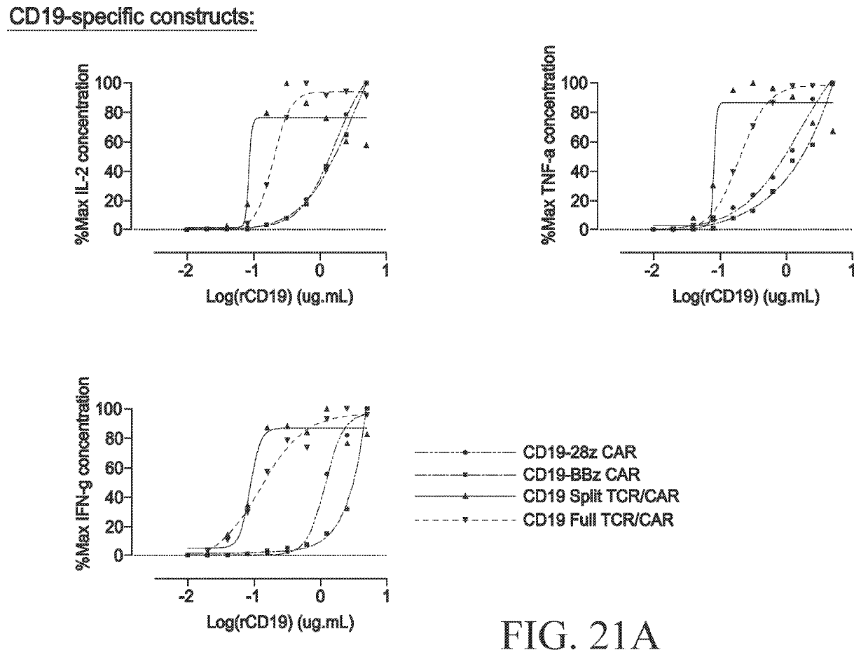

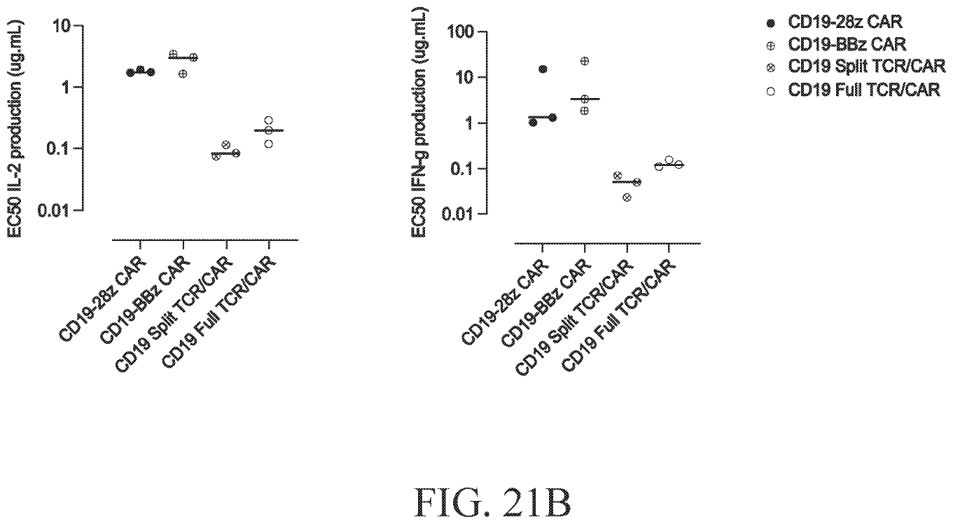

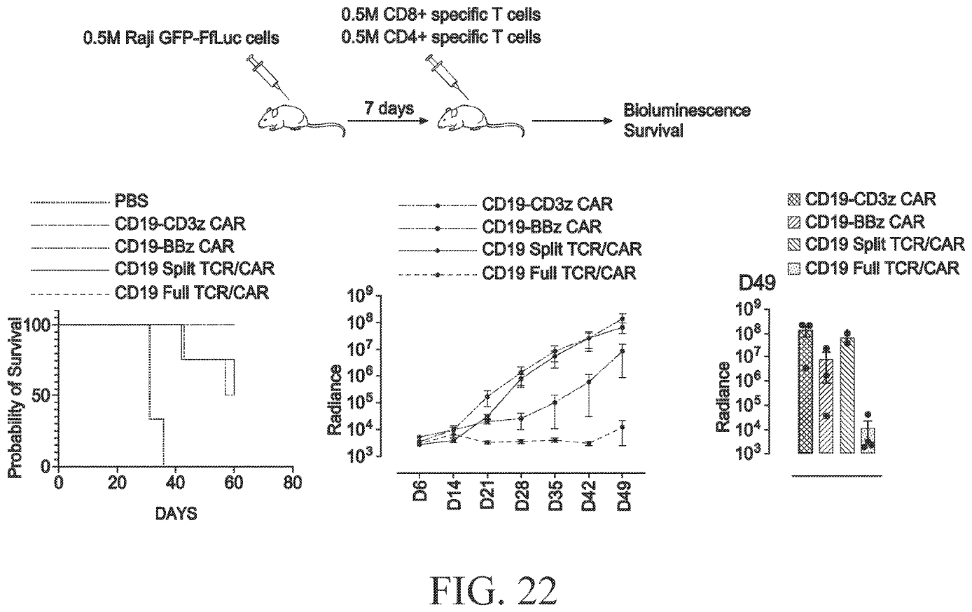

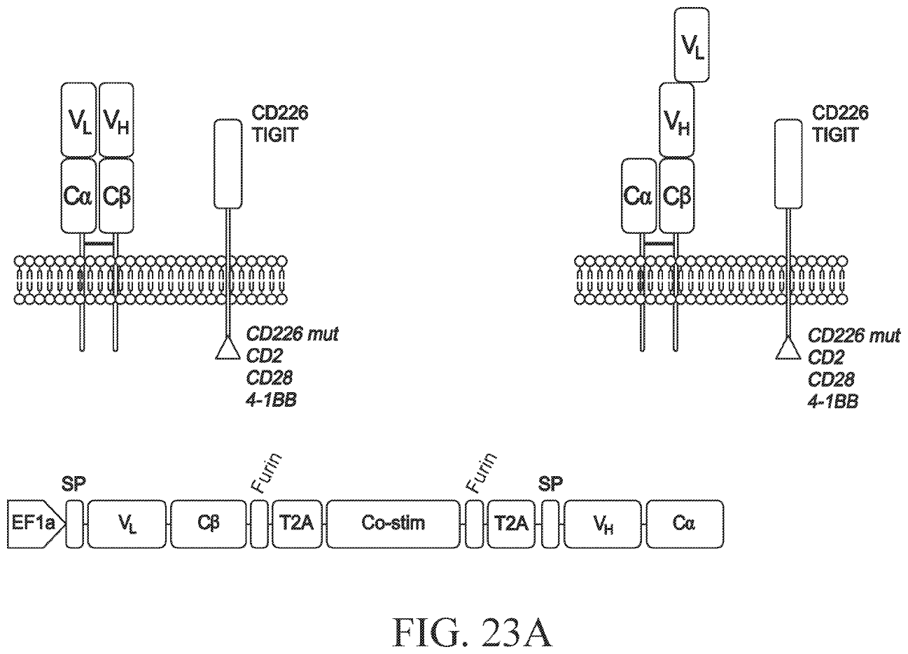

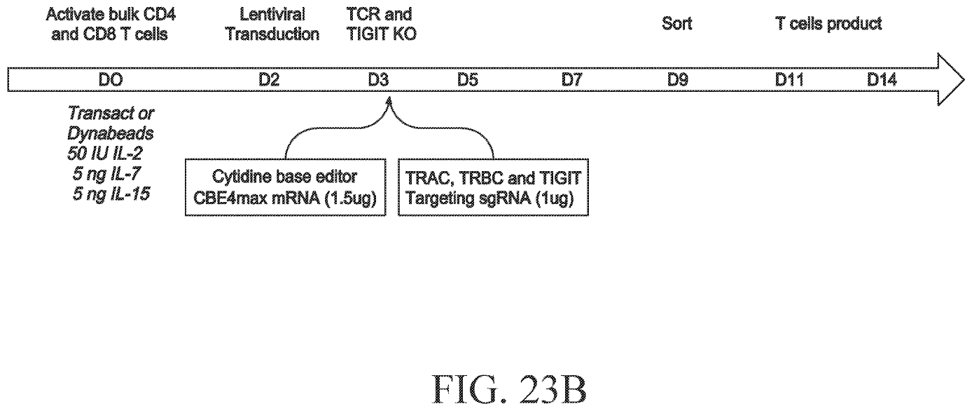

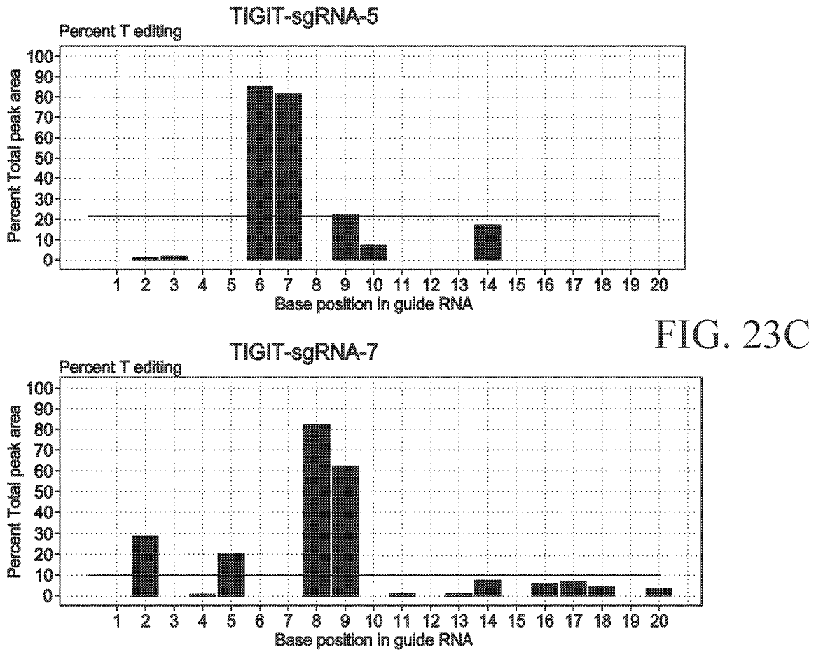

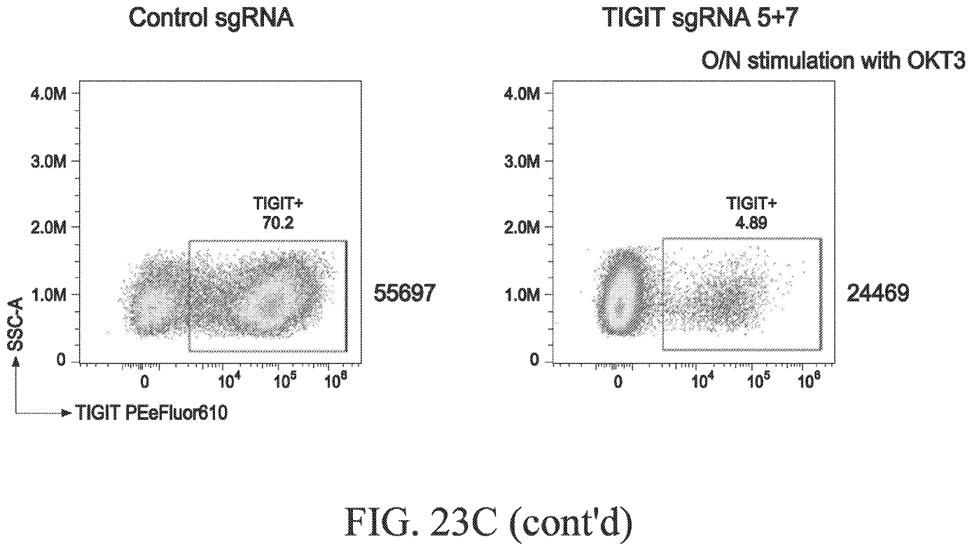

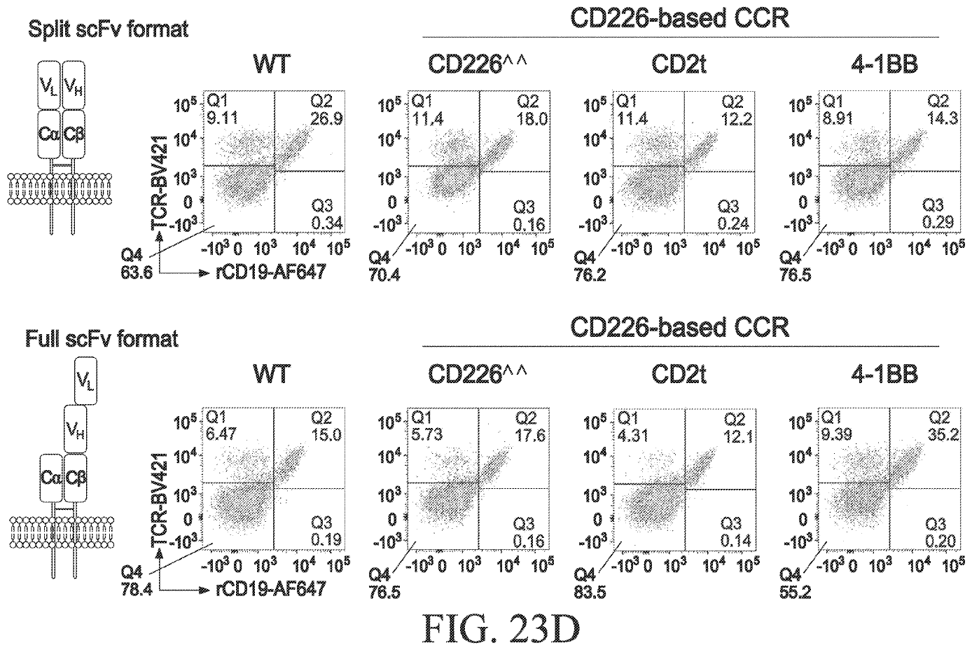

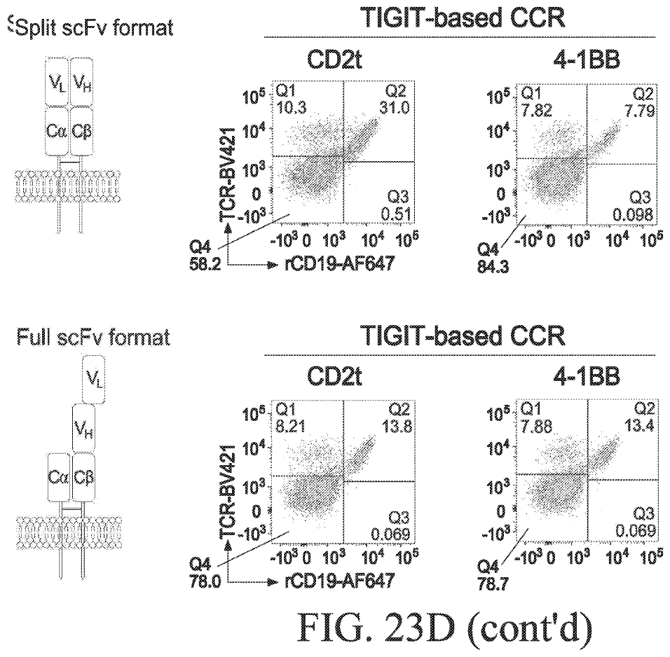

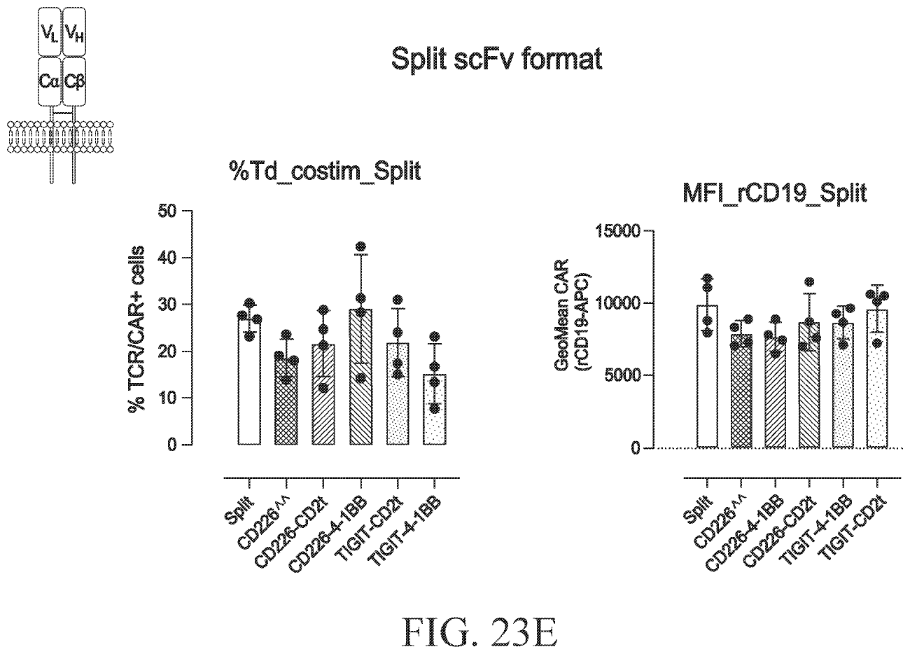

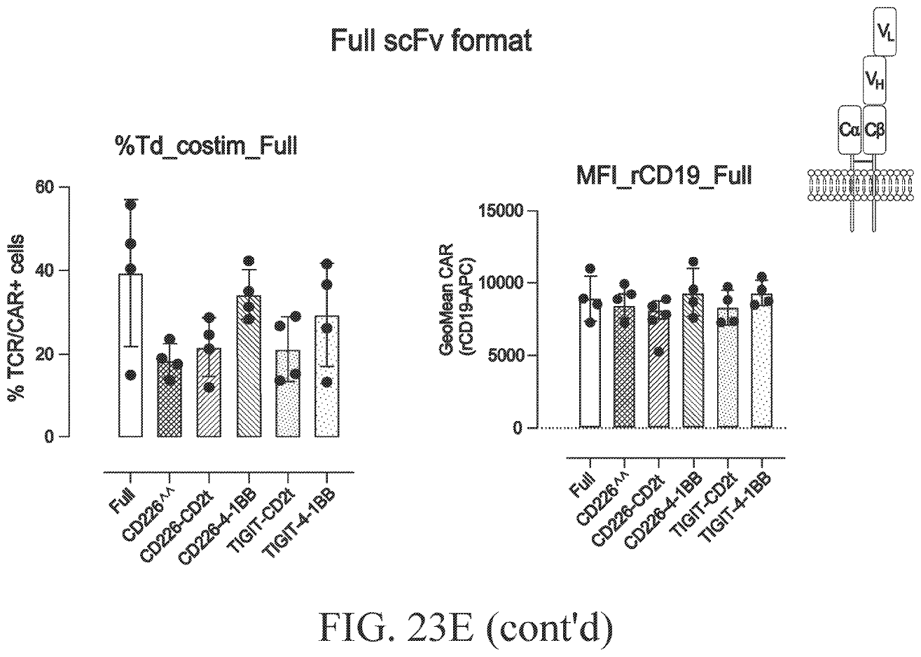

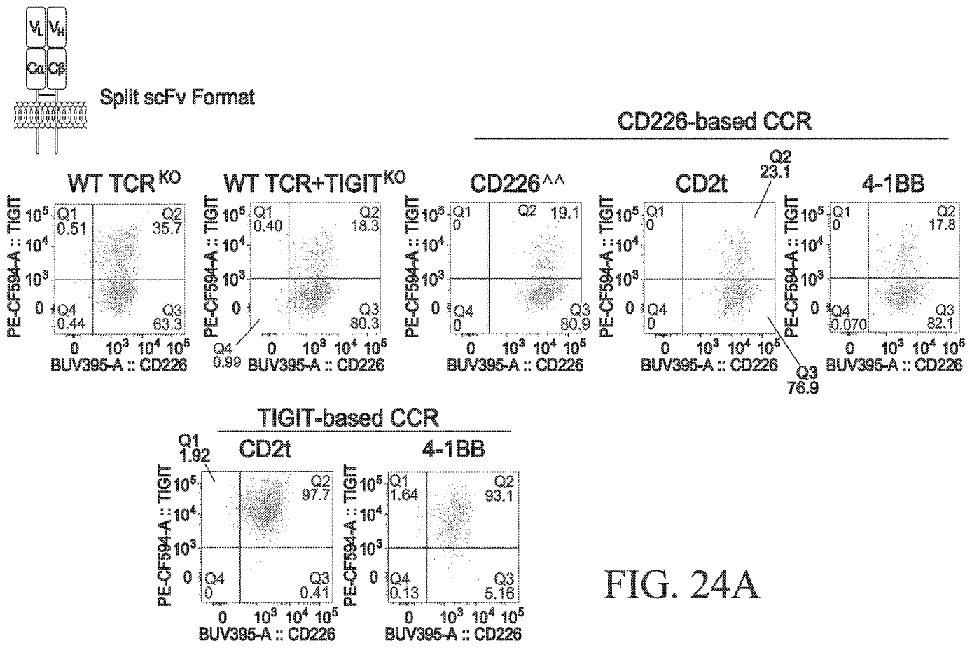

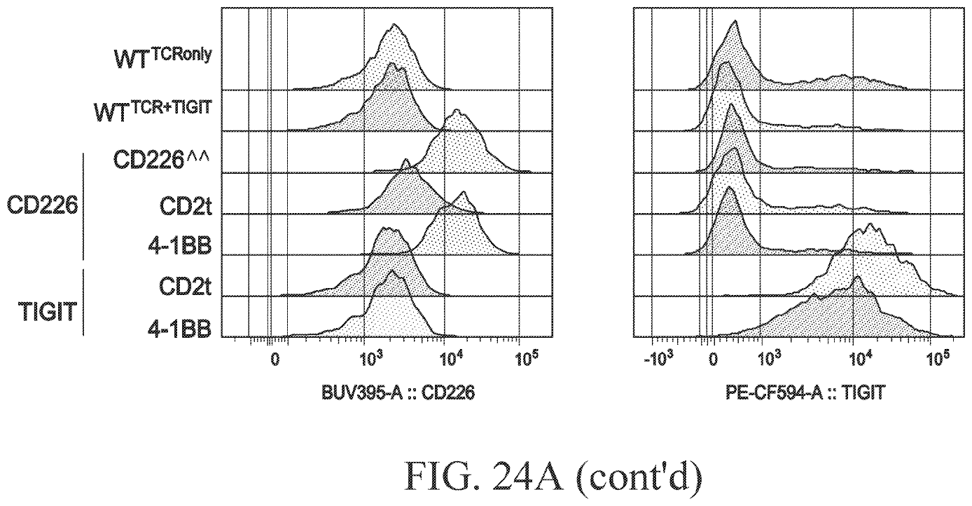

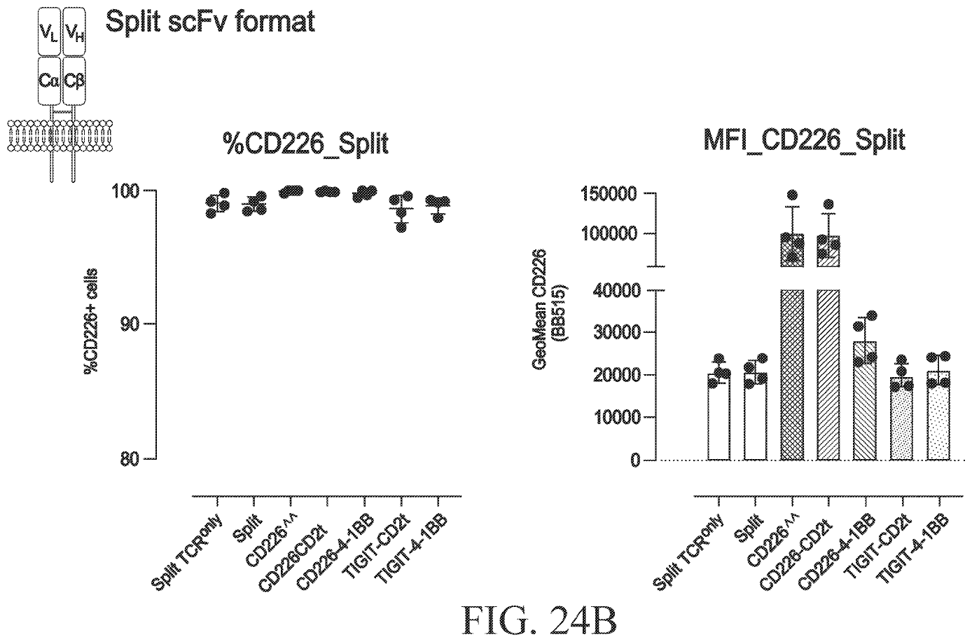

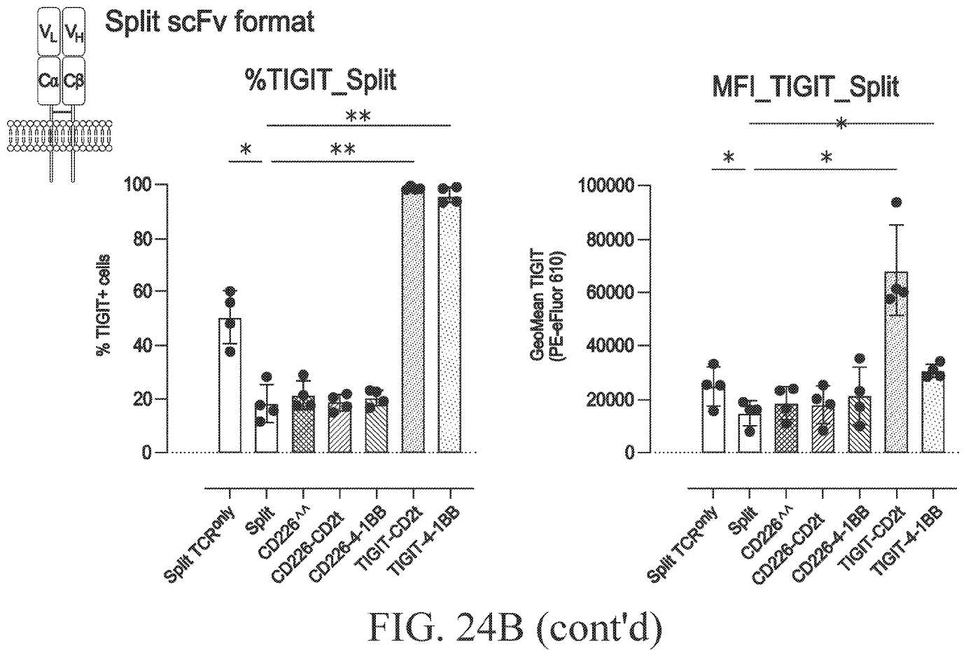

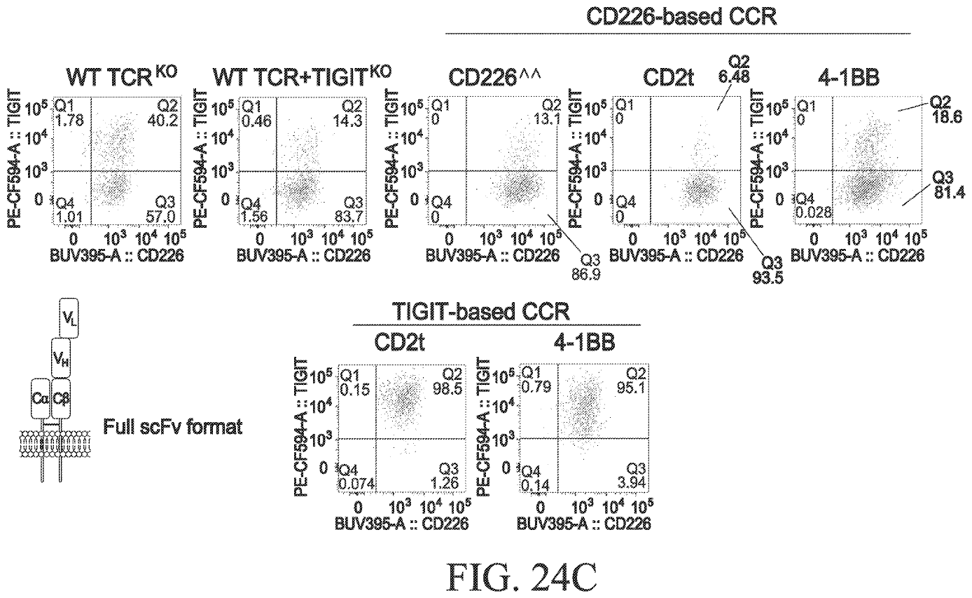

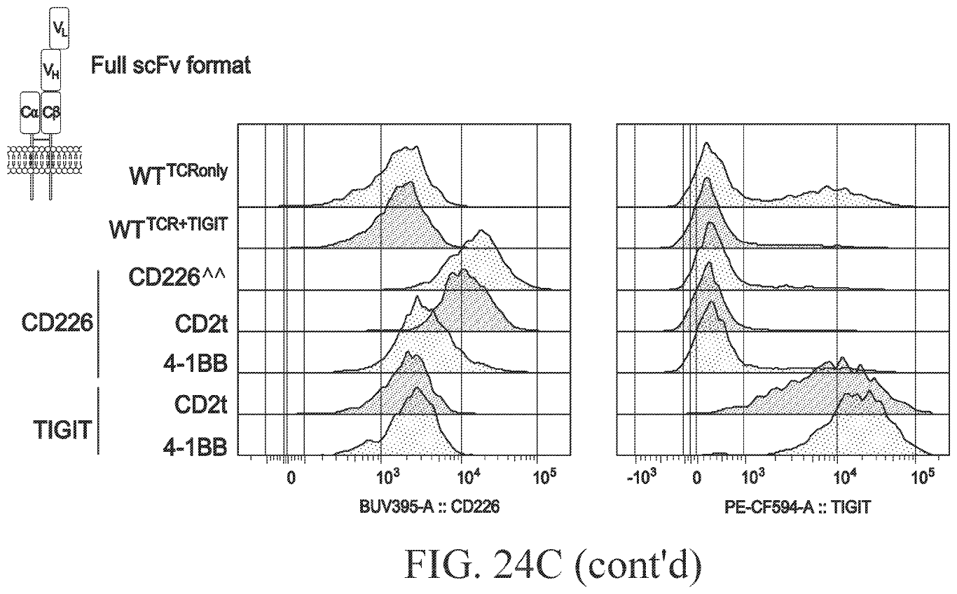

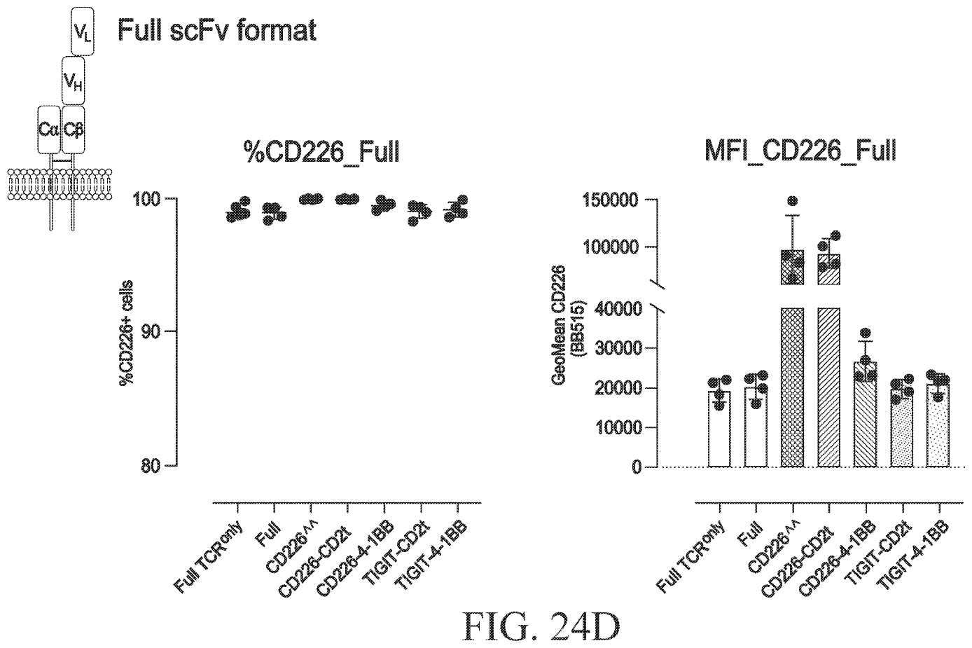

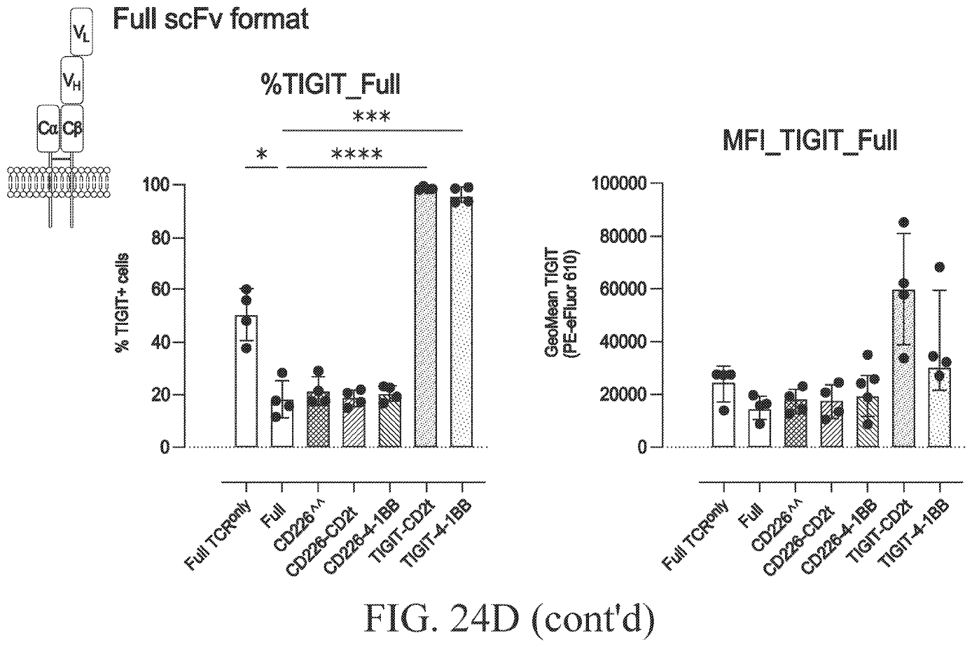

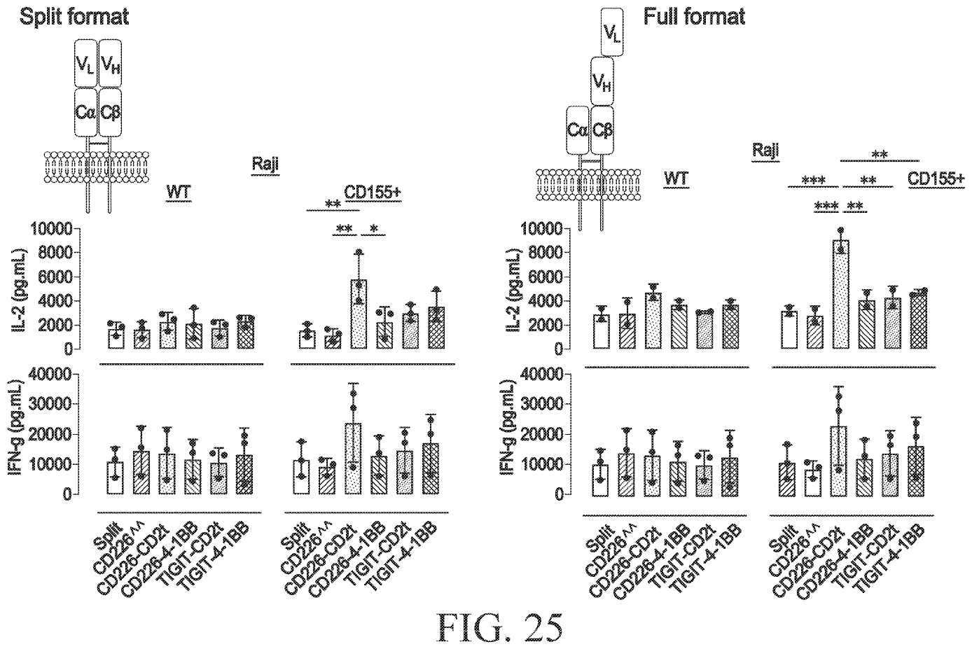

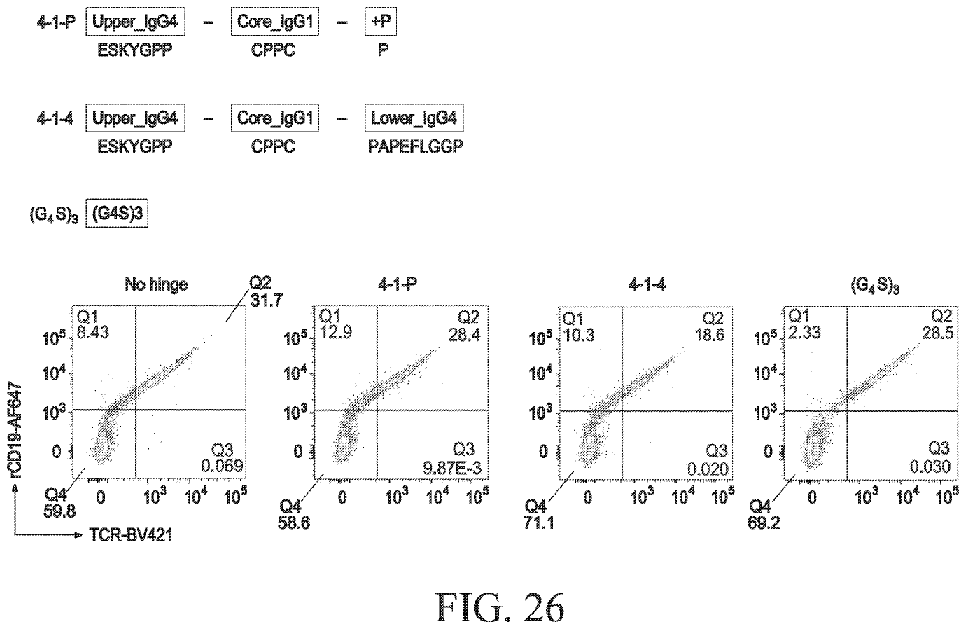

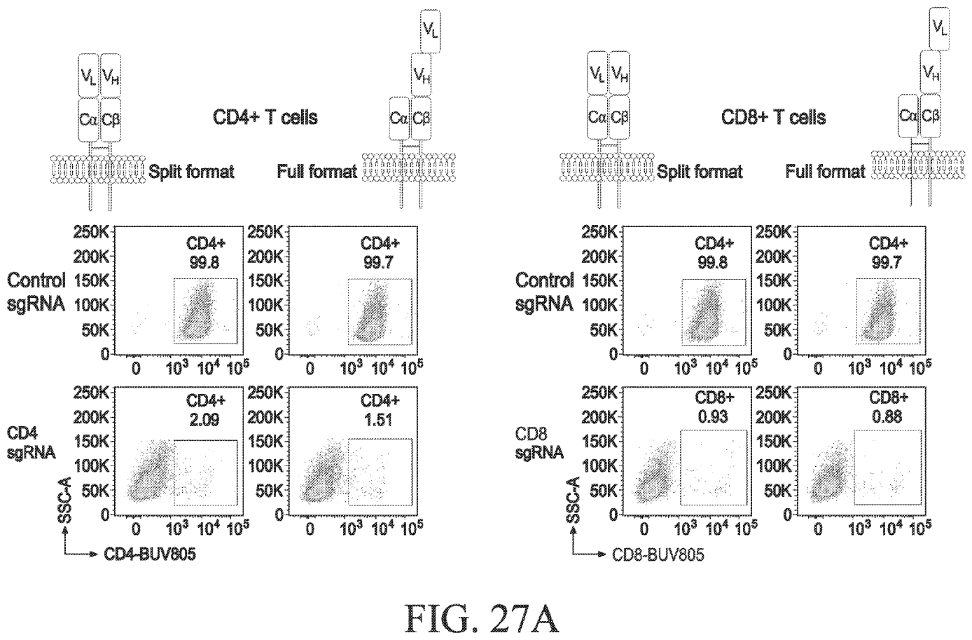

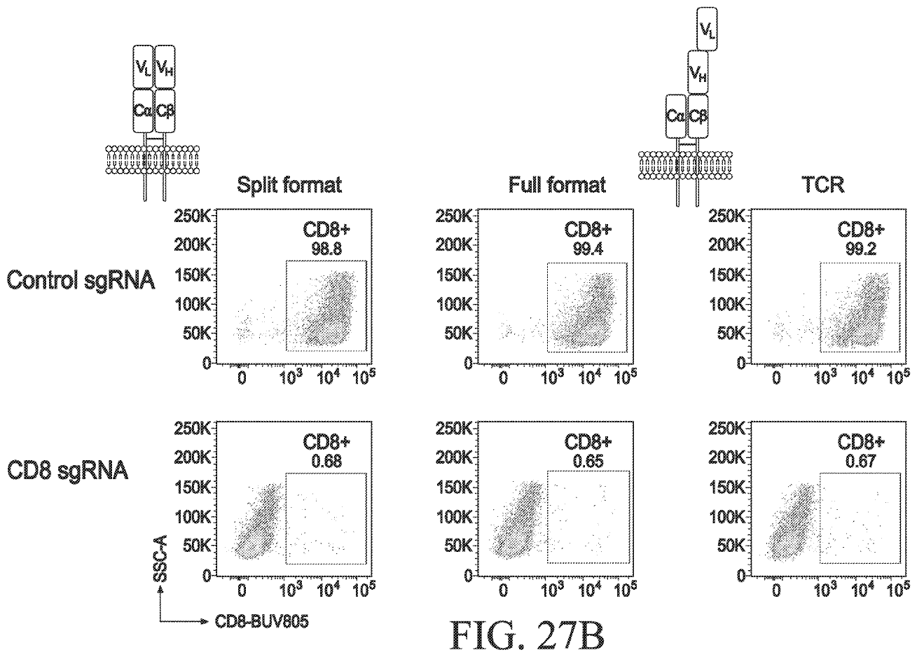

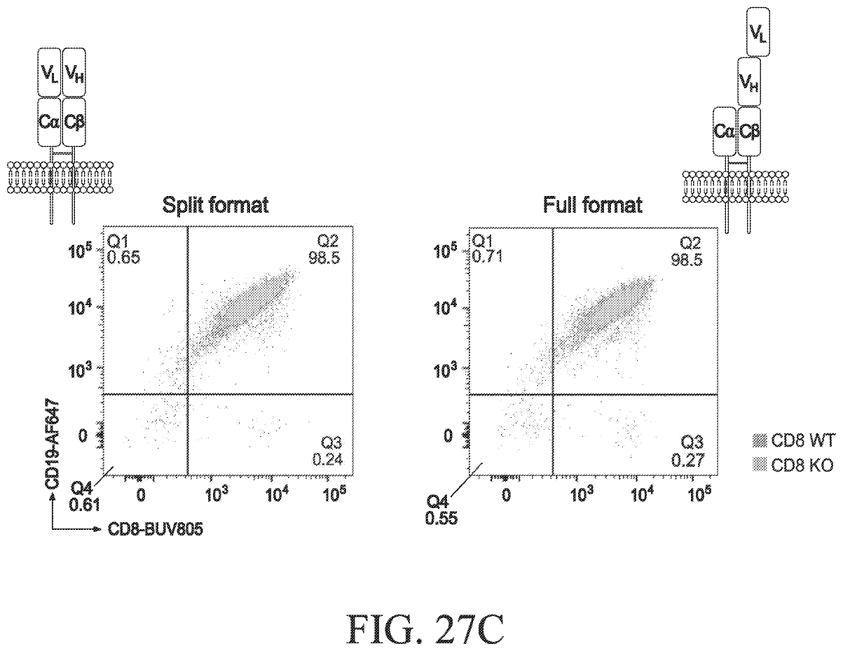

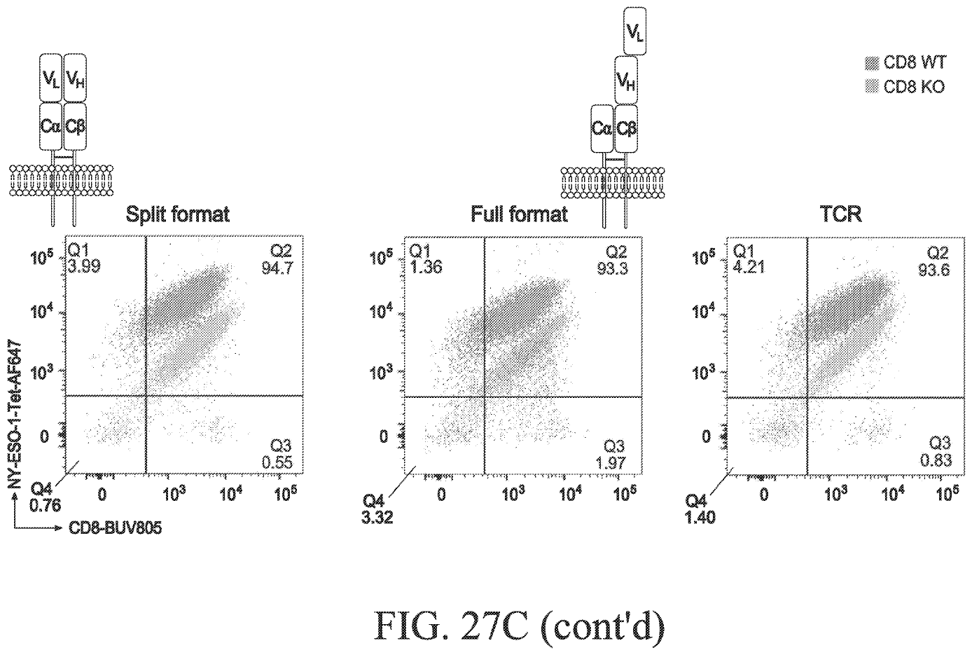

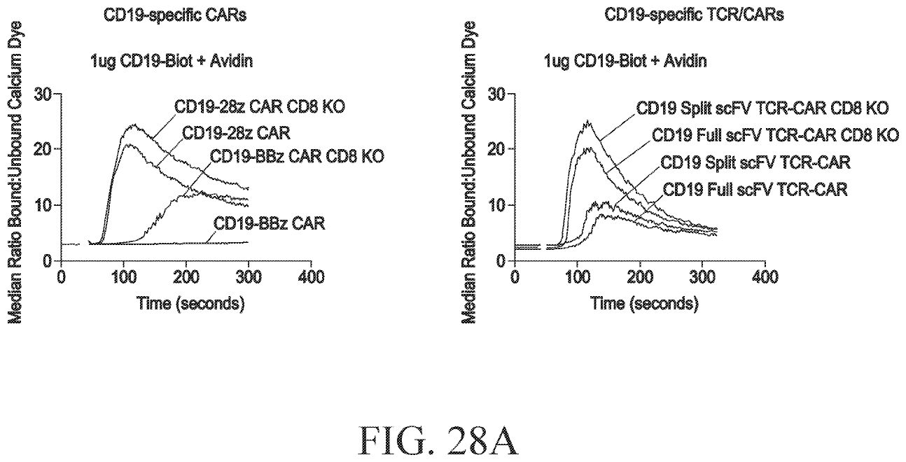

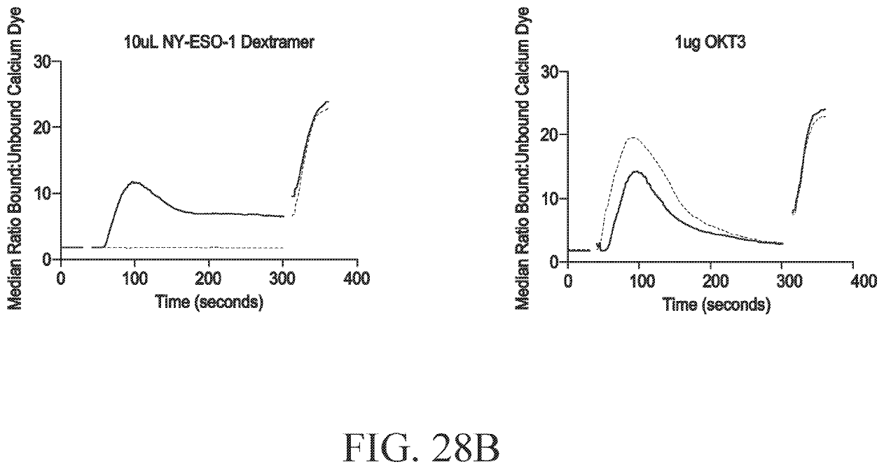

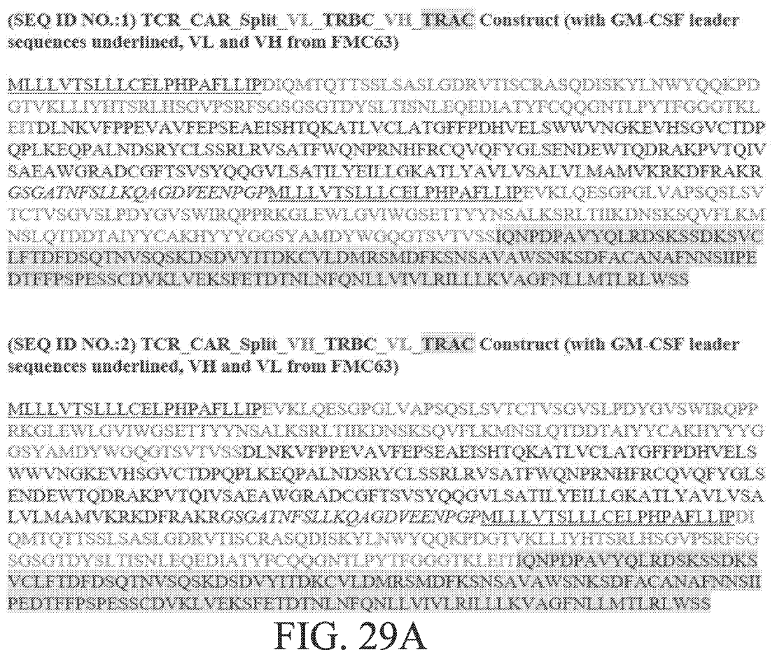

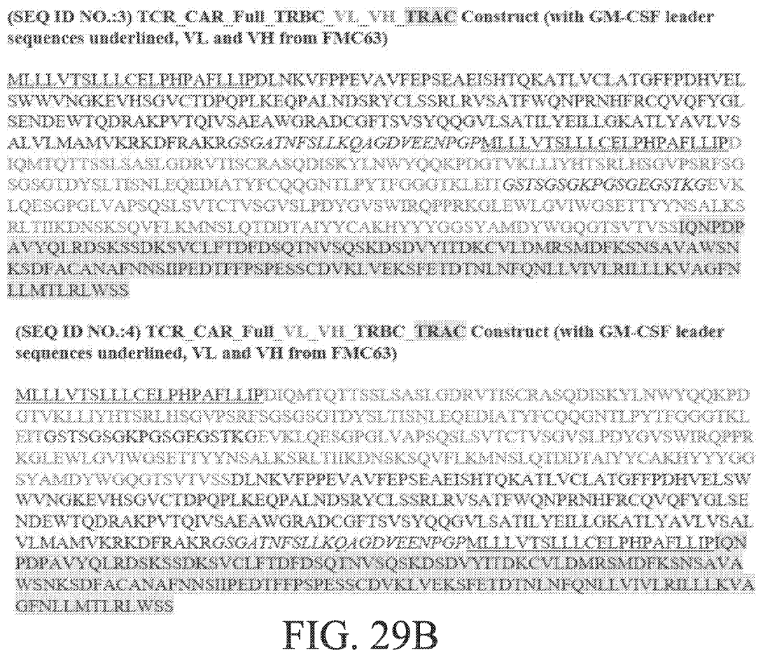

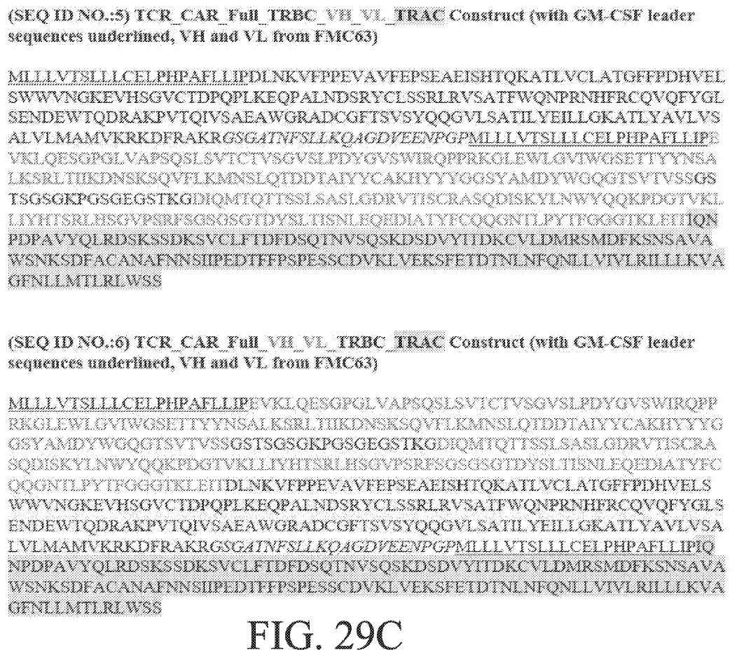

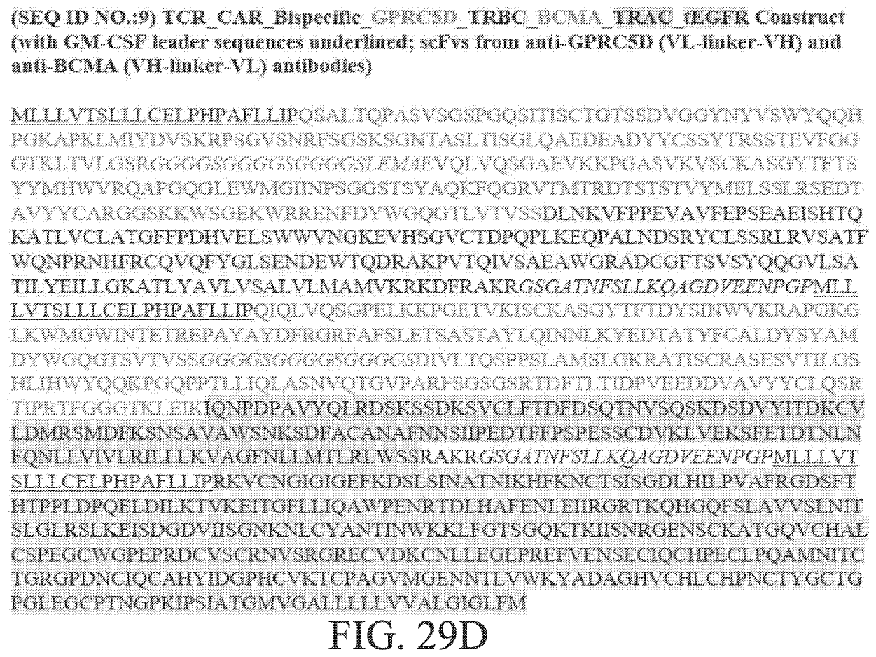

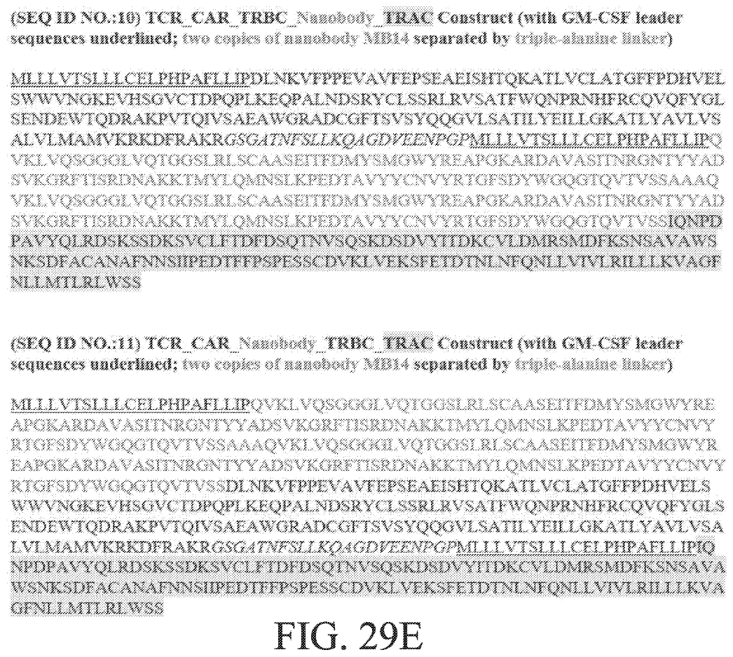

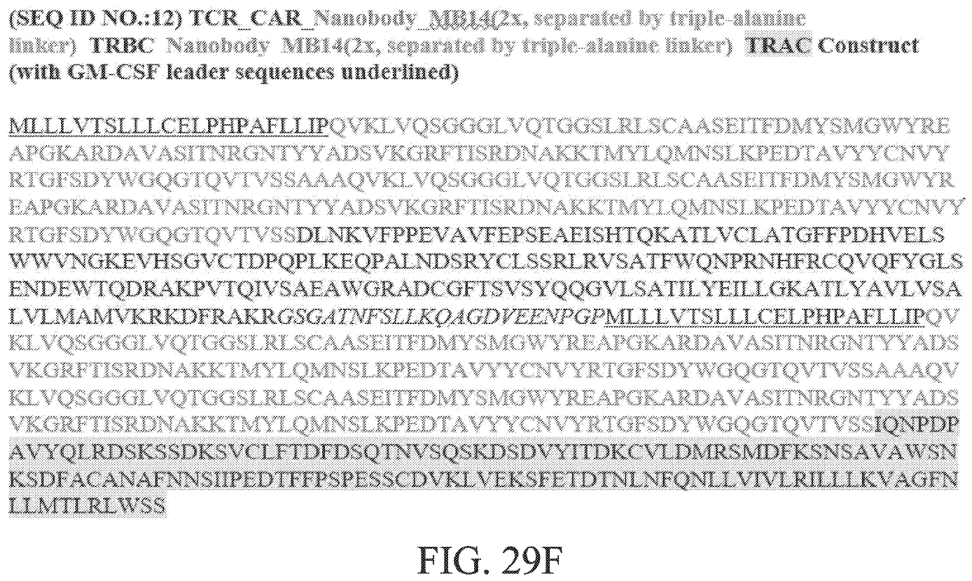

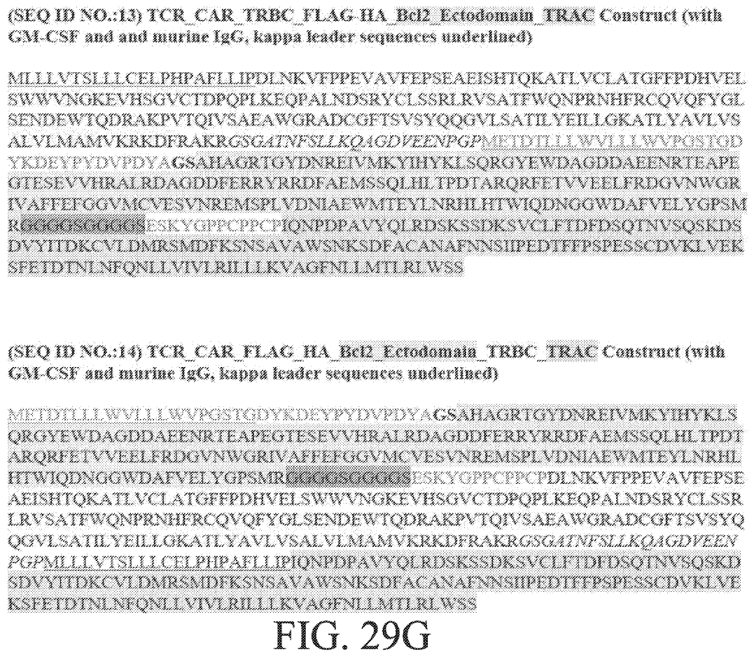

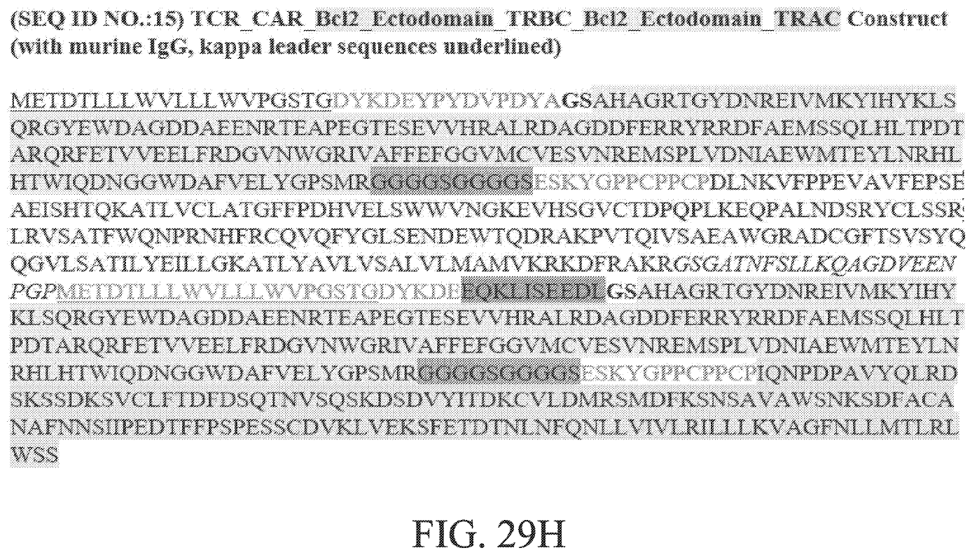

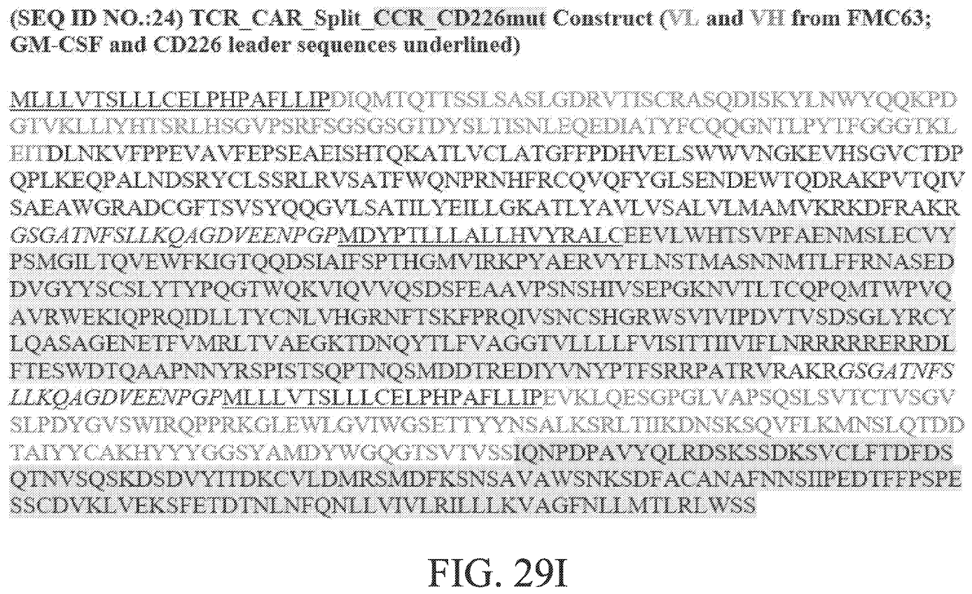

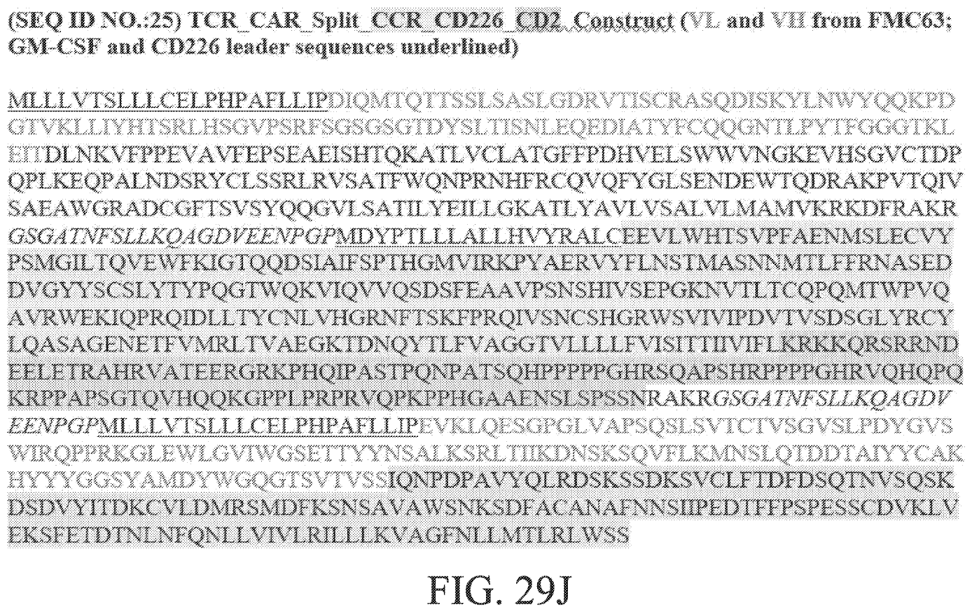

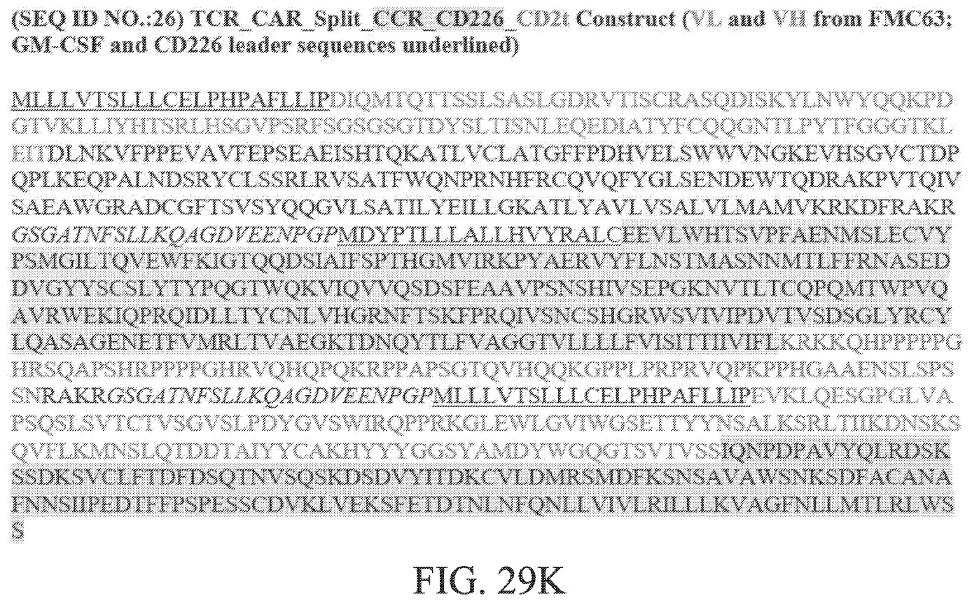

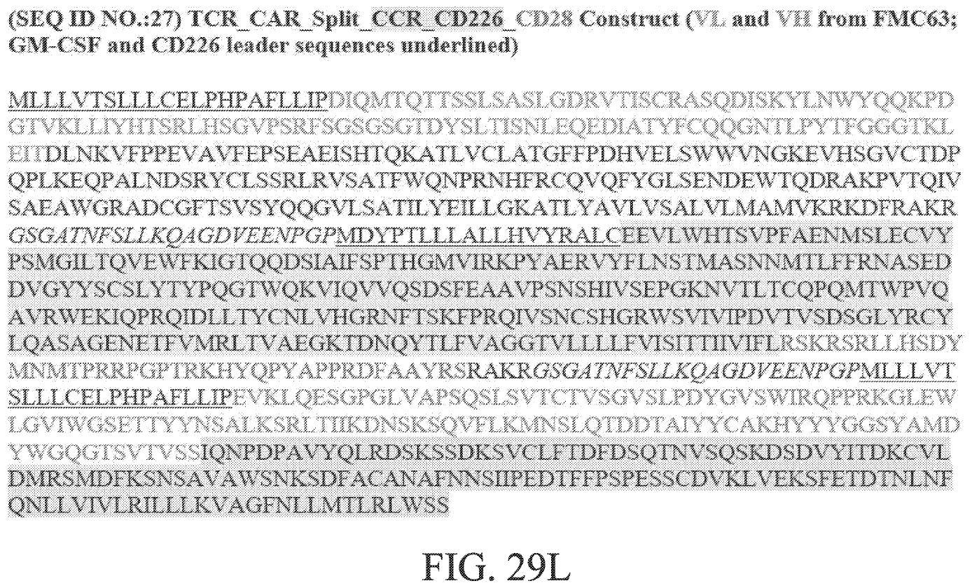

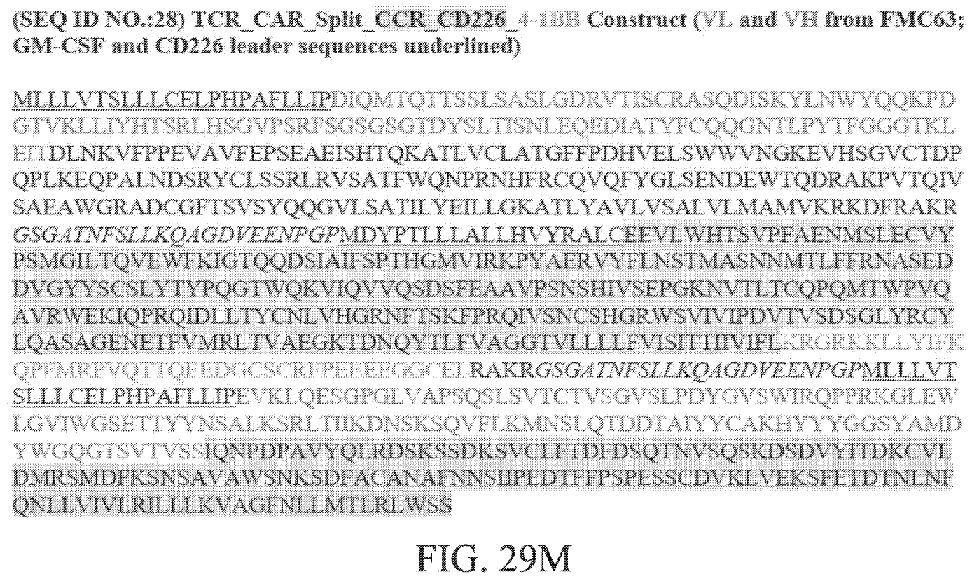

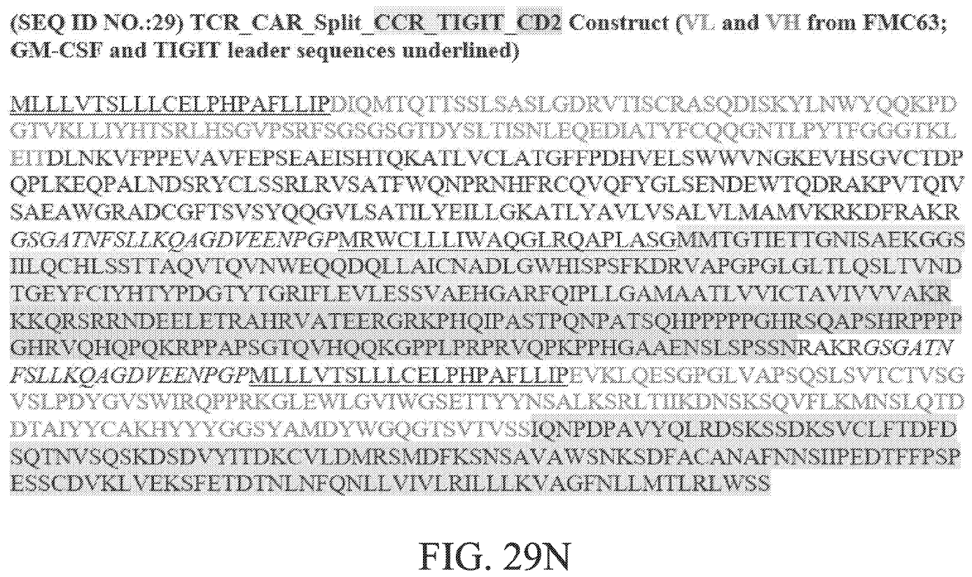

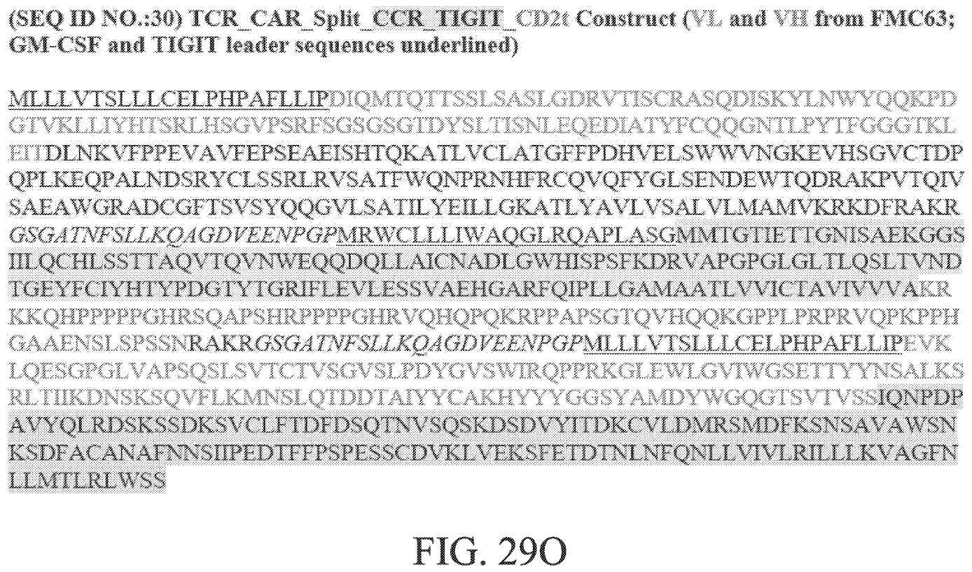

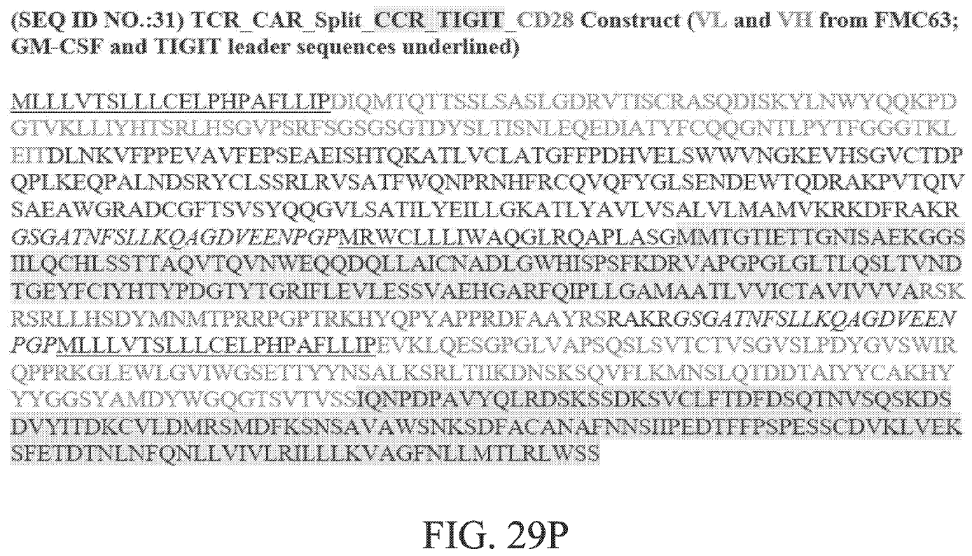

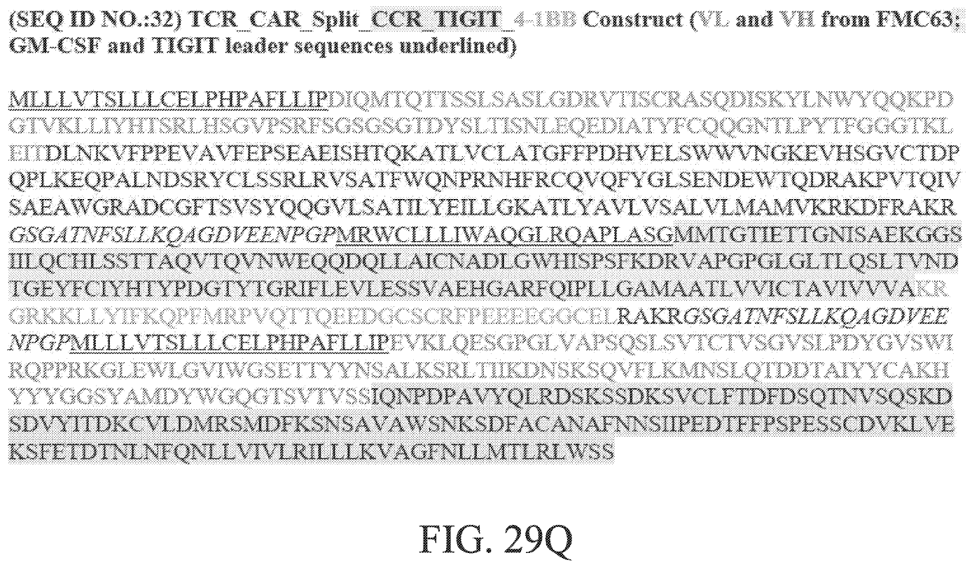

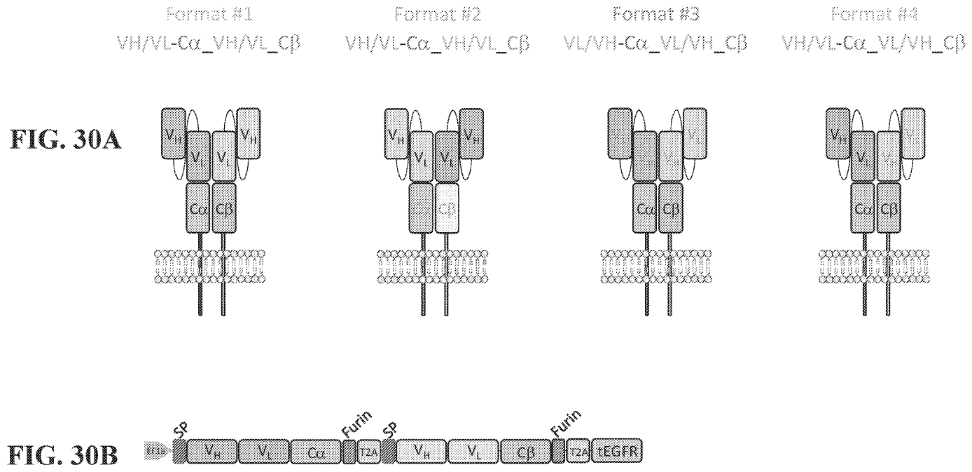

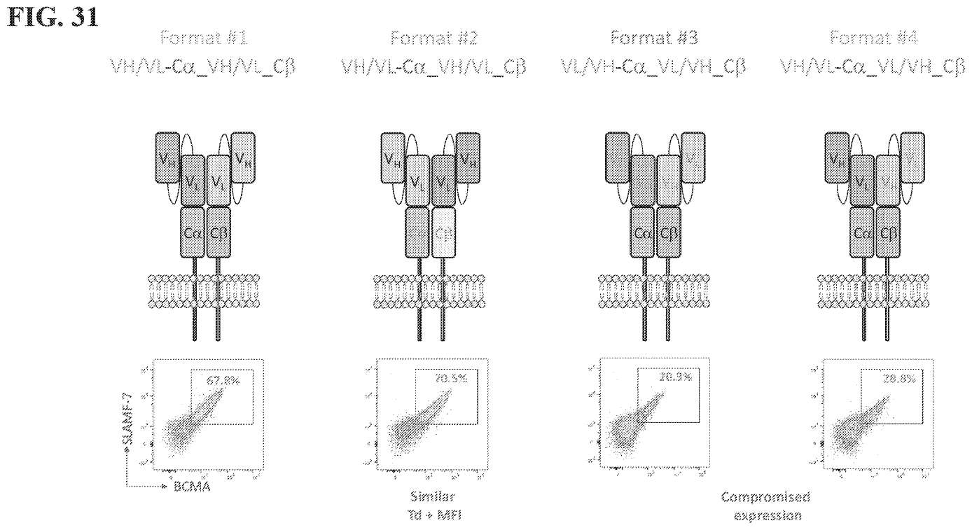

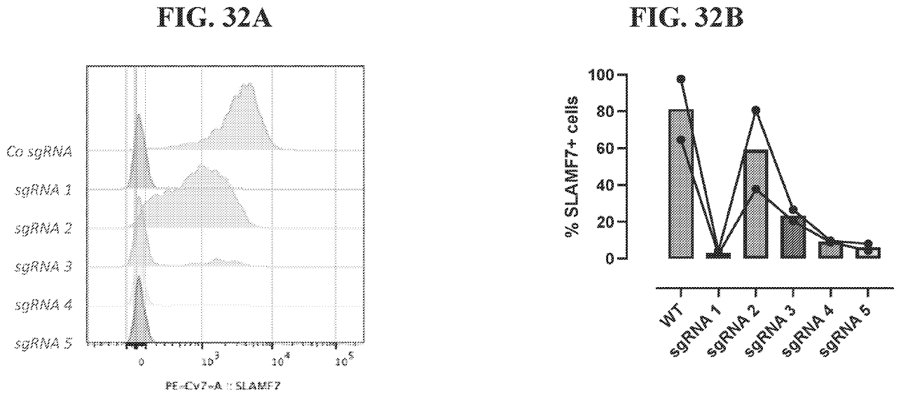

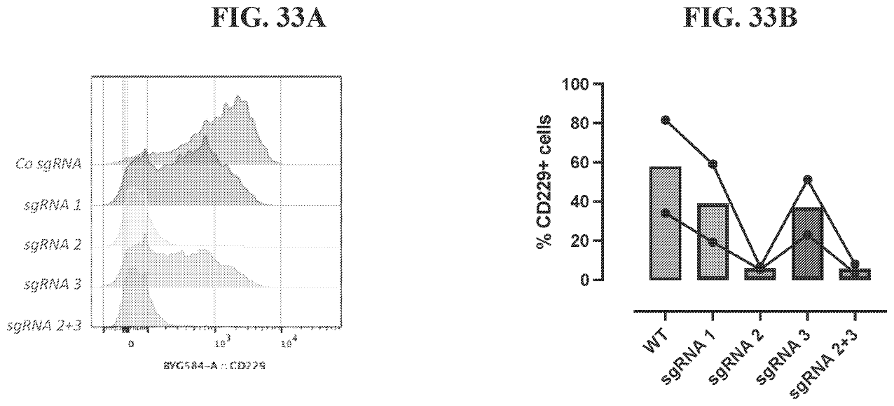

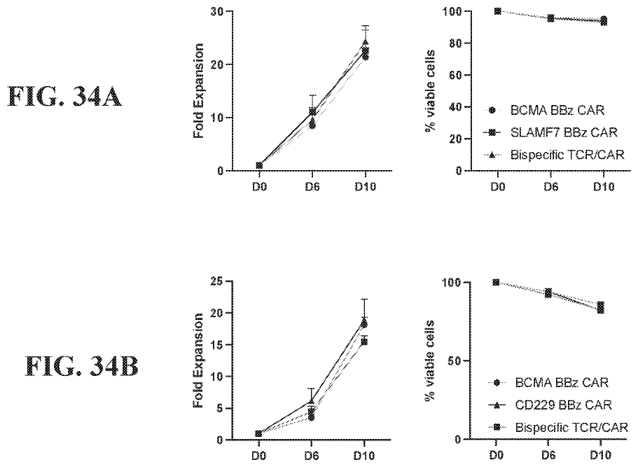

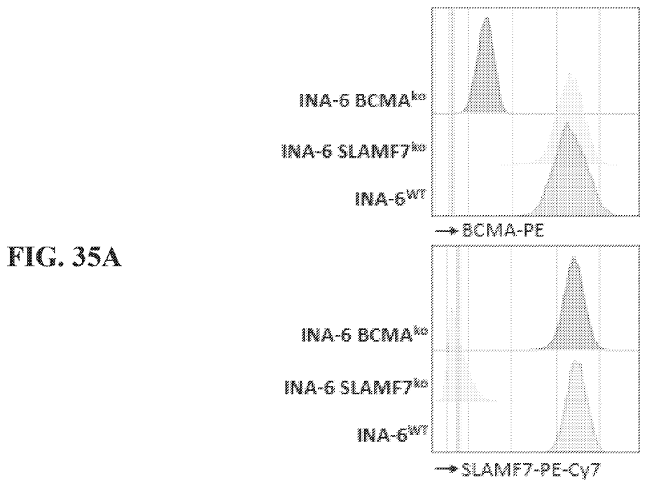

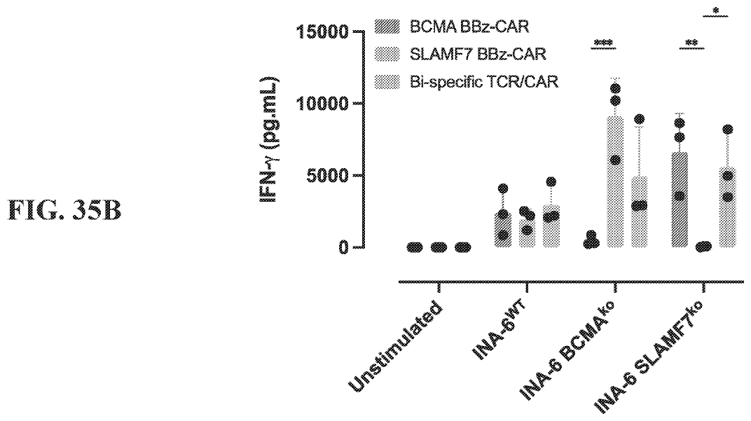

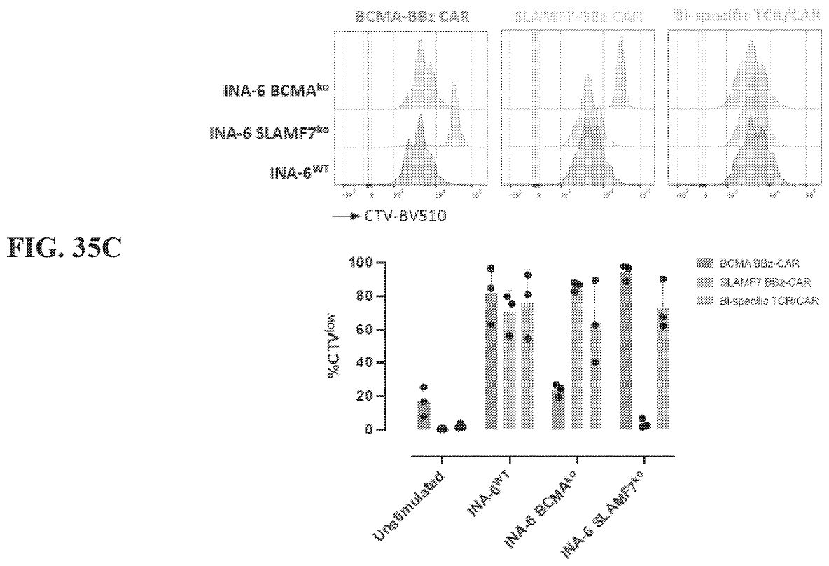

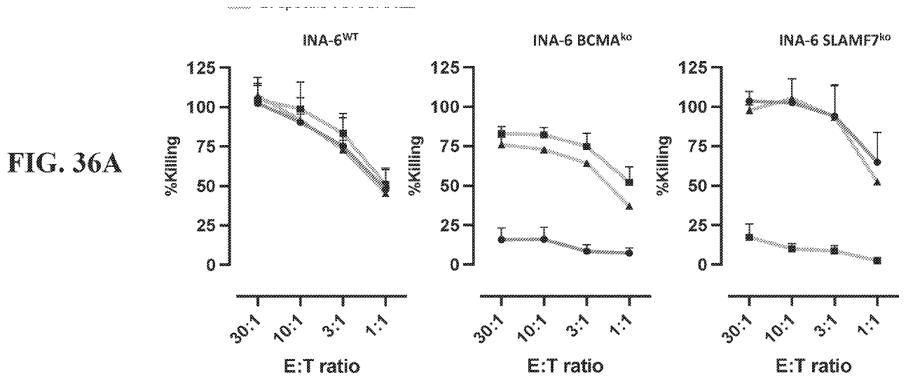

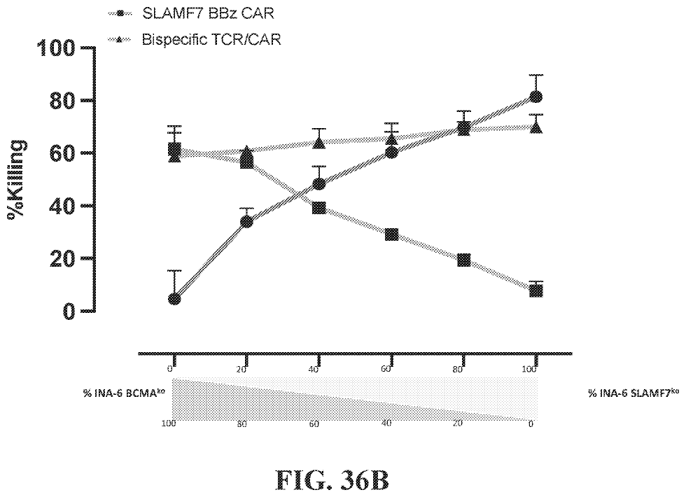

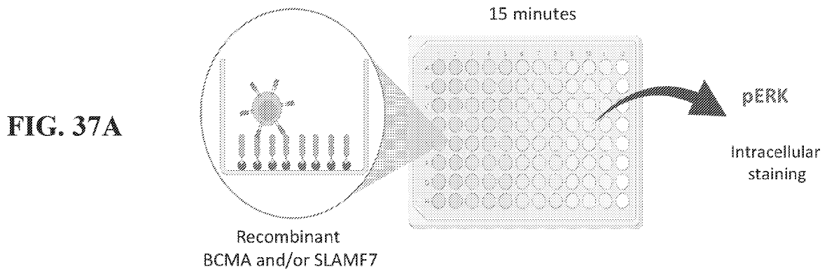

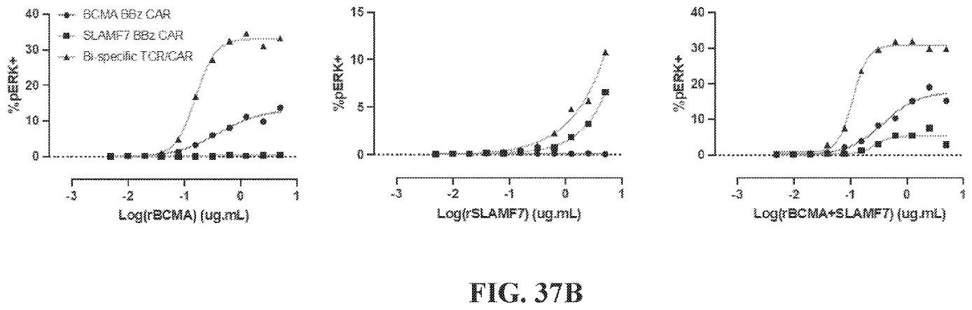

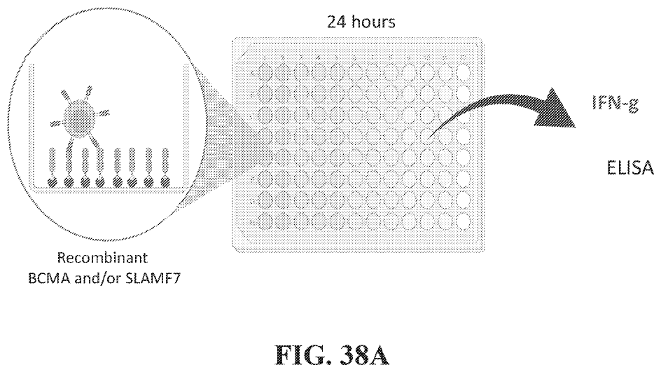

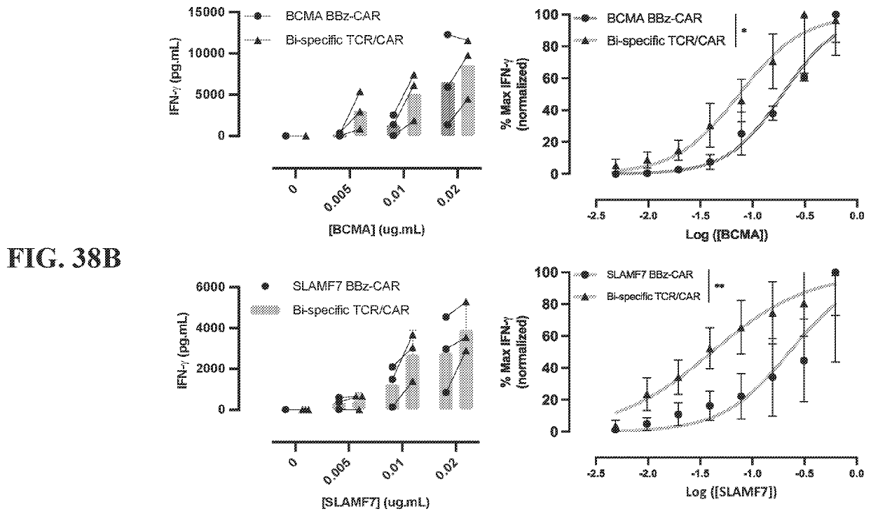

TCR/CARs reconstituted the full TCR signaling complex, conferred tumor specificity and exhibited superior antigen sensitivity in vitro, without tonic signaling (FIGS. 1A-1D and 2). The CD19 TCR/CAR shows marked anti-tumor efficacy in NSG mice.

Characterization of MM-specific TCR/CAR T cells: Lentiviral vectors encoding TCR/CARs specific for BCMA and GPRC5D multiple myeloma (MM) antigens (Ags) are constructed in split scFv and full scFv formats using available VH and VL sequences. The lentiviral vectors are used to transduce TCR-deficient Jurkats that express NFAT, NFkB and AP-1 reporters to allow identification and exclusion of constructs that tonically signal. TCR/CAR expression is determined by staining with TCRab mAb and recombinant BCMA and GPRCSD. TCR/CARs that express and lack tonic signaling are transduced into primary T cells. Endogenous TCR chain knock-out is performed using base editing with validated sgRNAs. TCR/CAR expression and cell phenotype is determined using spectral flow cytometry (Aurora, Cytek) with a monoclonal antibody (mAb) panel composed of CD3, TCR, recombinant protein for each scFv, CD4, CD8, differentiation markers (CCR7, CD45RA, CD62L, CD28, CD27 and CD95), activation markers (CD25 and CD69), inhibitory receptors (TIGIT, TIM-3, LAG-3, PD-1 and CD39), and transcription factors (TCF1, TOX, T-Bet and EOMES). All assays in Jurkat and primary T cells are compared with 4-1BB-bearing CARs specific for the same target Ags.

Specificity and function of TCR/CAR T cells: TCR/CAR cells for MM Ag are sort-purified and tested for recognition using cytotoxicity, proliferation and cytokine release assays after coculture with MMI. R, U266 and MOLP-8 MM cells²⁴ that naturally express the target Ags or are knocked out for Ag expression using CRISPR. Data are compared with conventional 4-1BB-bearing CAR-T cells specific for the same target Ags.

Antigen sensitivity: TCR/CARs may recognize lower levels of Ag than CARs because they engage the full TCR machinery. Cytokine (IL-2, IFN-y) production of TCR/CAR and CAR-T cells specific for each MM Ag is measured after stimulation with biotinylated recombinant BCMA and GPRCSD proteins adsorbed at various concentrations onto triplicate wells of avidin coated plates. EC50 is determined and compared between constructs.

Anti-tumor efficacy in vivo: The activity of TCR/CAR and CAR-T are determined in cohorts of NOD/SCID/γc^{-/-} (NSG) mice engrafted with MM. 1R/eGFP-ffluc MM cells as described²⁴. Mice receive TCR/CAR, CAR, or mock T cells at two dose levels. The frequency and phenotype (differentiation and exhaustion markers) of TCR/CAR and CAR T cells are determined in blood obtained at intervals after T cell infusion. Tumor engraftment is monitored by bioluminescence imaging (BLI). TCR/CAR or CAR T cells are flow-sorted from blood and bone marrow at day 10 and day 20 after infusion for RNA seq. Libraries are generated, sequenced, and. Tumor growth and tumor-free-survival are determined for each group.

TCR/CARs function in primary T cells, exhibit superior Ag sensitivity compared to CARs, retain greater proliferative capacity and be enriched for a transcriptional signature of memory rather than terminal effector differentiation in vitro and in vivo.

### Example 2

Design and Testing of Chimeric Costimulatory Molecules that Hijack the Tigit Inhibitor Pathway and Provide Positive Signaling to Antigen-Specific Tcr/Car T Cells

TCR/CARs that only provide for costimulation delivered through natural receptors may have limited efficacy against tumors that lack costimulatory ligands.

CD155 and CD112, the natural ligands for CD226 and TIGIT, are highly expressed by MM cells. Chimeric costimulatory receptor (CCRs) were designed that could be expressed with TCR/CARs to both provide co-stimulation in trans and subvert TIGIT inhibition. CCRs comprising CD226 or TIGIT extracellular domains and 4-1BB, CD2, or CD226 (mutant that resists degradation) endodomains were designed and conditions defined for high efficiency TCR/CAR transduction and base editing to disrupt both TCR chains and endogenous TIGIT. CCRs are readily co-expressed with TCR/CARs targeting CD19 (FIG. 3B).

Expression of CCRs in BCMA-specific TCR/CAR T cells: Studies are performed that assess design and function of various CFPs that convert TIGIT signaling to a costimulatory signal in BCMA-specific TCR/CAR T cells. Primary T cells are transduced with vectors encoding BCMA-specific TCR/CARs together with five different CFPs (CD226mut, CD226/CD2, CD226/4-1BB, TIGIT/CD2 and TIGIT/4-1BB). Base-editing the day after lentiviral transduction disrupts both the endogenous TCR and TIGIT. After 10-12 days, TCR/CAR expressing T cells are flow-sorted and phenotypes determined, as in Example 1. Transduction frequency, expression of TCR/CAR, CD226 and TIGIT are determined by flow cytometry (overexpression can delineate expression of the different CFPs) and compared to control T cells. Confirmatory immunoblot for CD226 and TIGIT is performed to detect CFPs.

Anti-tumor efficacy of BCMA-specific TCR/CAR expressing IFPs: i) In vitro function: BCMA TCR/CAR-T expressing each of the IFPs are sort-purified and evaluated in cytotoxicity, proliferation and cytokine release assays as described in Example 1 after coculture with CD155⁺ and CD155^{ko} MM1. R and U266 MM cells generated using CRISPR. This determines whether expression of IFPs augments function against CD155⁺ tumor cells and identifies constructs that confer superior function. ii) Effect on TEX: To determine whether CFPs augment T cell function in settings of chronic antigen stimulation, the development of TEX is mimicked in vitro using repetitive stimulation with CD155⁺ MM. IR cells. T cells are plated with tumor cells (ET ratio 1:1) and tumor cell killing monitored using Incucyte. Twenty-five percent (25%) of the cells are re-plated with fresh tumor every 3 days and killing, cytokine production and T cell phenotype (inhibitory receptors, TOX, TCF1, CD127, and CD27) are monitored after each stimulation. RNA seq is performed on each T cell population (n=6) after 3 and 5 tumor stimulations and compared with gene expression signatures of TEX, effector and memory T cells. iii. In vivo function and RNA seq: The antitumor activity of TCR/CAR T cells with and without each of the IFPs is evaluated as described in Example 1. The frequency and phenotype (differentiation and exhaustion markers) of T cells in blood obtained after T cell infusion is determined and tumor regression evaluated by BLI. Where TCR/CAR T cells with one or more IFPs are superior to the TCR/CAR alone, T cells from blood and bone marrow are flow-sorted at day 10 and day 20 after infusion for RNA seq to determine transcriptional signatures that correlate with superior efficacy. Development of TEX is associated with remodeling of chromatin accessibility. These in vitro or in vivo TEX models are useful to determine epigenetic changes using ATAC seq and Cut and Tag for specific transcription factors associated with TEX to understand how disruption of the TIGIT pathway and co-stimulation affects T cell fate and prevent exhaustion. All experiments are performed with T cells from at least 2 donors.

The TIGIT CCRs are well-expressed in primary T cells. The tested CD2 construct provides costimulation.

### Example 3

### Further Studies

Additional constructs were generated, and experiments were performed, as shown and described with respect to the Figures.

### REFERENCES

1. Raje N, Berdeja J, Lin Y, Siegel D, Jagannath S, Madduri D, Liedtke M, Rosenblatt J, Maus M V, Turka A, Lam L P, Morgan R A, Friedman K, Massaro M, Wang J, Russotti G, Yang Z, Campbell T, Hege K, Petrocca F, Quigley M T, Munshi N, Kochenderfer J N. Anti-BCMA CAR T-Cell Therapy bb2121 in Relapsed or Refractory Multiple Myeloma. N Engl J Med. 2019; 380 (18): 1726-37. Epub 2019 May 3. doi: 10.1056/NEJMoa1817226. PubMed PMID: 31042825.
2. Abramson J S, Palomba M L, Gordon L I, Lunning M A, Wang M, Arnason J, Mehta A, Purev E, Maloney D G, Andreadis C, Sehgal A, Solomon S R, Ghosh N, Albertson™, Garcia J, Kostic A, Mallaney M, Ogasawara K, Newhall K, Kim Y, Li D, Siddiqi T. Lisocabtagene maraleucel for patients with relapsed or refractory large B-cell lymphomas (TRANSCEND NHL 001): a multicentre seamless design study. Lancet. 2020; 396 (10254): 839-52. Epub 2020 Sep. 6. doi: 10.1016/S0140-6736 (20) 31366-0. PubMed PMID: 32888407.
3. Sadelain M, Riviere I, Riddell S. Therapeutic T cell engineering. Nature. 2017; 545 (7655): 423-31. Epub 2017 May 26. doi: 10.1038/nature22395. PubMed PMID: 28541315; PMCID: PMC5632949.
4. Majzner R G, Mackall C L. Tumor Antigen Escape from CAR T-cell Therapy. Cancer Discov. 2018; 8 (10): 1219-26. Epub 2018 Aug. 24. doi: 10.1158/2159-8290.CD-18-0442. PubMed PMID: 30135176.
5. Majzner R G, Mackall C L. Clinical lessons learned from the first leg of the CAR T cell journey. Nat Med. 2019, 25 (9): 1341-55. Epub 2019 Sep. 11. doi: 10.1038/s41591-019-0564-6. PubMed PMID: 31501612.
6. Gudipati V, Rydzek J, Doel-Perez I, Goncalves V D R, Scharf L, Konigsberger S, Lobner E, Kunert R, Einsele H, Stockinger H, Hudecek M, Huppa J B. Inefficient CAR-proximal signaling blunts antigen sensitivity. Nat Immunol. 2020; 21 (8): 848-56. Epub 2020 Jul. 8. doi: 10.1038/s41590-020-0719-0. PubMed PMID: 32632291.
7. Chen J, Lopez-Moyado I F, Seo H, Lio C J, Hempleman L J, Sekiya T, Yoshimura A, Scott-Browne J P, Rao A. NR4A transcription factors limit CAR T cell function in solid tumours. Nature. 2019; 567 (7749): 530-4. Epub 2019 Mar. 1. doi: 10.1038/s41586-019-0985-X. PubMed PMID: 30814732; PMCID: PMC6546093.
8. Feucht J, Sun J, Eyquem J, Ho Y J, Zhao Z, Leibold J, Dobrin A, Cabriolu A, Hamieh M, Sadelain M. Calibration of CAR activation potential directs alternative T cell fates and therapeutic potency. Nat Med. 2019; 25 (1): 82-8. Epub 2018 Dec. 19. doi: 10.1038/s41591-018-0290-5. PubMed PMID: 30559421; PMCID: PMC6532069.
9. Salter A I, Ivey R G, Kennedy J J, Voillet V, Rajan A, Alderman E J, Voytovich U J, Lin C, Sommermeyer D, Liu L, Whiteaker J R, Gottardo R, Paulovich A G, Riddell S R. Phosphoproteomic analysis of chimeric antigen receptor signaling reveals kinetic and quantitative differences that affect cell function. Sci Signal. 2018; 11 (544). Epub 2018 Aug. 23. doi: 10.1126/scisignal.aat6753. PubMed PMID: 30131370; PMCID: PMC6186424.
10. Guillerey C, Harjunpaa H, Carrie N, Kassem S, Teo T, Miles K, Krumeich S, Weulersse M, Cuisinier M, Stannard K, Yu Y, Minnie S A, Hill G R, Dougall W C, Avet-Loiseau H, Teng M W L, Nakamura K, Martinet L, Smyth M J. TIGIT immune checkpoint blockade restores CD8 (+) T-cell immunity against multiple myeloma. Blood. 2018; 132 (16): 1689-94. Epub 20180709. doi: 10.1182/blood-2018-01-825265. PubMed PMID: 29986909.
11. Minnie S A, Kuns R D, Gartlan K H, Zhang P, Wilkinson A N, Samson L, Guillerey C, Engwerda C, MacDonald KPA, Smyth M J, Markey K A, Vuckovic S, Hill G R. Myeloma escape after stem cell transplantation is a consequence of T-cell exhaustion and is prevented by TIGIT blockade. Blood. 2018; 132 (16): 1675-88. Epub 20180828. doi: 10.1182/blood-2018-01-825240. PubMed PMID: 30154111.
12. Munshi N C, Anderson L D, Jr., Shah N, Madduri D, Berdeja J, Lonial S, Raje N, Lin Y, Siegel D, Oriol A, Moreau P, Yakoub-Agha I, Delforge M, Cavo M, Einsele H, Goldschmidt H, Weisel K, Rambaldi A, Reece D, Petrocca F, Massaro M, Connamn J N, Kaiser S, Patel P, Huang L, Campbell T B, Hege K, San-Miguel J. Idecabtagene Vicleucel in Relapsed and Refractory Multiple Myeloma. N Engl J Med. 2021; 384 (8): 705-16. doi: 10.1056/NEJMoa2024850. PubMed PMID: 33626253.
13. Green D J P, M. Cowan, A. J. Cole, G. O. Sather, B. D. Nagengast, A. M. Song, X. Thomas, S. Wood, B. L. Blake, M. L. Works, M. G. Gooley, T. A. Maloney, D. G. Riddell, S. R. Response to BCMA CAR-T cells correlates with pretreatment antigen density and is improved by small molecule inhibition of gamma secretase2019; Blood (134): 1856.
14. Cowan A J, Pont M, Sather B D, Turtle C J, Till B G, Nagengast A M, Libby E N, III, Becker P S, Coffey D G, Tuazon S A, Wood B L, Blake M L, Works M G, Gooley T A, Wu Q V, Maloney D G, Riddell S R, Green D J. Efficacy and Safety of Fully Human Bcma CAR T Cells in Combination with a Gamma Secretase Inhibitor to Increase Bema Surface Expression in Patients with Relapsed or Refractory Multiple Myeloma. Blood. 2019; 134 (Supplement_1): 204-. doi: 10.1182/blood-2019-129405.
15. de Larrea C F, Stachr M, Lopez A V, Ng K Y, Chen Y, Godfrey W D, Purdon T J, Ponomarev V, Wendel H G, Brentjens R J, Smith E L. Defining an Optimal Dual-Targeted CAR T-cell Therapy Approach Simultaneously Targeting BCMA and GPRC5D to Prevent BCMA Escape-Driven Relapse in Multiple Myeloma. Blood Cancer Discov. 2020; 1 (2): 146-54. Epub 2020 Oct. 23. doi: 10.1158/2643-3230.bcd-20-0020. PubMed PMID: 33089218; PMCID: PMC7575057.
16. Srivastava S, Riddell S R. Engineering CAR-T cells: Design concepts. Trends Immunol. 2015; 36 (8): 494-502. Epub 20150711. doi: 10.1016/j.it. 2015.06.004. PubMed PMID: 26169254; PMCID: PMC4746114.
17. Sykulev Y, Joo M, Vturina I, Tsomides T J, Eisen H N. Evidence that a single peptide-MHC complex on a target cell can elicit a cytolytic T cell response. Immunity. 1996; 4 (6): 565-71. Epub 1996 Jun. 1. doi: 10.1016/s1074-7613 (00) 80483-5. PubMed PMID: 8673703.
18. Finco T S, Kadlecek T, Zhang W, Samelson L E, Weiss A. LAT is required for TCR-mediated activation of PLCgammal and the Ras pathway. Immunity. 1998; 9 (5): 617-26. Epub 1998 Dec. 10. doi: 10.1016/s1074-7613 (00) 80659-7. PubMed PMID: 9846483.
19. Salter A I, Rajan A, Kennedy J J, Ivey R G, Shelby S A, Leung I, Templeton M L, Muhunthan V, Voillet V, Sommermeyer D, Whiteaker J R, Gottardo R, Veatch S L, Paulovich A G, Riddell S R. Comparative analysis of TCR and CAR signaling informs CAR designs with superior antigen sensitivity and in vivo function. Sci Signal. 2021; 14 (697). Epub 20210824. doi: 10.1126/scisignal.abe2606. PubMed PMID: 34429382.
20. Watanabe K, Terakura S, Martens A C, van Meerten T, Uchiyama S, Imai M, Sakemura R, Goto T, Hanajiri R, Imahashi N, Shimada K, Tomita A, Kiyoi H, Nishida T, Naoe T, Murata M. Target antigen density governs the efficacy of anti-CD20-CD28-CD3 zeta chimeric antigen receptor-modified effector CD8⁺ T cells. J Immunol. 2015; 194 (3): 911-20. Epub 2014 Dec. 19. doi: 10.4049/jimmunol. 1402346. PubMed PMID: 25520398
21. Stengel K F, Harden-Bowles K, Yu X, Rouge L, Yin J, Comps-Agrar L, Wiesmann C, Bazan J F, Eaton D L, Grogan J L. Structure of TIGIT immunoreceptor bound to poliovirus receptor reveals a cell-cell adhesion and signaling mechanism that requires cis-trans receptor clustering. Proc Natl Acad Sci USA. 2012; 109 (14): 5399-404. Epub 2012 Mar. 17. doi: 10.1073/pnas.1120606109. PubMed PMID: 22421438; PMCID: PMC3325733.
22. Weulersse M, Asrir A, Pichler A C, Lemaitre L, Braun M, Carrie N, Joubert M V, Le Moine M, Do Souto L, Gaud G, Das I, Brauns E, Scarlata C M, Morandi E, Sundarrajan A, Cuisinier M, Buisson L, Maheo S, Kassem S, Agesta A, Peres M, Verhoeyen E, Martinez A, Mazieres J, Dupre L, Gossye T, Pancaldi V, Guillerey C, Ayyoub M, Dejean A S, Saoudi A, Goriely S, Avet-Loiseau H, Bald T, Smyth M J, Martinet L. Eomes-Dependent Loss of the Co-activating Receptor CD226 Restrains CD8 (+) T Cell Anti-tumor Functions and Limits the Efficacy of Cancer Immunotherapy. Immunity. 2020; 53 (4): 824-39 e10. Epub 2020 Oct. 15. doi: 10.1016/j.immuni.2020.09.006. PubMed PMID: 33053331.
23. Braun M, Aguilera A R, Sundarrajan A, Corvino D, Stannard K, Krumeich S, Das I, Lima L G, Meza Guzman L G, Li K, Li R, Salim N, Jorge M V, Ham S, Kelly G, Vari F, Lopletier A, Raghavendra A, Pearson S, Madore J, Jacquelin S, Effern M, Quine B, Koufariotis L T, Casey M, Nakamura K, Seo E Y, Holzel M, Geyer M, Kristiansen G, Taheri T, Ahern E, Hughes B G M, Wilmott J S, Long G V, Scolyer R A, Batstone M D, Landsberg J, Dietrich D, Pop O T, Flatz L, Dougall W C, Veillette A, Nicholson S E, Moller A, Johnston R J, Martinet L, Smyth M J, Bald T. CD155 on Tumor Cells Drives Resistance to Immunotherapy by Inducing the Degradation of the Activating Receptor CD226 in CD8 (+) T Cells. Immunity. 2020; 53 (4): 805-23 e15. Epub 2020 Oct. 15. doi: 10.1016/j.immuni.2020.09.010. PubMed PMID: 33053330.
24. Pont M J, Hill T, Cole G O, Abbott J J, Kelliher J, Salter A I, Hudecek M, Comstock M L, Rajan A, Patel B K R, Voutsinas J M, Wu Q, Liu L, Cowan A J, Wood B L, Green D J, Riddell S R. gamma-Secretase inhibition increases efficacy of BCMA-specific chimeric antigen receptor T cells in multiple myeloma. Blood. 2019; 134 (19): 1585-97. Epub 2019 Sep. 29. doi: 10.1182/blood.2019000050. PubMed PMID: 31558469; PMCID: PMC6871311.
25. Fraietta J A, Lacey S F, Orlando E J, Pruteanu-Malinici I, Gohil M, Lundh S, Boesteanu A C, Wang Y, O'Connor R S, Hwang W T, Pequignot E, Ambrose D E, Zhang C, Wilcox N, Bedoya F, Dorfmeier C, Chen F, Tian L, Parakandi H, Gupta M, Young R M, Johnson F B, Kulikovskaya I, Liu L, Xu J, Kassim S H, Davis M M, Levine B L, Frey N V, Siegel D L, Huang A C, Wherry E J, Bitter H, Brogdon J L, Porter D L, June C H, Melenhorst J J. Determinants of response and resistance to CD19 chimeric antigen receptor (CAR) T cell therapy of chronic lymphocytic leukemia. Nat Med. 2018; 24 (5): 563-71. Epub 2018 May 2. doi: 10.1038/s41591-018-0010-1. PubMed PMID: 29713085; PMCID: PMC6117613.
26. Sommermeyer D, Hudecek M, Kosasih P L, Gogishvili T, Maloney D G, Turtle C J, Riddell S R. Chimeric antigen receptor-modified T cells derived from defined CD8+ and CD4+ subsets confer superior antitumor reactivity in vivo. Leukemia. 2016; 30 (2): 492-500. Epub 2015 Sep. 16. doi: 10.1038/leu.2015.247. PubMed PMID: 26369987; PMCID: PMC4746098.
27 Hudecek M, Lupo-Stanghellini M T, Kosasih P L, Sommermeyer D, Jensen M C, Rader C, Riddell S R. Receptor affinity and extracellular domain modifications affect tumor recognition by RORI-specific chimeric antigen receptor T cells. Clin Cancer Res. 2013; 19 (12): 3153-64. Epub 2013 Apr. 27. doi: 10.1158/1078-0432.CCR-13-0330. PubMed PMID: 23620405; PMCID: PMC3804130.
28. horashian S, Kramer A M, Onuoha S, Wright G, Bartram J, Richardson R, Albon S J, Casanovas-Company J, Castro F, Popova B, Villanueva K, Yeung J, Vetharoy W, Guvenel A, Wawrzyniecka P A, Mekkaoui L, Cheung G W, Pinner D, Chu J, Lucchini G, Silva J, Ciocarlie O, Lazareva A, Inglott S, Gilmour K C, Ahsan G, Ferrari M, Manzoor S, Champion K, Brooks T,
Lopes A, Hackshaw A, Farzaneh F, Chiesa R, Rao K, Bonney D, Samarasinghe S, Goulden N, Vora A, Veys P, Hough R, Wynn R, Pule M A, Amrolia P J. Enhanced CAR T cell expansion and prolonged persistence in pediatric patients with ALL treated with a low-affinity CD19 CAR. Nat Med. 2019; 25 (9): 1408-14. Epub 2019 Sep. 4. doi: 10.1038/s41591-019-0549-5. PubMed PMID: 31477906.
29. Pires D E, Ascher D B. mCSM-AB: a web server for predicting antibody-antigen affinity changes upon mutation with graph-based signatures. Nucleic Acids Res. 2016; 44 (W1): W469-73. Epub 2016 May 25. doi: 10.1093/nar/gkw458. PubMed PMID: 27216816; PMCID: PMC4987957. Van Laethem et al., Cell 154 (6): 1326-41 (2013); doi: 10.1016/j.cell.2013.08.009
Wei et al., PNAS 117 (27): 15809-15817
Poorebrahim et al. Cancer Gene Therapy 28, 581-589 (2021)
Gross et al. PNAS 86 (24): 10024-10028; doi: 10.1073/pnas.86.24.10024
Becker et al. Cell 58 (5): 911-921 (1989); doi.org/10.1016/0092-8674 (89) 90943-4
Goverman et al. Cell 60 (6): 929-939 (1990); doi.org/10.1016/0092-8674 (90) 90341-B
Groettrup et al. EMBO J. 11 (7): 2735-2745 (1992) PCT Publication No. WO2018067993A1
PCT Publication No. WO2016187349A1
Wu et al. Cellular & Molecular Immunology 17:600-612 (2020); doi.org/10.1038/s41423-020-0470-3
US Patent Publication No. US 2015/183877
Semiya et al. J. Biochem. 113 (6): 687-691 (1993)
Ma et al. Front. Immunol. 12:626616 (2021); doi: 10.3389/fimmu.2021.626616

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/337,588, filed on May 2, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A polypeptide dimer comprising (i) a first polypeptide comprising a first T cell receptor (TCR) constant domain and (ii) a second polypeptide comprising a second TCR constant domain, wherein the first TCR constant domain and the second TCR constant domain associate with one another, and wherein first polypeptide, the second polypeptide, or both, further comprises a target-binding domain disposed amino-terminal to the first or the second TCR constant domain, respectively, wherein the target-binding domain does not comprise a TCR variable domain.

2. The polypeptide dimer of claim 1, wherein the target-binding domain comprises: (i) an antibody heavy chain variable domain (VI); (ii) an antibody light chain variable domain (VL); (iii) a single-chain variable fragment (scFv); (iv) a fragment antigen-binding region (Fab); (v) a single-chain Fab; (vi) an antigen-binding fragment of a heavy chain-only antibody (VHH); (vii) a designed ankyrin repeat protein (DARPin); (viii) a ¹⁰FNIII domain; (ix) a lectin binding domain; (x) a protein ligand-binding domain, such as a receptor ectodomain or a functional portion or fragment thereof; (xi) a killer immunoreceptor from a NK cell; (xii) a fibrinogen domain; (xiii) a cysteine-knot miniprotein; (xiv) a tetratricopeptide repeat domain; (xv) a leucine-rich repeat domain; (xvi) a lipocalin domain; (xvii) an armadillo repeat protein; (xviii) an affibody; (xix) an avimer; (xx) a knottin; (xxi) a fynomer; (xxii) an atrimer; (xxiii) CTLA4 (e.g., a ligand-binding domain of CTLA4); (xxiv) a synthetic protein designed to bind a natural ligand; (xxv) a ligand, such as for example a cytokine or a peptide tag; or (xxvi) any combination of (i)-(xxv).

3. The polypeptide dimer of claim 1 or claim 2, wherein: (a) the first TCR constant domain comprises a TCR alpha-chain constant domain (Cα) and the second TCR constant domain comprises a TCR beta-chain constant domain (Cβ); (b) the first TCR constant domain comprises a Cβ and the second TCR constant domain comprises a Cα; (c) the first TCR constant domain comprises a TCR gamma-chain constant domain (Cγ) and the second TCR constant domain comprises a TCR delta-chain constant domain (Cδ); or (d) the first TCR constant domain comprises a Cδ and the second TCR constant domain comprises a Cγ.

4. The polypeptide dimer of any one of claims 1-3, wherein the first TCR constant domain, the second TCR constant domain, or both, comprises an amino acid substitution mutation that promotes pairing between the first TCR constant domain and the second TCR constant domain.

5. The polypeptide dimer of claim 4, wherein the amino acid substitution mutation comprises a cysteine amino acid at a non-native position and the cysteine amino acid forms a disulfide bond with an amino acid at a corresponding position in the other of the second or the first TCR constant domain, respectively.

6. The polypeptide dimer of any one of claims 1-5, wherein the first polypeptide or the second polypeptide comprises a TCR Cα domain comprising one or more mutations to improve stability of the polypeptide dimer when expressed at a surface of a host cell, wherein, optionally, the one or more mutations comprise an L-V-L mutation.

7. The polypeptide dimer of any one of claims 1-6, wherein: (1) the first polypeptide comprises a TCR Co having at least 90%, at least 91%, at least 92%, at least 93%, at least 94%, at least 95%, at least 96%, at least 97%, at least 98%, at least 99%, or 100% identity to any one of SEQ ID NOs.: 56-57, and the second polypeptide comprises a TCR Cβ having at least 90%, at least 91%, at least 92%, at least 93%, at least 94%, at least 95%, at least 96%, at least 97%, at least 98%, at least 99%, or 100% identity to any one of SEQ ID NOs.: 58-61; or (2) the first polypeptide comprises a TCR Cβ having at least 90%, at least 91%, at least 92%, at least 93%, at least 94%, at least 95%, at least 96%, at least 97%, at least 98%, at least 99%, or 100% identity to any one of SEQ ID NOs.: 58-61; and the second polypeptide comprises a TCR Cα having at least 90%, at least 91%, at least 92%, at least 93%, at least 94%, at least 95%, at least 96%, at least 97%, at least 98%, at least 99%, or 100% identity to any one of SEQ ID NOs.: 56-57.

8. The polypeptide dimer of any one of claims 1-7, comprising a TCR Cα and a TCR Cβ having at least least 90%, at least 91%, at least 92%, at least 93%, at least 94%, at least 95%, at least 96%, at least 97%, at least 98%, or at least 99% to, or comprising or consisting of, the amino acid sequences set forth in SEQ ID NOs.: (i) 56 and 58, respectively; (ii) 56 and 59, respectively; (iii) 56 and 60, respectively; (iv) 56 and 61, respectively; (v) 57 and 58, respectively; (vi) 57 and 59, respectively; (vii) 57 and 60, respectively; or (viii) 57 and 61, respectively.

9. The polypeptide dimer of any one of claims 3-8, wherein the TCR Cβ comprises the intracellular sequence VKRKDF (SEQ ID NO.: 64) or MAMVKRKDSRG (SEQ ID NO.: 65).

10. The polypeptide dimer of any one of claims 1-9, wherein the first polypeptide, the second polypeptide, or both, comprises an intracellular portion that consists essentially of or consists of the intracellular portion of a native TCR constant domain (e.g. if the polypeptide comprises a TCR Cα, the intracellular portion of the first polypeptide consists essentially of or consists of the intracellular portion of a native TCR Cα; if the polypeptide comprises a TCR Cβ, the intracellular portion of the first polypeptide consists essentially of or consists of the intracellular portion of a native TCR Cβ).

11. The polypeptide dimer of any one of claims 1-10, wherein the first polypeptide, the second polypeptide, or both, does not comprise an intracellular signaling component from a CD3 protein, and/or does not comprise an intracellular costimulatory domain from a costimulatory protein.

12. The polypeptide dimer of any one of claims 1-11, comprising a design according to Table 1.

13. The polypeptide dimer of any one of claims 1-12, wherein the first polypeptide and the second polypeptide comprise, N-terminal to the first TCR constant domain and the second TCR constant domain, respectively: (i) a VH, and a cognate VL; (ii) a VL, and a cognate VH; (iii) a scFv, and no antigen-binding domain; (iv) no antigen-binding domain, and a scFv; (v) VH-CH1, and a cognate VL-CL; (vi) VL-CL, and a cognate VH-CH1; (vii) a scFab, and no antigen-binding domain; (viii) no antigen-binding domain, and a scFab; (ix) a scFv, and a scFv; (x) a scFab, and a scFv; (xi) a scFv, and a scFab; (xii) a VHH, and no antigen-binding domain; (xiii) no antigen-binding domain, and a VHH; (xiv) VHH-linker-VHH, and no binding domain; (xv) VHH-linker-VHH, and VHH-linker-VHH; (xvi) a scFv, and a VHH or a VHH-linker-VHH; (xvii) a VHH or a VHH-linker-VHH, and a scFv; (xvii) a scFab, and a VHH or a VHH-linker-VHH; (xviii) a VHH or a VHH-linker-VHH, and a scFab; (xix) a VHH, and a VHH; (xx) a natural or synthetic protein ligand-binding domain, and no antigen-binding domain; (xxi) no antigen-binding domain, and a natural or synthetic protein ligand-binding domain; (xxii) a scFv, and a natural or synthetic protein ligand-binding domain; (xxiii) a natural or synthetic protein ligand-binding domain, and a scFv; (xxiii) a scFab, and a natural or synthetic protein ligand-binding domain; (xxiv) a natural or synthetic protein ligand-binding domain, and a scFab; (xxv) a VHH or a VHH-linker-VHH, and a natural or synthetic protein ligand-binding domain; (xxvi) a natural or synthetic protein ligand-binding domain, and a VHH or a VHH-linker-VHH; or (xxvii) a natural or synthetic protein ligand-binding domain, and a protein ligand binding-domain.

14. The polypeptide dimer of any one of claims 1-13, wherein (1) the first polypeptide comprises a target-binding domain amino-terminal to the first TCR constant domain and the second polypeptide consists essentially of or consists of the second TCR constant domain; or (2) the second polypeptide comprises a target-binding domain amino-terminal to the second TCR constant domain and the first polypeptide consists essentially of or consists of the first TCR constant domain.

15. The polypeptide dimer of any one of claims 1-14, wherein the target is expressed on or by a cancer cell, or is expressed on or by a cell infected with a pathogen (e.g. virus, fungus, parasite, bacteria) or is otherwise associated with an an infection, or is associated with an autoimmune disease or a neurodegenerative disease (e.g., tau, amyloid-beta, alpha-synuclein), or is a cytokine (e.g. TNFα, IL-13, IL-10) or a chemokine.

16. The polypeptide dimer of any one of claims 1-15, wherein the target is expressed by a cancer cell, wherein, optionally, the cancer cell is associated with a solid tumor or a hematological malignancy.

17. The polypeptide dimer of any one of claims 1-16, wherein the target is or comprises a cancer antigen selected from BCMA, GPRC5D, CD19, RORI, SLAMF7, CD229, PNE, EGFR, EGFRVIII, EGP-2, EGP-40, GD2, GD3, HPV E6, HPV E7, Her2, L1-CAM, Lewis A, Lewis Y, MUC1, MUC16, PSCA, PSMA, CD20, CD22, CD56, CD23, CD24, CD30, CD33, CD37, CD44v7/8, CD3δ, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Carbonic anhydrase 9, BRAF, α-fetoprotein, MAGE-A3, MAGE-A4, SSX-2, PRAME, HA-1, B2M, ETA, tyrosinase, KRAS, NRAS, a peptide: MHC complex, and CEA.

18. The polypeptide dimer of any one of claims 1-17, wherein the polypeptide dimer comprises two or more target-binding domains and is multispecific.

19. The polypeptide dimer of any one of claims 1-18, wherein the polypeptide dimer comprises two or more target-binding domains and is bispecific.

20. The polypeptide dimer of claim 18 or 19, which binds to: (i) BCMA and GPRCSD; (ii) BCMA and SLAMF7; (iii) BCMA and CD229; (iv) GPRC5D and SLAMF7; (v) GPRC5D and CD229; (vi) SLAMF7 and CD229; (vii) CD19 and BCMA; or (viii) CD19 and CD229, wherein, optionally, the polypeptide dimer comprises two scFvs.

21. The polypeptide dimer of any one of claims 1-20, comprising a target-binding domain that comprises the VH, the VL, the HCDRs, and/or the LCDRs of: trastuzumab; pertuzumab; rituximab; erbituxumab; ublituxumab; 1.5.3; a BMCA-specific antibody such as J22.0-xi, J22.9-xi, J6M0, J6M1, J6M2, J9M0, J9M1, J9M2, CA8, A7D12.2, C11 D5.3, C12A3.2, C13F12.1, 13C2, 17A5, 83A10, 13A4, 13D2, 14B11, 14E1, 29B11, 29F3, 13A7, CA7, SG1, S307118G03, S332121F02, S332126E04, S322110D07, S336105A07, S335115G01, S335122F05, ET140-3, ET140-24, ET140-37, ET140-40, ET140-54, TBL-CLN1, C4.E2.1, Vicky-1, pSCHLI333, pSCHLI372, pSCHLI373, and those other BCMA-specific antibodies and antigen-binding fragments disclosed in PCT Publication Nos. WO 2002/066516, WO 2007/062090, WO 2010/104949, WO 2011/108008, WO 2012/163805, WO 2014/068079, WO 2015/166073, WO 2014/122143, WO 2014/089335, WO 2016/090327, WO 2016/079177, Ryan et al., Mol. Cancer. Ther. 6 (11): 3009, 2007, and Abbas et al., Blood 128:1688, 2016; a RORI-specific antibody such as R11, R12, Y4, Y13, Y27, or Y31; a CD19-specific antibody such as FMC63; a CD33-specific antibody such as gemtuzumab; an GPRC5D-specific antibody; a RORI-specific VHH; 3F8; alemtuzumab; XMAB-5574; a BCMA-specific VHH; pembrolizumab; nivolumab; a PD-1-specific antibody; elotuzomab; a SLAMF-specific antibody; a CD229-specific antibody; a PD-L1-specific antibody; or an (e.g. cancer antigen-specific, pathogen-specific, autoimmune disease antigen-specific, or neurodegenerative-disease-specific) antibody or antigen-binding fragment approved for therapeutic and/or diagnostic use in humans by the US Food and Drug Administration, the European Medicines Agency, or both.

22. The polypeptide dimer of any one of claims 1-21, comprising a target-binding domain that comprises a receptor ectodomain from Bcl2, or a portion or variant thereof that is functional to bind Bim.

23. The polypeptide dimer of any one of claims 1-22, comprising a target-binding domain comprising: (i) SEQ ID NO.: 97 and SEQ ID NO.: 98, optionally comprised in a scFv, such as having the sequence of SEQ ID NO.: 99 or SEQ ID NO.: 100; (ii) SEQ ID NO.: 101 and 102, optionally in a scFv, such as having the sequence of SEQ ID NO.: 103; (iii) SEQ ID NO.: 104 and SEQ ID NO.: 105, optionally in a scFv, such as having the sequence of SEQ ID NO.: 106; (iv) SEQ ID NO.: 107, optionally comprsed in SEQ ID NO.: 108; or (v) SEQ ID NO.: 109.

24. The polypeptide dimer of any one of claims 1-23, comprising a hinge sequence disposed between and connecting a target-binding domain and the first TCR constant domain or the second TCR constant domain, wherein, optionally, the hinge sequence comprises, consists essentially of, or consists of, the amino acid sequence set forth in any one of SEQ ID NOs.: 42-55 and 68-74, is a (GlyₓSer_{y})ₙ linker wherein x, y, and n are not zero, is a (A)ₙ linker wherein nis one or more, is a GPP linker, or any combination thereof.

25. The polypeptide dimer of any one of claims 1-24, wherein the first polypeptide and/or the second polypeptide does not comprise an immunoglobulin CH2 domain and/or an immunoglobulin CH3 domain and/or an immunoglobulin light chain constant domain disposed C-terminal to the TCR constant domain.

26. The polypeptide dimer of any one of claims 1-25, comprising a target-binding domain comprising (i) a VH comprised in the first polypeptide or the second polypeptide and (ii) a cognate VL comprised in the other of the first and the second polypeptide, wherein the target is not 2,4,6-trinitrophenyl (TNP), digoxin, or phosphorylcholine.

27. The polypeptide dimer of any one of claims 1-26, wherein the first polypeptide and the second polypeptide comprise, consist essentially of, or consist of, amino acid sequences having at least least 90%, at least 91%, at least 92%, at least 93%, at least 94%, at least 95%, at least 96%, at least 97%, at least 98%, or at least 99% to, or comprising or consisting of, the amino acid sequences set forth in SEQ ID NOs.: (i) 110 and 111, respectively; (ii) 112 and 113, respectively; or (iii) 118 and 119, respectively.

28. The polypeptide dimer of any one of claims 1-26, wherein the first polypeptide, the second polypeptide, or both, comprises an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 114, 115, 116, 117, 118, 119, 120, 121, 122, 123, 124, or 125.

29. The polypeptide dimer of any one of claims 1-28, wherein the target-binding domain has a Kd for the target in the range of about 1 nM to about 100 nM, as determined by surface plasmon resonance.

30. A polypeptide comprising the first or the second polypeptide of the polypeptide dimer of any one of claims 1-29, provided that the polypeptide comprises a target-binding domain disposed amino-terminal to the TCR constant domain.

31. A fusion polypeptide comprising an extracellular component, an intracellular component, and a transmembrane component disposed between and connecting the extracellular component and the intracellular component, wherein the extracellular component comprises a CD226 ectodomain or a portion or variant thereof that is functional to bind PVR, and wherein the intracellular component comprises: (i) a CD226 endodomain comprising one or more (e.g. substitution, e.g. non-conservative) mutation that (a) provides increased expression of the fusion polypeptide in a T cell exposed to PVR, as compared to expression of endogenous CD226 by the T cell exposed to PVR, and/or (b) disrupts a Sre kinase phosphorylation site on the fusion polypeptide, and/or (c) reduces ubiquitination of the fusion polypeptide by CBL-B, wherein, optionally, the mutated CD226 endodomain comprises a substitution mutation at a position corresponding to one or more of positions K295, and Y319, and K333, further comprising optionally K295A, Y319F, and/or K333A mutations; (ii) a CD2 intracellular domain sequence (e.g. comprising a co-stimulatory domain); (iii) a truncated CD2 intracellular domain sequence (e.g. comprising a co-stimulatory domain); (iv) a co-stimulatory domain sequence from any one or more of 4-1BB, CD28, OX40, CD27, CD3ε, CD3δ, CD3γ, CD3ζ, CD79A, CD79B, SLAMF1, ICOS, DAP10, GITR, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, PTCH2; or (v) any combination of (i)-(iv), wherein, optionally, the CD226 ectodomain or portion or variant thereof has least 90%, at least 91%, at least 92%, at least 93%, at least 94%, at least 95%, at least 96%, at least 97%, at least 98%, or at Icast 99% to, or comprises or consists of, the amino acid sequence set forth in SEQ ID NO.: 77.

32. The fusion polypeptide of claim 31, wherein the transmembrane component comprises a CD226 transmembrane domain.

33. The fusion polypeptide of claim 31, wherein the transmembrane component has 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 78.

34. A fusion polypeptide comprising an extracellular component, an intracellular component, and a transmembrane component disposed between and connecting the extracellular component and the intracellular component, wherein the extracellular component comprises a TIGIT ectodomain or a portion or variant thereof that is functional to bind PVR, and wherein the intracellular component comprises: (i) a CD2 intracellular domain sequence (e.g. comprising a co-stimulatory domain); (ii) a truncated CD2 intracellular domain sequence (e.g. comprising a co-stimulatory domain); (iii) a co-stimulatory domain sequence from any one or more of 4-1BB, CD28, OX40, CD27, CD3δ, CD3δ, CD3γ, CD3 (, CD79A, CD79B, SLAMF1, ICOS, DAP10, CD25, CARD11, FcRα, FcRβ, FcRγ, Fyn, HVEM, LIGHT, CD30, Lck, LAG3, LAT, LRP, NKG2D, NOTCH1, NOTCH2, NOTCH3, NOTCH4, ROR2, Ryk, Slp76, pTα, TCRα, TCRβ, TRIM, Zap70, PTCH2; or (iv) any combination of (i)-(iii).

35. The fusion polypeptide of claim 34, wherein the TIGIT ectodomain or portion or variant thereof has least 90%, at least 91%, at least 92%, at least 93%, at least 94%, at least 95%, at least 96%, at least 97%, at least 98%, or at least 99% to, or comprises or consists of, the amino acid sequence set forth in SEQ ID NO.: 82.

36. The fusion polypeptide of claim 34 or 35, wherein the transmembrane component comprises a TIGIT transmembrane domain.

37. The fusion polypeptide of claim 34 or 35, wherein the transmembrane component has 90%, at least 91%, at least 92%, at least 93%, at least 94%, at least 95%, at least 96%, at least 97%, at least 98%, or at least 99% to, or comprising or consisting of, the amino acid sequence set forth in SEQ ID NO.: 83.

38. The fusion polypeptide of any one of claims 31-37, wherein the intracellular component comprises an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 79, 84, 85, 86, 87, and 141-149.

39. The fusion polypeptide of any one of claims 31-38, wherein, when a host T cell expressing the fusion polypeptide binds to PVR, the host T cell produces: (i) a CD226 signal; (ii) a CD226 signal that is longer, more persistent, and/or more intense than the signal endogenous CD226 produces in a reference T cell expressing the endogenous CD226 and binding PVR; (iii) does not produce a TIGIT signal; and/or (iv) produces a TIGIT signal that is less intense, is attenuated, is shorter than, and/or is less persistent than the signal endogenous TIGIT produces in a reference T cell expressing the endogenous TIGIT and binding PVR.

40. The fusion polypeptide of any one of claims 31-39, comprising, consisting essentially of, or consisting of, an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 88-96.

41. A polynucleotide encoding (i) the polypeptide dimer of any one of claims 1-29, (ii) the polypeptide of claim 30, (iii) the fusion polypeptide of any one of claims 31-40, or (iv) any combination thereof.

42. The polynucleotide of claim 41, encoding the polypeptide dimer of any one of claims 1-29 and the fusion polypeptide of any one of claims 31-40.

43. The polynucleotide of claim 41 or 42, comprising:
(i) (i) (a) a sequence encoding the polypeptide dimer of any one of claims 1-29, wherein the polypeptide dimer binds to: BCMA, GPRCSD, BCMA and GPRC5D, BCMA and CD229, BCMA and SLAMF7, GPRC5D and CD229, GPRCSD and SLAMF7, or CD229 and SLAMF7, and/or (i) (b) a sequence encoding a chimeric antigen receptor (CAR) that binds to BCMA, GPRCSD, BCMA and GPRC5D, BCMA and CD229, BCMA and SLAMF7, GPRCSD and CD229, GPRCSD and SLAMF7, or CD229 and SLAMF7; and
(ii) a sequence encoding the fusion polypeptide of any one of claims 31-40.

44. The polynucleotide of any one of claims 41-43, further comprising: (i) a promoter, optionally a MNDu3 promoter or a EFla promoter; (ii) one or more sequence encoding a signal peptide; (iii) one or more sequence encoding a furin cleavage sequence; (iv) one or more sequence encoding a self-cleaving peptide; (v) one or more sequence encoding a tag peptide; (vi) one or more sequence encoding a transduction marker; or (vii) any combination thereof, wherein, optionally, the polynucleotide:
(a) comprises a sequence encoding a signal peptide disposed 5′ to: a sequence encoding the first polypeptide of the polypeptide dimer; a sequence encoding the second polypeptide of the polypeptide dimer; a sequence encoding the polypeptide of claim 30; and/or a sequence encoding the fusion polypeptide of any one of claims 31-40; and/or
(b) comprises (1) a sequence encoding a furin cleavage sequence site sequence and/or (2) a sequence encoding a self-cleaving peptide, disposed between: a sequence encoding the first polypeptide of the polypeptide dimer and a sequence encoding the second polypeptide of the polypeptide dimer; a sequence encoding the first polypeptide of the polypeptide dimer and a sequence encoding the fusion polypeptide; a sequence encoding the second polypeptide of the polypeptide dimer and a sequence encoding the fusion polypeptide; a sequence encoding the first polypeptide of the polypeptide dimer and a sequence encoding the polypeptide; a sequence encoding the second polypeptide of the polypeptide dimer and a sequence encoding the polypeptide; a sequence encoding the polypeptide and a sequence encoding the fusion polypeptide; or any combination thereof; and/or
(c) comprises a sequence encoding the fusion polypeptide of any one of claims 31-40 disposed between: (i) a sequence encoding the first polypeptide of the polypeptide dimer and (ii) a sequence encoding the second polypeptide of the polypeptide dimer; and/or
(d) is codon optimized for expression in a host cell, wherein the host cell is optionally a human host cell, further optionally a human immune system cell, still further optionally a human T cell (e.g. a CD4+ T cell, a CD8+ T cell, a CD4−CD8− double negative T cell, a γδ T cell, or any combination thereof), a human NK cell, or a human NK-T cell, wherein, further optionally, the polynucleotide is codon optimized for expression in a T cell (e.g. a CD4+ T cell, a CD8+ T cell, a CD4−CD8− double negative T cell, a γδ T cell, or any combination thereof), a NK cell, or a NK-T cell, wherein the cell is preferably human; and/or
(c) the polynucleotide encodes an amino acid sequence having at least 90%, at least 91%, at least 92%, at least 93%, at least 94%, at least 95%, at least 96%, at least 97%, at least 98%, or at least 99% to, or comprising or consisting of, the amino acid sequence set forth in any one of SEQ ID NOs.: 1-6, 9-15, and 24-32.

45. A vector comprising the polynucleotide of any one of claims 41-44.

46. The vector of claim 45, wherein the vector is capable of delivering the polynucleotide to a hematopoietic progenitor cell or a human immune system cell, wherein, optionally, the human immune system cell comprises a CD4+ T cell, a CD8+ T cell, a CD4−CD8− double negative T cell, a γδ T cell, a natural killer cell, a natural killer T cell, a dendritic cell, or any combination thereof, wherein, further optionally, the T cell comprises a naïve T cell, a central memory T cell, a stem cell memory T cell, an effector memory T cell, or any combination thereof.

47. The vector of claim 45 or 46, wherein the vector is a viral vector.

48. The vector of claim 47, wherein the viral vector is a lentiviral vector or a γ-retroviral vector.

49. A host cell expressing: (i) the polypeptide dimer of any one of claims 1-29; (ii) the polypeptide of claim 30; (iii) the fusion polypeptide of any one of claims 31-40; or (iv) any combination thereof.

50. A host cell comprising the polynucleotide of any one of claims 41-44.

51. A host cell comprising the vector of any one of claims 45-48.

52. The host cell of any one of claims 49-51, wherein the host cell comprises a hematopoietic progenitor cell or a human immune system cell.

53. The host cell of any one of claims 49-52, wherein the host cell comprises a CD4+ T cell, a CD8+ T cell, a CD4−CD8− double negative T cell, a γδ T cell, a natural killer cell, a natural killer T cell, a monocyte, or any combination thereof.

54. The host cell of claim any one of claims 49-53, wherein the host cell comprises a T cell.

55. The host cell of claim 54, wherein the T cell comprises a naïve T cell, a central memory T cell, a stem cell memory T cell, an effector memory T cell, or any combination thereof.

56. The host cell of any one of claims 49-55, comprising a chromosomal gene knockout or a mutation of: a TGFβR1 gene locus, a TGFβR2 gene locus, a PD-1 gene locus, a CTLA4 gene locus, a LAT gene locus, a TIM-3 gene locus, a PD-L1 gene locus, a TIGIT gene locus, an A2AR gene locus, a Fas locus, a FasL gene locus, a B7-H3 gene locus, a B7-H4 gene locus, an IDO gene locus, a VISTA gene locus, a SIGLEC7 gene locus, a SIGLEC9 gene locus, a TRAC gene locus, a TRBC gene locus, a T cell receptor gene locus, a MHC (e.g. HLA) gene locus, a CBLB gene locus, a RASA2 gene locus, a UBASH3A gene locus, a CISH gene locus, a CD4 gene locus, a CD8 gene locus, or any combination thereof, such as a TIGIT locus, a TRAC gene locus, a TRBC gene locus, and/or one or both of a CD4 gene locus and a CD8 gene locus.

57. The host cell of any one of claims 49-56, wherein the host cell is modified (e.g., having a chromosomal knockout mutation and/or a chromosomal missense mutation and/or a chromosomal splice junction mutation; encoding an inhibitory nucleic acid such as an siRNA or an antisense oligonucleotide) to have reduced protein expression (including null expression), of an endogenous TRAC, an endogenous TRBC, an endogenous TIGIT, endogenous CD4, endogenous CD8, or any combination thereof, as compared to the unmodified host cell.

58. The host cell of any one of claims 49-57, expressing the polypeptide dimer of any one of claims 1-30 and the fusion polypeptide of any one of claims 32-42, wherein, optionally, the polypeptide dimer binds to: BCMA, GPRCSD, BCMA and GPRCSD, BCMA and CD229, BCMA and SLAMF7, GPRC5D and CD229, GPRCSD and SLAMF7, or CD229 and SLAMF7.

59. A host cell comprising a T cell comprising a chromosomal gene knockout of a CD4 and/or of a CD8, wherein the T cell expresses a T cell receptor (TCR) or a chimeric antigen receptor CAR), wherein the TCR or CAR is capable of binding to a peptide: MHC complex in the absence of CD4, in the absence of CD8, or in the absence of both.

60. A composition comprising:
(i) the polypeptide dimer of any one of claims 1-29; and/or
(ii) the polypeptide of claim 30; and/or
(iii) the fusion polypeptide of any one of claims 31-40; and/or
(iv) the polynucleotide of any one of claims 41-44; and/or
(v) the vector of any one of claims 45-48; and/or
(vi) the host cell of any one of claims 49-59,
and a pharmaceutically acceptable carrier, excipient, or diluent.

61. The composition of claim 60, comprising (i) a composition comprising at least about 30% CD4+T host cells, combined with (ii) a composition comprising at least about 30% CD8+T host cells, in about a 1:1 ratio.

62. A method of treating a disease or condition in a subject, the method comprising administering to the subject an effective amount of:
(i) the polypeptide dimer of any one of claims 1-29; and/or
(ii) the polypeptide of claim 30; and/or
(iii) the fusion polypeptide of any one of claims 31-40; and/or
(iv) the polynucleotide of any one of claims 41-44; and/or
(v) the vector of any one of claims 45-48; and/or
(vi) the host cell of any one of claims 49-59; and/or
(vii) the composition of claim 60 or 61.

63. The polypeptide dimer of any one of claims 1-29, and/or the polypeptide of claim 30, and/or the fusion polypeptide of any one of claims 31-40, and/or the polynucleotide of any one of claims 41-44, and/or the vector of any one of claims 45-48, and/or the host cell of any one of claims 49-59, and/or the composition of claim 60 or 61, for use in a method of treating a disease or condition in a subject.

64. The polypeptide dimer of any one of claims 1-29, and/or the polypeptide of claim 30, and/or the fusion polypeptide of any one of claims 31-40, and/or the polynucleotide of any one of claims 41-44, and/or the vector of any one of claims 45-48, and/or the host cell of any one of claims 49-59, and/or the composition of claim 60 or 61, for use in a the preparation of a medicament for treating a disease or condition in a subject.

65. The method of claim 62, or the polypeptide dimer, polypeptide, fusion polypeptide, polynucleotide, vector, host cell, or composition for use of any one of claims 63-64, wherein the disease or condition comprises or is a hyperproliferative disease, a proliferative disease, an autoimmune disease, a neurodegenerative disease, or an infection.

66. The method of claim 62 or 65 or the polypeptide dimer, polypeptide, fusion polypeptide polynucleotide, vector, host cell, or composition for use of any one of claims 63-65, wherein the disease or condition is a cancer, such as a hematological cancer or a solid cancer, wherein, optionally:
(1) the cancer comprises a myeloma (e.g. multiple myeloma), a carcinoma, a sarcoma, a glioma, a lymphoma, a leukemia, a myeloma, or any combination thereof, and/or
(2) the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof; and/or
(3) the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

67. The method of claim 66 or the polypeptide dimer, polypeptide, fusion polypeptide, polynucleotide, vector, host cell, or composition for use of claim 66, wherein the cancer comprises a solid tumor, wherein, optionally, the solid tumor is a sarcoma or a carcinoma, wherein, further optionally, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

68. The method of claim 67 or the polypeptide dimer, polypeptide, fusion polypeptide, polynucleotide, vector, host cell, or composition for use of claim 67, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

69. The method of claim 67 or 68, or the polypeptide dimer, polypeptide, fusion polypeptide, polynucleotide, vector, host cell, or composition for use of claim 67 or 68, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

70. The method of claim 66 or the polypeptide dimer, polypeptide, fusion polypeptide polynucleotide, vector, host cell, or composition for use of claim 66, wherein the disease or condition is multiple myeloma.

71. The method of any one of claims 62 and 65-70, or the polypeptide dimer, polypeptide, fusion polypeptide, polynucleotide, vector, host cell, or composition for use of any one of claims 63-70, wherein the host cell is an allogeneic cell, a syngeneic cell, or an autologous cell.

72. The method of any one of claims 62 and 65-71, or the polypeptide dimer, polypeptide, fusion polypeptide, polynucleotide, vector, host cell, or composition for use of any one of claims 63-71, wherein the method comprises administering a plurality of doses of the fusion protein, polynucleotide, vector, host cell, or composition to the subject, wherein, optionally: (1) the plurality of doses are administered at intervals between administrations of about two, three, four, five, six, seven, eight, or more weeks; and/or (2) a dose of the host cell comprises about 10⁵ cells/m2 to about 10¹¹ cells/m2.

73. The method of any one of claims 62 and 65-72, or the polypeptide dimer, polypeptide, fusion polypeptide, polynucleotide, vector, host cell, or composition for use of any one of claims 63-72, wherein the subject is receiving, has received, or will receive one or more of:
(i) chemotherapy;
(ii) radiation therapy;
(iii) an inhibitor of an immune suppression component;
(iv) an agonist of a stimulatory immune checkpoint agent;
(v) RNAi;
(vi) a cytokine;
(vii) a surgery;
(viii) a monoclonal antibody and/or an antibody-drug conjugate; or
(ix) any combination of (i)-(viii), in any order.

74. A method comprising introducing into a host cell (i) a polynucleotide of any one of claims 41-44 or (ii) a vector of any one of claims 45-48, wherein, optionally, the host cell comprises a chromosomal gene knockout of TIGIT, CD226, TRAC, TRBC, CD8, CD4, or any combination thereof, and/or wherein, optionally:
(1) the host cell comprises a hematopoietic progenitor cell or an immune system cell, optionally a human immune system cell; and/or
(2) the host cell comprises a CD4+ T cell, a CD8+ T cell, a CD4−CD8− double negative T cell, a γδ T cell, a natural killer cell, a natural killer T cell, a monocyte, or any combination thereof; and/or
(3) the host cell comprises a T cell, wherein, further optionally, the T cell comprises a naïve T cell, a central memory T cell, a stem cell memory T cell, an effector memory T cell, or any combination thereof.

75. The method of claim 74, further comprising introducing a chromosomal gene knockout of TIGIT, CD226, TRAC, TRBC, CD8, CD4, or any combination thereof.
